(19) United States
(12) Patent Application Publication
(10) Pub. No.: US 2023/0159532 A1
(43) Pub. Date: May 25, 2023

(54) PARATHYROID HORMONE (PTH) RECEPTOR ANTAGONISTS AND USES THEREOF

(71) Applicant: Crinetics Pharmaceuticals, Inc., San Diego, CA (US)

(72) Inventors: Joseph PONTILLO, San Diego, CA (US); Shimiao WANG, San Diego, CA (US); Yunfei ZHU, San Diego, CA (US)

(21) Appl. No.: 17/936,508

(22) Filed: Sep. 29, 2022

Related U.S. Application Data
(60) Provisional application No. 63/250,827, filed on Sep. 30, 2021.

(51) Int. Cl. C07D 471/14; C07D 471/04

(52) U.S. Cl. CPC C07D 471/14; C07D 471/04

(57) ABSTRACT

Described herein are compounds that are parathyroid hormone (PTH) receptor modulators, methods of making such compounds, pharmaceutical compositions and medicaments comprising such compounds, and methods of using such compounds in the treatment of conditions, diseases, or disorders that would benefit from modulation of PTH receptor activity.

### CROSS-REFERENCE

This application claims the benefit of U.S. Provisional Application No. 63/250,827, filed Sep. 30, 2021, which is incorporated herein by reference in its entirety.

### FIELD OF THE INVENTION

Described herein are compounds that modulate the activity of one or more parathyroid hormone (PTH) receptors, methods of making such compounds, pharmaceutical compositions and medicaments comprising such compounds, and methods of using such compounds in the treatment of conditions, diseases, or disorders that would benefit from modulating parathyroid hormone (PTH) receptor activity.

### BACKGROUND OF THE INVENTION

Parathyroid hormone (PTH), parathyroid hormone-related protein (PTHrP), and tuberoinfundibular peptide of thirty-nine residues (TIP39) are endogenous ligands for parathyroid hormone 1 receptor (PTH1R) and parathyroid hormone 2 receptor (PTH2R). PTH is an endocrine hormone essential for mineral homeostasis. PTHrP is a widely distributed paracrine factor that plays a role in the development and remodeling of many tissues, in transepithelial calcium transport, and in smooth muscle relaxation. TIP39 was purified from bovine hypothalamus on the basis of its activation of PTH2R, and its physiological role(s) are largely unknown.

Two subtype PTH receptor proteins have been identified (PTH1R and PTH2R). PTH1 receptor expression is particularly high in skeletal tissues and kidney but it has a nearly ubiquitous tissue distribution. PTH2R is most abundant in the central nervous system, pancreas, testes, and placenta, and low levels have been reported in a number of other tissues. Modulation of a particular subtype PTH receptor, or combination thereof, is attractive for the treatment of conditions, diseases, or disorders that would benefit from modulating PTH receptor activity, such as, by way of example only, primary hyperparathryroidism (pHPT), humoral hypercalcemia of malignancy (HHM), and secondary hyperparathryroidism (secondary HPT).

### SUMMARY OF THE INVENTION

Compounds described herein are parathyroid hormone (PTH) receptor modulator compounds. In some embodiments, compounds described herein modulate one or more of the subtype PTH receptor proteins. In some embodiments, compounds described herein modulate PTH1R.

In one aspect, described herein is a compound of Formula (I′), or a pharmaceutically acceptable salt, or solvate thereof:

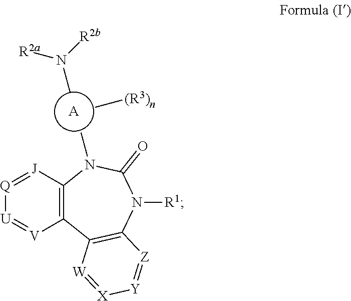

wherein:

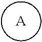

is selected from phenyl and a 6-membered heteroaryl ring comprising 1 or 2 nitrogen atoms;
J is C(R⁴) or N;
Q is C(R⁵) or N;
U is C(R⁶) or N;
V is C(R⁷) or N; wherein no more than two of J, Q, U, and V are N;
W is C(R⁸) or N;
X is C(R⁹) or N;
Y is C(R¹⁰) or N;
Z is C(R¹¹) or N; wherein no more than two of W, X, Y, and Z are N;
R¹ is hydrogen, C₁₋₆alkyl, C₂₋₆alkenyl, C₂₋₆alkynyl, C₃₋₆cycloalkyl, or C₂₋₉heterocycloalkyl, wherein C₁₋₆alkyl, C₂₋₆alkenyl, C₂₋₆alkynyl, C₃₋₆cycloalkyl, and C₂₋₉heterocycloalkyl are optionally substituted with one, two, three, four, or five R¹⁹;
R^{2a} is hydrogen, C₁₋₆alkyl, C₂₋₆alkenyl, C₂₋₆alkynyl, C₃₋₆cycloalkyl, or —(C(R¹⁶)(R¹⁷))ₘ—N(R^{18a})(R^{18b});
R^{2b} is hydrogen, C₁₋₆alkyl, C₂₋₆alkenyl, C₂₋₆alkynyl, or C₃₋₆cycloalkyl, wherein C₁₋₆alkyl, C₂₋₆alkenyl, C₂₋₆alkynyl, and C₃₋₆cycloalkyl are optionally substituted with one, two, three, four, or five R²⁰;
or R^{2a} and R^{2b} are taken together with the nitrogen atom to which they are attached to form a C₂₋₉heterocycloalkyl optionally substituted with one, two, three, four, or five R²⁰;
each R³ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —N(R¹⁴)C(O)R¹⁵, —S(O)₂R¹⁵, and —S(O)₂N(R¹²)(R¹³)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, —OR¹², —N(R¹²)(R¹³), and —C(O)OR¹²;
R⁴, R⁵, R⁶, R⁷, R⁸, R⁹, R¹⁰, and R¹¹ are each independently selected from hydrogen, halogen, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, —CN, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —N(R¹⁴)C(O)R¹⁵, —S(O)₂R¹⁵, and —S(O)₂N(R¹²)(R¹³)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, three, four, or five R²¹; each R¹² is independently selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, —C(O)OH, C₃₋₆cycloalkyl, C₂₋₉heterocycloalkyl, C₆₋₁₀aryl, and C₁₋₉heteroaryl;
each R¹³ is independently selected from hydrogen, C₁₋₆alkyl, and C₁₋₆haloalkyl;
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹⁶ and each R¹⁷ are each independently selected from hydrogen, halogen, hydroxy, C₁₋₆alkyl, C₁₋₆haloalkyl, and C₁₋₆alkoxy; or R¹⁶ and R¹⁷ are taken together to form a C₃₋₆cycloalkyl;
R^{18a} and R^{18b} are independently selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, three, four, or five R²⁰; or R^{18a} and R^{18b} are taken together with the nitrogen atom to which they are attached to form a C₂₋₉heterocycloalkyl optionally substituted with one, two, three, four, or five R²⁰;
each R¹⁹, R²⁰, and R²¹ are each independently selected from halogen, oxo, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —N(R¹⁴)C(O)R¹⁵, —S(O)₂R¹⁵, and —S(O)₂N(R¹²)(R¹³)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, oxo, C₁₋₆alkyl, C₁₋₆haloalkyl, —OR¹², —S(O)₂OH, —N(R¹²)(R¹³), and —C(O)OR¹²;
m is 2, 3, or 4; and
n is 0, 1, 2, 3, or 4.

In another aspect, described herein is a compound of Formula (I), or a pharmaceutically acceptable salt, or solvate thereof:

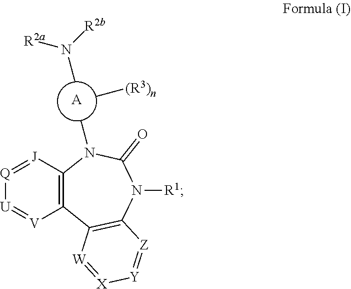

wherein:

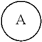

is selected from phenyl and a 6-membered heteroaryl ring comprising 1 or 2 nitrogen atoms;
J is C(R⁴) or N;
Q is C(R⁵) or N;
U is C(R⁶) or N;
V is C(R⁷) or N; wherein no more than two of J, Q, U, and V are N;
W is C(R⁸) or N;
X is C(R⁹) or N;
Y is C(R¹⁰) or N;
Z is C(R¹¹) or N; wherein no more than two of W, X, Y, and Z are N;
R¹ is hydrogen, C₁₋₆alkyl, C₂₋₆alkenyl, C₂₋₆alkynyl, C₃₋₆cycloalkyl, or C₂₋₉heterocycloalkyl, wherein C₁₋₆alkyl, C₂₋₆alkenyl, C₂₋₆alkynyl, C₃₋₆cycloalkyl, and C₂₋₉heterocycloalkyl are optionally substituted with one, two, three, four, or five R¹⁹;
R^{2a} is hydrogen, C₁₋₆alkyl, C₂₋₆alkenyl, C₂₋₆alkynyl, C₃₋₆cycloalkyl, or —(C(R¹⁶)(R¹⁷))ₘ—N(R^{18a})(R^{18b});
R^{2b} is hydrogen, C₁₋₆alkyl, C₂₋₆alkenyl, C₂₋₆alkynyl, or C₃₋₆cycloalkyl, wherein C₁₋₆alkyl, C₂₋₆alkenyl, C₂₋₆alkynyl, and C₃₋₆cycloalkyl are optionally substituted with one, two, three, four, or five R²⁰;
or R^{2a} and R^{2b} are taken together with the nitrogen atom to which they are attached to form a C₂₋₉heterocycloalkyl optionally substituted with one, two, three, four, or five R²⁰;
each R³ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —N(R¹⁴)C(O)R¹⁵, —S(O)₂R¹⁵, and —S(O)₂N(R¹²)(R¹³)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, —OR¹², —N(R¹²)(R¹³), and —C(O)OR¹²;
R⁴, R⁵, R⁶, R⁷, R⁸, R⁹, R¹⁰, and R¹¹ are each independently selected from hydrogen, halogen, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, —CN, —OR², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —N(R¹⁴)C(O)R¹⁵, —S(O)₂R¹⁵, and —S(O)₂N(R¹²)(R¹³)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, three, four, or five R²¹; each R¹² is independently selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹³ is independently selected from hydrogen, C₁₋₆alkyl, and C₁₋₆haloalkyl;
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹⁶ and each R¹⁷ are each independently selected from hydrogen, halogen, hydroxy, C₁₋₆alkyl, C₁₋₆haloalkyl, and C₁₋₆alkoxy; or R¹⁶ and R¹⁷ are taken together to form a C₃₋₆cycloalkyl;
R^{18a} and R^{18b} are independently selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, three, four, or five R²⁰; or R^{18a} and R^{18b} are taken together with the nitrogen atom to which they are attached to form a C₂₋₉heterocycloalkyl optionally substituted with one, two, three, four, or five R²⁰;
each R¹⁹, R²⁰, and R²¹ are each independently selected from halogen, oxo, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —N(R¹⁴)C(O)R¹⁵, —S(O)₂R¹⁵, and —S(O)₂N(R¹²)(R¹³)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, —OR¹², —N(R¹²)(R¹³), and —C(O)OR¹²;
m is 2, 3, or 4; and
n is 0, 1, 2, 3, or 4.

In some embodiments is a compound of Formula (I′) or (I), or a pharmaceutically acceptable salt or solvate thereof, wherein:
J is C(R⁴);
Q is C(R⁵);
U is C(R⁶); and
V is C(R⁷).

In some embodiments is a compound of Formula (I′) or (I), or a pharmaceutically acceptable salt or solvate thereof, wherein:
W is C(R⁸);
X is C(R⁹)
Y is C(R¹⁰); and
Z is N.

In some embodiments is a compound of Formula (I′) or (I), or a pharmaceutically acceptable salt or solvate thereof, wherein

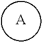

is phenyl. In some embodiments is a compound of Formula (I′) or (I), or a pharmaceutically acceptable salt or solvate thereof, wherein n is 1 or 2.

In some embodiments is a compound of Formula (I′) or (I), or a pharmaceutically acceptable salt or solvate thereof, having the structure of Formula (Ia):

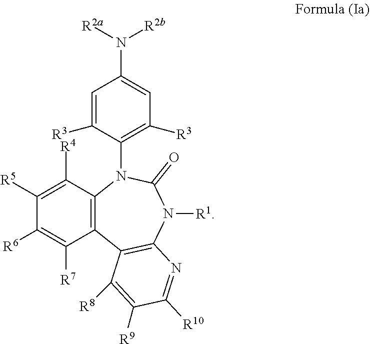

In some embodiments is a compound of Formula (I′), (I), or (Ia), or a pharmaceutically acceptable salt or solvate thereof, wherein R¹ is C₁₋₆alkyl optionally substituted with one, two, three, four, or five R¹⁹. In some embodiments is a compound of Formula (I′), (I), or (Ia), or a pharmaceutically acceptable salt or solvate thereof, wherein R¹ is C₁₋₆alkyl optionally substituted with one, two, three, four, or five R¹⁹, and each R¹⁹ is independently selected from halogen, C₁₋₆haloalkyl, C₃₋₆cycloalkyl, —OR¹², and —N(R¹²)(R¹³). In some embodiments is a compound of Formula (I′), (I), or (Ia), or a pharmaceutically acceptable salt or solvate thereof, wherein R¹ is C₁₋₆alkyl optionally substituted with one, two, three, four, or five R¹⁹, each R¹⁹ is independently selected from halogen, C₃₋₆cycloalkyl, and OR¹², and each R¹² is independently selected from hydrogen and C₁₋₆alkyl. In some embodiments is a compound of Formula (I′), (I), or (Ia), or a pharmaceutically acceptable salt or solvate thereof, wherein R¹ is —CF₃, —CH₂CF₃, —CH₂CH₂OH, —CH₂CH₂OCH₃, or

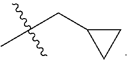 [SMILES: *CC1CC1]

In some embodiments is a compound of Formula (I′), (I), or (Ia), or a pharmaceutically acceptable salt or solvate thereof, wherein R¹ is an unsubstituted C₁₋₆alkyl. In some embodiments is a compound of Formula (I′), (I), or (Ia), or a pharmaceutically acceptable salt or solvate thereof, wherein R¹ is —CH₃, —CH₂CH₃, —CH(CH₃)₂, or —CH₂CH₂CH₃. In some embodiments is a compound of Formula (I′), (I), or (Ia), or a pharmaceutically acceptable salt or solvate thereof, wherein R¹ is C₃₋₆cycloalkyl optionally substituted with one, two, three, four, or five R¹⁹. In some embodiments is a compound of Formula (I′), (I), or (Ia), or a pharmaceutically acceptable salt or solvate thereof, wherein R¹ is

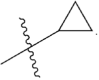 [SMILES: *C1CC1]

In some embodiments is a compound of Formula (I′), (I), or (Ia), or a pharmaceutically acceptable salt or solvate thereof, wherein R^{2a} is —(C(R¹⁶)(R¹⁷))ₘ—N(R^{18a})(R^{18b}). In some embodiments is a compound of Formula (I′), (I), or (Ia), or a pharmaceutically acceptable salt or solvate thereof, wherein each R¹⁶ and each R¹⁷ are each independently selected from hydrogen and C₁₋₆alkyl. In some embodiments is a compound of Formula (I′), (I), or (Ia), or a pharmaceutically acceptable salt or solvate thereof, wherein each R¹⁶ and each R¹⁷ are hydrogen. In some embodiments is a compound of Formula (I′), (I), or (Ia), or a pharmaceutically acceptable salt or solvate thereof, wherein m is 2. In some embodiments is a compound of Formula (I′), (I), or (Ia), or a pharmaceutically acceptable salt or solvate thereof, wherein m is 3. In some embodiments is a compound of Formula (I′), (I), or (Ia), or a pharmaceutically acceptable salt or solvate thereof, wherein R^{18a} is C₁₋₆alkyl optionally substituted with one, two, three, four, or five R²⁰. In some embodiments is a compound of Formula (I′), (I), or (Ia), or a pharmaceutically acceptable salt or solvate thereof, wherein R^{18a} is C₁₋₆alkyl optionally substituted with one, two, three, four, or five R²⁰, and each R²⁰ is independently selected from halogen, —OR¹², —N(R¹²)(R¹³), and —C(O)OR¹². In some embodiments is a compound of Formula (I′), (I), or (Ia), or a pharmaceutically acceptable salt or solvate thereof, wherein R^{18a} is C₁₋₆alkyl substituted with one, two, or three R²⁰, each R²⁰ is independently selected from halogen, —OR¹², —N(R¹²)(R¹³), and —C(O)OR¹², each R¹² is independently selected from hydrogen and C₁₋₆alkyl, and each R¹³ is independently selected from hydrogen and C₁₋₆alkyl. In some embodiments is a compound of Formula (I′), (I), or (Ia), or a pharmaceutically acceptable salt or solvate thereof, wherein R^{18a} is —CH₂CH₂OH, —CH₂CH₂OCH₃, —CH₂CH₂NH₂, —CH₂CH₂N(H)(CH₃), —CH₂CH₂C(O)OH, and —CH₂CH₂CH₂C(O)OH. In some embodiments is a compound of Formula (I′), (I), or (Ia), or a pharmaceutically acceptable salt or solvate thereof, wherein R^{18a} is an unsubstituted C₁₋₆alkyl. In some embodiments is a compound of Formula (I′), (I), or (Ia), or a pharmaceutically acceptable salt or solvate thereof, wherein R^{18a} is —CH₃. In some embodiments is a compound of Formula (I′), (I), or (Ia), or a pharmaceutically acceptable salt or solvate thereof, wherein R^{18a} is hydrogen. In some embodiments is a compound of Formula (I′), (I), or (Ia), or a pharmaceutically acceptable salt or solvate thereof, wherein R^{18b} is hydrogen. In some embodiments is a compound of Formula (I′), (I), or (Ia), or a pharmaceutically acceptable salt or solvate thereof, wherein R^{18b} is an unsubstituted C₁₋₆alkyl. In some embodiments is a compound of Formula (I′), (I), or (Ia), or a pharmaceutically acceptable salt or solvate thereof, wherein R^{18b}is —CH₃. In some embodiments is a compound of Formula (I′), (I), or (Ia), or a pharmaceutically acceptable salt or solvate thereof, wherein R^{2b} is hydrogen. In some embodiments is a compound of Formula (I′), (I), or (Ia), or a pharmaceutically acceptable salt or solvate thereof, wherein R^{2a} and R^{2b} are taken together with the nitrogen atom to which they are attached to form a C₂-9heterocycloalkyl optionally substituted with one, two, three, four, or five R²⁰. In some embodiments is a compound of Formula (I′), (I), or (Ia), or a pharmaceutically acceptable salt or solvate thereof, wherein R^{2a} and R^{2b} are taken together with the nitrogen atom to which they are attached to form a C₂₋₉heterocycloalkyl optionally substituted with one, two, three, four, or five R²⁰, and each R²⁰ is independently selected from halogen, —OR¹², —N(R¹²)(R¹³), and C₁₋₆alkyl optionally substituted with —N(R¹²)(R¹³). In some embodiments is a compound of Formula (I′), (I), or (Ia), or a pharmaceutically acceptable salt or solvate thereof, wherein R^{2a} and R^{2b} are taken together with the nitrogen atom to which they are attached to form a substituted C₂₋₉heterocycloalkyl selected from

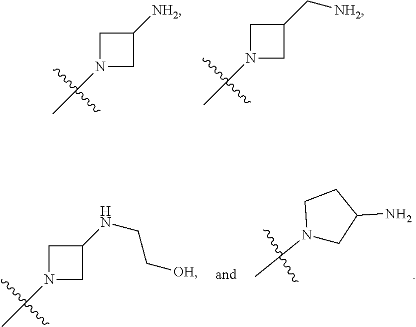 [SMILES: *N1CC(N)C1 ; *N1CC(CN)C1 ; *N1CC(NCCO)C1 ; *N1CCC(N)C1 ; "."]

and
In some embodiments is a compound of Formula (I′), (I), or (Ia), or a pharmaceutically acceptable salt or solvate thereof, wherein each R³ is independently selected from halogen, C₁₋₆alkyl, and —OR¹², wherein C₁₋₆alkyl is optionally substituted with one, two, three halogen, and R¹² is selected from hydrogen and C₁₋₆alkyl. In some embodiments is a compound of Formula (I′), (I), or (Ia), or a pharmaceutically acceptable salt or solvate thereof, wherein each R³ is independently selected from halogen. In some embodiments is a compound of Formula (I′), (I), or (Ia), or a pharmaceutically acceptable salt or solvate thereof, wherein each R³ is —F. In some embodiments is a compound of Formula (I′), (I), or (Ia), or a pharmaceutically acceptable salt or solvate thereof, wherein R⁵ is selected from hydrogen, halogen, C₁₋₆alkyl, C₃₋₆cycloalkyl, C₂₋₉heterocycloalkyl, —CN, —C(O)N(R¹²)(R¹³), and —OR¹², wherein C₁₋₆alkyl, C₃₋₆cycloalkyl, and C₂₋₉heterocycloalkyl are optionally substituted with one, two, three, four, or five R²¹. In some embodiments is a compound of Formula (I′), (I), or (Ia), or a pharmaceutically acceptable salt or solvate thereof, wherein R⁵ is selected from hydrogen, halogen, C₁₋₆alkyl, —CN, and —OR¹², wherein C₁₋₆alkyl is optionally substituted with one, two, three, four, or five R²¹. In some embodiments is a compound of Formula (I′), (I), or (Ia), or a pharmaceutically acceptable salt or solvate thereof, wherein R⁵ is selected from hydrogen, halogen, C₁₋₆alkyl, —CN, and —OR¹² wherein C₁₋₆alkyl is optionally substituted with one, two, three halogen, and R¹² is selected from hydrogen and C₁₋₆alkyl. In some embodiments is a compound of Formula (I′), (I), or (Ia), or a pharmaceutically acceptable salt or solvate thereof, wherein R⁵ is selected from hydrogen, —F, —Cl, —CH₃, —CF₃, —CN, and —OCH₃. In some embodiments is a compound of Formula (I′), (I), or (Ia), or a pharmaceutically acceptable salt or solvate thereof, wherein R⁷ is selected from hydrogen, halogen, C₁₋₆alkyl, —CN, and —OR¹², wherein C₁₋₆alkyl is optionally substituted with one, two, three, four, or five R²¹. In some embodiments is a compound of Formula (I′), (I), or (Ia), or a pharmaceutically acceptable salt or solvate thereof, wherein R⁷ is selected from hydrogen, halogen, C₁₋₆alkyl, —CN, and —OR¹², wherein C₁₋₆alkyl is optionally substituted with one, two, three halogen, and R¹² is selected from hydrogen and C₁₋₆alkyl. In some embodiments is a compound of Formula (I′), (I), or (Ia), or a pharmaceutically acceptable salt or solvate thereof, wherein R⁷ is selected from hydrogen, —F, —Cl, —CH₃, —CF₃, —CN, and —OCH₃. In some embodiments is a compound of Formula (I′), (I), or (Ia), or a pharmaceutically acceptable salt or solvate thereof, wherein R⁹ is selected from hydrogen, halogen, C₁₋₆alkyl, C₃₋₆cycloalkyl, —CN, and —OR¹², wherein C₁₋₆alkyl and C₃₋₆cycloalkyl are optionally substituted with one, two, three, four, or five R²¹. In some embodiments is a compound of Formula (I′), (I), or (Ia), or a pharmaceutically acceptable salt or solvate thereof, wherein R⁹ is selected from hydrogen, halogen, C₁₋₆alkyl, C₃₋₆cycloalkyl, —CN, and —OR¹², wherein C₁₋₆alkyl and C₃₋₆cycloalkyl are optionally substituted with one, two, three halogen, and R¹² is selected from hydrogen and C₁₋₆alkyl. In some embodiments is a compound of Formula (I′), (I), or (Ia), or a pharmaceutically acceptable salt or solvate thereof, wherein R⁹ is selected from hydrogen, —F, —Cl, —CH₃, —CF₃, —CN, —OCH₃, and

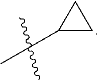 [SMILES: *C1CC1]

In some embodiments is a compound of Formula (I′), (I), or (Ia), or a pharmaceutically acceptable salt or solvate thereof, wherein R⁴, R⁶, R⁸, and R¹⁰ are each independently selected from hydrogen, halogen, and unsubstituted C₁₋₆alkyl. In some embodiments is a compound of Formula (I′), (I), or (Ia), or a pharmaceutically acceptable salt or solvate thereof, wherein R⁴, R⁶, R⁸, and R¹⁰ are each hydrogen.

Any combination of the groups described above for the various variables is contemplated herein. Throughout the specification, groups and substituents thereof are chosen by one skilled in the field to provide stable moieties and compounds.

Also described herein is a pharmaceutical composition comprising a compound described herein, or a pharmaceutically acceptable salt, or solvate thereof, and at least one pharmaceutically acceptable excipient. In some embodiments, the pharmaceutical composition is formulated for administration to a mammal by intravenous administration, subcutaneous administration, oral administration, inhalation, nasal administration, dermal administration, or ophthalmic administration. In some embodiments, the pharmaceutical composition is formulated for administration to a mammal by oral administration. In some embodiments, the pharmaceutical composition is in the form of a tablet, a pill, a capsule, a liquid, a suspension, a gel, a dispersion, a solution, an emulsion, an ointment, or a lotion. In some embodiments, the pharmaceutical composition is in the form of a tablet, a pill, or a capsule.

Also described herein is a method of treating a disease or condition in a mammal that would benefit from the modulation of parathyroid hormone (PTH) receptor activity comprising administering a compound described herein, or a pharmaceutically acceptable salt or solvate thereof, to the mammal in need thereof. In some embodiments, the disease or condition comprises oversecretion of parathyroid hormone (PTH), oversecretion of parathyroid hormone-related peptide (PTHrP), increased serum calcium levels, decreased bone mass, osteoporosis, kidney stones, cardiovascular disease, neurocognitive issues, or combinations thereof. In some embodiments, the disease or condition is selected from primary hyperparathyroidism (pHPT), humoral hypercalcemia of malignancy (HHM), or secondary hyperparathyroidism (secondary HPT).

Also described herein is a method of reducing serum calcium levels in a mammal in need thereof comprising administering a compound described herein, or a pharmaceutically acceptable salt or solvate thereof, to the mammal.

In any of the aforementioned aspects are further embodiments in which the effective amount of the compound of Formula (I′) or (I), or a pharmaceutically acceptable salt thereof, is: (a) systemically administered to the mammal; and/or (b) administered orally to the mammal; and/or (c) intravenously administered to the mammal; and/or (d) administered by inhalation; and/or (e) administered by nasal administration; or and/or (f) administered by injection to the mammal; and/or (g) administered topically to the mammal; and/or (h) administered by ophthalmic administration; and/or (i) administered rectally to the mammal; and/or (j) administered non-systemically or locally to the mammal.

In any of the aforementioned aspects are further embodiments comprising single administrations of the effective amount of the compound, including further embodiments in which the compound is administered once a day to the mammal or the compound is administered to the mammal multiple times over the span of one day. In some embodiments, the compound is administered on a continuous dosing schedule. In some embodiments, the compound is administered on a continuous daily dosing schedule.

In any of the embodiments disclosed herein, the mammal is a human.

In some embodiments, compounds provided herein are orally administered to a human.

Articles of manufacture, which include packaging material, a compound of Formula (I′) or (I), or a pharmaceutically acceptable salt thereof, within the packaging material, and a label that indicates that the compound or composition, or pharmaceutically acceptable salt, or solvate thereof, is used for modulating one or more subtype parathyroid hormone receptor proteins, or for the treatment, prevention or amelioration of one or more symptoms of a disease or condition that would benefit from modulating one or more subtype parathyroid hormone receptor proteins, are provided.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### DETAILED DESCRIPTION OF THE INVENTION

Parathyroid hormone (PTH) is an 84 amino acid peptide synthesized primarily by the chief cells of the parathyroid gland and functions as a prohormone acting in various tissues. However, most of the characterized biological activity of PTH can be mediated by its first 34 N-terminal residues. PTH is a key regulator of serum calcium concentration through its effects on bone, kidney, and intestine. Secretion of PTH is determined primarily by serum ionized calcium concentration through negative feedback. Parathyroid cells express calcium-sensing receptors on the cell surface; and PTH is secreted when calcium concentration is decreased. Serum phosphate levels also play an important role in calcium levels and PTH secretion due to the formation of calcium phosphate. An increase in serum phosphate results in a decrease in serum calcium and therefore an increase in PTH secretion. Likewise, a decrease in serum phosphate results in an increase in serum calcium.

In bone, PTH enhances the release of calcium from the large reservoir contained in the bones. PTH is secreted in response to low blood calcium (Ca²⁺) levels, and indirectly stimulates osteoclast activity, in an effort to release more ionic calcium (Ca²⁺) into the blood. Stimulation is indirect as osteoclasts do not express the PTH1 receptor. PTH instead binds PTH1R expressed in the osteoblasts, the cells responsible for creating bone, thus stimulating the cells to increase the expression of Receptor Activator of Nuclear Factor kappa B Ligand (RANKL) and inhibiting their secretion of osteoprotegerin (OPG). Free OPG competitively binds to RANKL as a decoy receptor, preventing RANKL from interacting with Receptor Activator of Nuclear Factor kB (RANK) (a receptor for RANKL). The binding of RANKL to RANK stimulates the osteoclast precursors to fuse, forming new osteoclasts, which ultimately enhances bone resorption, the normal destruction of bone, resulting in an increase of serum calcium.

In the kidney, PTH first reduces the reabsorption of phosphate (HPO₄²⁻) from the proximal tubule of the kidney, resulting in a decreased plasma phosphate concentration. As phosphate ions form water-insoluble salts with calcium, a decrease in the circulating phosphate concentration results in an increase in the amount of ionized calcium in blood. A second important PTH effect on the kidney is the stimulation of the conversion of 25-hydroxy vitamin D (25-hydroxycholecalciferol) into 1,25-dihydroxy vitamin D (calcitriol, 1,25-dihydroxycholecalciferol). In the kidney, PTH upregulates the activity of 1-α-hydroxylase enzyme, which converts 25-hydroxycholecalciferol, the major circulating form of inactive vitamin D, into calcitriol, the active form of vitamin D. Calcitriol is released into circulation to stimulate calcium uptake from the intestine. Finally, PTH also influences the reabsorption of calcium that occurs in the distal tubules and the renal collecting ducts of the kidney nephron.

The effects of PTH are mediated through the type 1 PTH receptor (PTH1 receptor, PTH1R, PTH/PTHrP receptor). PTH1R is expressed in high levels in bone and kidney and regulates calcium ion homeostasis through activation of adenylate cyclase and phospholipase C. In bone, PTH1R is expressed on the surface of osteoblasts and activated in response to PTH binding. PTH1R activation results in an increased RANKL expression. RANKL then binds RANK on the osteoclasts increasing the bone resorption rate. PTH1R is a class B member of the G protein-coupled receptors (GPCR) family. PTH1R activity is primarily mediated by Gus adenylyl cyclase-cAMP-protein kinase A (PKA) signaling pathway. The receptor can also activate the Gα_{q}-phospholipase C (PLC) β-inositol triphosphate-cytoplasmic Ca₂₊-protein kinase C (PKC) pathway, the Gα_{12/13}-phospholipase D-transforming protein RhoA pathway and the β-arrestin-extracellular signal-regulated kinase 1/2 (ERK1/2) pathway. In one aspect, compounds described herein are antagonists of PTH1R. Because of its critical role in regulation of calcium metabolism and bone growth and remodeling, PTH1R is of great interest in the treatment of osteoporosis and hypercalcemia.

Hyperparathyroidism is the abnormally high secretion of PTH. This occurs from a disorder either within the parathyroid glands (primary hyperparathyroidism, pHPT) or outside the parathyroid glands (secondary hyperparathyroidism, secondary HPT).

Primary hyperparathyroidism results from a hyperfunction of the parathyroid glands themselves, and most often occurs due to enlargement of one or more parathyroid glands. While over 100,000 people develop pHPT per year, patients are often asymptomatic, and when symptoms do arise, they are often due to elevated calcium levels in blood. Symptoms include constipation, chronic fatigue, bone aches, abdominal pain, neurocognitive issues, and kidney stones, among others. Primary HPT usually occurs randomly, and is most often diagnosed in people aged 50-60, with women being affected about three times more often than men. In 80% of cases, primary hyperparathyroidism is due to a single benign tumor known as a parathyroid adenoma. The remainder are due to adenomas of ≥2 glands, hyperplasia, and parathyroid cancer, although rarely. Primary HPT is associated with osteoporosis (from increased bone resorption), kidney stones (from increased urinary calcium levels), cardiovascular disease, and neurocognitive problems. Treatment options for pHPT are limited. About 85% of patients with pHPT are asymptomatic, and of these, about 31% maintain a “wait-and-see” approach. For the other 69% of asymptomatic patients and the 15% of patients showing symptoms, surgery to remove the overactive parathyroid glands is often the best course of action. In patients where surgery is unsuccessful, contraindicated, or delayed, medical therapy becomes necessary, to prevent progression of the disease, in mild patients, and to help restore or stabilize normal function in more severe patients. Current treatment options include the calcimimetic cinacalcet (Sensipar®) that enhances the sensitivity of the calcium sensing receptor in the parathyroid gland to extracellular calcium. Cinacalcet treatment results in a mild suppression of PTH secretion and a reduction in circulating calcium levels which leads to improved symptoms. However, this treatment does not impact overall bone mass density, and many patients (10-20%) report diarrhea and nausea due to the treatment. Bisphosphonates such as zolendronic acid (Zometa®) are sometimes used sequentially with calcimimetics to improve bone loss and bone mass density, but does not impact serum calcium or PTH, and can cause low blood pressure, fever, and vomiting in some patients. In one aspect, a PTH receptor antagonist is used in the treatment of pHPT. In some embodiments, a PTH1R antagonist is used in the treatment of pHPT. In some embodiments, serum calcium levels are decreased. In some embodiments, urinary calcium levels are decreased. In some embodiments, PTH levels are decreased. In some embodiments, bone mass density is stabilized.

Secondary hyperparathyroidism is a result of physiological secretion of PTH by the parathyroid glands in response to hypocalcemia, or low blood calcium levels. The most common causes of secondary HPT are vitamin D deficiency and chronic kidney failure. Vitamin D deficiency (for example, from lack of sunlight, diet, or malabsorption) leads to reduced calcium absorption by the intestine, hypocalcemia, and increased PTH secretion, and ultimately increased bone resorption. In cases of chronic kidney failure or chronic kidney disease, the kidneys can fail to convert vitamin D to its active form, resulting in hypocalcemia, increased PTH secretion, and ultimately increased bone resorption. Additionally, patients with chronic kidney disease also have higher blood phosphorous levels or severe calcium deficiencies which contribute to secondary HPT. Secondary HPT can also be associated with severe Crohn's or Celiac disease. In one aspect, a PTH receptor antagonist is used in the treatment of secondary HPT. In some embodiments, a PTH1R antagonist is used in the treatment of secondary HPT. In some embodiments, serum calcium levels are decreased. In some embodiments, urinary calcium levels are decreased. In some embodiments, PTH levels are decreased.

Tertiary hyperparathyroidism is seen in cases of long-term secondary HPT, and eventually leads to hyperplasia of the parathyroid glands and a loss of response to serum calcium levels. This disorder is most often seen in patients with end-stage kidney disease and as a result of autonomous release of parathyroid hormone while in a hypercalcaemic state. About 6-8% of kidney transplant recipients with chronic kidney disease develop tertiary HPT. In one aspect, a PTH receptor antagonist is used in the treatment of tertiary HPT. In some embodiments, a PTH1R antagonist is used in the treatment of tertiary HPT.

Parathyroid hormone-related protein (PTHrP) is a protein member of the parathyroid hormone family produced in low concentration in virtually all tissues. PTHrP acts in an autocrine/paracrine manner, exerting functions in cellular differentiation and apoptosis, playing a major role in pregnancy and fetal development but also in calcium transport and smooth muscle relaxation. In addition, PTHrP has been identified as a cause of hypercalcemia in cancer patients. PTHrP and PTH share structural similarities in their N-terminal parts (first 34 amino acid segments) that are sufficient for high affinity binding to PTH1R. Accordingly, both peptides are able to activate the PTH receptor with similar potency. PTHrP can mimic nearly all PTH functions including increases in bone resorption and distal tubular calcium reabsorption, activation of osteoclasts, and inhibition of proximal tubular phosphate transport. However, PTHrP lacks the normal feedback inhibition that PTH has. This increase in PTHrP-mediated PTH1R activity is responsible for most cases of humoral hypercalcemia of malignancy (HHM).

Humoral hypercalcemia of malignancy (HHM) is found in approximately 16% of cancer patients. HHM is most commonly seen in squamous cell carcinomas (head and neck, lung) and genitourinary tumors (renal cell carcinoma, ovarian). As cancer treatments get better and cancer patients are living longer, there is an increased need for long-term safe HHM therapies. Current treatment options include bisphosphonates such as zolendronic acid (Zometa®) and pamidronate (Aredia) for improving bone mass density. However, these treatments can cause osteonecrosis of jaw, nephrotoxicity, and hypocalcemia. RANKL antibodies such as denosumab (Xgeva®) have also been used to treat HHM, but require costly monthly injections and have long-term side effects including jaw necrosis. In one aspect, a PTH receptor antagonist is used in the treatment of HHM. In some embodiments, a PTH1R antagonist is used in the treatment of HHM. In some embodiments, serum calcium levels are decreased. In some embodiments, serum calcium levels are stabilized.

In addition to hyperparathyroidism (pHPT, secondary HPT, tertiary HPT) and humoral hypercalcemia of malignancy, it has also been hypothesized that there might be a role for an PTH1R antagonist in the treatment of familial hypocalciuric hypercalcemia (FHH), neonatal severe hyperparathyroidism (NSHPT), multiple endocrine neoplasia (including MEN1, MEN2a, MEN2b, and FMTC), Jansen's metaphyseal chondrodysplasia (JMC), and parathyroid carcinoma. In some embodiments, a PTH1R antagonist is used in the treatment of FHH. In some embodiments, a PTH1R antagonist is used in the treatment of NSHPT. In some embodiments, a PTH1R antagonist is used in the treatment of multiple endocrine neoplasia. In some embodiments, a PTH1R antagonist is used in the treatment of JMC. In some embodiments, a PTH1R antagonist is used in the treatment of parathyroid carcinoma.

In some embodiments, compounds described herein are amenable to administration to a mammal in need of treatment with an PTH1R antagonist.

### Compounds

Compounds of Formula (I′) or (I), including pharmaceutically acceptable salts, prodrugs, active metabolites, and pharmaceutically acceptable solvates thereof, are parathyroid hormone (PTH) receptor modulators. In some embodiments, the compounds of Formula (I′) or (I), including pharmaceutically acceptable salts, prodrugs, active metabolites, and pharmaceutically acceptable solvates thereof, are PTH1R modulators. In some embodiments, the PTH1R modulators are PTH1R antagonists.

In some embodiments, described herein is a compound of Formula (I′), or a pharmaceutically acceptable salt, or solvate thereof:

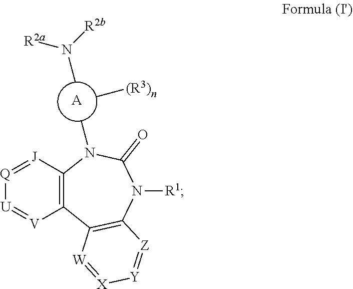

wherein:

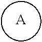

is selected from phenyl and a 6-membered heteroaryl ring comprising 1 or 2 nitrogen atoms;
J is C(R⁴) or N;
Q is C(R⁵) or N;
U is C(R⁶) or N;
V is C(R⁷) or N; wherein no more than two of J, Q, U, and V are N;
W is C(R⁸) or N;
X is C(R⁹) or N;
Y is C(R¹⁰) or N;
Z is C(R¹¹) or N; wherein no more than two of W, X, Y, and Z are N;
R¹ is hydrogen, C₁₋₆alkyl, C₂₋₆alkenyl, C₂₋₆alkynyl, C₃₋₆cycloalkyl, or C₂₋₉heterocycloalkyl, wherein C₁₋₆alkyl, C₂₋₆alkenyl, C₂₋₆alkynyl, C₃₋₆cycloalkyl, and C₂₋₉heterocycloalkyl are optionally substituted with one, two, three, four, or five R¹⁹;
R^{2a} is hydrogen, C₁₋₆alkyl, C₂₋₆alkenyl, C₂₋₆alkynyl, C₃₋₆cycloalkyl, or —(C(R¹⁶)(R¹⁷))ₘ-N(R^{18a})(R^{18b});
R^{2b} is hydrogen, C₁₋₆alkyl, C₂₋₆alkenyl, C₂₋₆alkynyl, or C₃₋₆cycloalkyl, wherein C₁₋₆alkyl, C₂₋₆alkenyl, C₂₋₆alkynyl, and C₃₋₆cycloalkyl are optionally substituted with one, two, three, four, or five R²⁰;
or R^{2a} and R^{2b} are taken together with the nitrogen atom to which they are attached to form a C₂₋₉heterocycloalkyl optionally substituted with one, two, three, four, or five R²⁰;
each R³ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —N(R¹⁴)C(O)R¹⁵, —S(O)₂R¹⁵, and —S(O)₂N(R¹²)(R¹³)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, —OR¹², —N(R¹²)(R¹³), and —C(O)OR¹²;
R⁴, R⁵, R⁶, R⁷, R⁸, R⁹, R¹⁰, and R¹¹ are each independently selected from hydrogen, halogen, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, —CN, —OR², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —N(R¹⁴)C(O)R¹⁵, —S(O)₂R¹⁵, and —S(O)₂N(R¹²)(R¹³)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, three, four, or five R²¹; each R¹² is independently selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, —C(O)OH, C₃₋₆cycloalkyl, C₂₋₉heterocycloalkyl, C₆₋₁₀aryl, and C₁₋₉heteroaryl;
each R¹³ is independently selected from hydrogen, C₁₋₆alkyl, and C₁₋₆haloalkyl;
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹⁶ and each R¹⁷ are each independently selected from hydrogen, halogen, hydroxy, C₁₋₆alkyl, C₁₋₆haloalkyl, and C₁₋₆alkoxy; or R¹⁶ and R¹⁷ are taken together to form a C₃₋₆cycloalkyl;
R^{18a} and R^{18b} are independently selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, three, four, or five R²⁰; or R^{18a} and R^{18b} are taken together with the nitrogen atom to which they are attached to form a C₂₋₉heterocycloalkyl optionally substituted with one, two, three, four, or five R²⁰;
each R¹⁹, R²⁰, and R²¹ are each independently selected from halogen, oxo, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —N(R¹⁴)C(O)R¹⁵, —S(O)₂R¹⁵, and —S(O)₂N(R¹²)(R¹³)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, oxo, C₁₋₆alkyl, C₁₋₆haloalkyl, —OR¹², —S(O)₂OH, —N(R¹²)(R¹³), and —C(O)OR¹²;
m is 2, 3, or 4; and
n is 0, 1, 2, 3, or 4.

In some embodiments is a compound of Formula (I′), or a pharmaceutically acceptable salt or solvate thereof, wherein J is C(R⁴), Q is C(R⁵), U is C(R⁶), and V is C(R⁷). In some embodiments is a compound of Formula (I′), or a pharmaceutically acceptable salt or solvate thereof, wherein J is C(R⁴) or N, Q is C(R⁵) or N, U is C(R⁶) or N, and V is C(R⁷) or N. In some embodiments is a compound of Formula (I′), or a pharmaceutically acceptable salt or solvate thereof, wherein J is N, Q is C(R⁵) or N, U is C(R⁶), and V is C(R⁷). In some embodiments is a compound of Formula (I′), or a pharmaceutically acceptable salt or solvate thereof, wherein J is C(R⁴), Q is N, U is C(R⁶), and V is C(R⁷). In some embodiments is a compound of Formula (I′), or a pharmaceutically acceptable salt or solvate thereof, wherein J is C(R⁴), Q is C(R⁵), U is N, and V is C(R⁷). In some embodiments is a compound of Formula (I′), or a pharmaceutically acceptable salt or solvate thereof, wherein J is C(R⁴), Q is C(R⁵), U is C(R⁶), and V is N.

In some embodiments is a compound of Formula (I′), or a pharmaceutically acceptable salt or solvate thereof, wherein W is C(R⁸), X is C(R⁹), Y is C(R¹⁰), and Z is N. In some embodiments is a compound of Formula (I′), or a pharmaceutically acceptable salt or solvate thereof, wherein W is C(R⁵), X is C(R⁹), Y is C(R¹⁰), and Z is C(R¹¹). In some embodiments is a compound of Formula (I′), or a pharmaceutically acceptable salt or solvate thereof, wherein W is N, X is C(R⁹), Y is C(R¹⁰), and Z is C(R¹¹). In some embodiments is a compound of Formula (I′), or a pharmaceutically acceptable salt or solvate thereof, wherein W is C(R⁸), X is N, Y is C(R¹⁰), and Z is C(R¹¹). In some embodiments is a compound of Formula (I′), or a pharmaceutically acceptable salt or solvate thereof, wherein W is C(R⁸), X is C(R⁹), Y is N, and Z is C(R¹¹).

In some embodiments is a compound of Formula (I′), or a pharmaceutically acceptable salt or solvate thereof, wherein R⁵ is selected from hydrogen, halogen, C₁₋₆alkyl, C₃₋₆cycloalkyl, C₂₋₉heterocycloalkyl, —CN, —C(O)N(R¹²)(R¹³), and —OR¹², wherein C₁₋₆alkyl, C₃₋₆cycloalkyl, and C₂₋₉heterocycloalkyl are optionally substituted with one, two, three, four, or five R²¹. In some embodiments is a compound of Formula (I′), or a pharmaceutically acceptable salt or solvate thereof, wherein R⁵ is selected from hydrogen, halogen, C₁₋₆alkyl, —CN, and —OR¹², wherein C₁₋₆alkyl is optionally substituted with one, two, three, four, or five R²¹. In some embodiments is a compound of Formula (I′), or a pharmaceutically acceptable salt or solvate thereof, wherein R⁵ is selected from hydrogen, halogen, C₁₋₆alkyl, —CN, and —OR¹², wherein C₁₋₆alkyl is optionally substituted with one, two, three halogen, and R¹² is selected from hydrogen and C₁₋₆alkyl. In some embodiments is a compound of Formula (I′), or a pharmaceutically acceptable salt or solvate thereof, wherein R⁵ is selected from hydrogen, —F, —Cl, —CH₃, —CF₃, —CN, and —OCH₃. In some embodiments is a compound of Formula (I′), or a pharmaceutically acceptable salt or solvate thereof, wherein R⁵ is hydrogen. In some embodiments is a compound of Formula (I′), or a pharmaceutically acceptable salt or solvate thereof, wherein R⁵ is —F. In some embodiments is a compound of Formula (I′), or a pharmaceutically acceptable salt or solvate thereof, wherein R⁵ is —Cl. In some embodiments is a compound of Formula (I′), or a pharmaceutically acceptable salt or solvate thereof, wherein R⁵ is —CH₃. In some embodiments is a compound of Formula (I′), or a pharmaceutically acceptable salt or solvate thereof, wherein R⁵ is —CF₃. In some embodiments is a compound of Formula (I′), or a pharmaceutically acceptable salt or solvate thereof, wherein R⁵ is —CN. In some embodiments is a compound of Formula (I′), or a pharmaceutically acceptable salt or solvate thereof, wherein R⁵ is —OCH₃.

In some embodiments is a compound of Formula (I′), or a pharmaceutically acceptable salt or solvate thereof, wherein R⁷ is selected from hydrogen, halogen, C₁₋₆alkyl, —CN, and —OR¹² wherein C₁₋₆alkyl is optionally substituted with one, two, three, four, or five R²¹. In some embodiments is a compound of Formula (I′), or a pharmaceutically acceptable salt or solvate thereof, wherein R⁷ is selected from hydrogen, halogen, C₁₋₆alkyl, —CN, and —OR¹², wherein C₁₋₆alkyl is optionally substituted with one, two, three halogen, and R¹² is selected from hydrogen and C₁₋₆alkyl. In some embodiments is a compound of Formula (I′), or a pharmaceutically acceptable salt or solvate thereof, wherein R⁷ is selected from hydrogen, —F, —Cl, —CH₃, —CF₃, —CN, and —OCH₃. In some embodiments is a compound of Formula (I′), or a pharmaceutically acceptable salt or solvate thereof, wherein R⁷ is hydrogen. In some embodiments is a compound of Formula (I′), or a pharmaceutically acceptable salt or solvate thereof, wherein R⁷ is —F. In some embodiments is a compound of Formula (I′), or a pharmaceutically acceptable salt or solvate thereof, wherein R⁷ is —Cl. In some embodiments is a compound of Formula (I′), or a pharmaceutically acceptable salt or solvate thereof, wherein R⁷ is —CH₃. In some embodiments is a compound of Formula (I′), or a pharmaceutically acceptable salt or solvate thereof, wherein R⁷ is —CF₃. In some embodiments is a compound of Formula (I′), or a pharmaceutically acceptable salt or solvate thereof, wherein R⁷ is —CN. In some embodiments is a compound of Formula (I′), or a pharmaceutically acceptable salt or solvate thereof, wherein R⁷ is —OCH₃.

In some embodiments is a compound of Formula (I′), or a pharmaceutically acceptable salt or solvate thereof, wherein R⁹ is selected from hydrogen, halogen, C₁₋₆alkyl, C₃₋₆cycloalkyl, —CN, and —OR¹², wherein C₁₋₆alkyl and C₃₋₆cycloalkyl are optionally substituted with one, two, three, four, or five R²¹. In some embodiments is a compound of Formula (I′), or a pharmaceutically acceptable salt or solvate thereof, wherein R⁹ is selected from hydrogen, halogen, C₁₋₆alkyl, C₃₋₆cycloalkyl, —CN, and —OR¹², wherein C₁₋₆alkyl and C₃₋₆cycloalkyl are optionally substituted with one, two, three halogen, and R¹² is selected from hydrogen and C₁₋₆alkyl. In some embodiments is a compound of Formula (I′), or a pharmaceutically acceptable salt or solvate thereof, wherein R⁹ is selected from hydrogen, —F, —Cl, —CH₃, —CF₃, —CN, —OCH₃, and

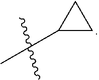 [SMILES: *C1CC1]

In some embodiments is a compound of Formula (I′), or a pharmaceutically acceptable salt or solvate thereof, wherein R⁹ is hydrogen. In some embodiments is a compound of Formula (I′), or a pharmaceutically acceptable salt or solvate thereof, wherein R⁹ is —F. In some embodiments is a compound of Formula (I′), or a pharmaceutically acceptable salt or solvate thereof, wherein R⁹ is —Cl. In some embodiments is a compound of Formula (I′), or a pharmaceutically acceptable salt or solvate thereof, wherein R⁹ is —CH₃. In some embodiments is a compound of Formula (I′), or a pharmaceutically acceptable salt or solvate thereof, wherein R⁹ is —CF₃. In some embodiments is a compound of Formula (I′), or a pharmaceutically acceptable salt or solvate thereof, wherein R⁹ is —CN. In some embodiments is a compound of Formula (I′), or a pharmaceutically acceptable salt or solvate thereof, wherein R⁹ is —OCH₃. In some embodiments is a compound of Formula (I′), or a pharmaceutically acceptable salt or solvate thereof, wherein R⁹ is

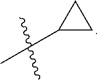 [SMILES: *C1CC1]

In some embodiments is a compound of Formula (I′), or a pharmaceutically acceptable salt or solvate thereof, wherein R⁴, R⁶, R⁸, and R¹⁰ are each independently selected from hydrogen, halogen, and unsubstituted C₁₋₆alkyl. In some embodiments is a compound of Formula (I′), or a pharmaceutically acceptable salt or solvate thereof, wherein R⁴, R⁶, R⁸, and R¹⁰ are each independently selected from hydrogen and halogen. In some embodiments is a compound of Formula (I′), or a pharmaceutically acceptable salt or solvate thereof, wherein R⁴, R⁶, R⁸, and R¹⁰ are each independently selected from hydrogen and unsubstituted C₁₋₆alkyl. In some embodiments is a compound of Formula (I′), or a pharmaceutically acceptable salt or solvate thereof, wherein R⁴, R⁶, R⁸, and R¹⁰ are each hydrogen. In some embodiments is a compound of Formula (I′), or a pharmaceutically acceptable salt or solvate thereof, wherein R¹¹ is hydrogen. In some embodiments is a compound of Formula (I′), or a pharmaceutically acceptable salt or solvate thereof, wherein R¹¹ is halogen.

In some embodiments is a compound of Formula (I′), or a pharmaceutically acceptable salt or solvate thereof, wherein

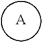

is phenyl. In some embodiments is a compound of Formula (I′), or a pharmaceutically acceptable salt or solvate thereof, wherein

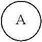

is a 6-membered heteroaryl ring comprising 1 or 2 nitrogen atoms. In some embodiments is a compound of Formula (I′), or a pharmaceutically acceptable salt or solvate thereof, wherein

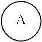

is a 6-membered heteroaryl ring comprising 1 nitrogen atom. In some embodiments is a compound of Formula (I′), or a pharmaceutically acceptable salt or solvate thereof, wherein

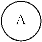

is a 6-membered heteroaryl ring comprising 2 nitrogen atoms.

In some embodiments is a compound of Formula (I′), or a pharmaceutically acceptable salt or solvate thereof, wherein R¹ is C₁₋₆alkyl optionally substituted with one, two, three, four, or five R¹⁹. In some embodiments is a compound of Formula (I′), or a pharmaceutically acceptable salt or solvate thereof, wherein R¹ is C₁₋₆alkyl optionally substituted with one, two, three, four, or five R¹⁹, and each R¹⁹ is independently selected from halogen, C₁₋₆haloalkyl, C₃₋₆cycloalkyl, —OR¹², and —N(R¹²)(R¹³). In some embodiments is a compound of Formula (I′), or a pharmaceutically acceptable salt or solvate thereof, wherein R¹ is C₁₋₆alkyl optionally substituted with one, two, three, four, or five R¹⁹, each R¹⁹ is independently selected from halogen, C₃₋₆cycloalkyl, and OR¹², and each R¹² is independently selected from hydrogen and C₁₋₆alkyl. In some embodiments is a compound of Formula (I′), or a pharmaceutically acceptable salt or solvate thereof, wherein R¹ is —CF₃, —CH₂CF₃, —CH₂CH₂OH, —CH₂CH₂OCH₃, or

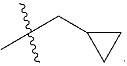 [SMILES: *CC1CC1]

In some embodiments is a compound of Formula (I′), or a pharmaceutically acceptable salt or solvate thereof, wherein R¹ is —CF₃. In some embodiments is a compound of Formula (I′), or a pharmaceutically acceptable salt or solvate thereof, wherein R¹ is —CH₂CF₃. In some embodiments is a compound of Formula (I′), or a pharmaceutically acceptable salt or solvate thereof, wherein R¹ is —CH₂CH₂OH. In some embodiments is a compound of Formula (I′), or a pharmaceutically acceptable salt or solvate thereof, wherein R¹ is —CH₂CH₂OCH₃. In some embodiments is a compound of Formula (I′), or a pharmaceutically acceptable salt or solvate thereof, wherein R¹ is

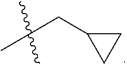 [SMILES: *CC1CC1]

In some embodiments is a compound of Formula (I′), or a pharmaceutically acceptable salt or solvate thereof, wherein R¹ is an unsubstituted C₁₋₆alkyl. In some embodiments is a compound of Formula (I′), or a pharmaceutically acceptable salt or solvate thereof, wherein R¹ is —CH₃, —CH₂CH₃, —CH(CH₃)₂, or —CH₂CH₂CH₃. In some embodiments is a compound of Formula (I′), or a pharmaceutically acceptable salt or solvate thereof, wherein R¹ is —CH₃. In some embodiments is a compound of Formula (I′), or a pharmaceutically acceptable salt or solvate thereof, wherein R¹ is —CH₂CH₃. In some embodiments is a compound of Formula (I′), or a pharmaceutically acceptable salt or solvate thereof, wherein R¹ is —CH(CH₃)₂. In some embodiments is a compound of Formula (I′), or a pharmaceutically acceptable salt or solvate thereof, wherein R¹ is —CH₂CH₂CH₃. In some embodiments is a compound of Formula (I′), or a pharmaceutically acceptable salt or solvate thereof, wherein R¹ is C₃₋₆cycloalkyl optionally substituted with one, two, three, four, or five R¹⁹. In some embodiments is a compound of Formula (I′), or a pharmaceutically acceptable salt or solvate thereof, wherein R¹ is

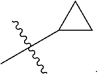 [SMILES: *C1CC1]

In some embodiments is a compound of Formula (I′), or a pharmaceutically acceptable salt or solvate thereof, wherein R^{2a} is —(C(R¹⁶)(R¹⁷))ₘ-N(R^{18a})(R^{18b}). In some embodiments is a compound of Formula (I′), or a pharmaceutically acceptable salt or solvate thereof, wherein each R¹⁶ and each R¹⁷ are each independently selected from hydrogen and C₁₋₆alkyl. In some embodiments is a compound of Formula (I′), or a pharmaceutically acceptable salt or solvate thereof, wherein each R¹⁶ and each R¹⁷ are hydrogen. In some embodiments is a compound of Formula (I′), or a pharmaceutically acceptable salt or solvate thereof, wherein m is 2. In some embodiments is a compound of Formula (I′), or a pharmaceutically acceptable salt or solvate thereof, wherein m is 3. In some embodiments is a compound of Formula (I′), or a pharmaceutically acceptable salt or solvate thereof, wherein m is 4. In some embodiments is a compound of Formula (I′), or a pharmaceutically acceptable salt or solvate thereof, wherein R^{18a} is C₁₋₆alkyl optionally substituted with one, two, three, four, or five R²⁰. In some embodiments is a compound of Formula (I′), or a pharmaceutically acceptable salt or solvate thereof, wherein R^{18a} is C₁₋₆alkyl optionally substituted with one, two, three, four, or five R²⁰, and each R²⁰ is independently selected from halogen, —OR¹², —N(R¹²)(R¹³), and —C(O)OR¹². In some embodiments is a compound of Formula (I′), or a pharmaceutically acceptable salt or solvate thereof, wherein R^{18a} is C₁₋₆alkyl substituted with one, two, or three R²⁰, each R²⁰ is independently selected from halogen, —OR¹², —N(R¹²)(R¹³), and —C(O)OR¹², each R¹² is independently selected from hydrogen and C₁₋₆alkyl, and each R¹³ is independently selected from hydrogen and C₁₋₆alkyl. In some embodiments is a compound of Formula (I′), or a pharmaceutically acceptable salt or solvate thereof, wherein R^{18a} is —CH₂CH₂OH, —CH₂CH₂OCH₃, —CH₂CH₂NH₂, —CH₂CH₂N(H)(CH₃), —CH₂CH₂C(O)OH, and —CH₂CH₂CH₂C(O)OH. In some embodiments is a compound of Formula (I′), or a pharmaceutically acceptable salt or solvate thereof, wherein R^{18a} is —CH₂CH₂OH. In some embodiments is a compound of Formula (I′), or a pharmaceutically acceptable salt or solvate thereof, wherein R^{18a} is —CH₂CH₂OCH₃. In some embodiments is a compound of Formula (I′), or a pharmaceutically acceptable salt or solvate thereof, wherein R^{18a} is —CH₂CH₂NH₂. In some embodiments is a compound of Formula (I′), or a pharmaceutically acceptable salt or solvate thereof, wherein R^{18a} is —CH₂CH₂N(H)(CH₃). In some embodiments is a compound of Formula (I′), or a pharmaceutically acceptable salt or solvate thereof, wherein R^{18a} is —CH₂CH₂C(O)OH. In some embodiments is a compound of Formula (I′), or a pharmaceutically acceptable salt or solvate thereof, wherein R^{18a} is —CH₂CH₂CH₂C(O)OH. In some embodiments is a compound of Formula (I′), or a pharmaceutically acceptable salt or solvate thereof, wherein R^{18a} is an unsubstituted C₁₋₆alkyl. In some embodiments is a compound of Formula (I′), or a pharmaceutically acceptable salt or solvate thereof, wherein R^{18a} is —CH₃. In some embodiments is a compound of Formula (I′), or a pharmaceutically acceptable salt or solvate thereof, wherein R^{18a} is hydrogen. In some embodiments is a compound of Formula (I′), or a pharmaceutically acceptable salt or solvate thereof, wherein R^{18b} is hydrogen. In some embodiments is a compound of Formula (I′), or a pharmaceutically acceptable salt or solvate thereof, wherein R^{18a} is an unsubstituted C₁₋₆alkyl. In some embodiments is a compound of Formula (I′), or a pharmaceutically acceptable salt or solvate thereof, wherein R^{18b} is —CH₃. In some embodiments is a compound of Formula (I′), or a pharmaceutically acceptable salt or solvate thereof, wherein R^{2b} is hydrogen.

In some embodiments is a compound of Formula (I′), or a pharmaceutically acceptable salt or solvate thereof, wherein R^{2a} and R^{2b} are taken together with the nitrogen atom to which they are attached to form a C₂₋₉heterocycloalkyl optionally substituted with one, two, three, four, or five R²⁰. In some embodiments is a compound of Formula (I′), or a pharmaceutically acceptable salt or solvate thereof, wherein R^{2a} and R^{2b} are taken together with the nitrogen atom to which they are attached to form a C₂₋₉heterocycloalkyl optionally substituted with one, two, three, four, or five R²⁰, and each R²⁰ is independently selected from halogen, —OR¹², —N(R¹²)(R¹³), and C₁₋₆alkyl optionally substituted with —N(R¹²)(R¹³). In some embodiments is a compound of Formula (I′), or a pharmaceutically acceptable salt or solvate thereof, wherein R^{2a} and R^{2b} are taken together with the nitrogen atom to which they are attached to form a substituted C₂₋₉heterocycloalkyl selected from

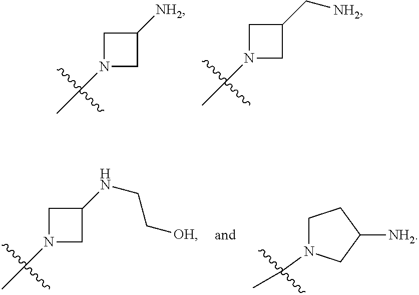 [SMILES: *N1CC(N)C1 ; *N1CC(CN)C1 ; *N1CC(NCCO)C1 ; *N1CCC(N)C1]

In some embodiments is a compound of Formula (I′), or a pharmaceutically acceptable salt or solvate thereof, wherein R^{2a} and R^{2b} are taken together with the nitrogen atom to which they are attached to form

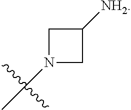 [SMILES: *N1CC(N)C1]

In some embodiments is a compound of Formula (I′), or a pharmaceutically acceptable salt or solvate thereof, wherein R^{2a} and R^{2b} are taken together with the nitrogen atom to which they are attached to form

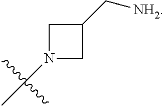 [SMILES: *N1CC(CN)C1]

In some embodiments is a compound of Formula (I′), or a pharmaceutically acceptable salt or solvate thereof, wherein R^{2a} and R^{2b} are taken together with the nitrogen atom to which they are attached to form

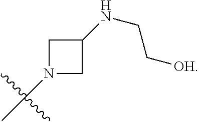 [SMILES: *N1CC(NCCO)C1]

In some embodiments is a compound of Formula (I′), or a pharmaceutically acceptable salt or solvate thereof, wherein R^{2a} and R^{2b} are taken together with the nitrogen atom to which they are attached to form

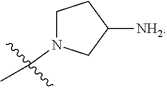 [SMILES: *N1CCC(N)C1]

In some embodiments is a compound of Formula (I′), or a pharmaceutically acceptable salt or solvate thereof, wherein each R³ is independently selected from halogen, C₁₋₆alkyl, and —OR¹², wherein C₁₋₆alkyl is optionally substituted with one, two, three halogen, and R¹² is selected from hydrogen and C₁₋₆alkyl. In some embodiments is a compound of Formula (I′), or a pharmaceutically acceptable salt or solvate thereof, wherein each R³ is independently selected from halogen. In some embodiments is a compound of Formula (I′), or a pharmaceutically acceptable salt or solvate thereof, wherein each R³ is —F.

In some embodiments is a compound of Formula (I′), or a pharmaceutically acceptable salt or solvate thereof, wherein n is 1 or 2. In some embodiments is a compound of Formula (I′), or a pharmaceutically acceptable salt or solvate thereof, wherein n is 1. In some embodiments is a compound of Formula (I′), or a pharmaceutically acceptable salt or solvate thereof, wherein n is 2. In some embodiments is a compound of Formula (I′), or a pharmaceutically acceptable salt or solvate thereof, wherein n is 3. In some embodiments is a compound of Formula (I′), or a pharmaceutically acceptable salt or solvate thereof, wherein n is 4. In some embodiments is a compound of Formula (I′), or a pharmaceutically acceptable salt or solvate thereof, wherein n is 0.

In some embodiments, described herein is a compound of Formula (I), or a pharmaceutically acceptable salt, or solvate thereof:

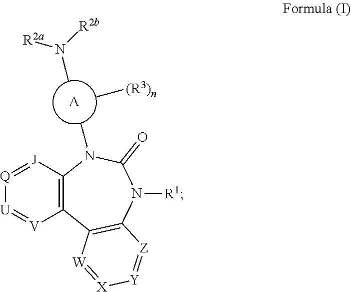

wherein:

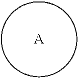

is selected from phenyl and a 6-membered heteroaryl ring comprising 1 or 2 nitrogen atoms;
J is C(R⁴) or N;
Q is C(R⁵) or N;
U is C(R⁶) or N;
V is C(R⁷) or N; wherein no more than two of J, Q, U, and V are N;
W is C(R⁸) or N;
X is C(R⁹) or N;
Y is C(R¹⁰) or N;
Z is C(R¹¹) or N; wherein no more than two of W, X, Y, and Z are N;
R¹ is hydrogen, C₁₋₆alkyl, C₂₋₆alkenyl, C₂₋₆alkynyl, C₃₋₆cycloalkyl, or C₂₋₉heterocycloalkyl, wherein C₁₋₆alkyl, C₂₋₆alkenyl, C₂₋₆alkynyl, C₃₋₆cycloalkyl, and C₂₋₉heterocycloalkyl are optionally substituted with one, two, three, four, or five R¹⁹;
R^{2a} is hydrogen, C₁₋₆alkyl, C₂₋₆alkenyl, C₂₋₆alkynyl, C₃₋₆cycloalkyl, or —(C(R¹⁶)(R¹⁷))ₘ-N(R^{18a})(R^{18b});
R^{2b} is hydrogen, C₁₋₆alkyl, C₂₋₆alkenyl, C₂₋₆alkynyl, or C₃₋₆cycloalkyl, wherein C₁₋₆alkyl, C₂₋₆alkenyl, C₂₋₆alkynyl, and C₃₋₆cycloalkyl are optionally substituted with one, two, three, four, or five R²⁰;
or R^{2a} and R^{2b} are taken together with the nitrogen atom to which they are attached to form a C₂₋₉heterocycloalkyl optionally substituted with one, two, three, four, or five R²⁰;
each R³ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —N(R¹⁴)C(O)R¹⁵, —S(O)₂R¹⁵, and —S(O)₂N(R¹²)(R¹³)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, —OR¹², —N(R¹²)(R¹³), and —C(O)OR¹²;
R⁴, R⁵, R⁶, R⁷, R⁸, R⁹, R¹⁰, and R¹¹ are each independently selected from hydrogen, halogen, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, —CN, —OR², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —N(R¹⁴)C(O)R¹⁵, —S(O)₂R¹⁵, and —S(O)₂N(R¹²)(R¹³)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, three, four, or five R²¹;
each R¹² is independently selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹³ is independently selected from hydrogen, C₁₋₆alkyl, and C₁₋₆haloalkyl;
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹⁶ and each R¹⁷ are each independently selected from hydrogen, halogen, hydroxy, C₁₋₆alkyl, C₁₋₆haloalkyl, and C₁₋₆alkoxy; or R¹⁶ and R¹⁷ are taken together to form a C₃₋₆cycloalkyl;
R^{18a} and R^{18b} are independently selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, three, four, or five R²⁰; or R^{18a} and R^{18b} are taken together with the nitrogen atom to which they are attached to form a C₂₋₉heterocycloalkyl optionally substituted with one, two, three, four, or five R²⁰;
each R¹⁹, R²⁰, and R²¹ are each independently selected from halogen, oxo, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —N(R¹⁴)C(O)R¹⁵, —S(O)₂R¹⁵, and —S(O)₂N(R¹²)(R¹³)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, —OR¹², —N(R¹²)(R¹³), and —C(O)OR¹²;
m is 2, 3, or 4; and
n is 0, 1, 2, 3, or 4.

In some embodiments is a compound of Formula (I), or a pharmaceutically acceptable salt or solvate thereof, wherein J is C(R⁴), Q is C(R⁵), U is C(R⁶), and V is C(R⁷). In some embodiments is a compound of Formula (I), or a pharmaceutically acceptable salt or solvate thereof, wherein J is C(R⁴) or N, Q is C(R⁵) or N, U is C(R⁶) or N, and V is C(R⁷) or N. In some embodiments is a compound of Formula (I), or a pharmaceutically acceptable salt or solvate thereof, wherein J is N, Q is C(R⁵) or N, U is C(R⁶), and V is C(R⁷). In some embodiments is a compound of Formula (I), or a pharmaceutically acceptable salt or solvate thereof, wherein J is C(R⁴), Q is N, U is C(R⁶), and V is C(R⁷). In some embodiments is a compound of Formula (I), or a pharmaceutically acceptable salt or solvate thereof, wherein J is C(R⁴), Q is C(R⁵), U is N, and V is C(R⁷). In some embodiments is a compound of Formula (I), or a pharmaceutically acceptable salt or solvate thereof, wherein J is C(R⁴), Q is C(R⁵), U is C(R⁶), and V is N.

In some embodiments is a compound of Formula (I), or a pharmaceutically acceptable salt or solvate thereof, wherein W is C(R⁸), X is C(R⁹), Y is C(R¹⁰), and Z is N. In some embodiments is a compound of Formula (I), or a pharmaceutically acceptable salt or solvate thereof, wherein W is C(R⁸), X is C(R⁹), Y is C(R¹⁰), and Z is C(R¹¹). In some embodiments is a compound of Formula (I), or a pharmaceutically acceptable salt or solvate thereof, wherein W is N, X is C(R⁹), Y is C(R¹⁰), and Z is C(R¹¹). In some embodiments is a compound of Formula (I), or a pharmaceutically acceptable salt or solvate thereof, wherein W is C(R⁸), X is N, Y is C(R¹⁰), and Z is C(R¹¹). In some embodiments is a compound of Formula (I), or a pharmaceutically acceptable salt or solvate thereof, wherein W is C(R⁸), X is C(R⁹), Y is N, and Z is C(R¹¹).

In some embodiments is a compound of Formula (I), or a pharmaceutically acceptable salt or solvate thereof, wherein R⁵ is selected from hydrogen, halogen, C₁₋₆alkyl, C₃₋₆cycloalkyl, C₂₋₉heterocycloalkyl, —CN, —C(O)N(R¹²)(R¹³), and —OR¹², wherein C₁₋₆alkyl, C₃₋₆cycloalkyl, and C₂₋₉heterocycloalkyl are optionally substituted with one, two, three, four, or five R²¹. In some embodiments is a compound of Formula (I), or a pharmaceutically acceptable salt or solvate thereof, wherein R⁵ is selected from hydrogen, halogen, C₁₋₆alkyl, —CN, and —OR¹², wherein C₁₋₆alkyl is optionally substituted with one, two, three, four, or five R²¹. In some embodiments is a compound of Formula (I), or a pharmaceutically acceptable salt or solvate thereof, wherein R⁵ is selected from hydrogen, halogen, C₁₋₆alkyl, —CN, and —OR¹², wherein C₁₋₆alkyl is optionally substituted with one, two, three halogen, and R¹² is selected from hydrogen and C₁₋₆alkyl. In some embodiments is a compound of Formula (I), or a pharmaceutically acceptable salt or solvate thereof, wherein R⁵ is selected from hydrogen, —F, —Cl, —CH₃, —CF₃, —CN, and —OCH₃. In some embodiments is a compound of Formula (I), or a pharmaceutically acceptable salt or solvate thereof, wherein R⁵ is hydrogen. In some embodiments is a compound of Formula (I), or a pharmaceutically acceptable salt or solvate thereof, wherein R⁵ is —F. In some embodiments is a compound of Formula (I), or a pharmaceutically acceptable salt or solvate thereof, wherein R⁵ is —Cl. In some embodiments is a compound of Formula (I), or a pharmaceutically acceptable salt or solvate thereof, wherein R⁵ is —CH₃. In some embodiments is a compound of Formula (I), or a pharmaceutically acceptable salt or solvate thereof, wherein R⁵ is —CF₃. In some embodiments is a compound of Formula (I), or a pharmaceutically acceptable salt or solvate thereof, wherein R⁵ is —CN. In some embodiments is a compound of Formula (I), or a pharmaceutically acceptable salt or solvate thereof, wherein R⁵ is —OCH₃.

In some embodiments is a compound of Formula (I), or a pharmaceutically acceptable salt or solvate thereof, wherein R⁷ is selected from hydrogen, halogen, C₁₋₆alkyl, —CN, and —OR¹², wherein C₁₋₆alkyl is optionally substituted with one, two, three, four, or five R²¹. In some embodiments is a compound of Formula (I), or a pharmaceutically acceptable salt or solvate thereof, wherein R⁷ is selected from hydrogen, halogen, C₁₋₆alkyl, —CN, and —OR¹², wherein C₁₋₆alkyl is optionally substituted with one, two, three halogen, and R¹² is selected from hydrogen and C₁₋₆alkyl. In some embodiments is a compound of Formula (I), or a pharmaceutically acceptable salt or solvate thereof, wherein R⁷ is selected from hydrogen, —F, —Cl, —CH₃, —CF₃, —CN, and —OCH₃. In some embodiments is a compound of Formula (I), or a pharmaceutically acceptable salt or solvate thereof, wherein R⁷ is hydrogen. In some embodiments is a compound of Formula (I), or a pharmaceutically acceptable salt or solvate thereof, wherein R⁷ is —F. In some embodiments is a compound of Formula (I), or a pharmaceutically acceptable salt or solvate thereof, wherein R⁷ is —Cl. In some embodiments is a compound of Formula (I), or a pharmaceutically acceptable salt or solvate thereof, wherein R⁷ is —CH₃. In some embodiments is a compound of Formula (I), or a pharmaceutically acceptable salt or solvate thereof, wherein R⁷ is —CF₃. In some embodiments is a compound of Formula (I), or a pharmaceutically acceptable salt or solvate thereof, wherein R⁷ is —CN. In some embodiments is a compound of Formula (I), or a pharmaceutically acceptable salt or solvate thereof, wherein R⁷ is —OCH₃.

In some embodiments is a compound of Formula (I), or a pharmaceutically acceptable salt or solvate thereof, wherein R⁹ is selected from hydrogen, halogen, C₁₋₆alkyl, C₃₋₆cycloalkyl, —CN, and —OR¹², wherein C₁₋₆alkyl and C₃₋₆cycloalkyl are optionally substituted with one, two, three, four, or five R²¹. In some embodiments is a compound of Formula (I), or a pharmaceutically acceptable salt or solvate thereof, wherein R⁹ is selected from hydrogen, halogen, C₁₋₆alkyl, C₃₋₆cycloalkyl, —CN, and —OR¹², wherein C₁₋₆alkyl and C₃₋₆cycloalkyl are optionally substituted with one, two, three halogen, and R¹² is selected from hydrogen and C₁₋₆alkyl. In some embodiments is a compound of Formula (I), or a pharmaceutically acceptable salt or solvate thereof, wherein R⁹ is selected from hydrogen, —F, —Cl, —CH₃, —CF₃, —CN, —OCH₃, and

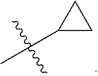 [SMILES: *C1CC1]

In some embodiments is a compound of Formula (I), or a pharmaceutically acceptable salt or solvate thereof, wherein R⁹ is hydrogen. In some embodiments is a compound of Formula (I), or a pharmaceutically acceptable salt or solvate thereof, wherein R⁹ is —F. In some embodiments is a compound of Formula (I), or a pharmaceutically acceptable salt or solvate thereof, wherein R⁹ is —Cl. In some embodiments is a compound of Formula (I), or a pharmaceutically acceptable salt or solvate thereof, wherein R⁹ is —CH₃. In some embodiments is a compound of Formula (I), or a pharmaceutically acceptable salt or solvate thereof, wherein R⁹ is —CF₃. In some embodiments is a compound of Formula (I), or a pharmaceutically acceptable salt or solvate thereof, wherein R⁹ is —CN. In some embodiments is a compound of Formula (I), or a pharmaceutically acceptable salt or solvate thereof, wherein R⁹ is —OCH₃. In some embodiments is a compound of Formula (I), or a pharmaceutically acceptable salt or solvate thereof, wherein R⁹ is

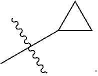 [SMILES: *C1CC1]

In some embodiments is a compound of Formula (I), or a pharmaceutically acceptable salt or solvate thereof, wherein R⁴, R⁶, R⁸, and R¹⁰ are each independently selected from hydrogen, halogen, and unsubstituted C₁₋₆alkyl. In some embodiments is a compound of Formula (I), or a pharmaceutically acceptable salt or solvate thereof, wherein R⁴, R⁶, R⁸, and R¹⁰ are each independently selected from hydrogen and halogen. In some embodiments is a compound of Formula (I), or a pharmaceutically acceptable salt or solvate thereof, wherein R⁴, R⁶, R⁸, and R¹⁰ are each independently selected from hydrogen and unsubstituted C₁₋₆alkyl. In some embodiments is a compound of Formula (I), or a pharmaceutically acceptable salt or solvate thereof, wherein R⁴, R⁶, R⁸, and R¹⁰ are each hydrogen. In some embodiments is a compound of Formula (I), or a pharmaceutically acceptable salt or solvate thereof, wherein R¹¹ is hydrogen. In some embodiments is a compound of Formula (I), or a pharmaceutically acceptable salt or solvate thereof, wherein R¹¹ is halogen.

In some embodiments is a compound of Formula (I), or a pharmaceutically acceptable salt or solvate thereof, wherein

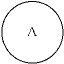

is phenyl. In some embodiments is a compound of Formula (I), or a pharmaceutically acceptable salt or solvate thereof, wherein

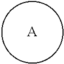

is a 6-membered heteroaryl ring comprising 1 or 2 nitrogen atoms. In some embodiments is a compound of Formula (I), or a pharmaceutically acceptable salt or solvate thereof, wherein

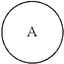

is a 6-membered heteroaryl ring comprising 1 nitrogen atom. In some embodiments is a compound of Formula (I), or a pharmaceutically acceptable salt or solvate thereof, wherein

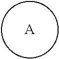

is a 6-membered heteroaryl ring comprising 2 nitrogen atoms.

In some embodiments is a compound of Formula (I), or a pharmaceutically acceptable salt or solvate thereof, wherein R¹ is C₁₋₆alkyl optionally substituted with one, two, three, four, or five R¹⁹. In some embodiments is a compound of Formula (I), or a pharmaceutically acceptable salt or solvate thereof, wherein R¹ is C₁₋₆alkyl optionally substituted with one, two, three, four, or five R¹⁹, and each R¹⁹ is independently selected from halogen, C₁₋₆haloalkyl, C₃₋₆cycloalkyl, —OR¹², and —N(R¹²)(R¹³). In some embodiments is a compound of Formula (I), or a pharmaceutically acceptable salt or solvate thereof, wherein R¹ is C₁₋₆alkyl optionally substituted with one, two, three, four, or five R¹⁹, each R¹⁹ is independently selected from halogen, C₃₋₆cycloalkyl, and OR¹², and each R¹² is independently selected from hydrogen and C₁₋₆alkyl. In some embodiments is a compound of Formula (I), or a pharmaceutically acceptable salt or solvate thereof, wherein R¹ is —CF₃, —CH₂CF₃, —CH₂CH₂OH, —CH₂CH₂OCH₃, or

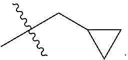 [SMILES: *CC1CC1]

In some embodiments is a compound of Formula (I), or a pharmaceutically acceptable salt or solvate thereof, wherein R¹ is —CF₃. In some embodiments is a compound of Formula (I), or a pharmaceutically acceptable salt or solvate thereof, wherein R¹ is —CH₂CF₃. In some embodiments is a compound of Formula (I), or a pharmaceutically acceptable salt or solvate thereof, wherein R¹ is —CH₂CH₂OH. In some embodiments is a compound of Formula (I), or a pharmaceutically acceptable salt or solvate thereof, wherein R¹ is —CH₂CH₂OCH₃. In some embodiments is a compound of Formula (I), or a pharmaceutically acceptable salt or solvate thereof, wherein R¹ is

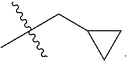 [SMILES: *CC1CC1]

In some embodiments is a compound of Formula (I), or a pharmaceutically acceptable salt or solvate thereof, wherein R¹ is an unsubstituted C₁₋₆alkyl. In some embodiments is a compound of Formula (I), or a pharmaceutically acceptable salt or solvate thereof, wherein R¹ is —CH₃, —CH₂CH₃, —CH(CH₃)₂, or —CH₂CH₂CH₃. In some embodiments is a compound of Formula (I), or a pharmaceutically acceptable salt or solvate thereof, wherein R¹ is —CH₃. In some embodiments is a compound of Formula (I), or a pharmaceutically acceptable salt or solvate thereof, wherein R¹ is —CH₂CH₃. In some embodiments is a compound of Formula (I), or a pharmaceutically acceptable salt or solvate thereof, wherein R¹ is —CH(CH₃)₂. In some embodiments is a compound of Formula (I), or a pharmaceutically acceptable salt or solvate thereof, wherein R¹ is —CH₂CH₂CH₃. In some embodiments is a compound of Formula (I), or a pharmaceutically acceptable salt or solvate thereof, wherein R¹ is C₃₋₆cycloalkyl optionally substituted with one, two, three, four, or five R¹⁹. In some embodiments is a compound of Formula (I), or a pharmaceutically acceptable salt or solvate thereof, wherein R¹ is

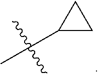 [SMILES: *C1CC1]

In some embodiments is a compound of Formula (I), or a pharmaceutically acceptable salt or solvate thereof, wherein R^{2a} is —(C(R¹⁶)(R¹⁷))ₘ-N(R^{18a})(R^{18b}). In some embodiments is a compound of Formula (I), or a pharmaceutically acceptable salt or solvate thereof, wherein each R¹⁶ and each R¹⁷ are each independently selected from hydrogen and C₁₋₆alkyl. In some embodiments is a compound of Formula (I), or a pharmaceutically acceptable salt or solvate thereof, wherein each R¹⁶ and each R¹⁷ are hydrogen. In some embodiments is a compound of Formula (I), or a pharmaceutically acceptable salt or solvate thereof, wherein m is 2. In some embodiments is a compound of Formula (I), or a pharmaceutically acceptable salt or solvate thereof, wherein m is 3. In some embodiments is a compound of Formula (I), or a pharmaceutically acceptable salt or solvate thereof, wherein m is 4. In some embodiments is a compound of Formula (I), or a pharmaceutically acceptable salt or solvate thereof, wherein R^{18a} is C₁₋₆alkyl optionally substituted with one, two, three, four, or five R²⁰. In some embodiments is a compound of Formula (I), or a pharmaceutically acceptable salt or solvate thereof, wherein R^{18a} is C₁₋₆alkyl optionally substituted with one, two, three, four, or five R²⁰, and each R²⁰ is independently selected from halogen, —OR¹², —N(R¹²)(R¹³), and —C(O)OR¹². In some embodiments is a compound of Formula (I), or a pharmaceutically acceptable salt or solvate thereof, wherein R^{18a} is C₁₋₆alkyl substituted with one, two, or three R²⁰, each R²⁰ is independently selected from halogen, —OR¹², —N(R¹²)(R¹³), and —C(O)OR¹², each R¹² is independently selected from hydrogen and C₁₋₆alkyl, and each R¹³ is independently selected from hydrogen and C₁₋₆alkyl. In some embodiments is a compound of Formula (I), or a pharmaceutically acceptable salt or solvate thereof, wherein R^{18a} is —CH₂CH₂OH, —CH₂CH₂OCH₃, —CH₂CH₂NH₂, —CH₂CH₂N(H)(CH₃), —CH₂CH₂C(O)OH, and —CH₂CH₂CH₂C(O)OH. In some embodiments is a compound of Formula (I), or a pharmaceutically acceptable salt or solvate thereof, wherein R^{18a} is —CH₂CH₂OH. In some embodiments is a compound of Formula (I), or a pharmaceutically acceptable salt or solvate thereof, wherein R^{18a} is —CH₂CH₂OCH₃. In some embodiments is a compound of Formula (I), or a pharmaceutically acceptable salt or solvate thereof, wherein R^{18a} is —CH₂CH₂NH₂. In some embodiments is a compound of Formula (I), or a pharmaceutically acceptable salt or solvate thereof, wherein R^{18a} is —CH₂CH₂N(H)(CH₃). In some embodiments is a compound of Formula (I), or a pharmaceutically acceptable salt or solvate thereof, wherein R^{18a} is —CH₂CH₂C(O)OH. In some embodiments is a compound of Formula (I), or a pharmaceutically acceptable salt or solvate thereof, wherein R^{18a} is —CH₂CH₂CH₂C(O)OH. In some embodiments is a compound of Formula (I), or a pharmaceutically acceptable salt or solvate thereof, wherein R^{18a} is an unsubstituted C₁₋₆alkyl. In some embodiments is a compound of Formula (I), or a pharmaceutically acceptable salt or solvate thereof, wherein R^{18a} is —CH₃. In some embodiments is a compound of Formula (I), or a pharmaceutically acceptable salt or solvate thereof, wherein R^{18a} is hydrogen. In some embodiments is a compound of Formula (I), or a pharmaceutically acceptable salt or solvate thereof, wherein R^{18b} is hydrogen. In some embodiments is a compound of Formula (I), or a pharmaceutically acceptable salt or solvate thereof, wherein R^{18b} is an unsubstituted C₁₋₆alkyl. In some embodiments is a compound of Formula (I), or a pharmaceutically acceptable salt or solvate thereof, wherein R^{18b} is —CH₃. In some embodiments is a compound of Formula (I), or a pharmaceutically acceptable salt or solvate thereof, wherein R^{2b} is hydrogen.

In some embodiments is a compound of Formula (I), or a pharmaceutically acceptable salt or solvate thereof, wherein R^{2a} and R^{2b} are taken together with the nitrogen atom to which they are attached to form a C₂₋₉heterocycloalkyl optionally substituted with one, two, three, four, or five R²⁰. In some embodiments is a compound of Formula (I), or a pharmaceutically acceptable salt or solvate thereof, wherein R^{2a} and R^{2b} are taken together with the nitrogen atom to which they are attached to form a C₂₋₉heterocycloalkyl optionally substituted with one, two, three, four, or five R²⁰, and each R²⁰ is independently selected from halogen, —OR¹², —N(R¹²)(R¹³), and C₁₋₆alkyl optionally substituted with —N(R¹²)(R¹³). In some embodiments is a compound of Formula (I), or a pharmaceutically acceptable salt or solvate thereof, wherein R^{2a} and R^{2b} are taken together with the nitrogen atom to which they are attached to form a substituted C₂₋₉heterocycloalkyl selected from

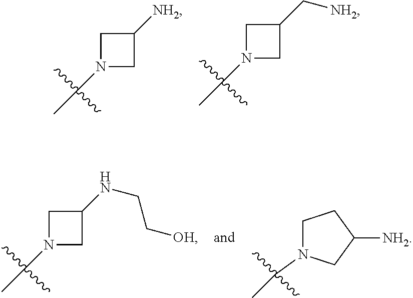 [SMILES: *N1CC(N)C1 ; *N1CC(CN)C1 ; *N1CC(NCCO)C1 ; *N1CCC(N)C1]

In some embodiments is a compound of Formula (I), or a pharmaceutically acceptable salt or solvate thereof, wherein R^{2a} and R^{2b} are taken together with the nitrogen atom to which they are attached to form

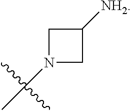 [SMILES: *N1CC(N)C1]

In some embodiments is a compound of Formula (I), or a pharmaceutically acceptable salt or solvate thereof, wherein R^{2a} and R^{2b} are taken together with the nitrogen atom to which they are attached to form

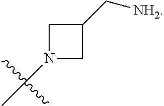 [SMILES: *N1CC(CN)C1]

In some embodiments is a compound of Formula (I), or a pharmaceutically acceptable salt or solvate thereof, wherein R^{2a} and R^{2b} are taken together with the nitrogen atom to which they are attached to form

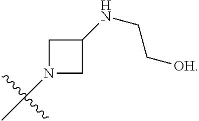 [SMILES: *N1CC(NCCO)C1]

In some embodiments is a compound of Formula (I), or a pharmaceutically acceptable salt or solvate thereof, wherein R^{2a} and R^{2b} are taken together with the nitrogen atom to which they are attached to form

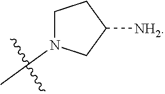 [SMILES: *N1CC[C@H](N)C1]

In some embodiments is a compound of Formula (I), or a pharmaceutically acceptable salt or solvate thereof, wherein each R³ is independently selected from halogen, C₁₋₆alkyl, and —OR¹², wherein C₁₋₆alkyl is optionally substituted with one, two, three halogen, and R¹² is selected from hydrogen and C₁₋₆alkyl. In some embodiments is a compound of Formula (I), or a pharmaceutically acceptable salt or solvate thereof, wherein each R³ is independently selected from halogen. In some embodiments is a compound of Formula (I), or a pharmaceutically acceptable salt or solvate thereof, wherein each R³ is —F.

In some embodiments is a compound of Formula (I), or a pharmaceutically acceptable salt or solvate thereof, wherein n is 1 or 2. In some embodiments is a compound of Formula (I), or a pharmaceutically acceptable salt or solvate thereof, wherein n is 1. In some embodiments is a compound of Formula (I), or a pharmaceutically acceptable salt or solvate thereof, wherein n is 2. In some embodiments is a compound of Formula (I), or a pharmaceutically acceptable salt or solvate thereof, wherein n is 3. In some embodiments is a compound of Formula (I), or a pharmaceutically acceptable salt or solvate thereof, wherein n is 4. In some embodiments is a compound of Formula (I), or a pharmaceutically acceptable salt or solvate thereof, wherein n is 0.

In some embodiments, described herein is a compound of Formula (Ia), or a pharmaceutically acceptable salt, or solvate thereof:

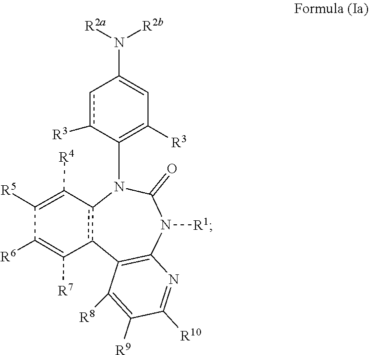

wherein:
R¹ is hydrogen, C₁₋₆alkyl, C₂₋₆alkenyl, C₂₋₆alkynyl, C₃₋₆cycloalkyl, or C₂₋₉heterocycloalkyl, wherein C₁₋₆alkyl, C₂₋₆alkenyl, C₂₋₆alkynyl, C₃₋₆cycloalkyl, and C₂₋₉heterocycloalkyl are optionally substituted with one, two, three, four, or five R¹⁹;
R^{2a} is hydrogen, C₁₋₆alkyl, C₂₋₆alkenyl, C₂₋₆alkynyl, C₃₋₆cycloalkyl, or —(C(R¹⁶)(R¹⁷))ₘ-N(R^{18a})(R^{18b});
R^{2b} is hydrogen, C₁₋₆alkyl, C₂₋₆alkenyl, C₂₋₆alkynyl, or C₃₋₆cycloalkyl, wherein C₁₋₆alkyl, C₂₋₆alkenyl, C₂₋₆alkynyl, and C₃₋₆cycloalkyl are optionally substituted with one, two, three, four, or five R²⁰;
or R^{2a} and R^{2b} are taken together with the nitrogen atom to which they are attached to form a C₂₋₉heterocycloalkyl optionally substituted with one, two, three, four, or five R²⁰;
each R³ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —N(R¹⁴)C(O)R¹⁵, —S(O)₂R¹⁵, and —S(O)₂N(R¹²)(R¹³)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, —OR¹², —N(R¹²)(R¹³), and —C(O)OR¹²;
R⁴, R⁵, R⁶, R⁷, R⁸, R⁹, and R¹⁰ are each independently selected from hydrogen, halogen, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, —CN, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —N(R¹⁴)C(O)R¹⁵, —S(O)₂R¹⁵, and —S(O)₂N(R¹²)(R¹³)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, three, four, or five R²¹; each R¹² is independently selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹³ is independently selected from hydrogen, C₁₋₆alkyl, and C₁₋₆haloalkyl;
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹⁶ and each R¹⁷ are each independently selected from hydrogen, halogen, hydroxy, C₁₋₆alkyl, C₁₋₆haloalkyl, and C₁₋₆alkoxy; or R¹⁶ and R¹⁷ are taken together to form a C₃₋₆cycloalkyl;
R^{18a} and R^{18b} are independently selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, three, four, or five R²⁰; or R^{18a} and R^{18b} are taken together with the nitrogen atom to which they are attached to form a C₂₋₉heterocycloalkyl optionally substituted with one, two, three, four, or five R²⁰;
each R¹⁹, R²⁰, and R²¹ are each independently selected from halogen, oxo, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —N(R¹⁴)C(O)R¹⁵, —S(O)₂R¹⁵, and —S(O)₂N(R¹²)(R¹³)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, —OR¹², —N(R¹²)(R¹³), and —C(O)OR¹²; and
m is 2, 3, or 4.

In some embodiments is a compound of Formula (Ia), or a pharmaceutically acceptable salt or solvate thereof, wherein R⁵ is selected from hydrogen, halogen, C₁₋₆alkyl, C₃₋₆cycloalkyl, C₂₋₉heterocycloalkyl, —CN, —C(O)N(R¹²)(R¹³), and —OR¹², wherein C₁₋₆alkyl, C₃₋₆cycloalkyl, and C₂₋₉heterocycloalkyl are optionally substituted with one, two, three, four, or five R²¹. In some embodiments is a compound of Formula (Ia), or a pharmaceutically acceptable salt or solvate thereof, wherein R⁵ is selected from hydrogen, halogen, C₁₋₆alkyl, —CN, and —OR¹², wherein C₁₋₆alkyl is optionally substituted with one, two, three, four, or five R²¹. In some embodiments is a compound of Formula (Ia), or a pharmaceutically acceptable salt or solvate thereof, wherein R⁵ is selected from hydrogen, halogen, C₁₋₆alkyl, —CN, and —OR¹², wherein C₁₋₆alkyl is optionally substituted with one, two, three halogen, and R¹² is selected from hydrogen and C₁₋₆alkyl. In some embodiments is a compound of Formula (Ia), or a pharmaceutically acceptable salt or solvate thereof, wherein R⁵ is selected from hydrogen, —F, —Cl, —CH₃, —CF₃, —CN, and —OCH₃. In some embodiments is a compound of Formula (Ia), or a pharmaceutically acceptable salt or solvate thereof, wherein R⁵ is hydrogen. In some embodiments is a compound of Formula (Ia), or a pharmaceutically acceptable salt or solvate thereof, wherein R⁵ is —F. In some embodiments is a compound of Formula (Ia), or a pharmaceutically acceptable salt or solvate thereof, wherein R⁵ is —Cl. In some embodiments is a compound of Formula (Ia), or a pharmaceutically acceptable salt or solvate thereof, wherein R⁵ is —CH₃. In some embodiments is a compound of Formula (Ia), or a pharmaceutically acceptable salt or solvate thereof, wherein R⁵ is —CF₃. In some embodiments is a compound of Formula (Ia), or a pharmaceutically acceptable salt or solvate thereof, wherein R⁵ is —CN. In some embodiments is a compound of Formula (Ia), or a pharmaceutically acceptable salt or solvate thereof, wherein R⁵ is —OCH₃.

In some embodiments is a compound of Formula (Ia), or a pharmaceutically acceptable salt or solvate thereof, wherein R⁷ is selected from hydrogen, halogen, C₁₋₆alkyl, —CN, and —OR¹² wherein C₁₋₆alkyl is optionally substituted with one, two, three, four, or five R²¹. In some embodiments is a compound of Formula (Ia), or a pharmaceutically acceptable salt or solvate thereof, wherein R⁷ is selected from hydrogen, halogen, C₁₋₆alkyl, —CN, and —OR¹², wherein C₁₋₆alkyl is optionally substituted with one, two, three halogen, and R¹² is selected from hydrogen and C₁₋₆alkyl. In some embodiments is a compound of Formula (Ia), or a pharmaceutically acceptable salt or solvate thereof, wherein R⁷ is selected from hydrogen, —F, —Cl, —CH₃, —CF₃, —CN, and —OCH₃. In some embodiments is a compound of Formula (Ia), or a pharmaceutically acceptable salt or solvate thereof, wherein R⁷ is hydrogen. In some embodiments is a compound of Formula (Ia), or a pharmaceutically acceptable salt or solvate thereof, wherein R⁷ is —F. In some embodiments is a compound of Formula (Ia), or a pharmaceutically acceptable salt or solvate thereof, wherein R⁷ is —Cl. In some embodiments is a compound of Formula (Ia), or a pharmaceutically acceptable salt or solvate thereof, wherein R⁷ is —CH₃. In some embodiments is a compound of Formula (Ia), or a pharmaceutically acceptable salt or solvate thereof, wherein R⁷ is —CF₃. In some embodiments is a compound of Formula (Ia), or a pharmaceutically acceptable salt or solvate thereof, wherein R⁷ is —CN. In some embodiments is a compound of Formula (Ia), or a pharmaceutically acceptable salt or solvate thereof, wherein R⁷ is —OCH₃.

In some embodiments is a compound of Formula (Ia), or a pharmaceutically acceptable salt or solvate thereof, wherein R⁹ is selected from hydrogen, halogen, C₁₋₆alkyl, C₃₋₆cycloalkyl, —CN, and —OR¹², wherein C₁₋₆alkyl and C₃₋₆cycloalkyl are optionally substituted with one, two, three, four, or five R²¹. In some embodiments is a compound of Formula (Ia), or a pharmaceutically acceptable salt or solvate thereof, wherein R⁹ is selected from hydrogen, halogen, C₁₋₆alkyl, C₃₋₆cycloalkyl, —CN, and —OR¹², wherein C₁₋₆alkyl and C₃₋₆cycloalkyl are optionally substituted with one, two, three halogen, and R¹² is selected from hydrogen and C₁₋₆alkyl. In some embodiments is a compound of Formula (Ia), or a pharmaceutically acceptable salt or solvate thereof, wherein R⁹ is selected from hydrogen, —F, —Cl, —CH₃, —CF₃, —CN, —OCH₃, and

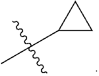 [SMILES: *C1CC1]

In some embodiments is a compound of Formula (Ia), or a pharmaceutically acceptable salt or solvate thereof, wherein R⁹ is hydrogen. In some embodiments is a compound of Formula (Ia), or a pharmaceutically acceptable salt or solvate thereof, wherein R⁹ is —F. In some embodiments is a compound of Formula (Ia), or a pharmaceutically acceptable salt or solvate thereof, wherein R⁹ is —Cl. In some embodiments is a compound of Formula (Ia), or a pharmaceutically acceptable salt or solvate thereof, wherein R⁹ is —CH₃. In some embodiments is a compound of Formula (Ia), or a pharmaceutically acceptable salt or solvate thereof, wherein R⁹ is —CF₃. In some embodiments is a compound of Formula (Ia), or a pharmaceutically acceptable salt or solvate thereof, wherein R⁹ is —CN. In some embodiments is a compound of Formula (Ia), or a pharmaceutically acceptable salt or solvate thereof, wherein R⁹ is —OCH₃. In some embodiments is a compound of Formula (Ia), or a pharmaceutically acceptable salt or solvate thereof, wherein R⁹ is

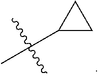 [SMILES: *C1CC1]

In some embodiments is a compound of Formula (Ia), or a pharmaceutically acceptable salt or solvate thereof, wherein R⁴, R⁶, R⁸, and R¹⁰ are each independently selected from hydrogen, halogen, and unsubstituted C₁₋₆alkyl. In some embodiments is a compound of Formula (Ia), or a pharmaceutically acceptable salt or solvate thereof, wherein R⁴, R⁶, R⁸, and R¹⁰ are each independently selected from hydrogen and halogen. In some embodiments is a compound of Formula (Ia), or a pharmaceutically acceptable salt or solvate thereof, wherein R⁴, R⁶, R⁸, and R¹⁰ are each independently selected from hydrogen and unsubstituted C₁₋₆alkyl. In some embodiments is a compound of Formula (Ia), or a pharmaceutically acceptable salt or solvate thereof, wherein R⁴, R⁶, R⁸, and R¹⁰ are each hydrogen.

In some embodiments is a compound of Formula (Ia), or a pharmaceutically acceptable salt or solvate thereof, wherein R¹ is C₁₋₆alkyl optionally substituted with one, two, three, four, or five R¹⁹. In some embodiments is a compound of Formula (Ia), or a pharmaceutically acceptable salt or solvate thereof, wherein R¹ is C₁₋₆alkyl optionally substituted with one, two, three, four, or five R¹⁹, and each R¹⁹ is independently selected from halogen, C₁₋₆haloalkyl, C₃₋₆cycloalkyl, —OR¹², and —N(R¹²)(R¹³). In some embodiments is a compound of Formula (Ia), or a pharmaceutically acceptable salt or solvate thereof, wherein R¹ is C₁₋₆alkyl optionally substituted with one, two, three, four, or five R¹⁹, each R¹⁹ is independently selected from halogen, C₃₋₆cycloalkyl, and OR¹², and each R¹² is independently selected from hydrogen and C₁₋₆alkyl. In some embodiments is a compound of Formula (Ia), or a pharmaceutically acceptable salt or solvate thereof, wherein R¹ is —CF₃, —CH₂CF₃, —CH₂CH₂OH, —CH₂CH₂OCH₃, or

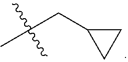 [SMILES: *CC1CC1]

In some embodiments is a compound of Formula (Ia), or a pharmaceutically acceptable salt or solvate thereof, wherein R¹ is —CF₃. In some embodiments is a compound of Formula (Ia), or a pharmaceutically acceptable salt or solvate thereof, wherein R¹ is —CH₂CF₃. In some embodiments is a compound of Formula (Ia), or a pharmaceutically acceptable salt or solvate thereof, wherein R¹ is —CH₂CH₂OH. In some embodiments is a compound of Formula (Ia), or a pharmaceutically acceptable salt or solvate thereof, wherein R¹ is —CH₂CH₂OCH₃. In some embodiments is a compound of Formula (Ia), or a pharmaceutically acceptable salt or solvate thereof, wherein R¹ is

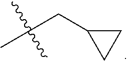 [SMILES: *CC1CC1]

In some embodiments is a compound of Formula (Ia), or a pharmaceutically acceptable salt or solvate thereof, wherein R¹ is an unsubstituted C₁₋₆alkyl. In some embodiments is a compound of Formula (Ia), or a pharmaceutically acceptable salt or solvate thereof, wherein R¹ is —CH₃, —CH₂CH₃, —CH(CH₃)₂, or —CH₂CH₂CH₃. In some embodiments is a compound of Formula (Ia), or a pharmaceutically acceptable salt or solvate thereof, wherein R¹ is —CH₃. In some embodiments is a compound of Formula (Ia), or a pharmaceutically acceptable salt or solvate thereof, wherein R¹ is —CH₂CH₃. In some embodiments is a compound of Formula (Ia), or a pharmaceutically acceptable salt or solvate thereof, wherein R¹ is —CH(CH₃)₂. In some embodiments is a compound of Formula (Ia), or a pharmaceutically acceptable salt or solvate thereof, wherein R¹ is —CH₂CH₂CH₃. In some embodiments is a compound of Formula (Ia), or a pharmaceutically acceptable salt or solvate thereof, wherein R¹ is C₃₋₆cycloalkyl optionally substituted with one, two, three, four, or five R¹⁹. In some embodiments is a compound of Formula (Ia), or a pharmaceutically acceptable salt or solvate thereof, wherein R¹ is

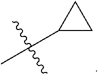 [SMILES: *C1CC1]

In some embodiments is a compound of Formula (Ia), or a pharmaceutically acceptable salt or solvate thereof, wherein R^{2a} is —(C(R¹⁶)(R¹⁷))ₘ-N(R^{18a})(R^{18b}). In some embodiments is a compound of Formula (Ia), or a pharmaceutically acceptable salt or solvate thereof, wherein each R¹⁶ and each R¹⁷ are each independently selected from hydrogen and C₁₋₆alkyl. In some embodiments is a compound of Formula (Ia), or a pharmaceutically acceptable salt or solvate thereof, wherein each R¹⁶ and each R¹⁷ are hydrogen. In some embodiments is a compound of Formula (Ia), or a pharmaceutically acceptable salt or solvate thereof, wherein m is 2. In some embodiments is a compound of Formula (Ia), or a pharmaceutically acceptable salt or solvate thereof, wherein m is 3. In some embodiments is a compound of Formula (Ia), or a pharmaceutically acceptable salt or solvate thereof, wherein m is 4. In some embodiments is a compound of Formula (Ia), or a pharmaceutically acceptable salt or solvate thereof, wherein R^{18a} is C₁₋₆alkyl optionally substituted with one, two, three, four, or five R²⁰. In some embodiments is a compound of Formula (Ia), or a pharmaceutically acceptable salt or solvate thereof, wherein R^{18a} is C₁₋₆alkyl optionally substituted with one, two, three, four, or five R²⁰, and each R²⁰ is independently selected from halogen, —OR¹², —N(R¹²)(R¹³), and —C(O)OR¹². In some embodiments is a compound of Formula (Ia), or a pharmaceutically acceptable salt or solvate thereof, wherein R^{18a} is C₁₋₆alkyl substituted with one, two, or three R²⁰, each R²⁰ is independently selected from halogen, —OR¹², —N(R¹²)(R¹³), and —C(O)OR¹², each R¹² is independently selected from hydrogen and C₁₋₆alkyl, and each R¹³ is independently selected from hydrogen and C₁₋₆alkyl. In some embodiments is a compound of Formula (Ia), or a pharmaceutically acceptable salt or solvate thereof, wherein R^{18a} is —CH₂CH₂OH, —CH₂CH₂OCH₃, —CH₂CH₂NH₂, —CH₂CH₂N(H)(CH₃), —CH₂CH₂C(O)OH, and —CH₂CH₂CH₂C(O)OH. In some embodiments is a compound of Formula (Ia), or a pharmaceutically acceptable salt or solvate thereof, wherein R^{18a} is —CH₂CH₂OH. In some embodiments is a compound of Formula (Ia), or a pharmaceutically acceptable salt or solvate thereof, wherein R^{18a} is —CH₂CH₂OCH₃. In some embodiments is a compound of Formula (Ia), or a pharmaceutically acceptable salt or solvate thereof, wherein R^{18a} is —CH₂CH₂NH₂. In some embodiments is a compound of Formula (Ia), or a pharmaceutically acceptable salt or solvate thereof, wherein R^{18a} is —CH₂CH₂N(H)(CH₃). In some embodiments is a compound of Formula (Ia), or a pharmaceutically acceptable salt or solvate thereof, wherein R^{18a} is —CH₂CH₂C(O)OH. In some embodiments is a compound of Formula (Ia), or a pharmaceutically acceptable salt or solvate thereof, wherein R^{18a} is —CH₂CH₂CH₂C(O)OH. In some embodiments is a compound of Formula (Ia), or a pharmaceutically acceptable salt or solvate thereof, wherein R^{18a} is an unsubstituted C₁₋₆alkyl. In some embodiments is a compound of Formula (Ia), or a pharmaceutically acceptable salt or solvate thereof, wherein R^{18a} is —CH₃. In some embodiments is a compound of Formula (Ia), or a pharmaceutically acceptable salt or solvate thereof, wherein R^{18a} is hydrogen. In some embodiments is a compound of Formula (Ia), or a pharmaceutically acceptable salt or solvate thereof, wherein R^{18b} is hydrogen. In some embodiments is a compound of Formula (Ia), or a pharmaceutically acceptable salt or solvate thereof, wherein R^{18b} is an unsubstituted C₁₋₆alkyl. In some embodiments is a compound of Formula (Ia), or a pharmaceutically acceptable salt or solvate thereof, wherein R^{18b} is —CH₃. In some embodiments is a compound of Formula (Ia), or a pharmaceutically acceptable salt or solvate thereof, wherein R^{2b} is hydrogen.

In some embodiments is a compound of Formula (Ia), or a pharmaceutically acceptable salt or solvate thereof, wherein R^{2a} and R^{2b} are taken together with the nitrogen atom to which they are attached to form a C₂₋₉heterocycloalkyl optionally substituted with one, two, three, four, or five R²⁰. In some embodiments is a compound of Formula (Ia), or a pharmaceutically acceptable salt or solvate thereof, wherein R^{2a} and R^{2b} are taken together with the nitrogen atom to which they are attached to form a C₂₋₉heterocycloalkyl optionally substituted with one, two, three, four, or five R²⁰, and each R²⁰ is independently selected from halogen, —OR¹², —N(R¹²)(R¹³) and C₁₋₆alkyl optionally substituted with —N(R¹²)(R¹³). In some embodiments is a compound of Formula (Ia), or a pharmaceutically acceptable salt or solvate thereof, wherein R^{2a} and R^{2b} are taken together with the nitrogen atom to which they are attached to form a substituted C₂₋₉heterocycloalkyl selected from

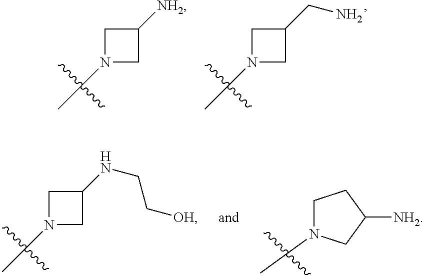 [SMILES: *N1CC(N)C1 ; *N1CC(CN)C1 ; "," ; *N1CC(NCCO)C1 ; "and" ; *N1CCC(N)C1]

In some embodiments is a compound of Formula (Ia), or a pharmaceutically acceptable salt or solvate thereof, wherein R^{2a} and R^{2b} are taken together with the nitrogen atom to which they are attached to form

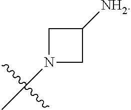 [SMILES: *N1CC(N)C1]

In some embodiments is a compound of Formula (Ia), or a pharmaceutically acceptable salt or solvate thereof, wherein R^{2a} and R^{2b} are taken together with the nitrogen atom to which they are attached to form

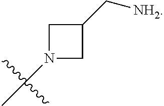 [SMILES: *N1CC(CN)C1]

In some embodiments is a compound of Formula (Ia), or a pharmaceutically acceptable salt or solvate thereof, wherein R^{2a} and R^{2b} are taken together with the nitrogen atom to which they are attached to form

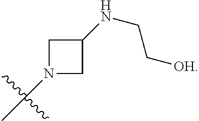 [SMILES: *N1CC(NCCO)C1]

In some embodiments is a compound of Formula (Ia), or a pharmaceutically acceptable salt or solvate thereof, wherein R^{2a} and R^{2b} are taken together with the nitrogen atom to which they are attached to form

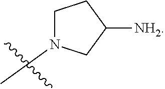 [SMILES: *N1CCC(N)C1]

In some embodiments is a compound of Formula (Ia), or a pharmaceutically acceptable salt or solvate thereof, wherein each R³ is independently selected from halogen, C₁₋₆alkyl, and —OR¹², wherein C₁₋₆alkyl is optionally substituted with one, two, three halogen, and R¹² is selected from hydrogen and C₁₋₆alkyl. In some embodiments is a compound of Formula (Ia), or a pharmaceutically acceptable salt or solvate thereof, wherein each R³ is independently selected from halogen. In some embodiments is a compound of Formula (Ia), or a pharmaceutically acceptable salt or solvate thereof, wherein each R³ is —F.

Exemplary compounds of Formula (I′) or (I) include the compounds described in the following Tables:

TABLE 1

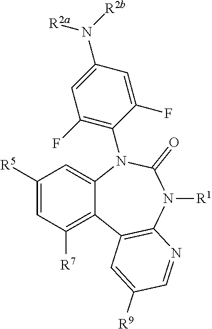

| Cpd No. | R⁹ | R⁷ | R⁵ | R¹ | 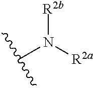 |
|---|---|---|---|---|---|
| 1-1 | H | H | Cl | 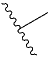 | 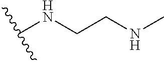 [SMILES: *NCCNC] |
| 1-2 | H | H | Cl | 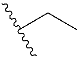 [SMILES: *CC] | 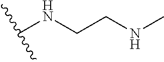 [SMILES: *NCCNC] |
| 1-3 | H | H | Cl | 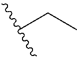 [SMILES: *CC] | 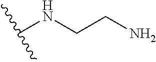 [SMILES: *NCCN] |
| 1-4 | H | H | Cl | 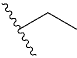 [SMILES: *CC] | 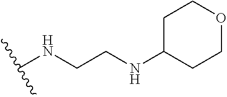 [SMILES: *NCCNC1CCOCC1] |
| 1-5 | H | H | Cl | 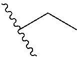 [SMILES: *CC] | 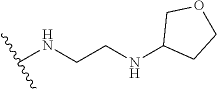 [SMILES: *NCCNC1CCOC1] |
| 1-6 | H | H | Cl | 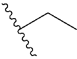 [SMILES: *CC] | 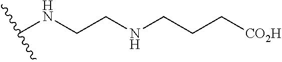 [SMILES: *NCCNCCCC(=O)O] |
| 1-7 | H | H | Cl | 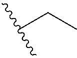 [SMILES: *CC] | 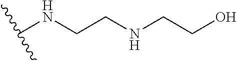 [SMILES: *NCCNCCO] |
| 1-8 | Cl | H | Cl | 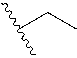 [SMILES: *CC] | 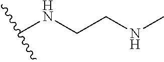 [SMILES: *NCCNC] |
| 1-9 | Cl | H | Cl | 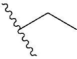 [SMILES: *CC] | 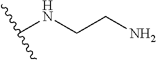 [SMILES: *NCCN] |
| 1-10 | Cl | H | Cl | 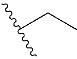 [SMILES: *CC] | 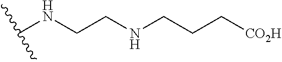 [SMILES: *NCCNCCCC(=O)O] |
| 1-12 | Cl | H | Cl | 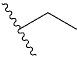 [SMILES: *CC] | 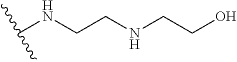 [SMILES: *NCCNCCO] |
| 1-13 | Cl | H | Cl | 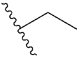 [SMILES: *CC] | 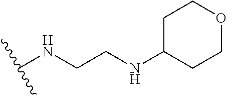 [SMILES: *NCCNC1CCOCC1] |
| 1-14 | F | H | Cl | 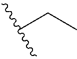 [SMILES: *CC] | 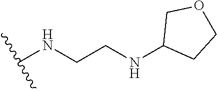 [SMILES: *NCCNC1CCOC1] |
| 1-15 | F | H | Cl | 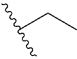 [SMILES: *CC] | 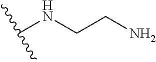 [SMILES: *NCCN] |
| 1-16 | F | H | Cl | 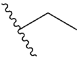 [SMILES: *CC] | 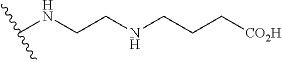 [SMILES: *NCCNCCCC(=O)O] |
| 1-17 | F | H | Cl | 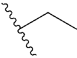 [SMILES: *CC] | 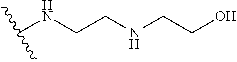 [SMILES: *NCCNCCO] |
| 1-18 | -CH₃ | H | Cl | 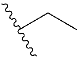 [SMILES: *CC] | 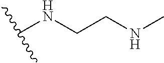 [SMILES: *NCCNC] |
| 1-19 | F | H | Cl | 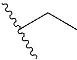 [SMILES: *CC] | 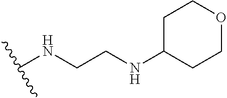 [SMILES: *NCCNC1CCOCC1] |
| 1-20 | F | H | Cl | 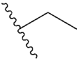 [SMILES: *CC] | 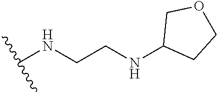 [SMILES: *NCCNC1CCOC1] |
| 1-21 | Cl | -CH₃ | H | 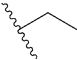 [SMILES: *CC] | 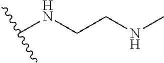 [SMILES: *NCCNC] |
| 1-22 | -CH₃ | H | Cl | 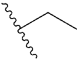 [SMILES: *CC] | 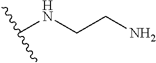 [SMILES: *NCCN] |
| 1-23 | H | -CH₃ | Cl | 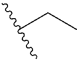 [SMILES: *CC] | 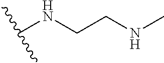 [SMILES: *NCCNC] |
| 1-24 | 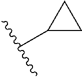 [SMILES: *C1CC1] | H | Cl | 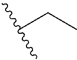 [SMILES: *CC] | 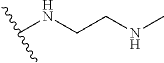 [SMILES: *NCCNC] |
| 1-25 | -CH₃ | H | Cl | 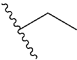 [SMILES: *CC] | 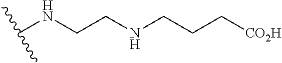 [SMILES: *NCCNCCCC(=O)O] |
| 1-26 | -CH₃ | H | Cl | 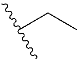 [SMILES: *CC] | 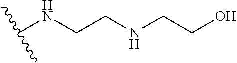 [SMILES: *NCCNCCO] |
| 1-27 | -CH₃ | H | Cl | 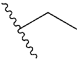 [SMILES: *CC] | 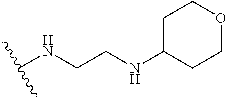 [SMILES: *NCCNC1CCOCC1] |
| 1-28 | -CH₃ | H | Cl | 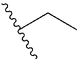 [SMILES: *CC] | 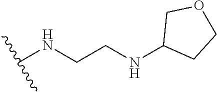 [SMILES: *NCCNC1CCOC1] |
| 1-29 | Cl | H | -CH₃ | 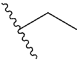 [SMILES: *CC] | 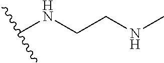 [SMILES: *NCCNC] |
| 1-30 | Cl | H | Cl | 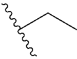 [SMILES: *CC] | 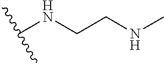 [SMILES: *NCCNC] |
| 1-31 | F | -CH₃ | Cl | 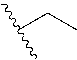 [SMILES: *CC] | 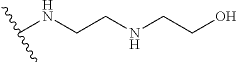 [SMILES: *NCCNCCO] |
| 1-32 | F | -CH₃ | Cl | 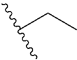 [SMILES: *CC] | 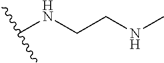 [SMILES: *NCCNC] |
| 1-33 | Cl | H | Cl | 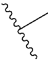 | 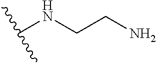 [SMILES: *NCCN] |
| 1-34 | Cl | H | Cl | 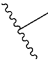 | 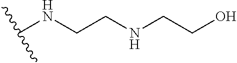 [SMILES: *NCCNCCO] |
| 1-35 | Cl | H | Cl | 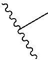 | 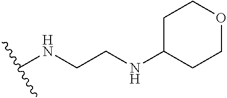 [SMILES: *NCCNC1CCOCC1] |
| 1-36 | Cl | -CH₃ | Cl | 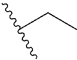 [SMILES: *CC] | 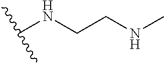 [SMILES: *NCCNC] |
| 1-37 | F | Cl | Cl | 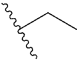 [SMILES: *CC] | 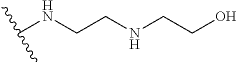 [SMILES: *NCCNCCO] |
| 1-38 | F | Cl | Cl | 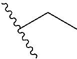 [SMILES: *CC] | 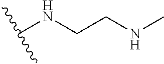 [SMILES: *NCCNC] |
| 1-39 | Cl | H | Cl | 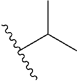 [SMILES: *C(C)C] | 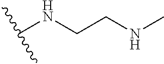 [SMILES: *NCCNC] |
| 1-40 | Cl | H | Cl | 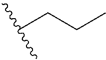 [SMILES: *CCC] | 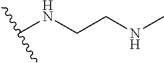 [SMILES: *NCCNC] |
| 1-41 | Cl | H | Cl | 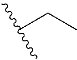 [SMILES: *CC] | 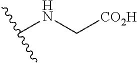 [SMILES: *NCC(=O)O] |
| 1-42 | Cl | H | Cl | 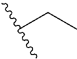 [SMILES: *CC] | 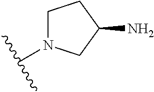 [SMILES: *N1CC[C@@H](N)C1] |
| 1-43 | Cl | H | Cl | 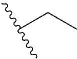 [SMILES: *CC] | 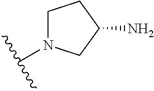 [SMILES: *N1CC[C@H](N)C1] |
| 1-44 | Cl | H | Cl | 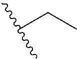 [SMILES: *CC] | 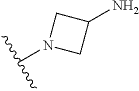 [SMILES: *N1CC(N)C1] |
| 1-45 | Cl | H | Cl | 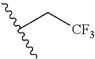 [SMILES: *CC(F)(F)F] | 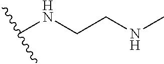 [SMILES: *NCCNC] |
| 1-46 | Cl | H | Cl | 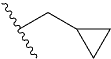 [SMILES: *CC1CC1] | 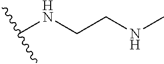 [SMILES: *NCCNC] |
| 1-47 | Cl | H | Cl | 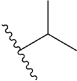 [SMILES: *C(C)C] | 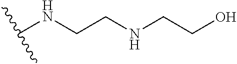 [SMILES: *NCCNCCO] |
| 1-48 | Cl | H | Cl | 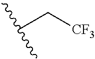 [SMILES: *CC(F)(F)F] | 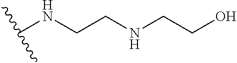 [SMILES: *NCCNCCO] |
| 1-49 | Cl | H | Cl | 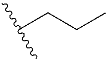 [SMILES: *CCC] | 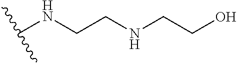 [SMILES: *NCCNCCO] |
| 1-50 | Cl | H | Cl | 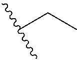 [SMILES: *CC] | 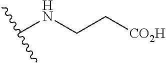 [SMILES: *NCCC(=O)O] |
| 1-51 | Cl | -CH₃ | Cl | 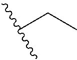 [SMILES: *CC] | 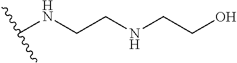 [SMILES: *NCCNCCO] |
| 1-52 | Cl | H | Cl | 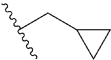 [SMILES: *CC1CC1] | 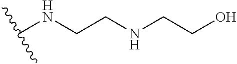 [SMILES: *NCCNCCO] |
| 1-53 | Cl | H | Cl | 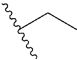 [SMILES: *CC] | 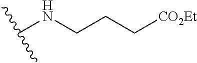 [SMILES: *NCCCC(=O)OCC] |
| 1-54 | Cl | H | CN | 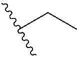 [SMILES: *CC] | 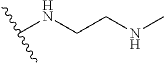 [SMILES: *NCCNC] |
| 1-55 | Cl | H | CN | 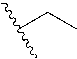 [SMILES: *CC] | 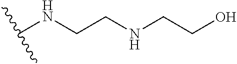 [SMILES: *NCCNCCO] |
| 1-56 | CF3 | H | Cl | 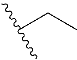 [SMILES: *CC] | 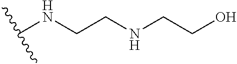 [SMILES: *NCCNCCO] |
| 1-57 | CF3 | H | Cl | 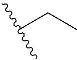 [SMILES: *CC] | 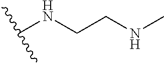 [SMILES: *NCCNC] |
| 1-58 | Cl | H | CN | 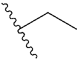 [SMILES: *CC] | 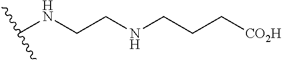 [SMILES: *NCCNCCCC(=O)O] |
| 1-59 | Cl | H | Cl | 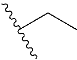 [SMILES: *CC] | 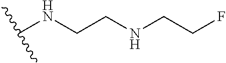 [SMILES: *NCCNCCF] |
| 1-60 | Cl | F | Cl | 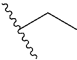 [SMILES: *CC] | 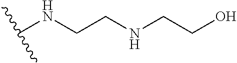 [SMILES: *NCCNCCO] |
| 1-61 | Cl | F | Cl | 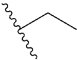 [SMILES: *CC] | 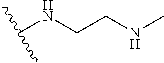 [SMILES: *NCCNC] |
| 1-62 | F | -CH₃ | -CN | 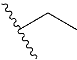 [SMILES: *CC] | 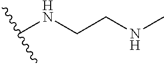 [SMILES: *NCCNC] |
| 1-63 | F | -CH₃ | -CN | 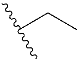 [SMILES: *CC] | 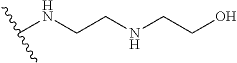 [SMILES: *NCCNCCO] |
| 1-64 | F | -CH₃ | Cl | 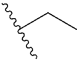 [SMILES: *CC] | 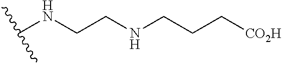 [SMILES: *NCCNCCCC(=O)O] |
| 1-65 | F | -CH₃ | Cl | 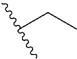 [SMILES: *CC] | 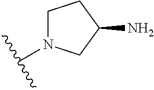 [SMILES: *N1CC[C@@H](N)C1] |
| 1-66 | F | H | -cf 3 | 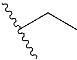 [SMILES: *CC] | 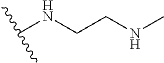 [SMILES: *NCCNC] |
| 1-67 | F | H | -cf 3 | 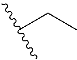 [SMILES: *CC] | 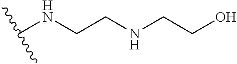 [SMILES: *NCCNCCO] |
| 1-68 | F | -CH₃ | -CN | 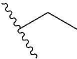 [SMILES: *CC] | 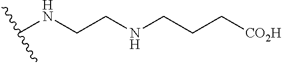 [SMILES: *NCCNCCCC(=O)O] |
| 1-69 | F | H | -CF3 | 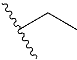 [SMILES: *CC] | 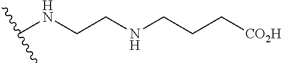 [SMILES: *NCCNCCCC(=O)O] |
| 1-70 | Cl | H | Cl | 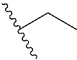 [SMILES: *CC] | 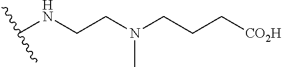 [SMILES: *NCCN(C)CCCC(=O)O] |
| 1-71 | Cl | H | Cl | 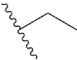 [SMILES: *CC] | 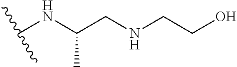 [SMILES: *N[C@@H](C)CNCCO] |
| 1-72 | Cl | H | Cl | 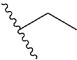 [SMILES: *CC] | 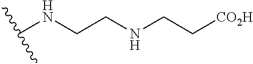 [SMILES: *NCCNCCC(=O)O] |
| 1-73 | Cl | H | Cl | 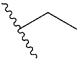 [SMILES: *CC] | 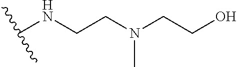 [SMILES: *NCCN(C)CCO] |
| 1-74 | Cl | H | Cl | 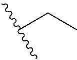 [SMILES: *CC] | 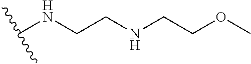 [SMILES: *NCCNCCOC] |
| 1-75 | F | -CH₃ | Cl | 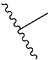 | 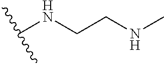 [SMILES: *NCCNC] |
| 1-76 | F | -CH₃ | Cl | 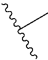 | 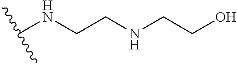 [SMILES: *NCCNCCO] |
| 1-77 | F | -CH₃ | Cl | 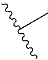 | 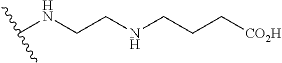 [SMILES: *NCCNCCCC(=O)O] |
| 1-78 | F | -CH₃ | Cl | 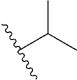 [SMILES: *C(C)C] | 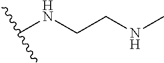 [SMILES: *NCCNC] |
| 1-79 | F | -CH₃ | Cl | 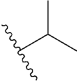 [SMILES: *C(C)C] | 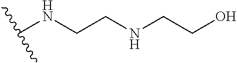 [SMILES: *NCCNCCO] |
| 1-80 | F | -CH₃ | Cl | 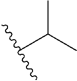 [SMILES: *C(C)C] | 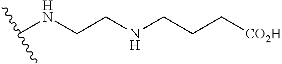 [SMILES: *NCCNCCCC(=O)O] |
| 1-81 | F | H | -CN | 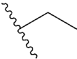 [SMILES: *CC] | 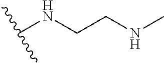 [SMILES: *NCCNC] |
| 1-82 | F | H | -CN | 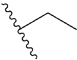 [SMILES: *CC] | 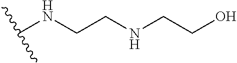 [SMILES: *NCCNCCO] |
| 1-83 | F | H | -CN | 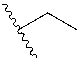 [SMILES: *CC] | 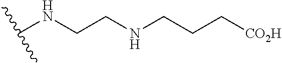 [SMILES: *NCCNCCCC(=O)O] |
| 1-84 | F | H | -CN | 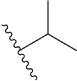 [SMILES: *C(C)C] | 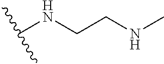 [SMILES: *NCCNC] |
| 1-85 | F | H | -CN | 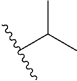 [SMILES: *C(C)C] | 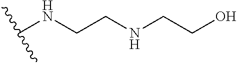 [SMILES: *NCCNCCO] |
| 1-86 | F | H | -CN | 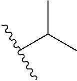 [SMILES: *C(C)C] | 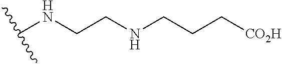 [SMILES: *NCCNCCCC(=O)O] |
| 1-87 | F | -OCH₃ | Cl | 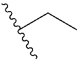 [SMILES: *CC] | 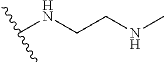 [SMILES: *NCCNC] |
| 1-88 | F | -OCH₃ | Cl | 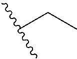 [SMILES: *CC] | 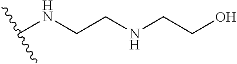 [SMILES: *NCCNCCO] |
| 1-89 | CN | H | Cl | 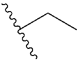 [SMILES: *CC] | 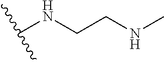 [SMILES: *NCCNC] |
| 1-90 | CN | H | Cl | 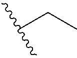 [SMILES: *CC] | 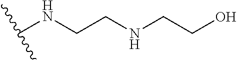 [SMILES: *NCCNCCO] |
| 1-91 | Cl | H | -CN | 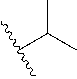 [SMILES: *C(C)C] | 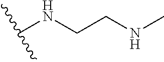 [SMILES: *NCCNC] |
| 1-92 | Cl | H | -CN | 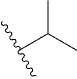 [SMILES: *C(C)C] | 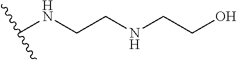 [SMILES: *NCCNCCO] |
| 1-93 | Cl | H | -CN | 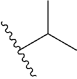 [SMILES: *C(C)C] | 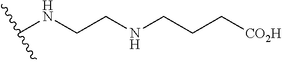 [SMILES: *NCCNCCCC(=O)O] |
| 1-94 | F | F | Cl | 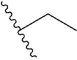 [SMILES: *CC] | 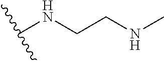 [SMILES: *NCCNC] |
| 1-95 | F | F | Cl | 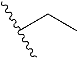 [SMILES: *CC] | 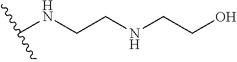 [SMILES: *NCCNCCO] |
| 1-96 | F | F | Cl | 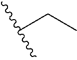 [SMILES: *CC] | 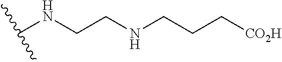 [SMILES: *NCCNCCCC(=O)O] |
| 1-97 | -CF₃ | H | -CN | 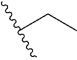 [SMILES: *CC] | 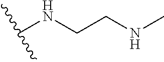 [SMILES: *NCCNC] |
| 1-98 | -CF₃ | H | -CN | 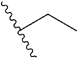 [SMILES: *CC] | 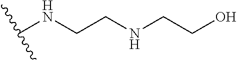 [SMILES: *NCCNCCO] |
| 1-99 | Cl | F | -CN | 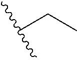 [SMILES: *CC] | 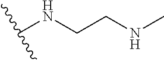 [SMILES: *NCCNC] |
| 1-100 | Cl | F | -CN | 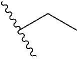 [SMILES: *CC] | 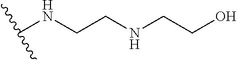 [SMILES: *NCCNCCO] |
| 1-101 | Cl | F | -CN | 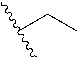 [SMILES: *CC] | 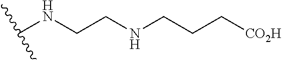 [SMILES: *NCCNCCCC(=O)O] |
| 1-102 | F | F | -CN | 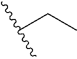 [SMILES: *CC] | 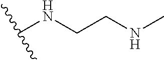 [SMILES: *NCCNC] |
| 1-103 | F | F | -CN | 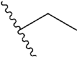 [SMILES: *CC] | 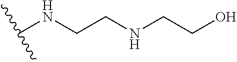 [SMILES: *NCCNCCO] |
| 1-104 | F | F | -CN | 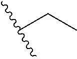 [SMILES: *CC] | 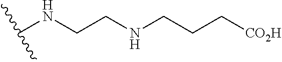 [SMILES: *NCCNCCCC(=O)O] |
| 1-105 | F | H | Cl | 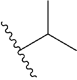 [SMILES: *C(C)C] | 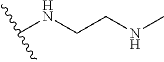 [SMILES: *NCCNC] |
| 1-106 | F | H | Cl | 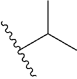 [SMILES: *C(C)C] | 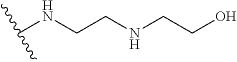 [SMILES: *NCCNCCO] |
| 1-107 | F | F | -CN | 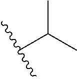 [SMILES: *C(C)C] | 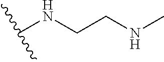 [SMILES: *NCCNC] |
| 1-108 | F | F | -CN | 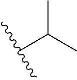 [SMILES: *C(C)C] | 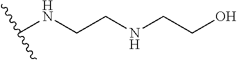 [SMILES: *NCCNCCO] |
| 1-109 | F | F | -CN | 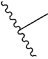 | 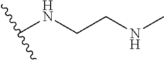 [SMILES: *NCCNC] |
| 1-110 | F | F | -CN | 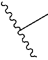 | 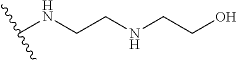 [SMILES: *NCCNCCO] |
| 1-111 | F | -CH₃ | -CN | 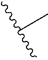 | 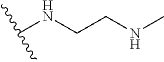 [SMILES: *NCCNC] |
| 1-112 | F | -CH₃ | -CN | 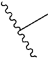 | 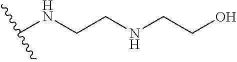 [SMILES: *NCCNCCO] |
| 1-113 | F | F | Cl | 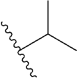 [SMILES: *C(C)C] | 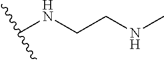 [SMILES: *NCCNC] |
| 1-114 | F | F | Cl | 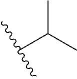 [SMILES: *C(C)C] | 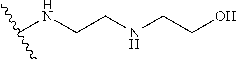 [SMILES: *NCCNCCO] |
| 1-115 | Cl | F | -CN | 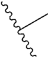 | 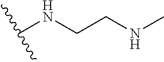 [SMILES: *NCCNC] |
| 1-116 | Cl | F | -CN | 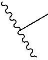 | 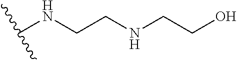 [SMILES: *NCCNCCO] |
| 1-117 | F | H | -CN | 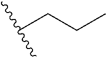 [SMILES: *CCC] | 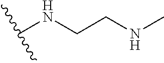 [SMILES: *NCCNC] |
| 1-118 | F | H | -CN | 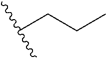 [SMILES: *CCC] | 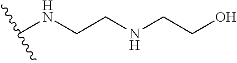 [SMILES: *NCCNCCO] |
| 1-119 | Cl | H | -CN | 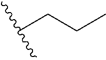 [SMILES: *CCC] | 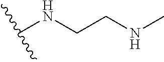 [SMILES: *NCCNC] |
| 1-120 | Cl | H | -CN | 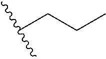 [SMILES: *CCC] | 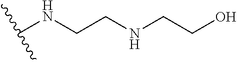 [SMILES: *NCCNCCO] |
| 1-121 | F | H | -CN | 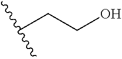 [SMILES: *CCO] | 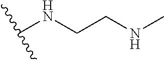 [SMILES: *NCCNC] |
| 1-122 | F | H | -CN | 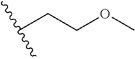 [SMILES: *CCOC] | 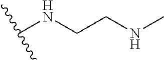 [SMILES: *NCCNC] |
| 1-123 | F | H | -CN | 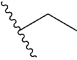 [SMILES: *CC] | 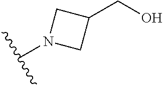 [SMILES: *N1CC(CO)C1] |
| 1-124 | F | H | -CN | 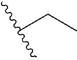 [SMILES: *CC] | 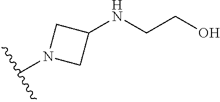 [SMILES: *N1CC(NCCO)C1] |
| 1-125 | F | F | -CN | 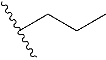 [SMILES: *CCC] | 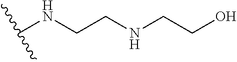 [SMILES: *NCCNCCO] |
| 1-126 | Cl | H | -CN | 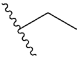 [SMILES: *CC] | 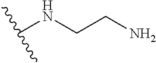 [SMILES: *NCCN] |
| 1-127 | Cl | H | -CN | 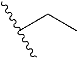 [SMILES: *CC] | 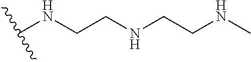 [SMILES: *NCCNCCNC] |
| 1-128 | Cl | H | -CN | 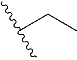 [SMILES: *CC] | 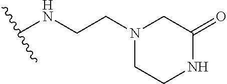 [SMILES: *NCCN1CCNC(=O)C1] |
| 1-129 | Cl | H | F | 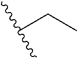 [SMILES: *CC] | 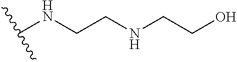 [SMILES: *NCCNCCO] |
| 1-130 | Cl | H | F | 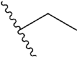 [SMILES: *CC] | 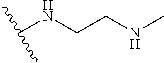 [SMILES: *NCCNC] |
| 1-131 | Cl | H | -CN | 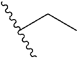 [SMILES: *CC] | 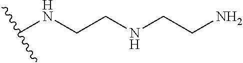 [SMILES: *NCCNCCN] |
| 1-132 | Cl | H | -CN | 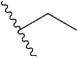 [SMILES: *CC] | 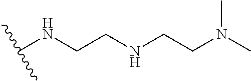 [SMILES: *NCCNCCN(C)C] |
| 1-133 | Cl | H | -CN | 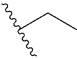 [SMILES: *CC] | 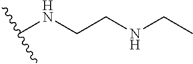 [SMILES: *NCCNCC] |
| 1-134 | F | -CN | Cl | 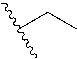 [SMILES: *CC] | 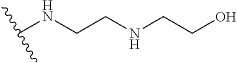 [SMILES: *NCCNCCO] |
| 1-135 | Cl | H | H | 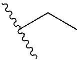 [SMILES: *CC] | 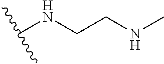 [SMILES: *NCCNC] |
| 1-136 | Cl | H | -CN | 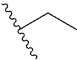 [SMILES: *CC] | 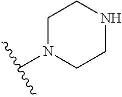 [SMILES: *N1CCNCC1] |
| 1-137 | Cl | H | -CN | 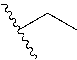 [SMILES: *CC] | 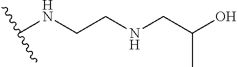 [SMILES: *NCCNCC(C)O] |
| 1-138 | Cl | H | -CN | 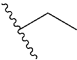 [SMILES: *CC] | 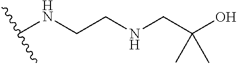 [SMILES: *NCCNCC(C)(C)O] |
| 1-139 | Cl | H | -CN | 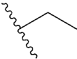 [SMILES: *CC] | 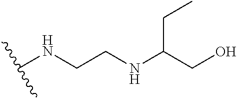 [SMILES: *NCCNC(CC)CO] |
| 1-140 | Cl | H | -CN | 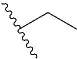 [SMILES: *CC] | 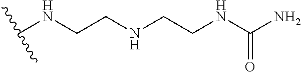 [SMILES: *NCCNCCNC(N)=O] |
| 1-141 | Cl | H | -CN | 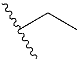 [SMILES: *CC] | 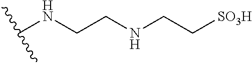 [SMILES: *NCCNCCS(=O)(=O)O] |
| 1-142 | Cl | H | H | 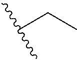 [SMILES: *CC] | 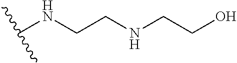 [SMILES: *NCCNCCO] |
| 1-143 | Cl | H | -CN | 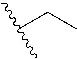 [SMILES: *CC] | 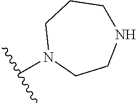 [SMILES: *N1CCCNCC1] |
| 1-144 | Cl | H | -CN | 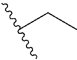 [SMILES: *CC] | 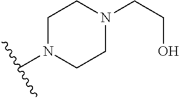 [SMILES: *N1CCN(CCO)CC1] |
| 1-145 | Cl | H | -CN | 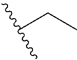 [SMILES: *CC] | 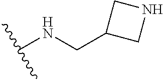 [SMILES: *NCC1CNC1] |
| 1-146 | Cl | H | -CN | 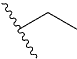 [SMILES: *CC] | 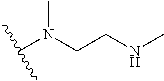 [SMILES: *N(C)CCNC] |
| 1-147 | Cl | H | -CN | 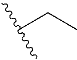 [SMILES: *CC] | 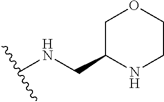 [SMILES: *NC[C@H]1COCCN1] |
| 1-148 | Cl | H | -CN | 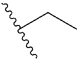 [SMILES: *CC] | 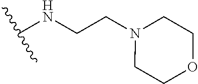 [SMILES: *NCCN1CCOCC1] |
| 1-149 | Cl | H | -CN | 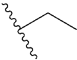 [SMILES: *CC] | 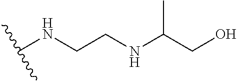 [SMILES: *NCCNC(C)CO] |
| 1-150 | Cl | H | -CN | 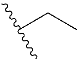 [SMILES: *CC] | 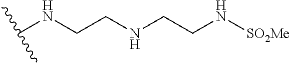 [SMILES: *NCCNCCNS(C)(=O)=O] |
| 1-151 | Cl | H | -CN | 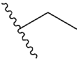 [SMILES: *CC] | 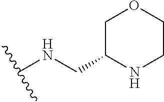 [SMILES: *NC[C@@H]1COCCN1] |
| 1-152 | Cl | H | -CN | 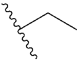 [SMILES: *CC] | 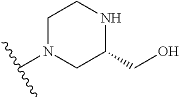 [SMILES: *N1CCN[C@H](CO)C1] |
| 1-153 | Cl | H | -CN | 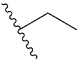 [SMILES: *CC] | 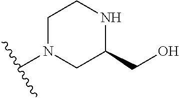 [SMILES: *N1CCN[C@@H](CO)C1] |
| 1-154 | Cl | H | -CN | 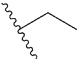 [SMILES: *CC] | 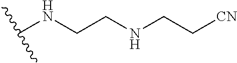 [SMILES: *NCCNCCC#N] |
| 1-155 | Cl | H | -CN | 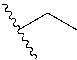 [SMILES: *CC] | 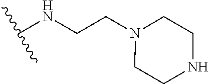 [SMILES: *NCCN1CCNCC1] |
| 1-156 | Cl | H | -CN | 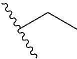 [SMILES: *CC] | 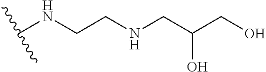 [SMILES: *NCCNCC(O)CO] |
| 1-157 | Cl | H | -CN | 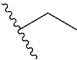 [SMILES: *CC] | 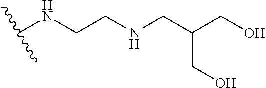 [SMILES: *NCCNCC(CO)CO] |
| 1-158 | Cl | H | -CN | 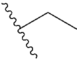 [SMILES: *CC] | 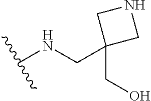 [SMILES: *NCC1(CO)CNC1] |
| 1-159 | Cl | H | -CN | 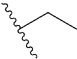 [SMILES: *CC] | 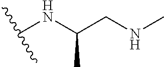 [SMILES: *N[C@H](C)CNC] |
| 1-160 | Cl | H | -CN | 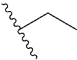 [SMILES: *CC] | 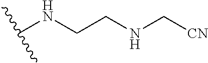 [SMILES: *NCCNCC#N] |
| 1-161 | Cl | H | -CN | 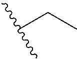 [SMILES: *CC] | 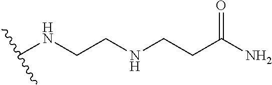 [SMILES: *NCCNCCC(N)=O] |
| 1-162 | Cl | H | -CN | 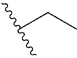 [SMILES: *CC] | 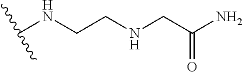 [SMILES: *NCCNCC(N)=O] |
| 1-163 | F | F | -CN | 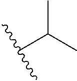 [SMILES: *C(C)C] | 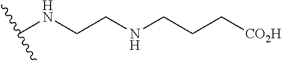 [SMILES: *NCCNCCCC(=O)O] |
| 1-164 | Cl | H | -CN | 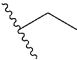 [SMILES: *CC] | 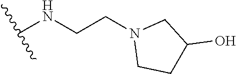 [SMILES: *NCCN1CCC(O)C1] |
| 1-165 | Cl | H | -CN | 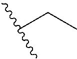 [SMILES: *CC] | 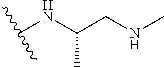 [SMILES: *N[C@@H](C)CNC] |
| 1-166 | Cl | H | -CN | 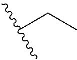 [SMILES: *CC] | 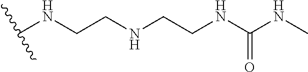 [SMILES: *NCCNCCNC(=O)NC] |
| 1-167 | Cl | H | -CN | 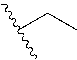 [SMILES: *CC] | 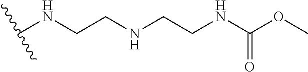 [SMILES: *NCCNCCNC(=O)OC] |
| 1-168 | Cl | H | -CN | 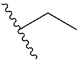 [SMILES: *CC] | 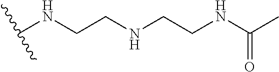 [SMILES: *NCCNCCNC(C)=O] |
| 1-169 | F | H | -CN | 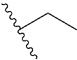 [SMILES: *CC] | 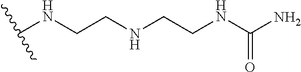 [SMILES: *NCCNCCNC(N)=O] |
| 1-170 | Cl | H | -CN | 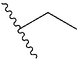 [SMILES: *CC] | 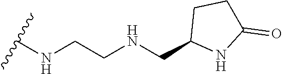 [SMILES: *NCCNC[C@H]1CCC(=O)N1] |
| 1-171 | Cl | H | CN | 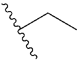 [SMILES: *CC] | 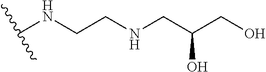 [SMILES: *NCCNC[C@H](O)CO] |
| 1-172 | F | H | -CN | 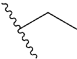 [SMILES: *CC] | 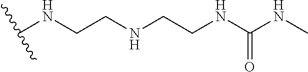 [SMILES: *NCCNCCNC(=O)NC] |
| 1-173 | Cl | H | -CN | 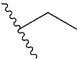 [SMILES: *CC] | 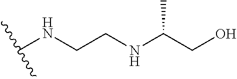 [SMILES: *NCCN[C@H](C)CO] |
| 1-174 | Cl | H | -CN | 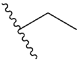 [SMILES: *CC] | 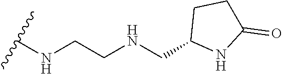 [SMILES: *NCCNC[C@@H]1CCC(=O)N1] |
| 1-175 | Cl | H | -CN | 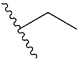 [SMILES: *CC] | 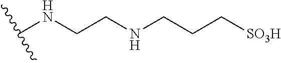 [SMILES: *NCCNCCCS(=O)(=O)O] |
| 1-176 | Cl | H | -CN | 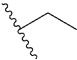 [SMILES: *CC] | 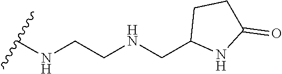 [SMILES: *NCCNCC1CCC(=O)N1] |
| 1-177 | Cl | H | -CN | 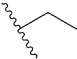 [SMILES: *CC] | 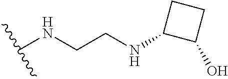 [SMILES: *NCCN[C@@H]1CC[C@@H]1O] |
| 1-178 | Cl | H | -CN | 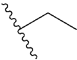 [SMILES: *CC] | 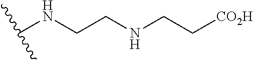 [SMILES: *NCCNCCC(=O)O] |
| 1-179 | Cl | H | -CN | 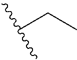 [SMILES: *CC] | 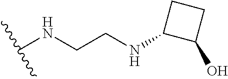 [SMILES: *NCCN[C@@H]1CC[C@H]1O] |
| 1-180 | Cl | H | -CN | 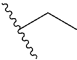 [SMILES: *CC] | 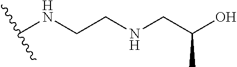 [SMILES: *NCCNC[C@H](C)O] |
| 1-181 | Cl | H | -CN | 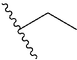 [SMILES: *CC] | 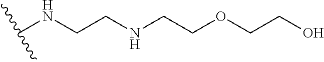 [SMILES: *NCCNCCOCCO] |
| 1-182 | Cl | H | -CN | 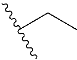 [SMILES: *CC] | 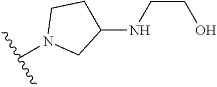 [SMILES: *N1CCC(NCCO)C1] |
| 1-183 | Cl | H | -CN | 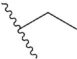 [SMILES: *CC] | 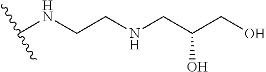 [SMILES: *NCCNC[C@@H](O)CO] |
| 1-184 | -CF₃ | H | -CN | 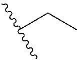 [SMILES: *CC] | 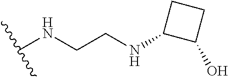 [SMILES: *NCCN[C@@H]1CC[C@@H]1O] |
| 1-185 | Cl | H | -CN | 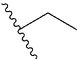 [SMILES: *CC] | 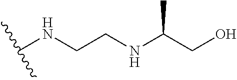 [SMILES: *NCCN[C@@H](C)CO] |
| 1-186 | Cl | H | -CN | 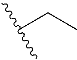 [SMILES: *CC] | 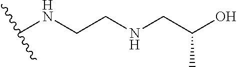 [SMILES: *NCCNC[C@@H](C)O] |
| 1-187 | Cl | H | -CN | 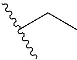 [SMILES: *CC] | 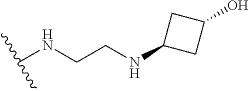 [SMILES: *NCCN[C@H]1C[C@H](O)C1] |
| 1-188 | Cl | H | -CN | 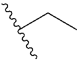 [SMILES: *CC] | 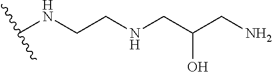 [SMILES: *NCCNCC(O)CN] |
| 1-189 | Cl | H | -CN | 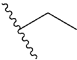 [SMILES: *CC] | 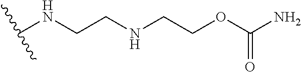 [SMILES: *NCCNCCOC(N)=O] |
| 1-190 | Cl | H | -CN | 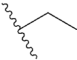 [SMILES: *CC] | 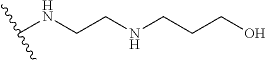 [SMILES: *NCCNCCCO] |
| 1-191 | Cl | H | -CN | 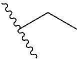 [SMILES: *CC] | 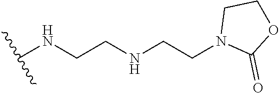 [SMILES: *NCCNCCN1CCOC1=O] |
| 1-192 | Cl | H | -CN | 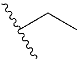 [SMILES: *CC] | 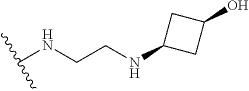 [SMILES: *NCCN[C@H]1C[C@@H](O)C1] |
| 1-193 | Cl | H | -CN | 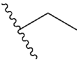 [SMILES: *CC] | 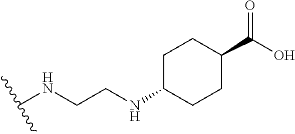 [SMILES: *NCCN[C@H]1CC[C@H](C(=O)O)CC1] |
| 1-194 | F | H | -CN | 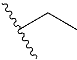 [SMILES: *CC] | 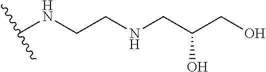 [SMILES: *NCCNC[C@@H](O)CO] |
| 1-195 | Cl | H | -CN | 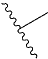 | 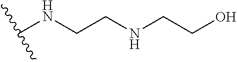 [SMILES: *NCCNCCO] |
| 1-196 | -CF₃ | H | -CN | 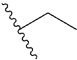 [SMILES: *CC] | 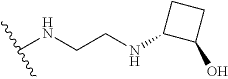 [SMILES: *NCCN[C@@H]1CC[C@H]1O] |
| 1-197 | Cl | H | -CN | 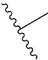 | 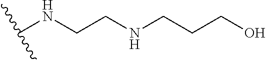 [SMILES: *NCCNCCCO] |
| 1-198 | Cl | H | -CN | 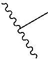 | 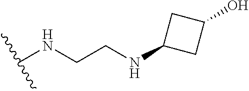 [SMILES: *NCCN[C@H]1C[C@H](O)C1] |
| 1-199 | F | H | -CN | 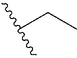 [SMILES: *CC] | 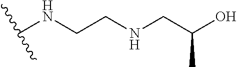 [SMILES: *NCCNC[C@H](C)O] |
| 1-200 | F | H | -CN | 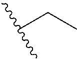 [SMILES: *CC] | 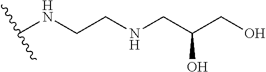 [SMILES: *NCCNC[C@H](O)CO] |
| 1-201 | Cl | H | -CN | 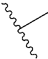 | 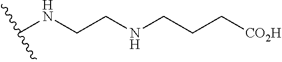 [SMILES: *NCCNCCCC(=O)O] |
| 1-202 | Cl | H | -CN | 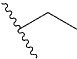 [SMILES: *CC] | 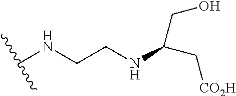 [SMILES: *NCCN[C@@H](CO)CC(=O)O] |
| 1-203 | F | H | -CN | 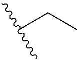 [SMILES: *CC] | 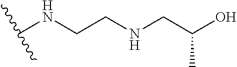 [SMILES: *NCCNC[C@@H](C)O] |
| 1-204 | F | H | -CN | 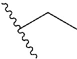 [SMILES: *CC] | 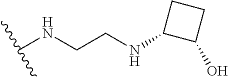 [SMILES: *NCCN[C@@H]1CC[C@@H]1O] |
| 1-205 | Cl | H | -CN | 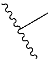 | 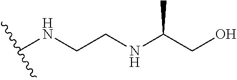 [SMILES: *NCCN[C@@H](C)CO] |
| 1-206 | Cl | H | -CN | 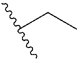 [SMILES: *CC] | 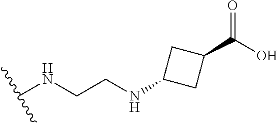 [SMILES: *NCCN[C@H]1C[C@H](C(=O)O)C1] |
| 1-207 | -CF₃ | H | -CN | 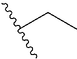 [SMILES: *CC] | 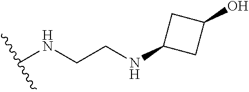 [SMILES: *NCCN[C@H]1C[C@@H](O)C1] |
| 1-208 | -CH₃ | H | -CN | 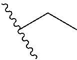 [SMILES: *CC] | 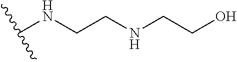 [SMILES: *NCCNCCO] |
| 1-209 | Cl | H | -CN | 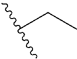 [SMILES: *CC] | 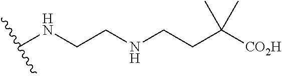 [SMILES: *NCCNCCC(C)(C)C(=O)O] |
| 1-210 | Cl | H | -CN | 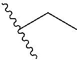 [SMILES: *CC] | 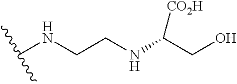 [SMILES: *NCCN[C@@H](CO)C(=O)O] |
| 1-211 | Cl | H | -CN | 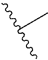 | 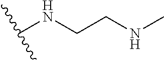 [SMILES: *NCCNC] |
| 1-212 | F | F | -CN | 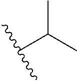 [SMILES: *C(C)C] | 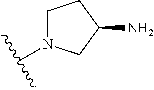 [SMILES: *N1CC[C@@H](N)C1] |
| 1-213 | F | H | -CN | 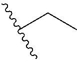 [SMILES: *CC] | 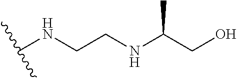 [SMILES: *NCCN[C@@H](C)CO] |
| 1-214 | F | H | -CN | 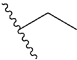 [SMILES: *CC] | 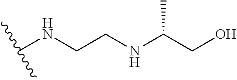 [SMILES: *NCCN[C@H](C)CO] |
| 1-215 | Cl | H | -CN | 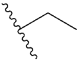 [SMILES: *CC] | 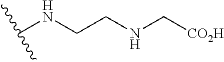 [SMILES: *NCCNCC(=O)O] |
| 1-216 | F | H | -CN | 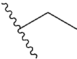 [SMILES: *CC] | 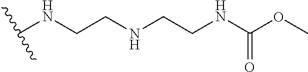 [SMILES: *NCCNCCNC(=O)OC] |
| 1-217 | Cl | H | -CN | 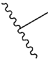 | 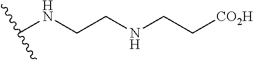 [SMILES: *NCCNCCC(=O)O] |
| 1-218 | F | H | -CN | 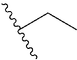 [SMILES: *CC] | 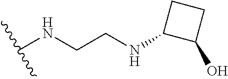 [SMILES: *NCCN[C@@H]1CC[C@H]1O] |
| 1-219 | Cl | H | -CN | 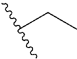 [SMILES: *CC] | 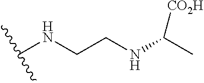 [SMILES: *NCCN[C@@H](C)C(=O)O] |
| 1-220 | Cl | H | -CN | 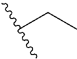 [SMILES: *CC] | 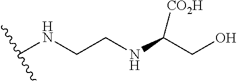 [SMILES: *NCCN[C@H](CO)C(=O)O] |
| 1-221 | F | H | -CN | 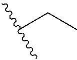 [SMILES: *CC] | 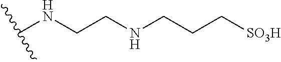 [SMILES: *NCCNCCCS(=O)(=O)O] |
| 1-222 | F | H | -CN | 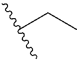 [SMILES: *CC] | 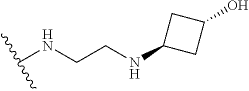 [SMILES: *NCCN[C@H]1C[C@H](O)C1] |
| 1-223 | Cl | H | -CN | 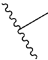 | 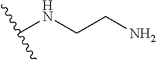 [SMILES: *NCCN] |
| 1-224 | -CF₃ | H | -CN | 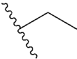 [SMILES: *CC] | 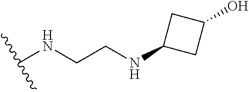 [SMILES: *NCCN[C@H]1C[C@H](O)C1] |
| 1-225 | Cl | H | -CN | 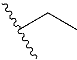 [SMILES: *CC] | 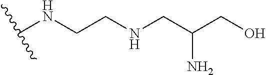 [SMILES: *NCCNCC(N)CO] |
| 1-226 | F | H | -CH₃ | 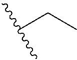 [SMILES: *CC] | 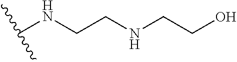 [SMILES: *NCCNCCO] |
| 1-227 | -CF₃ | H | -CN | 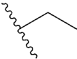 [SMILES: *CC] | 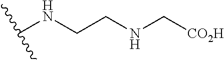 [SMILES: *NCCNCC(=O)O] |
| 1-228 | Cl | H | -CN | 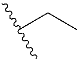 [SMILES: *CC] | 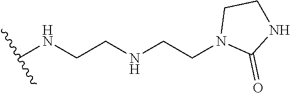 [SMILES: *NCCNCCN1CCNC1=O] |
| 1-229 | Cl | H | -CN | 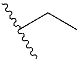 [SMILES: *CC] | 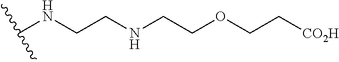 [SMILES: *NCCNCCOCCC(=O)O] |
| 1-230 | Cl | H | -CN | 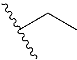 [SMILES: *CC] | 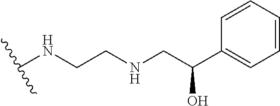 [SMILES: *NCCNC[C@H](O)c1ccccc1] |
| 1-231 | Cl | H | -CN | 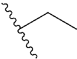 [SMILES: *CC] | 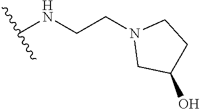 [SMILES: *NCCN1CC[C@@H](O)C1] |
| 1-232 | F | H | -CN | 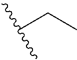 [SMILES: *CC] | 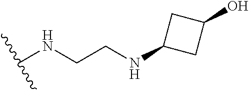 [SMILES: *NCCN[C@H]1C[C@@H](O)C1] |
| 1-233 | Cl | H | -CN | 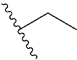 [SMILES: *CC] | 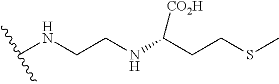 [SMILES: *NCCN[C@@H](CCSC)C(=O)O] |
| 1-234 | Cl | H | -CN | 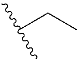 [SMILES: *CC] | 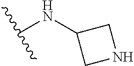 [SMILES: *NC1CNC1] |
| 1-235 | Cl | H | -CN | 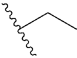 [SMILES: *CC] | 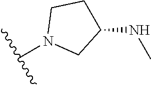 [SMILES: *N1CC[C@H](NC)C1] |
| 1-236 | Cl | H | -CN | 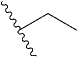 [SMILES: *CC] | 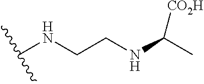 [SMILES: *NCCN[C@H](C)C(=O)O] |
| 1-237 | F | H | -CN | 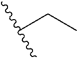 [SMILES: *CC] | 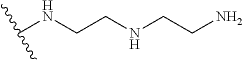 [SMILES: *NCCNCCN] |
| 1-238 | Cl | H | -CN | 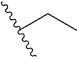 [SMILES: *CC] | 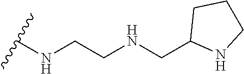 [SMILES: *NCCNCC1CCCN1] |
| 1-239 | Cl | H | -CN | 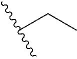 [SMILES: *CC] | 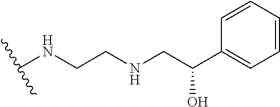 [SMILES: *NCCNC[C@@H](O)c1ccccc1] |
| 1-240 | Cl | H | -CN | 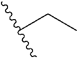 [SMILES: *CC] | 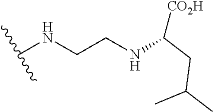 [SMILES: *NCCN[C@@H](CC(C)C)C(=O)O] |
| 1-241 | Cl | H | -CN | 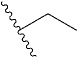 [SMILES: *CC] | 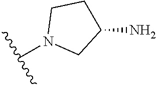 [SMILES: *N1CC[C@H](N)C1] |
| 1-242 | Cl | H | Cl | 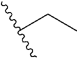 [SMILES: *CC] | 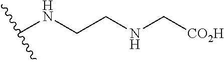 [SMILES: *NCCNCC(=O)O] |

Compounds in Table 1 are named:
1-1: 13-chloro-10-(2,6-difluoro-4-{[2-(methylamino)ethyl]amino}phenyl)-8-methyl-6,8,10-triazatricyclo[9.4.0.0^{2,7}]pentadeca-1(11),2(7),3,5,12,14-hexaen-9-one;
1-2: 13-chloro-10-(2,6-difluoro-4-{[2-(methylamino)ethyl]amino}phenyl)-8-ethyl-6,8,10-triazatricyclo[9.4.0.0^{2,7}]pentadeca-1(11),2(7),3,5,12,14-hexaen-9-one;
1-3: 10-{4-[(2-aminoethyl)amino]-2,6-difluorophenyl}-13-chloro-8-ethyl-6,8,10-triazatricyclo[9.4.0.0^{2,7}]pentadeca-1(11),2(7),3,5,12,14-hexaen-9-one;
1-4: 13-chloro-10-[2,6-difluoro-4-({2-[(oxan-4-yl)amino]ethyl}amino)phenyl]-8-ethyl-6,8,10-triazatricyclo[9.4.0.0^{2,7}]pentadeca-1(11),2(7),3,5,12,14-hexaen-9-one;
1-5: 13-chloro-10-[2,6-difluoro-4-({2-[(oxolan-3-yl)amino]ethyl}amino)phenyl]-8-ethyl-6,8,10-triazatricyclo[9.4.0.0^{2,7}]pentadeca-1(11),2(7),3,5,12,14-hexaen-9-one;
1-6: 4-({2-[(4-{13-chloro-8-ethyl-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)butanoic acid;
1-7: 13-chloro-10-[2,6-difluoro-4-({2-[(2-hydroxyethyl)amino]ethyl}amino)phenyl]-8-ethyl-6,8,10-triazatricyclo[9.4.0.0^{2,7}]pentadeca-1(11),2(7),3,5,12,14-hexaen-9-one;
1-8: 4,13-dichloro-10-(2,6-difluoro-4-{[2-(methylamino)ethyl]amino}phenyl)-8-ethyl-6,8,10-triazatricyclo[9.4.0.0^{2,7}]pentadeca-1(11),2(7),3,5,12,14-hexaen-9-one;
1-9: 10-{4-[(2-aminoethyl)amino]-2,6-difluorophenyl}-4,13-dichloro-8-ethyl-6,8,10-triazatricyclo[9.4.0.0^{2,7}]pentadeca-1(11),2(7),3,5,12,14-hexaen-9-one;
1-10: 4-({2-[(4-{4,13-dichloro-8-ethyl-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)butanoic acid;
1-11: 4,13-dichloro-10-[2,6-difluoro-4-({2-[(2-hydroxyethyl)amino]ethyl}amino)phenyl]-8-ethyl-6,8,10-triazatricyclo[9.4.0.0^{2,7}]pentadeca-1(11),2(7),3,5,12,14-hexaen-9-one;
1-12: 4,13-dichloro-10-[2,6-difluoro-4-({2-[(oxan-4-yl)amino]ethyl}amino)phenyl]-8-ethyl-6,8,10-triazatricyclo[9.4.0.0^{2,7}]pentadeca-1(11),2(7),3,5,12,14-hexaen-9-one;
1-13: 4,13-dichloro-10-[2,6-difluoro-4-({2-[(oxolan-3-yl)amino]ethyl}amino)phenyl]-8-ethyl-6,8,10-triazatricyclo[9.4.0.0^{2,7}]pentadeca-1(11),2(7),3,5,12,14-hexaen-9-one;
1-14: 13-chloro-10-(2,6-difluoro-4-{[2-(methylamino)ethyl]amino}phenyl)-8-ethyl-4-fluoro-6,8,10-triazatricyclo[9.4.0.0^{2,7}]pentadeca-1(11),2(7),3,5,12,14-hexaen-9-one;
1-15: 10-{4-[(2-aminoethyl)amino]-2,6-difluorophenyl}-13-chloro-8-ethyl-4-fluoro-6,8,10-triazatricyclo[9.4.0.0^{2,7}]pentadeca-1(11),2(7),3,5,12,14-hexaen-9-one;
1-16: 4-({2-[(4-{13-chloro-8-ethyl-4-fluoro-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)butanoic acid;
1-17: 13-chloro-10-[2,6-difluoro-4-({2-[(2-hydroxyethyl)amino]ethyl}amino)phenyl]-8-ethyl-4-fluoro-6,8,10-triazatricyclo[9.4.0.0^{2,7}]pentadeca-1(11),2(7),3,5,12,14-hexaen-9-one;
1-18: 13-chloro-10-(2,6-difluoro-4-{[2-(methylamino)ethyl]amino}phenyl)-8-ethyl-4-methyl-6,8,10-triazatricyclo[9.4.0.0^{2,7}]pentadeca-1(11),2(7),3,5,12,14-hexaen-9-one;
1-19: 13-chloro-10-[2,6-difluoro-4-({2-[(oxan-4-yl)amino]ethyl}amino)phenyl]-8-ethyl-4-fluoro-6,8,10-triazatricyclo[9.4.0.0^{2,7}]pentadeca-1(11),2(7),3,5,12,14-hexaen-9-one;
1-20: 13-chloro-10-[2,6-difluoro-4-({2-[(oxolan-3-yl)amino]ethyl}amino)phenyl]-8-ethyl-4-fluoro-6,8,10-triazatricyclo[9.4.0.0^{2,7}]pentadeca-1(11),2(7),3,5,12,14-hexaen-9-one;
1-21: 4-chloro-10-(2,6-difluoro-4-{[2-(methylamino)ethyl]amino}phenyl)-8-ethyl-15-methyl-6,8,10-triazatricyclo[9.4.0.0^{2,7}]pentadeca-1(11),2(7),3,5,12,14-hexaen-9-one;
1-22: 10-{4-[(2-aminoethyl)amino]-2,6-difluorophenyl}-13-chloro-8-ethyl-4-methyl-6,8,10-triazatricyclo[9.4.0.0^{2,7}]pentadeca-1(11),2(7),3,5,12,14-hexaen-9-one;
1-23: 13-chloro-10-(2,6-difluoro-4-{[2-(methylamino)ethyl]amino}phenyl)-8-ethyl-15-methyl-6,8,10-triazatricyclo[9.4.0.0^{2,7}]pentadeca-1(11),2(7),3,5,12,14-hexaen-9-one;
1-24: 13-chloro-4-cyclopropyl-10-(2,6-difluoro-4-{[2-(methylamino)ethyl]amino}phenyl)-8-ethyl-6,8,10-triazatricyclo[9.4.0.0^{2,7}]pentadeca-1(11),2(7),3,5,12,14-hexaen-9-one;
1-25: 4-({2-[(4-{13-chloro-8-ethyl-4-methyl-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)butanoic acid;
1-26: 13-chloro-10-[2,6-difluoro-4-({2-[(2-hydroxyethyl)amino]ethyl}amino)phenyl]-8-ethyl-4-methyl-6,8,10-triazatricyclo[9.4.0.0^{2,7}]pentadeca-1(11),2(7),3,5,12,14-hexaen-9-one;
1-27: 13-chloro-10-[2,6-difluoro-4-({2-[(oxan-4-yl)amino]ethyl}amino)phenyl]-8-ethyl-4-methyl-6,8,10-triazatricyclo[9.4.0.0^{2,7}]pentadeca-1(11),2(7),3,5,12,14-hexaen-9-one;
1-28: 13-chloro-10-[2,6-difluoro-4-({2-[(oxolan-3-yl)amino]ethyl}amino)phenyl]-8-ethyl-4-methyl-6,8,10-triazatricyclo[9.4.0.0^{2,7}]pentadeca-1(11),2(7),3,5,12,14-hexaen-9-one;
1-29: 4-chloro-10-(2,6-difluoro-4-{[2-(methylamino)ethyl]amino}phenyl)-8-ethyl-13-methoxy-6,8,10-triazatricyclo[9.4.0.0^{2,7}]pentadeca-1(11),2(7),3,5,12,14-hexaen-9-one;
1-30: 4,13-dichloro-10-(2,6-difluoro-4-{[2-(methylamino)ethyl]amino}phenyl)-8-methyl-6,8,10-triazatricyclo[9.4.0.0^{2,7}]pentadeca-1(11),2(7),3,5,12,14-hexaen-9-one;
1-31: 13-chloro-10-[2,6-difluoro-4-({2-[(2-hydroxyethyl)amino]ethyl}amino)phenyl]-8-ethyl-4-fluoro-15-methyl-6,8,10-triazatricyclo[9.4.0.0^{2,7}]pentadeca-1(11),2(7),3,5,12,14-hexaen-9-one;
1-32: 13-chloro-10-(2,6-difluoro-4-{[2-(methylamino)ethyl]amino}phenyl)-8-ethyl-4-fluoro-15-methyl-6,8,10-triazatricyclo[9.4.0.0^{2,7}]pentadeca-1(11),2(7),3,5,12,14-hexaen-9-one;
1-33: 10-{4-[(2-aminoethyl)amino]-2,6-difluorophenyl}-4,13-dichloro-8-methyl-6,8,10-triazatricyclo[9.4.0.0^{2,7}]pentadeca-1(11),2(7),3,5,12,14-hexaen-9-one;
1-34: 4,13-dichloro-10-[2,6-difluoro-4-({2-[(2-hydroxyethyl)amino]ethyl}amino)phenyl]-8-methyl-6,8,10-triazatricyclo[9.4.0.0^{2,7}]pentadeca-1(11),2(7),3,5,12,14-hexaen-9-one;
1-35: 4,13-dichloro-10-[2,6-difluoro-4-({2-[(oxan-4-yl)amino]ethyl}amino)phenyl]-8-methyl-6,8,10-triazatricyclo[9.4.0.0^{2,7}]pentadeca-1(11),2(7),3,5,12,14-hexaen-9-one;
1-36: 4,13-dichloro-10-(2,6-difluoro-4-{[2-(methylamino)ethyl]amino}phenyl)-8-ethyl-15-methyl-6,8,10-triazatricyclo[9.4.0.0^{2,7}]pentadeca-1(11),2(7),3,5,12,14-hexaen-9-one;
1-37: 13,15-dichloro-10-[2,6-difluoro-4-({2-[(2-hydroxyethyl)amino]ethyl}amino)phenyl]-8-ethyl-4-fluoro-6,8,10-triazatricyclo[9.4.0.0^{2,7}]pentadeca-1(11),2(7),3,5,12,14-hexaen-9-one;
1-38: 13,15-dichloro-10-(2,6-difluoro-4-{[2-(methylamino)ethyl]amino}phenyl)-8-ethyl-4-fluoro-6,8,10-triazatricyclo[9.4.0.0^{2,7}]pentadeca-1(11),2(7),3,5,12,14-hexaen-9-one;
1-39: 4,13-dichloro-10-(2,6-difluoro-4-{[2-(methylamino)ethyl]amino}phenyl)-8-(propan-2-yl)-6,8,10-triazatricyclo[9.4.0.0^{2,7}]pentadeca-1(11),2(7),3,5,12,14-hexaen-9-one;
1-40: 4,13-dichloro-10-(2,6-difluoro-4-{[2-(methylamino)ethyl]amino}phenyl)-8-propyl-6,8,10-triazatricyclo[9.4.0.0^{2,7}]pentadeca-1(11),2(7),3,5,12,14-hexaen-9-one;
1-41: 2-[(4-{4,13-dichloro-8-ethyl-9-oxo-6,8,10-triazatricyclo[9.4.0.0^{2,7}]pentadeca-1(11),2(7),3,5,12,14-hexaen-10-yl}-3,5-difluorophenyl)amino]acetic acid;
1-42: 10-{4-[(3R)-3-aminopyrrolidin-1-yl]-2,6-difluorophenyl}-4,13-dichloro-8-ethyl-6,8,10-triazatricyclo[9.4.0.0^{2,7}]pentadeca-1(11),2(7),3,5,12,14-hexaen-9-one;
1-43: 10-{4-[(3S)-3-aminopyrrolidin-1-yl]-2,6-difluorophenyl}-4,13-dichloro-8-ethyl-6,8,10-triazatricyclo[9.4.0.0^{2,7}]pentadeca-1(11),2(7),3,5,12,14-hexaen-9-one;
1-44: 10-[4-(3-aminoazetidin-1-yl)-2,6-difluorophenyl]-4,13-dichloro-8-ethyl-6,8,10-triazatricyclo[9.4.0.0^{2,7}]pentadeca-1(11),2(7),3,5,12,14-hexaen-9-one;
1-45: 4,13-dichloro-10-(2,6-difluoro-4-{[2-(methylamino)ethyl]amino}phenyl)-8-(2,2,2-trifluoroethyl)-6,8,10-triazatricyclo[9.4.0.0^{2,7}]pentadeca-1(11),2(7),3,5,12,14-hexaen-9-one;
1-46: 4,13-dichloro-8-(cyclopropylmethyl)-10-(2,6-difluoro-4-{[2-(methylamino)ethyl]amino}phenyl)-6,8,10-triazatricyclo[9.4.0.0^{2,7}]pentadeca-1(11),2(7),3,5,12,14-hexaen-9-one;
1-47: 4,13-dichloro-10-[2,6-difluoro-4-({2-[(2-hydroxyethyl)amino]ethyl}amino)phenyl]-8-(propan-2-yl)-6,8,10-triazatricyclo[9.4.0.0^{2,7}]pentadeca-1(11),2(7),3,5,12,14-hexaen-9-one;
1-48: 4,13-dichloro-10-[2,6-difluoro-4-({2-[(2-hydroxyethyl)amino]ethyl}amino)phenyl]-8-(2,2,2-trifluoroethyl)-6,8,10-triazatricyclo[9.4.0.0^{2,7}]pentadeca-1(11),2(7),3,5,12,14-hexaen-9-one;
1-49: 4,13-dichloro-10-[2,6-difluoro-4-({2-[(2-hydroxyethyl)amino]ethyl}amino)phenyl]-8-propyl-6,8,10-triazatricyclo[9.4.0.0^{2,7}]pentadeca-1(11),2(7),3,5,12,14-hexaen-9-one;
1-50: 3-[(4-{4,13-dichloro-8-ethyl-9-oxo-6,8,10-triazatricyclo[9.4.0.0^{2,7}]pentadeca-1(11),2(7),3,5,12,14-hexaen-10-yl}-3,5-difluorophenyl)amino]propanoic acid;
1-51: 4,13-dichloro-10-[2,6-difluoro-4-({2-[(2-hydroxyethyl)amino]ethyl}amino)phenyl]-8-ethyl-15-methyl-6,8,10-triazatricyclo[9.4.0.0^{2,7}]pentadeca-1(11),2(7),3,5,12,14-hexaen-9-one;
1-52: 4,13-dichloro-8-(cyclopropylmethyl)-10-[2,6-difluoro-4-({2-[(2-hydroxyethyl)amino]ethyl}amino)phenyl]-6,8,10-triazatricyclo[9.4.0.0^{2,7}]pentadeca-1(11),2(7),3,5,12,14-hexaen-9-one;
1-53: ethyl 4-[(4-{4,13-dichloro-8-ethyl-9-oxo-6,8,10-triazatricyclo[9.4.0.0^{2,7}]pentadeca-1(11),2(7),3,5,12,14-hexaen-10-yl}-3,5-difluorophenyl)amino]butanoate;
1-54: 4-chloro-10-(2,6-difluoro-4-{[2-(methylamino)ethyl]amino}phenyl)-8-ethyl-9-oxo-6,8,10-triazatricyclo[9.4.0.0^{2,7}]pentadeca-1(11),2(7),3,5,12,14-hexaene-13-carbonitrile;
1-55: 4-chloro-10-[2,6-difluoro-4-({2-[(2-hydroxyethyl)amino]ethyl}amino)phenyl]-8-ethyl-9-oxo-6,8,10-triazatricyclo[9.4.0.0^{2,7}]pentadeca-1(11),2(7),3,5,12,14-hexaene-13-carbonitrile;
1-56: 13-chloro-10-[2,6-difluoro-4-({2-[(2-hydroxyethyl)amino]ethyl}amino)phenyl]-8-ethyl-4-(trifluoromethyl)-6,8,10-triazatricyclo[9.4.0.0^{2,7}]pentadeca-1(11),2(7),3,5,12,14-hexaen-9-one;
1-57: 13-chloro-10-(2,6-difluoro-4-{[2-(methylamino)ethyl]amino}phenyl)-8-ethyl-4-(trifluoromethyl)-6,8,10-triazatricyclo[9.4.0.0^{2,7}]pentadeca-1(11),2(7),3,5,12,14-hexaen-9-one;
1-58: 4-({2-[(4-{4-chloro-13-cyano-8-ethyl-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)butanoic acid;
1-59: 4,13-dichloro-10-[2,6-difluoro-4-({2-[(2-fluoroethyl)amino]ethyl}amino)phenyl]-8-ethyl-6,8,10-triazatricyclo[9.4.0.0^{2,7}]pentadeca-1(11),2(7),3,5,12,14-hexaen-9-one;
1-60: 4,13-dichloro-10-[2,6-difluoro-4-({2-[(2-hydroxyethyl)amino]ethyl}amino)phenyl]-8-ethyl-15-fluoro-6,8,10-triazatricyclo[9.4.0.0^{2,7}]pentadeca-1(11),2(7),3,5,12,14-hexaen-9-one;
1-61: 4,13-dichloro-10-(2,6-difluoro-4-{[2-(methylamino)ethyl]amino}phenyl)-8-ethyl-15-fluoro-6,8,10-triazatricyclo[9.4.0.0^{2,7}]pentadeca-1(11),2(7),3,5,12,14-hexaen-9-one;
1-62: 10-(2,6-difluoro-4-{[2-(methylamino)ethyl]amino}phenyl)-8-ethyl-4-fluoro-15-methyl-9-oxo-6,8,10-triazatricyclo[9.4.0.0^{2,7}]pentadeca-1(11),2(7),3,5,12,14-hexaene-13-carbonitrile;
1-63: 10-[2,6-difluoro-4-({2-[(2-hydroxyethyl)amino]ethyl}amino)phenyl]-8-ethyl-4-fluoro-15-methyl-9-oxo-6,8,10-triazatricyclo[9.4.0.0^{2,7}]pentadeca-1(11),2(7),3,5,12,14-hexaene-13-carbonitrile;
1-64: 4-({2-[(4-{13-chloro-8-ethyl-4-fluoro-15-methyl-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)butanoic acid;
1-65: 10-{4-[(3R)-3-aminopyrrolidin-1-yl]-2,6-difluorophenyl}-13-chloro-8-ethyl-4-fluoro-15-methyl-6,8,10-triazatricyclo[9.4.0.0^{2,7}]pentadeca-1(11),2(7),3,5,12,14-hexaen-9-one;
1-66: 10-(2,6-difluoro-4-{[2-(methylamino)ethyl]amino}phenyl)-8-ethyl-4-fluoro-13-(trifluoromethyl)-6,8,10-triazatricyclo[9.4.0.0^{2,7}]pentadeca-1(11),2(7),3,5,12,14-hexaen-9-one;
1-67: 10-[2,6-difluoro-4-({2-[(2-hydroxyethyl)amino]ethyl}amino)phenyl]-8-ethyl-4-fluoro-13-(trifluoromethyl)-6,8,10-triazatricyclo[9.4.0.0^{2,7}]pentadeca-1(11),2(7),3,5,12,14-hexaen-9-one;
1-68: 4-({2-[(4-{13-cyano-8-ethyl-4-fluoro-15-methyl-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)butanoic acid;
1-69: 4-{[2-({4-[8-ethyl-4-fluoro-9-oxo-13-(trifluoromethyl)-6,8,10-triazatricyclo[9.4.0.0^{2,7}]pentadeca-1(11),2(7),3,5,12,14-hexaen-10-yl]-3,5-difluorophenyl}amino)ethyl]amino}butanoic acid;
1-70: 4-({2-[(4-{4,13-dichloro-8-ethyl-9-oxo-6,8,10-triazatricyclo[9.4.0.0^{2,7}]pentadeca-1(11),2(7),3,5,12,14-hexaen-10-yl}-3,5-difluorophenyl)amino]ethyl}(methyl)amino)butanoic acid;
1-71: 4,13-dichloro-10-(2,6-difluoro-4-{[(2S)-1-[(2-hydroxyethyl)amino]propan-2-yl]amino}phenyl)-8-ethyl-6,8,10-triazatricyclo[9.4.0.0^{2,7}]pentadeca-1(11),2(7),3,5,12,14-hexaen-9-one;
1-72: 3-({2-[(4-{4,13-dichloro-8-ethyl-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)propanoic acid;
1-73: 4,13-dichloro-10-[2,6-difluoro-4-({2-[(2-hydroxyethyl)(methyl)amino]ethyl}amino)phenyl]-8-ethyl-6,8,10-triazatricyclo[9.4.0.0^{2,7}]pentadeca-1(11),2(7),3,5,12,14-hexaen-9-one;
1-74: 4,13-dichloro-10-[2,6-difluoro-4-({2-[(2-methoxyethyl)amino]ethyl}amino)phenyl]-8-ethyl-6,8,10-triazatricyclo[9.4.0.0^{2,7}]pentadeca-1(11),2(7),3,5,12,14-hexaen-9-one;
1-75: 13-chloro-10-(2,6-difluoro-4-{[2-(methylamino)ethyl]amino}phenyl)-4-fluoro-8,15-dimethyl-6,8,10-triazatricyclo[9.4.0.0^{2,7}]pentadeca-1(11),2(7),3,5,12,14-hexaen-9-one;
1-76: 13-chloro-10-[2,6-difluoro-4-({2-[(2-hydroxyethyl)amino]ethyl}amino)phenyl]-4-fluoro-8,15-dimethyl-6,8,10-triazatricyclo[9.4.0.0^{2,7}]pentadeca-1(11),2(7),3,5,12,14-hexaen-9-one;
1-77: 4-({2-[(4-{13-chloro-4-fluoro-8,15-dimethyl-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)butanoic acid;
1-78: 13-chloro-10-(2,6-difluoro-4-{[2-(methylamino)ethyl]amino}phenyl)-4-fluoro-15-methyl-8-(propan-2-yl)-6,8,10-triazatricyclo[9.4.0.0^{2,7}]pentadeca-1(11),2(7),3,5,12,14-hexaen-9-one;
1-79: 13-chloro-10-[2,6-difluoro-4-({2-[(2-hydroxyethyl)amino]ethyl}amino)phenyl]-4-fluoro-15-methyl-8-(propan-2-yl)-6,8,10-triazatricyclo[9.4.0.0^{2,7}]pentadeca-1(11),2(7),3,5,12,14-hexaen-9-one;
1-80: 4-{[2-({4-[13-chloro-4-fluoro-15-methyl-9-oxo-8-(propan-2-yl)-6,8,10-triazatricyclo[9.4.0.0^{2,7}]pentadeca-1(11),2(7),3,5,12,14-hexaen-10-yl]-3,5-difluorophenyl}amino)ethyl]amino}butanoic acid;
1-81: 10-(2,6-difluoro-4-{[2-(methylamino)ethyl]amino}phenyl)-8-ethyl-4-fluoro-9-oxo-6,8,10-triazatricyclo[9.4.0.0^{2,7}]pentadeca-1(11),2(7),3,5,12,14-hexaene-13-carbonitrile;
1-82: 10-[2,6-difluoro-4-({2-[(2-hydroxyethyl)amino]ethyl}amino)phenyl]-8-ethyl-4-fluoro-9-oxo-6,8,10-triazatricyclo[9.4.0.0^{2,7}]pentadeca-1(11),2(7),3,5,12,14-hexaene-13-carbonitrile;
1-83: 4-({2-[(4-{13-cyano-8-ethyl-4-fluoro-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)butanoic acid;
1-84: 10-(2,6-difluoro-4-{[2-(methylamino)ethyl]amino}phenyl)-4-fluoro-9-oxo-8-(propan-2-yl)-6,8,10-triazatricyclo[9.4.0.0^{2,7}]pentadeca-1(11),2(7),3,5,12,14-hexaene-13-carbonitrile;
1-85: 10-[2,6-difluoro-4-({2-[(2-hydroxyethyl)amino]ethyl}amino)phenyl]-4-fluoro-9-oxo-8-(propan-2-yl)-6,8,10-triazatricyclo[9.4.0.0^{2,7}]pentadeca-1(11),2(7),3,5,12,14-hexaene-13-carbonitrile;
1-86: 4-{[2-({4-[13-cyano-4-fluoro-9-oxo-8-(propan-2-yl)-6,8,10-triazatricyclo[9.4.0.0^{2,7}]pentadeca-1(11),2(7),3,5,12,14-hexaen-10-yl]-3,5-difluorophenyl}amino)ethyl]amino}butanoic acid;
1-87: 13-chloro-10-(2,6-difluoro-4-{[2-(methylamino)ethyl]amino}phenyl)-8-ethyl-4-fluoro-15-methoxy-6,8,10-triazatricyclo[9.4.0.0^{2,7}]pentadeca-1(11),2(7),3,5,12,14-hexaen-9-one;
1-88: 13-chloro-10-[2,6-difluoro-4-({2-[(2-hydroxyethyl)amino]ethyl}amino)phenyl]-8-ethyl-4-fluoro-15-methoxy-6,8,10-triazatricyclo[9.4.0.0^{2,7}]pentadeca-1(11),2(7),3,5,12,14-hexaen-9-one;
1-89: 13-chloro-10-(2,6-difluoro-4-{[2-(methylamino)ethyl]amino}phenyl)-8-ethyl-9-oxo-6,8,10-triazatricyclo[9.4.0.0^{2,7}]pentadeca-1(11),2(7),3,5,12,14-hexaene-4-carbonitrile;
1-90: 13-chloro-10-[2,6-difluoro-4-({2-[(2-hydroxyethyl)amino]ethyl}amino)phenyl]-8-ethyl-9-oxo-6,8,10-triazatricyclo[9.4.0.0^{2,7}]pentadeca-1(11),2(7),3,5,12,14-hexaene-4-carbonitrile;
1-91: 4-chloro-10-(2,6-difluoro-4-{[2-(methylamino)ethyl]amino}phenyl)-9-oxo-8-(propan-2-yl)-6,8,10-triazatricyclo[9.4.0.0^{2,7}]pentadeca-1(11),2(7),3,5,12,14-hexaene-13-carbonitrile;
1-92: 4-chloro-10-[2,6-difluoro-4-({2-[(2-hydroxyethyl)amino]ethyl}amino)phenyl]-9-oxo-8-(propan-2-yl)-6,8,10-triazatricyclo[9.4.0.0^{2,7}]pentadeca-1(11),2(7),3,5,12,14-hexaene-13-carbonitrile;
1-93: 4-{[2-({4-[4-chloro-13-cyano-9-oxo-8-(propan-2-yl)-6,8,10-triazatricyclo[9.4.0.0^{2,7}]pentadeca-1(11),2(7),3,5,12,14-hexaen-10-yl]-3,5-difluorophenyl}amino)ethyl]amino}butanoic acid;
1-94: 13-chloro-10-(2,6-difluoro-4-{[2-(methylamino)ethyl]amino}phenyl)-8-ethyl-4,15-difluoro-6,8,10-triazatricyclo[9.4.0.0^{2,7}]pentadeca-1(11),2(7),3,5,12,14-hexaen-9-one;
1-95: 13-chloro-10-[2,6-difluoro-4-({2-[(2-hydroxyethyl)amino]ethyl}amino)phenyl]-8-ethyl-4,15-difluoro-6,8,10-triazatricyclo[9.4.0.0^{2,7}]pentadeca-1(11),2(7),3,5,12,14-hexaen-9-one;
1-96: 4-({2-[(4-{13-chloro-8-ethyl-4,15-difluoro-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)butanoic acid;
1-97: 10-(2,6-difluoro-4-{[2-(methylamino)ethyl]amino}phenyl)-8-ethyl-9-oxo-4-(trifluoromethyl)-6,8,10-triazatricyclo[9.4.0.0^{2,7}]pentadeca-1(11),2(7),3,5,12,14-hexaene-13-carbonitrile;
1-98: 10-[2,6-difluoro-4-({2-[(2-hydroxyethyl)amino]ethyl}amino)phenyl]-8-ethyl-9-oxo-4-(trifluoromethyl)-6,8,10-triazatricyclo[9.4.0.0^{2,7}]pentadeca-1(11),2(7),3,5,12,14-hexaene-13-carbonitrile;
1-99: 4-chloro-10-(2,6-difluoro-4-{[2-(methylamino)ethyl]amino}phenyl)-8-ethyl-15-fluoro-9-oxo-6,8,10-triazatricyclo[9.4.0.0^{2,7}]pentadeca-1(11),2(7),3,5,12,14-hexaene-13-carbonitrile;
1-100: 4-chloro-10-[2,6-difluoro-4-({2-[(2-hydroxyethyl)amino]ethyl}amino)phenyl]-8-ethyl-15-fluoro-9-oxo-6,8,10-triazatricyclo[9.4.0.0^{2,7}]pentadeca-1(11),2(7),3,5,12,14-hexaene-13-carbonitrile;
1-101: 4-({2-[(4-{4-chloro-13-cyano-8-ethyl-15-fluoro-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)butanoic acid;
1-102: 10-(2,6-difluoro-4-{[2-(methylamino)ethyl]amino}phenyl)-8-ethyl-4,15-difluoro-9-oxo-6,8,10-triazatricyclo[9.4.0.0^{2,7}]pentadeca-1(11),2(7),3,5,12,14-hexaene-13-carbonitrile;
1-103: 10-[2,6-difluoro-4-({2-[(2-hydroxyethyl)amino]ethyl}amino)phenyl]-8-ethyl-4,15-difluoro-9-oxo-6,8,10-triazatricyclo[9.4.0.0^{2,7}]pentadeca-1(11),2(7),3,5,12,14-hexaene-13-carbonitrile;
1-104: 4-({2-[(4-{13-cyano-8-ethyl-4,15-difluoro-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)butanoic acid;
1-105: 13-chloro-10-(2,6-difluoro-4-{[2-(methylamino)ethyl]amino}phenyl)-4-fluoro-8-(propan-2-yl)-6,8,10-triazatricyclo[9.4.0.0^{2,7}]pentadeca-1(11),2(7),3,5,12,14-hexaen-9-one;
1-106: 13-chloro-10-[2,6-difluoro-4-({2-[(2-hydroxyethyl)amino]ethyl}amino)phenyl]-4-fluoro-8-(propan-2-yl)-6,8,10-triazatricyclo[9.4.0.0^{2,7}]pentadeca-1(11),2(7),3,5,12,14-hexaen-9-one;
1-107: 10-(2,6-difluoro-4-{[2-(methylamino)ethyl]amino}phenyl)-4,15-difluoro-9-oxo-8-(propan-2-yl)-6,8,10-triazatricyclo[9.4.0.0^{2,7}]pentadeca-1(11),2(7),3,5,12,14-hexaene-13-carbonitrile;
1-108: 10-[2,6-difluoro-4-({2-[(2-hydroxyethyl)amino]ethyl}amino)phenyl]-4,15-difluoro-9-oxo-8-(propan-2-yl)-6,8,10-triazatricyclo[9.4.0.0^{2,7}]pentadeca-1(11),2(7),3,5,12,14-hexaene-13-carbonitrile;
1-109: 10-(2,6-difluoro-4-{[2-(methylamino)ethyl]amino}phenyl)-4,15-difluoro-8-methyl-9-oxo-6,8,10-triazatricyclo[9.4.0.0^{2,7}]pentadeca-1(11),2(7),3,5,12,14-hexaene-13-carbonitrile;
1-110: 10-[2,6-difluoro-4-({2-[(2-hydroxyethyl)amino]ethyl}amino)phenyl]-4,15-difluoro-8-methyl-9-oxo-6,8,10-triazatricyclo[9.4.0.0^{2,7}]pentadeca-1(11),2(7),3,5,12,14-hexaene-13-carbonitrile;
1-111: 10-(2,6-difluoro-4-{[2-(methylamino)ethyl]amino}phenyl)-4-fluoro-8,15-dimethyl-9-oxo-6,8,10-triazatricyclo[9.4.0.0^{2,7}]pentadeca-1(11),2(7),3,5,12,14-hexaene-13-carbonitrile;
1-112: 10-[2,6-difluoro-4-({2-[(2-hydroxyethyl)amino]ethyl}amino)phenyl]-4-fluoro-8,15-dimethyl-9-oxo-6,8,10-triazatricyclo[9.4.0.0^{2,7}]pentadeca-1(11),2(7),3,5,12,14-hexaene-13-carbonitrile;
1-113: 13-chloro-10-(2,6-difluoro-4-{[2-(methylamino)ethyl]amino}phenyl)-4,15-difluoro-8-(propan-2-yl)-6,8,10-triazatricyclo[9.4.0.0^{2,7}]pentadeca-1(11),2(7),3,5,12,14-hexaen-9-one;
1-114: 13-chloro-10-[2,6-difluoro-4-({2-[(2-hydroxyethyl)amino]ethyl}amino)phenyl]-4,15-difluoro-8-(propan-2-yl)-6,8,10-triazatricyclo[9.4.0.0^{2,7}]pentadeca-1(11),2(7),3,5,12,14-hexaen-9-one;
1-115: 4-chloro-10-(2,6-difluoro-4-{[2-(methylamino)ethyl]amino}phenyl)-15-fluoro-8-methyl-9-oxo-6,8,10-triazatricyclo[9.4.0.0^{2,7}]pentadeca-1(11),2(7),3,5,12,14-hexaene-13-carbonitrile;
1-116: 4-chloro-10-[2,6-difluoro-4-({2-[(2-hydroxyethyl)amino]ethyl}amino)phenyl]-15-fluoro-8-methyl-9-oxo-6,8,10-triazatricyclo[9.4.0.0^{2,7}]pentadeca-1(11),2(7),3,5,12,14-hexaene-13-carbonitrile;
1-117: 10-(2,6-difluoro-4-{[2-(methylamino)ethyl]amino}phenyl)-4-fluoro-9-oxo-8-propyl-6,8,10-triazatricyclo[9.4.0.0^{2,7}]pentadeca-1(11),2(7),3,5,12,14-hexaene-13-carbonitrile;
1-118: 10-[2,6-difluoro-4-({2-[(2-hydroxyethyl)amino]ethyl}amino)phenyl]-4-fluoro-9-oxo-8-propyl-6,8,10-triazatricyclo[9.4.0.0^{2,7}]pentadeca-1(11),2(7),3,5,12,14-hexaene-13-carbonitrile;
1-119: 4-chloro-10-(2,6-difluoro-4-{[2-(methylamino)ethyl]amino}phenyl)-9-oxo-8-propyl-6,8,10-triazatricyclo[9.4.0.0^{2,7}]pentadeca-1(11),2(7),3,5,12,14-hexaene-13-carbonitrile;
1-120: 4-chloro-10-[2,6-difluoro-4-({2-[(2-hydroxyethyl)amino]ethyl}amino)phenyl]-9-oxo-8-propyl-6,8,10-triazatricyclo[9.4.0.0^{2,7}]pentadeca-1(11),2(7),3,5,12,14-hexaene-13-carbonitrile;
1-121: 10-(2,6-difluoro-4-{[2-(methylamino)ethyl]amino}phenyl)-4-fluoro-8-(2-hydroxyethyl)-9-oxo-6,8,10-triazatricyclo[9.4.0.0^{2,7}]pentadeca-1(11),2(7),3,5,12,14-hexaene-13-carbonitrile;
1-122: 10-(2,6-difluoro-4-{[2-(methylamino)ethyl]amino}phenyl)-4-fluoro-8-(2-methoxyethyl)-9-oxo-6,8,10-triazatricyclo[9.4.0.0^{2,7}]pentadeca-1(11),2(7),3,5,12,14-hexaene-13-carbonitrile;
1-123: 10-{4-[3-(aminomethyl)azetidin-1-yl]-2,6-difluorophenyl}-8-ethyl-4-fluoro-9-oxo-6,8,10-triazatricyclo[9.4.0.0^{2,7}]pentadeca-1(11),2(7),3,5,12,14-hexaene-13-carbonitrile;
1-124: 10-(2,6-difluoro-4-{3-[(2-hydroxyethyl)amino]azetidin-1-yl}phenyl)-8-ethyl-4-fluoro-9-oxo-6,8,10-triazatricyclo[9.4.0.0^{2,7}]pentadeca-1(11),2(7),3,5,12,14-hexaene-13-carbonitrile;
1-125: 10-[2,6-difluoro-4-({2-[(2-hydroxyethyl)amino]ethyl}amino)phenyl]-4,15-difluoro-9-oxo-8-propyl-6,8,10-triazatricyclo[9.4.0.0^{2,7}]pentadeca-1(11),2(7),3,5,12,14-hexaene-13-carbonitrile;
1-126: 10-{4-[(2-aminoethyl)amino]-2,6-difluorophenyl}-4-chloro-8-ethyl-9-oxo-6,8,10-triazatricyclo[9.4.0.0^{2,7}]pentadeca-1(11),2(7),3,5,12,14-hexaene-13-carbonitrile;
1-127: 4-chloro-10-{2,6-difluoro-4-[(2-{[2-(methylamino)ethyl]amino}ethyl)amino]phenyl}-8-ethyl-9-oxo-6,8,10-triazatricyclo[9.4.0.0^{2,7}]pentadeca-1(11),2(7),3,5,12,14-hexaene-13-carbonitrile;
1-128: 4-chloro-10-(2,6-difluoro-4-{[2-(3-oxopiperazin-1-yl)ethyl]amino}phenyl)-8-ethyl-9-oxo-6,8,10-triazatricyclo[9.4.0.0^{2,7}]pentadeca-1(11),2(7),3,5,12,14-hexaene-13-carbonitrile;
1-129: 4-chloro-10-[2,6-difluoro-4-({2-[(2-hydroxyethyl)amino]ethyl}amino)phenyl]-8-ethyl-13-fluoro-6,8,10-triazatricyclo[9.4.0.0^{2,7}]pentadeca-1(11),2(7),3,5,12,14-hexaen-9-one;
1-130: 4-chloro-10-(2,6-difluoro-4-{[2-(methylamino)ethyl]amino}phenyl)-8-ethyl-13-fluoro-6,8,10-triazatricyclo[9.4.0.0^{2,7}]pentadeca-1(11),2(7),3,5,12,14-hexaen-9-one;
1-131: 10-[4-({2-[(2-aminoethyl)amino]ethyl}amino)-2,6-difluorophenyl]-4-chloro-8-ethyl-9-oxo-6,8,10-triazatricyclo[9.4.0.0^{2,7}]pentadeca-1(11),2(7),3,5,12,14-hexaene-13-carbonitrile;
1-132: 4-chloro-10-{4-[(2-{[2-(dimethylamino)ethyl]amino}ethyl)amino]-2,6-difluorophenyl}-8-ethyl-9-oxo-6,8,10-triazatricyclo[9.4.0.0^{2,7}]pentadeca-1(11),2(7),3,5,12,14-hexaene-13-carbonitrile;
1-133: 4-chloro-8-ethyl-10-(4-{[2-(ethylamino)ethyl]amino}-2,6-difluorophenyl)-9-oxo-6,8,10-triazatricyclo[9.4.0.0^{2,7}]pentadeca-1(11),2(7),3,5,12,14-hexaene-13-carbonitrile;
1-134: 13-chloro-10-[2,6-difluoro-4-({2-[(2-hydroxyethyl)amino]ethyl}amino)phenyl]-8-ethyl-4-fluoro-9-oxo-6,8,10-triazatricyclo[9.4.0.0^{2,7}]pentadeca-1(11),2(7),3,5,12,14-hexaene-15-carbonitrile;
1-135: 4-chloro-10-(2,6-difluoro-4-{[2-(methylamino)ethyl]amino}phenyl)-8-ethyl-6,8,10-triazatricyclo[9.4.0.0^{2,7}]pentadeca-1(11),2(7),3,5,12,14-hexaen-9-one;
1-136: 4-chloro-10-[2,6-difluoro-4-(piperazin-1-yl)phenyl]-8-ethyl-9-oxo-6,8,10-triazatricyclo[9.4.0.0^{2,7}]pentadeca-1(11),2(7),3,5,12,14-hexaene-13-carbonitrile;
1-137: 4-chloro-10-[2,6-difluoro-4-({2-[(2-hydroxypropyl)amino]ethyl}amino)phenyl]-8-ethyl-9-oxo-6,8,10-triazatricyclo[9.4.0.0^{2,7}]pentadeca-1(11),2(7),3,5,12,14-hexaene-13-carbonitrile;
1-138: 4-chloro-10-[2,6-difluoro-4-({2-[(2-hydroxy-2-methylpropyl)amino]ethyl}amino)phenyl]-8-ethyl-9-oxo-6,8,10-triazatricyclo[9.4.0.0^{2,7}]pentadeca-1(11),2(7),3,5,12,14-hexaene-13-carbonitrile;
1-139: 4-chloro-10-[2,6-difluoro-4-({2-[(1-hydroxybutan-2-yl)amino]ethyl}amino)phenyl]-8-ethyl-9-oxo-6,8,10-triazatricyclo[9.4.0.0^{2,7}]pentadeca-1(11),2(7),3,5,12,14-hexaene-13-carbonitrile;
1-140: [2-({2-[(4-{4-chloro-13-cyano-8-ethyl-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)ethyl]urea;
1-141: 2-({2-[(4-{4-chloro-13-cyano-8-ethyl-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)ethane-1-sulfonic acid;
1-142: 4-chloro-10-[2,6-difluoro-4-({2-[(2-hydroxyethyl)amino]ethyl}amino)phenyl]-8-ethyl-6,8,10-triazatricyclo[9.4.0.0^{2,7}]pentadeca-1(11),2(7),3,5,12,14-hexaen-9-one;
1-143: 4-chloro-10-[4-(1,4-diazepan-1-yl)-2,6-difluorophenyl]-8-ethyl-9-oxo-6,8,10-triazatricyclo[9.4.0.0^{2,7}]pentadeca-1(11),2(7),3,5,12,14-hexaene-13-carbonitrile;
1-144: 4-chloro-10-{2,6-difluoro-4-[4-(2-hydroxyethyl)piperazin-1-yl]phenyl}-8-ethyl-9-oxo-6,8,10-triazatricyclo[9.4.0.0^{2,7}]pentadeca-1(11),2(7),3,5,12,14-hexaene-13-carbonitrile;
1-145: 10-(4-{[(azetidin-3-yl)methyl]amino}-2,6-difluorophenyl)-4-chloro-8-ethyl-9-oxo-6,8,10-triazatricyclo[9.4.0.0^{2,7}]pentadeca-1(11),2(7),3,5,12,14-hexaene-13-carbonitrile;
1-146: 4-chloro-10-(2,6-difluoro-4-{methyl[2-(methylamino)ethyl]amino}phenyl)-8-ethyl-9-oxo-6,8,10-triazatricyclo[9.4.0.0^{2,7}]pentadeca-1(11),2(7),3,5,12,14-hexaene-13-carbonitrile;
1-147: 4-chloro-10-[2,6-difluoro-4-({[(3S)-morpholin-3-yl]methyl}amino)phenyl]-8-ethyl-9-oxo-6,8,10-triazatricyclo[9.4.0.0^{2,7}]pentadeca-1(11),2(7),3,5,12,14-hexaene-13-carbonitrile;
1-148: 4-chloro-10-(2,6-difluoro-4-{[2-(morpholin-4-yl)ethyl]amino}phenyl)-8-ethyl-9-oxo-6,8,10-triazatricyclo[9.4.0.0^{2,7}]pentadeca-1(11),2(7),3,5,12,14-hexaene-13-carbonitrile;
1-149: 4-chloro-10-[2,6-difluoro-4-({2-[(1-hydroxypropan-2-yl)amino]ethyl}amino)phenyl]-8-ethyl-9-oxo-6,8,10-triazatricyclo[9.4.0.0^{2,7}]pentadeca-1(11),2(7),3,5,12,14-hexaene-13-carbonitrile;
1-150: N-[2-({2-[(4-{4-chloro-13-cyano-8-ethyl-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)ethyl]methanesulfonamide;
1-151: 4-chloro-10-[2,6-difluoro-4-({[(3R)-morpholin-3-yl]methyl}amino)phenyl]-8-ethyl-9-oxo-6,8,10-triazatricyclo[9.4.0.0^{2,7}]pentadeca-1(11),2(7),3,5,12,14-hexaene-13-carbonitrile;
1-152: 4-chloro-10-{2,6-difluoro-4-[(3S)-3-(hydroxymethyl)piperazin-1-yl]phenyl}-8-ethyl-9-oxo-6,8,10-triazatricyclo[9.4.0.0^{2,7}]pentadeca-1(11),2(7),3,5,12,14-hexaene-13-carbonitrile;
1-153: 4-chloro-10-{2,6-difluoro-4-[(3R)-3-(hydroxymethyl)piperazin-1-yl]phenyl}-8-ethyl-9-oxo-6,8,10-triazatricyclo[9.4.0.0^{2,7}]pentadeca-1(11),2(7),3,5,12,14-hexaene-13-carbonitrile;
1-154: 4-chloro-10-[4-({2-[(2-cyanoethyl)amino]ethyl}amino)-2,6-difluorophenyl]-8-ethyl-9-oxo-6,8,10-triazatricyclo[9.4.0.0^{2,7}]pentadeca-1(11),2(7),3,5,12,14-hexaene-13-carbonitrile;
1-155: 4-chloro-10-(2,6-difluoro-4-{[2-(piperazin-1-yl)ethyl]amino}phenyl)-8-ethyl-9-oxo-6,8,10-triazatricyclo[9.4.0.0^{2,7}]pentadeca-1(11),2(7),3,5,12,14-hexaene-13-carbonitrile;
1-156: 4-chloro-10-[4-({2-[(2,3-dihydroxypropyl)amino]ethyl}amino)-2,6-difluorophenyl]-8-ethyl-9-oxo-6,8,10-triazatricyclo[9.4.0.0^{2,7}]pentadeca-1(11),2(7),3,5,12,14-hexaene-13-carbonitrile;
1-157: 4-chloro-10-[4-({2-[(1,3-dihydroxypropan-2-yl)amino]ethyl}amino)-2,6-difluorophenyl]-8-ethyl-9-oxo-6,8,10-triazatricyclo[9.4.0.0^{2,7}]pentadeca-1(11),2(7),3,5,12,14-hexaene-13-carbonitrile;
1-158: 4-chloro-10-[2,6-difluoro-4-({[3-(hydroxymethyl)azetidin-3-yl]methyl}amino)phenyl]-8-ethyl-9-oxo-6,8,10-triazatricyclo[9.4.0.0^{2,7}]pentadeca-1(11),2(7),3,5,12,14-hexaene-13-carbonitrile;
1-159: 4-chloro-10-(2,6-difluoro-4-{[(2R)-1-(methylamino)propan-2-yl]amino}phenyl)-8-ethyl-9-oxo-6,8,10-triazatricyclo[9.4.0.0^{2,7}]pentadeca-1(11),2(7),3,5,12,14-hexaene-13-carbonitrile;
1-160: 4-chloro-10-[4-({2-[(cyanomethyl)amino]ethyl}amino)-2,6-difluorophenyl]-8-ethyl-9-oxo-6,8,10-triazatricyclo[9.4.0.0^{2,7}]pentadeca-1(11),2(7),3,5,12,14-hexaene-13-carbonitrile;
1-161: 3-({2-[(4-{4-chloro-13-cyano-8-ethyl-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)propanamide;
1-162: 2-({2-[(4-{4-chloro-13-cyano-8-ethyl-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)acetamide;
1-163: 4-{[2-({4-[13-cyano-4,15-difluoro-9-oxo-8-(propan-2-yl)-6,8,10-triazatricyclo[9.4.0.0^{2,7}]pentadeca-1(15),2(7),3,5,11,13-hexaen-10-yl]-3,5-difluorophenyl}amino)ethyl]amino}butanoic acid;
1-164: 4-chloro-10-(2,6-difluoro-4-{[2-(3-hydroxypyrrolidin-1-yl)ethyl]amino}phenyl)-8-ethyl-9-oxo-6,8,10-triazatricyclo[9.4.0.0^{2,7}]pentadeca-1(11),2(7),3,5,12,14-hexaene-13-carbonitrile;
1-165: 4-chloro-10-(2,6-difluoro-4-{[(2S)-1-(methylamino)propan-2-yl]amino}phenyl)-8-ethyl-9-oxo-6,8,10-triazatricyclo[9.4.0.0^{2,7}]pentadeca-1(11),2(7),3,5,12,14-hexaene-13-carbonitrile;
1-166: 1-[2-({2-[(4-{4-chloro-13-cyano-8-ethyl-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)ethyl]-3-methylurea;
1-167: methyl N-[2-({2-[(4-{4-chloro-13-cyano-8-ethyl-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)ethyl]carbamate;
1-168: N-[2-({2-[(4-{4-chloro-13-cyano-8-ethyl-9-oxo-6,8,10-triazatricyclo[9.4.0.0^{2,7}]pentadeca-1(15),2(7),3,5,11,13-hexaen-10-yl}-3,5-difluorophenyl)amino]ethyl}amino)ethyl]acetamide;
1-169: [2-({2-[(4-{13-cyano-8-ethyl-4-fluoro-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)ethyl]urea;
1-170: 4-chloro-10-(2,6-difluoro-4-{[2-({[(2R)-5-oxopyrrolidin-2-yl]methyl}amino)ethyl]amino}phenyl)-8-ethyl-9-oxo-6,8,10-triazatricyclo[9.4.0.0^{2,7}]pentadeca-1(11),2(7),3,5,12,14-hexaene-13-carbonitrile;
1-171: 4-chloro-10-{4-[(2-{[(2S)-2,3-dihydroxypropyl]amino}ethyl)amino]-2,6-difluorophenyl}-8-ethyl-9-oxo-6,8,10-triazatricyclo[9.4.0.0^{2,7}]pentadeca-1(11),2(7),3,5,12,14-hexaene-13-carbonitrile;
1-172: 1-[2-({2-[(4-{13-cyano-8-ethyl-4-fluoro-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)ethyl]-3-methylurea;
1=173: 4-chloro-10-{2,6-difluoro-4-[(2-{[(2R)-1-hydroxypropan-2-yl]amino}ethyl)amino]phenyl}-8-ethyl-9-oxo-6,8,10-triazatricyclo[9.4.0.0^{2,7}]pentadeca-1(11),2(7),3,5,12,14-hexaene-13-carbonitrile;
1-174: 4-chloro-10-(2,6-difluoro-4-{[2-({[(2S)-5-oxopyrrolidin-2-yl]methyl}amino)ethyl]amino}phenyl)-8-ethyl-9-oxo-6,8,10-triazatricyclo[9.4.0.0^{2,7}]pentadeca-1(11),2(7),3,5,12,14-hexaene-13-carbonitrile;
1-175: 3-({2-[(4-{4-chloro-13-cyano-8-ethyl-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)propane-1-sulfonic acid;
1-176: 4-chloro-10-{2,6-difluoro-4-[(2-{[(5-oxopyrrolidin-2-yl)methyl]amino}ethyl)amino]phenyl}-8-ethyl-9-oxo-6,8,10-triazatricyclo[9.4.0.0^{2,7}]pentadeca-1(15),2(7),3,5,11,13-hexaene-13-carbonitrile;
1-177: 4-chloro-10-{2,6-difluoro-4-[(2-{[(1R,2S)-2-hydroxycyclobutyl]amino}ethyl)amino]phenyl}-8-ethyl-9-oxo-6,8,10-triazatricyclo[9.4.0.0^{2,7}]pentadeca-1(11),2(7),3,5,12,14-hexaene-13-carbonitrile;
1-178: 3-({2-[(4-{4-chloro-13-cyano-8-ethyl-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)propanoic acid;
1-179: 4-chloro-10-{2,6-difluoro-4-[(2-{[(1R,2R)-2-hydroxycyclobutyl]amino}ethyl)amino]phenyl}-8-ethyl-9-oxo-6,8,10-triazatricyclo[9.4.0.0^{2,7}]pentadeca-1(11),2(7),3,5,12,14-hexaene-13-carbonitrile;
1-180: 4-chloro-10-{2,6-difluoro-4-[(2-{[(2S)-2-hydroxypropyl]amino}ethyl)amino]phenyl}-8-ethyl-9-oxo-6,8,10-triazatricyclo[9.4.0.0^{2,7}]pentadeca-1(11),2(7),3,5,12,14-hexaene-13-carbonitrile;
1-181: 4-chloro-10-{2,6-difluoro-4-[(2-{[2-(2-hydroxyethoxy)ethyl]amino}ethyl)amino]phenyl}-8-ethyl-9-oxo-6,8,10-triazatricyclo[9.4.0.0^{2,7}]pentadeca-1(11),2(7),3,5,12,14-hexaene-13-carbonitrile;
1-182: 4-chloro-10-(2,6-difluoro-4-{3-[(2-hydroxyethyl)amino]pyrrolidin-1-yl}phenyl)-8-ethyl-9-oxo-6,8,10-triazatricyclo[9.4.0.0^{2,7}]pentadeca-1(11),2(7),3,5,12,14-hexaene-13-carbonitrile;
1-183: 4-chloro-10-{4-[(2-{[(2R)-2,3-dihydroxypropyl]amino}ethyl)amino]-2,6-difluorophenyl}-8-ethyl-9-oxo-6,8,10-triazatricyclo[9.4.0.0^{2,7}]pentadeca-1(11),2(7),3,5,12,14-hexaene-13-carbonitrile;
1-184: 10-{2,6-difluoro-4-[(2-{[(1R,2S)-2-hydroxycyclobutyl]amino}ethyl)amino]phenyl}-8-ethyl-9-oxo-4-(trifluoromethyl)-6,8,10-triazatricyclo[9.4.0.0^{2,7}]pentadeca-1(11),2(7),3,5,12,14-hexaene-13-carbonitrile;
1-185: 4-chloro-10-{2,6-difluoro-4-[(2-{[(2S)-1-hydroxypropan-2-yl]amino}ethyl)amino]phenyl}-8-ethyl-9-oxo-6,8,10-triazatricyclo[9.4.0.0^{2,7}]pentadeca-1(11),2(7),3,5,12,14-hexaene-13-carbonitrile;
1-186: 4-chloro-10-{2,6-difluoro-4-[(2-{[(2R)-2-hydroxypropyl]amino}ethyl)amino]phenyl}-8-ethyl-9-oxo-6,8,10-triazatricyclo[9.4.0.0^{2,7}]pentadeca-1(11),2(7),3,5,12,14-hexaene-13-carbonitrile;
1-187: 4-chloro-10-{2,6-difluoro-4-[(2-{[(1r,3r)-3-hydroxycyclobutyl]amino}ethyl)amino]phenyl}-8-ethyl-9-oxo-6,8,10-triazatricyclo[9.4.0.0^{2,7}]pentadeca-1(11),2(7),3,5,12,14-hexaene-13-carbonitrile;
1-188: 10-[4-({2-[(3-amino-2-hydroxypropyl)amino]ethyl}amino)-2,6-difluorophenyl]-4-chloro-8-ethyl-9-oxo-6,8,10-triazatricyclo[9.4.0.0^{2,7}]pentadeca-1(11),2(7),3,5,12,14-hexaene-13-carbonitrile;
1-189: 2-({2-[(4-{4-chloro-13-cyano-8-ethyl-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)ethyl carbamate;
1-190: 4-chloro-10-[2,6-difluoro-4-({2-[(3-hydroxypropyl)amino]ethyl}amino)phenyl]-8-ethyl-9-oxo-6,8,10-triazatricyclo[9.4.0.0^{2,7}]pentadeca-1(11),2(7),3,5,12,14-hexaene-13-carbonitrile;
1-191: 4-chloro-10-{2,6-difluoro-4-[(2-{[2-(2-oxo-1,3-oxazolidin-3-yl)ethyl]amino}ethyl)amino]phenyl}-8-ethyl-9-oxo-6,8,10-triazatricyclo[9.4.0.0^{2,7}]pentadeca-1(11),2(7),3,5,12,14-hexaene-13-carbonitrile;
1-192: 4-chloro-10-{2,6-difluoro-4-[(2-{1[(1s,3s)-3-hydroxycyclobutyl]amino}ethyl)amino]phenyl}-8-ethyl-9-oxo-6,8,10-triazatricyclo[9.4.0.0^{2,7}]pentadeca-1(11),2(7),3,5,12,14-hexaene-13-carbonitrile;
1-193: (1r,4r)-4-({2-[(4-{4-chloro-13-cyano-8-ethyl-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)cyclohexane-1-carboxylic acid;
1-194: 10-{4-[(2-{[(2R)-2,3-dihydroxypropyl]amino}ethyl)amino]-2,6-difluorophenyl}-8-ethyl-4-fluoro-9-oxo-6,8,10-triazatricyclo[9.4.0.0^{2,7}]pentadeca-1(11),2(7),3,5,12,14-hexaene-13-carbonitrile;
1-195: 4-chloro-10-[2,6-difluoro-4-({2-[(2-hydroxyethyl)amino]ethyl}amino)phenyl]-8-methyl-9-oxo-6,8,10-triazatricyclo[9.4.0.0^{2,7}]pentadeca-1(11),2(7),3,5,12,14-hexaene-13-carbonitrile;
1-196: 10-{2,6-difluoro-4-[(2-{[(1R,2R)-2-hydroxycyclobutyl]amino}ethyl)amino]phenyl}-8-ethyl-9-oxo-4-(trifluoromethyl)-6,8,10-triazatricyclo[9.4.0.0^{2,7}]pentadeca-1(11),2(7),3,5,12,14-hexaene-13-carbonitrile;
1-197: 4-chloro-10-[2,6-difluoro-4-({2-[(3-hydroxypropyl)amino]ethyl}amino)phenyl]-8-methyl-9-oxo-6,8,10-triazatricyclo[9.4.0.0^{2,7}]pentadeca-1(11),2(7),3,5,12,14-hexaene-13-carbonitrile;
1-198: 4-chloro-10-{2,6-difluoro-4-[(2-{[(1r,3r)-3-hydroxycyclobutyl]amino}ethyl)amino]phenyl}-8-methyl-9-oxo-6,8,10-triazatricyclo[9.4.0.0^{2,7}]pentadeca-1(11),2(7),3,5,12,14-hexaene-13-carbonitrile;
1-199: 10-{2,6-difluoro-4-[(2-{[(2S)-2-hydroxypropyl]amino}ethyl)amino]phenyl}-8-ethyl-4-fluoro-9-oxo-6,8,10-triazatricyclo[9.4.0.0^{2,7}]pentadeca-1(11),2(7),3,5,12,14-hexaene-13-carbonitrile;
1-200: 10-{4-[(2-{[(2S)-2,3-dihydroxypropyl]amino}ethyl)amino]-2,6-difluorophenyl}-8-ethyl-4-fluoro-9-oxo-6,8,10-triazatricyclo[9.4.0.0^{2,7}]pentadeca-1(11),2(7),3,5,12,14-hexaene-13-carbonitrile;
1-201: 4-({2-[(4-{4-chloro-13-cyano-8-methyl-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)butanoic acid;
1-202: (3R)-3-({2-[(4-{4-chloro-13-cyano-8-ethyl-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)-4-hydroxybutanoic acid;
1-203: 10-{2,6-difluoro-4-[(2-{[(2R)-2-hydroxypropyl]amino}ethyl)amino]phenyl}-8-ethyl-4-fluoro-9-oxo-6,8,10-triazatricyclo[9.4.0.0^{2,7}]pentadeca-1(11),2(7),3,5,12,14-hexaene-13-carbonitrile;
1-204: 10-{2,6-difluoro-4-[(2-{[(1R,2S)-2-hydroxycyclobutyl]amino}ethyl)amino]phenyl}-8-ethyl-4-fluoro-9-oxo-6,8,10-triazatricyclo[9.4.0.0^{2,7}]pentadeca-1(11),2(7),3,5,12,14-hexaene-13-carbonitrile;
1-205: 4-chloro-10-{2,6-difluoro-4-[(2-{[(2S)-1-hydroxypropan-2-yl]amino}ethyl)amino]phenyl}-8-methyl-9-oxo-6,8,10-triazatricyclo[9.4.0.0^{2,7}]pentadeca-1(11),2(7),3,5,12,14-hexaene-13-carbonitrile;
1-206: (1r,3r)-3-({2-[(4-{4-chloro-13-cyano-8-ethyl-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)cyclobutane-1-carboxylic acid;
1-207: 10-{2,6-difluoro-4-[(2-{[(1s,3s)-3-hydroxycyclobutyl]amino}ethyl)amino]phenyl}-8-ethyl-9-oxo-4-(trifluoromethyl)-6,8,10-triazatricyclo[9.4.0.0^{2,7}]pentadeca-1(11),2(7),3,5,12,14-hexaene-13-carbonitrile;
1-208: 10-[2,6-difluoro-4-({2-[(2-hydroxyethyl)amino]ethyl}amino)phenyl]-8-ethyl-4-methyl-9-oxo-6,8,10-triazatricyclo[9.4.0.0^{2,7}]pentadeca-1(11),2(7),3,5,12,14-hexaene-13-carbonitrile;
1-209: 4-({2-[(4-{4-chloro-13-cyano-8-ethyl-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)-2,2-dimethylbutanoic acid;
1-210: (2S)-2-({2-[(4-{4-chloro-13-cyano-8-ethyl-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)-3-hydroxypropanoic acid;
1-211: 4-chloro-10-(2,6-difluoro-4-{[2-(methylamino)ethyl]amino}phenyl)-8-methyl-9-oxo-6,8,10-triazatricyclo[9.4.0.0^{2,7}]pentadeca-1(11),2(7),3,5,12,14-hexaene-13-carbonitrile;
1-212: 10-{4-[(3R)-3-aminopyrrolidin-1-yl]-2,6-difluorophenyl}-4,15-difluoro-9-oxo-8-(propan-2-yl)-6,8,10-triazatricyclo[9.4.0.0^{2,7}]pentadeca-1(15),2(7),3,5,11,13-hexaene-13-carbonitrile;
1-213: 10-{2,6-difluoro-4-[(2-{[(2S)-1-hydroxypropan-2-yl]amino}ethyl)amino]phenyl}-8-ethyl-4-fluoro-9-oxo-6,8,10-triazatricyclo[9.4.0.0^{2,7}]pentadeca-1(11),2(7),3,5,12,14-hexaene-13-carbonitrile;
1-214: 10-{2,6-difluoro-4-[(2-{[(2R)-1-hydroxypropan-2-yl]amino}ethyl)amino]phenyl}-8-ethyl-4-fluoro-9-oxo-6,8,10-triazatricyclo[9.4.0.0^{2,7}]pentadeca-1(11),2(7),3,5,12,14-hexaene-13-carbonitrile;
1-215: 2-({2-[(4-{4-chloro-13-cyano-8-ethyl-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)acetic acid;
1-216: methyl N-[2-({2-[(4-{13-cyano-8-ethyl-4-fluoro-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)ethyl]carbamate;
1-217: 3-({2-[(4-{4-chloro-13-cyano-8-methyl-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)propanoic acid;
1-218: 10-{2,6-difluoro-4-[(2-{[(1R,2R)-2-hydroxycyclobutyl]amino}ethyl)amino]phenyl}-8-ethyl-4-fluoro-9-oxo-6,8,10-triazatricyclo[9.4.0.0^{2,7}]pentadeca-1(11),2(7),3,5,12,14-hexaene-13-carbonitrile;
1-219: (2S)-2-({2-[(4-{4-chloro-13-cyano-8-ethyl-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)propanoic acid;
1-220: (2R)-2-({2-[(4-{4-chloro-13-cyano-8-ethyl-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)-3-hydroxypropanoic acid;
1-221: 3-({2-[(4-{13-cyano-8-ethyl-4-fluoro-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)propane-1-sulfonic acid;
1-222: 10-{2,6-difluoro-4-[(2-{[(1r,3r)-3-hydroxycyclobutyl]amino}ethyl)amino]phenyl}-8-ethyl-4-fluoro-9-oxo-6,8,10-triazatricyclo[9.4.0.0^{2,7}]pentadeca-1(11),2(7),3,5,12,14-hexaene-13-carbonitrile;
1-223: 10-{4-[(2-aminoethyl)amino]-2,6-difluorophenyl}-4-chloro-8-methyl-9-oxo-6,8,10-triazatricyclo[9.4.0.0^{2,7}]pentadeca-1(11),2(7),3,5,12,14-hexaene-13-carbonitrile;
1-224: 10-{2,6-difluoro-4-[(2-{[(1r,3r)-3-hydroxycyclobutyl]amino}ethyl)amino]phenyl}-8-ethyl-9-oxo-4-(trifluoromethyl)-6,8,10-triazatricyclo[9.4.0.0^{2,7}]pentadeca-1(11),2(7),3,5,12,14-hexaene-13-carbonitrile;
1-225: 10-[4-({2-[(2-amino-3-hydroxypropyl)amino]ethyl}amino)-2,6-difluorophenyl]-4-chloro-8-ethyl-9-oxo-6,8,10-triazatricyclo[9.4.0.0^{2,7}]pentadeca-1(11),2(7),3,5,12,14-hexaene-13-carbonitrile;
1-226: 10-[2,6-difluoro-4-({2-[(2-hydroxyethyl)amino]ethyl}amino)phenyl]-8-ethyl-4-fluoro-13-methyl-6,8,10-triazatricyclo[9.4.0.0^{2,7}]pentadeca-1(11),2(7),3,5,12,14-hexaen-9-one;
1-227: 2-{[2-({4-[13-cyano-8-ethyl-9-oxo-4-(trifluoromethyl)-6,8,10-triazatricyclo[9.4.0.0^{2,7}]pentadeca-1(11),2(7),3,5,12,14-hexaen-10-yl]-3,5-difluorophenyl}amino)ethyl]amino}acetic acid;
1-228: 4-chloro-10-{2,6-difluoro-4-[(2-{[2-(2-oxoimidazolidin-1-yl)ethyl]amino}ethyl)amino]phenyl}-8-ethyl-9-oxo-6,8,10-triazatricyclo[9.4.0.0^{2,7}]pentadeca-1(11),2(7),3,5,12,14-hexaene-13-carbonitrile;
1-229: 3-[2-({2-[(4-{4-chloro-13-cyano-8-ethyl-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)ethoxy]propanoic acid;
1-230: 4-chloro-10-{2,6-difluoro-4-[(2-{[(2R)-2-hydroxy-2-phenylethyl]amino}ethyl)amino]phenyl}-8-ethyl-9-oxo-6,8,10-triazatricyclo[9.4.0.0^{2,7}]pentadeca-1(11),2(7),3,5,12,14-hexaene-13-carbonitrile;
1-231: 4-chloro-10-[2,6-difluoro-4-({2-[(3R)-3-hydroxypyrrolidin-1-yl]ethyl}amino)phenyl]-8-ethyl-9-oxo-6,8,10-triazatricyclo[9.4.0.0^{2,7}]pentadeca-1(11),2(7),3,5,12,14-hexaene-13-carbonitrile;
1-232: 10-{2,6-difluoro-4-[(2-{[(1s,3s)-3-hydroxycyclobutyl]amino}ethyl)amino]phenyl}-8-ethyl-4-fluoro-9-oxo-6,8,10-triazatricyclo[9.4.0.0^{2,7}]pentadeca-1(11),2(7),3,5,12,14-hexaene-13-carbonitrile;
1-233: (2S)-2-({2-[(4-{4-chloro-13-cyano-8-ethyl-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)-4-(methylsulfanyl)butanoic acid;
1-234: 10-{4-[(azetidin-3-yl)amino]-2,6-difluorophenyl}-4-chloro-8-ethyl-9-oxo-6,8,10-triazatricyclo[9.4.0.0^{2,7}]pentadeca-1(11),2(7),3,5,12,14-hexaene-13-carbonitrile;
1-235: 4-chloro-10-{2,6-difluoro-4-[(3S)-3-(methylamino)pyrrolidin-1-yl]phenyl}-8-ethyl-9-oxo-6,8,10-triazatricyclo[9.4.0.0^{2,7}]pentadeca-1(11),2(7),3,5,12,14-hexaene-13-carbonitrile;
1-236: (2R)-2-({2-[(4-{4-chloro-13-cyano-8-ethyl-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)propanoic acid;
1-237: 10-[4-({2-[(2-aminoethyl)amino]ethyl}amino)-2,6-difluorophenyl]-8-ethyl-4-fluoro-9-oxo-6,8,10-triazatricyclo[9.4.0.0^{2,7}]pentadeca-1(11),2(7),3,5,12,14-hexaene-13-carbonitrile;
1-238: 4-chloro-10-{2,6-difluoro-4-[(2-{[(pyrrolidin-2-yl)methyl]amino}ethyl)amino]phenyl}-8-ethyl-9-oxo-6,8,10-triazatricyclo[9.4.0.0^{2,7}]pentadeca-1(15),2(7),3,5,11,13-hexaene-13-carbonitrile;
1-239: 4-chloro-10-{2,6-difluoro-4-[(2-{[(2S)-2-hydroxy-2-phenylethyl]amino}ethyl)amino]phenyl}-8-ethyl-9-oxo-6,8,10-triazatricyclo[9.4.0.0^{2,7}]pentadeca-1(11),2(7),3,5,12,14-hexaene-13-carbonitrile;
1-240: (2S)-2-({2-[(4-{4-chloro-13-cyano-8-ethyl-9-oxo-6,8,10-triazatricyclo[l9.4.0.0^{2,7}]pentadeca-1(11),2(7),3,5,12,14-hexaen-10-yl}-3,5-difluorophenyl)amino]ethyl}amino)-4-methylpentanoic acid;
1-241: 10-{4-[(3 S)-3-aminopyrrolidin-1-yl]-2,6-difluorophenyl}-4-chloro-8-ethyl-9-oxo-6,8,10-triazatricyclo[9.4.0.0^{2,7}]pentadeca-1(11),2(7),3,5,12,14-hexaene-13-carbonitrile; and
1-242: 2-({2-[(4-{4,13-dichloro-8-ethyl-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)acetic acid.

In some embodiments, provided herein is a pharmaceutically acceptable salt of a compound that is described in Table 1.

TABLE 2

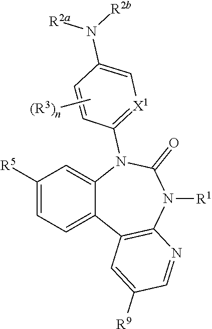

| Cpd | | | | | |
|---|---|---|---|---|---|
| No. | R9 | R5 | R1 | 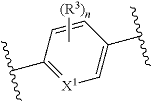 | 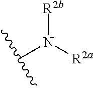 |
| 2-1 | H | Cl | 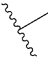 | 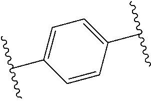 [SMILES: *c1ccc(*)cc1] | 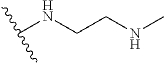 [SMILES: *NCCNC] |
| 2-2 | H | Cl | 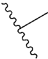 | 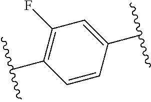 [SMILES: *c1ccc(*)c(F)c1] | 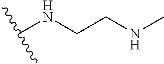 [SMILES: *NCCNC] |
| 2-3 | Cl | Cl | 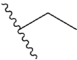 [SMILES: *CC] | 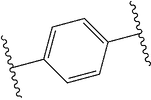 [SMILES: *c1ccc(*)cc1] | 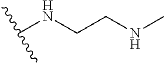 [SMILES: *NCCNC] |
| 2-4 | F | Cl | 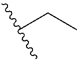 [SMILES: *CC] | 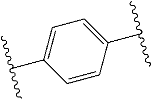 [SMILES: *c1ccc(*)cc1] | 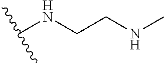 [SMILES: *NCCNC] |
| 2-5 | -CH₃ | Cl | 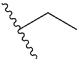 [SMILES: *CC] | 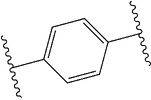 [SMILES: *c1ccc(*)cc1] | 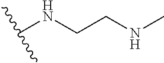 [SMILES: *NCCNC] |
| 2-6 | Cl | -OCH₃ | 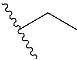 [SMILES: *CC] | 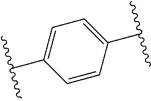 [SMILES: *c1ccc(*)cc1] | 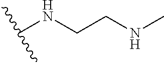 [SMILES: *NCCNC] |
| 2-7 | Cl | Cl | 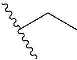 [SMILES: *CC] | 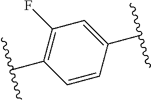 [SMILES: *c1ccc(*)c(F)c1] | 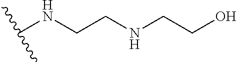 [SMILES: *NCCNCCO] |
| 2-8 | Cl | Cl | 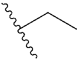 [SMILES: *CC] | 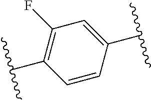 [SMILES: *c1ccc(*)c(F)c1] | 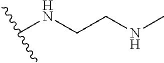 [SMILES: *NCCNC] |
| 2-9 | Cl | Cl | 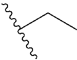 [SMILES: *CC] | 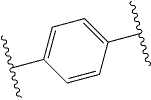 [SMILES: *c1ccc(*)cc1] | 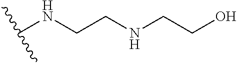 [SMILES: *NCCNCCO] |
| 2-10 | Cl | Cl | 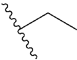 [SMILES: *CC] | 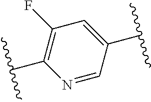 [SMILES: *c1cnc(*)c(F)c1] | 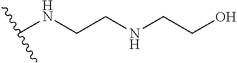 [SMILES: *NCCNCCO] |
| 2-11 | Cl | Cl | 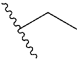 [SMILES: *CC] | 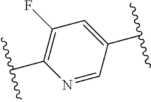 [SMILES: *c1cnc(*)c(F)c1] | 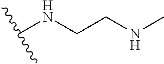 [SMILES: *NCCNC] |
| 2-12 | Cl | Cl | 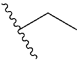 [SMILES: *CC] | 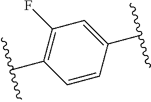 [SMILES: *c1ccc(*)c(F)c1] | 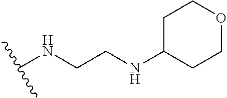 [SMILES: *NCCNC1CCOCC1] |
| 2-13 | Cl | Cl | 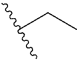 [SMILES: *CC] | 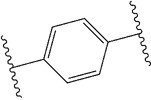 [SMILES: *c1ccc(*)cc1] | 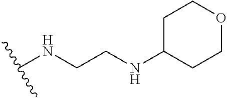 [SMILES: *NCCNC1CCOCC1] |
| 2-14 | Cl | Cl | 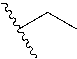 [SMILES: *CC] | 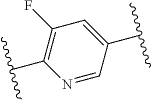 [SMILES: *c1cnc(*)c(F)c1] | 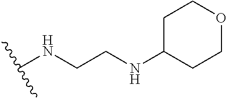 [SMILES: *NCCNC1CCOCC1] |

Compounds in Table 2 are named:
2-1: 13-chloro-8-methyl-10-(4-{[2-(methylamino)ethyl]amino}phenyl)-6,8,10-triazatricyclo[9.4.0.0^{2,7}]pentadeca-1(11),2(7),3,5,12,14-hexaen-9-one;
2-2: 13-chloro-10-(2-fluoro-4-{[2-(methylamino)ethyl]amino}phenyl)-8-methyl-6,8,10-triazatricyclo[9.4.0.0^{2,7}]pentadeca-1(11),2(7),3,5,12,14-hexaen-9-one;
2-3: 4,13-dichloro-8-ethyl-10-(4-{[2-(methylamino)ethyl]amino}phenyl)-6,8,10-triazatricyclo[9.4.0.0^{2,7}]pentadeca-1(11),2(7),3,5,12,14-hexaen-9-one;
2-4: 13-chloro-8-ethyl-4-fluoro-10-(4-{[2-(methylamino)ethyl]amino}phenyl)-6,8,10-triazatricyclo[9.4.0.0^{2,7}]pentadeca-1(11),2(7),3,5,12,14-hexaen-9-one;
2-5: 13-chloro-8-ethyl-4-methyl-10-(4-{[2-(methylamino)ethyl]amino}phenyl)-6,8,10-triazatricyclo[9.4.0.0^{2,7}]pentadeca-1(11),2(7),3,5,12,14-hexaen-9-one;
2-6: 4-chloro-8-ethyl-13-methoxy-10-(4-{[2-(methylamino)ethyl]amino}phenyl)-6,8,10-triazatricyclo[9.4.0.0^{2,7}]pentadeca-1(11),2(7),3,5,12,14-hexaen-9-one;
2-7: 4,13-dichloro-8-ethyl-10-[2-fluoro-4-({2-[(2-hydroxyethyl)amino]ethyl}amino)phenyl]-6,8,10-triazatricyclo[9.4.0.0^{2,7}]pentadeca-1(11),2(7),3,5,12,14-hexaen-9-one;
2-8: 4,13-dichloro-8-ethyl-10-(2-fluoro-4-{[2-(methylamino)ethyl]amino}phenyl)-6,8,10-triazatricyclo[9.4.0.0^{2,7}]pentadeca-1(11),2(7),3,5,12,14-hexaen-9-one;
2-9: 4,13-dichloro-8-ethyl-10-[4-({2-[(2-hydroxyethyl)amino]ethyl}amino)phenyl]-6,8,10-triazatricyclo[9.4.0.0^{2,7}]pentadeca-1(11),2(7),3,5,12,14-hexaen-9-one;
2-10: 4,13-dichloro-8-ethyl-10-[3-fluoro-5-({2-[(2-hydroxyethyl)amino]ethyl}amino)pyridin-2-yl]-6,8,10-triazatricyclo[9.4.0.0^{2,7}]pentadeca-1(11),2(7),3,5,12,14-hexaen-9-one;
2-11: 4,13-dichloro-8-ethyl-10-(3-fluoro-5-{[2-(methylamino)ethyl]amino}pyridin-2-yl)-6,8,10-triazatricyclo[9.4.0.0^{2,7}]pentadeca-1(11),2(7),3,5,12,14-hexaen-9-one;
2-12: 4,13-dichloro-8-ethyl-10-[2-fluoro-4-({2-[(oxan-4-yl)amino]ethyl}amino)phenyl]-6,8,10-triazatricyclo[9.4.0.0^{2,7}]pentadeca-1(11),2(7),3,5,12,14-hexaen-9-one;
2-13: 4,13-dichloro-8-ethyl-10-[4-({2-[(oxan-4-yl)amino]ethyl}amino)phenyl]-6,8,10-triazatricyclo[9.4.0.0^{2,7}]pentadeca-1(11),2(7),3,5,12,14-hexaen-9-one;
2-14: 4,13-dichloro-8-ethyl-10-[3-fluoro-5-({2-[(oxan-4-yl)amino]ethyl}amino)pyridin-2-yl]-6,8,10-triazatricyclo[9.4.0.0^{2,7}]pentadeca-1(11),2(7),3,5,12,14-hexaen-9-one.

In some embodiments, provided herein is a pharmaceutically acceptable salt of a compound that is described in Table 2.

TABLE 3

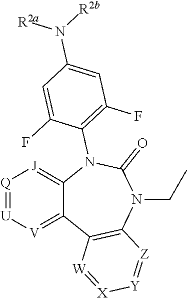

| Cpd No. | 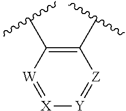 | 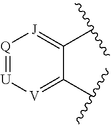 | 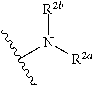 |
|---|---|---|---|
| 3-1 | 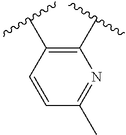 [SMILES: *c1ccc(C)nc1*] | 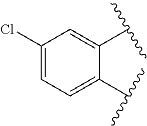 [SMILES: *c1ccc(Cl)cc1*] | 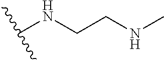 [SMILES: *NCCNC] |
| 3-2 | 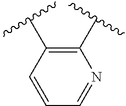 [SMILES: *c1cccnc1*] | 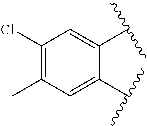 [SMILES: *c1cc(C)c(Cl)cc1*] | 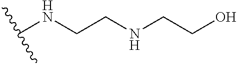 [SMILES: *NCCNCCO] |
| 3-3 | 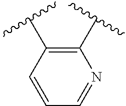 [SMILES: *c1cccnc1*] | 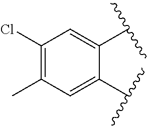 [SMILES: *c1cc(C)c(Cl)cc1*] | 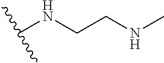 [SMILES: *NCCNC] |
| 3-4 | 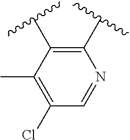 [SMILES: *c1ncc(Cl)c(C)c1*] | 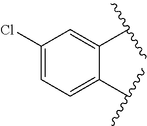 [SMILES: *c1ccc(Cl)cc1*] | 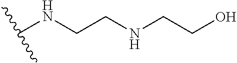 [SMILES: *NCCNCCO] |
| 3-5 | 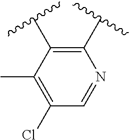 [SMILES: *c1ncc(Cl)c(C)c1*] | 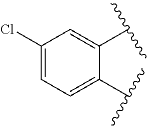 [SMILES: *c1ccc(Cl)cc1*] | 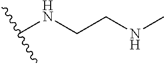 [SMILES: *NCCNC] |
| 3-6 | 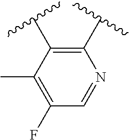 [SMILES: *c1ncc(F)c(C)c1*] | 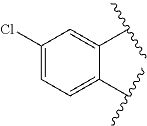 [SMILES: *c1ccc(Cl)cc1*] | 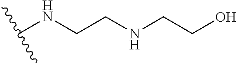 [SMILES: *NCCNCCO] |
| 3-7 | 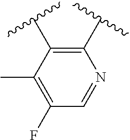 [SMILES: *c1ncc(F)c(C)c1*] | 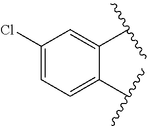 [SMILES: *c1ccc(Cl)cc1*] | 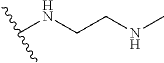 [SMILES: *NCCNC] |
| 3-8 | 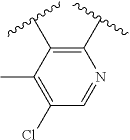 [SMILES: *c1ncc(Cl)c(C)c1*] | 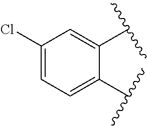 [SMILES: *c1ccc(Cl)cc1*] | 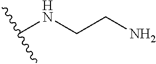 [SMILES: *NCCN] |
| 3-9 | 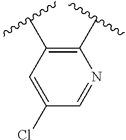 [SMILES: *c1cc(Cl)cnc1*] | 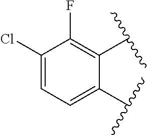 [SMILES: *c1ccc(Cl)c(F)c1*] | 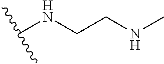 [SMILES: *NCCNC] |
| 3-10 | 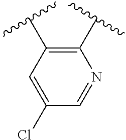 [SMILES: *c1cc(Cl)cnc1*] | 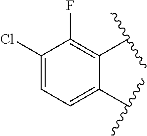 [SMILES: *c1ccc(Cl)c(F)c1*] | 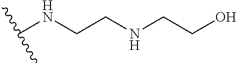 [SMILES: *NCCNCCO] |
| 3-11 | 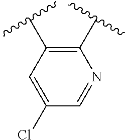 [SMILES: *c1cc(Cl)cnc1*] | 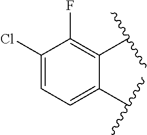 [SMILES: *c1ccc(Cl)c(F)c1*] | 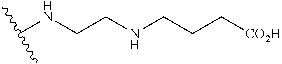 [SMILES: *NCCNCCCC(=O)O] |
| 3-12 | 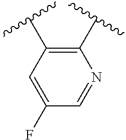 [SMILES: *c1cc(F)cnc1*] | 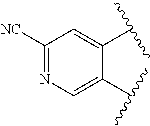 [SMILES: *c1cnc(C#N)cc1*] | 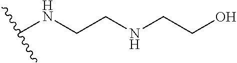 [SMILES: *NCCNCCO] |
| 3-13 | 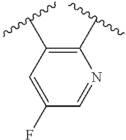 [SMILES: *c1cc(F)cnc1*] | 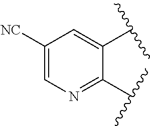 [SMILES: *c1cc(C#N)cnc1*] | 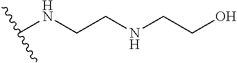 [SMILES: *NCCNCCO] |
| 3-14 | 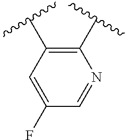 [SMILES: *c1cc(F)cnc1*] | 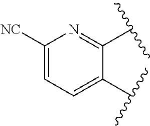 [SMILES: *c1ccc(C#N)nc1*] | 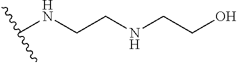 [SMILES: *NCCNCCO] |
| 3-15 | 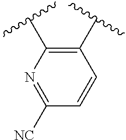 [SMILES: *c1ccc(C#N)nc1*] | 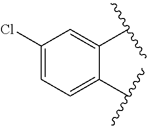 [SMILES: *c1ccc(Cl)cc1*] | 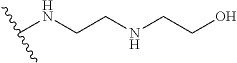 [SMILES: *NCCNCCO] |
| 3-16 | 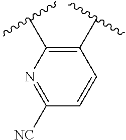 [SMILES: *c1ccc(C#N)nc1*] | 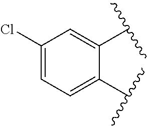 [SMILES: *c1ccc(Cl)cc1*] | 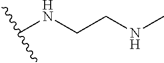 [SMILES: *NCCNC] |
| 3-17 | 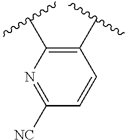 [SMILES: *c1ccc(C#N)nc1*] | 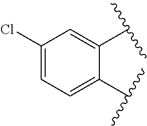 [SMILES: *c1ccc(Cl)cc1*] | 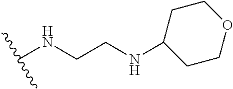 [SMILES: *NCCNC1CCOCC1] |
| 3-18 | 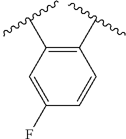 [SMILES: *c1ccc(F)cc1*] | 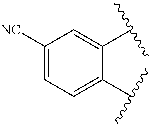 [SMILES: *c1ccc(C#N)cc1*] | 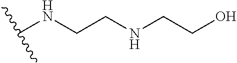 [SMILES: *NCCNCCO] |
| 3-19 | 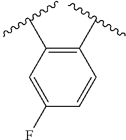 [SMILES: *c1ccc(F)cc1*] | 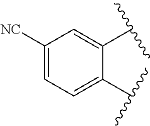 [SMILES: *c1ccc(C#N)cc1*] | 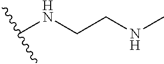 [SMILES: *NCCNC] |
| 3-20 | 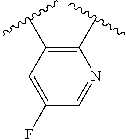 [SMILES: *c1cc(F)cnc1*] | 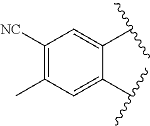 [SMILES: *c1cc(C)c(C#N)cc1*] | 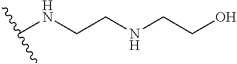 [SMILES: *NCCNCCO] |
| 3-21 | 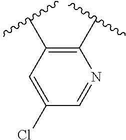 [SMILES: *c1cc(Cl)cnc1*] | 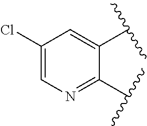 [SMILES: *c1cc(Cl)cnc1*] | 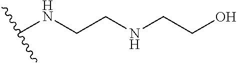 [SMILES: *NCCNCCO] |
| 3-22 | 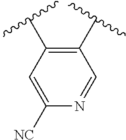 [SMILES: *c1cnc(C#N)cc1*] | 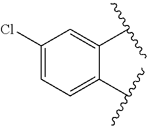 [SMILES: *c1ccc(Cl)cc1*] | 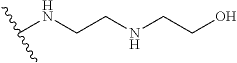 [SMILES: *NCCNCCO] |
| 3-23 | 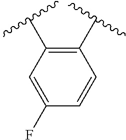 [SMILES: *c1ccc(F)cc1*] | 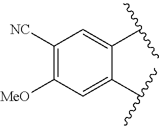 [SMILES: *c1cc(C#N)c(OC)cc1*] | 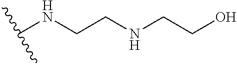 [SMILES: *NCCNCCO] |
| 3-24 | 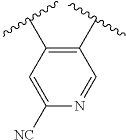 [SMILES: *c1cnc(C#N)cc1*] | 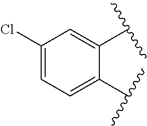 [SMILES: *c1ccc(Cl)cc1*] | 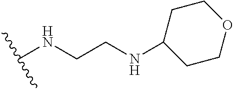 [SMILES: *NCCNC1CCOCC1] |
| 3-25 | 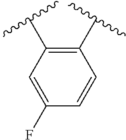 [SMILES: *c1ccc(F)cc1*] | 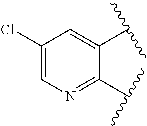 [SMILES: *c1cc(Cl)cnc1*] | 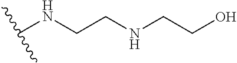 [SMILES: *NCCNCCO] |

Compounds in Table 3 are named:
3-1: 13-chloro-10-(2,6-difluoro-4-{[2-(methylamino)ethyl]amino}phenyl)-8-ethyl-5-methyl-6,8,10-triazatricyclo[9.4.0.0^{2,7}]pentadeca-1(11),2(7),3,5,12,14-hexaen-9-one;
3-2: 13-chloro-10-[2,6-difluoro-4-({2-[(2-hydroxyethyl)amino]ethyl}amino)phenyl]-8-ethyl-14-methyl-6,8,10-triazatricyclo[9.4.0.0^{2,7}]pentadeca-1(11),2(7),3,5,12,14-hexaen-9-one;
3-3: 13-chloro-10-(2,6-difluoro-4-{[2-(methylamino)ethyl]amino}phenyl)-8-ethyl-14-methyl-6,8,10-triazatricyclo[9.4.0.0^{2,7}]pentadeca-1(11),2(7),3,5,12,14-hexaen-9-one;
3-4: 4,13-dichloro-10-[2,6-difluoro-4-({2-[(2-hydroxyethyl)amino]ethyl}amino)phenyl]-8-ethyl-3-methyl-6,8,10-triazatricyclo[9.4.0.0^{2,7}]pentadeca-1(11),2(7),3,5,12,14-hexaen-9-one;
3-5: 4,13-dichloro-10-(2,6-difluoro-4-{[2-(methylamino)ethyl]amino}phenyl)-8-ethyl-3-methyl-6,8,10-triazatricyclo[9.4.0.0^{2,7}]pentadeca-1(11),2(7),3,5,12,14-hexaen-9-one;
3-6: 13-chloro-10-[2,6-difluoro-4-({2-[(2-hydroxyethyl)amino]ethyl}amino)phenyl]-8-ethyl-4-fluoro-3-methyl-6,8,10-triazatricyclo[9.4.0.0^{2,7}]pentadeca-1(11),2(7),3,5,12,14-hexaen-9-one;
3-7: 13-chloro-10-(2,6-difluoro-4-{[2-(methylamino)ethyl]amino}phenyl)-8-ethyl-4-fluoro-3-methyl-6,8,10-triazatricyclo[9.4.0.0^{2,7}]pentadeca-1(11),2(7),3,5,12,14-hexaen-9-one;
3-8: 10-{4-[(2-aminoethyl)amino]-2,6-difluorophenyl}-4,13-dichloro-8-ethyl-3-methyl-6,8,10-triazatricyclo[9.4.0.0^{2,7}]pentadeca-1(11),2(7),3,5,12,14-hexaen-9-one;
3-9: 4,13-dichloro-10-(2,6-difluoro-4-{[2-(methylamino)ethyl]amino}phenyl)-8-ethyl-12-fluoro-6,8,10-triazatricyclo[9.4.0.0^{2,7}]pentadeca-1(11),2(7),3,5,12,14-hexaen-9-one;
3-10: 4,13-dichloro-10-[2,6-difluoro-4-({2-[(2-hydroxyethyl)amino]ethyl}amino)phenyl]-8-ethyl-12-fluoro-6,8,10-triazatricyclo[9.4.0.0^{2,7}]pentadeca-1(11),2(7),3,5,12,14-hexaen-9-one;
3-11: 4-({2-[(4-{4,13-dichloro-8-ethyl-12-fluoro-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)butanoic acid;
3-12: 8-[2,6-difluoro-4-({2-[(2-hydroxyethyl)amino]ethyl}amino)phenyl]-10-ethyl-14-fluoro-9-oxo-4,8,10,12-tetraazatricyclo[9.4.0.0^{2,7}]pentadeca-1(11),2(7),3,5,12,14-hexaene-5-carbonitrile;
3-13: 8-[2,6-difluoro-4-({2-[(2-hydroxyethyl)amino]ethyl}amino)phenyl]-10-ethyl-14-fluoro-9-oxo-3,8,10,12-tetraazatricyclo[9.4.0.0^{2,7}]pentadeca-1(11),2(7),3,5,12,14-hexaene-5-carbonitrile;
3-14: 8-[2,6-difluoro-4-({2-[(2-hydroxyethyl)amino]ethyl}amino)phenyl]-10-ethyl-14-fluoro-9-oxo-6,8,10,12-tetraazatricyclo[9.4.0.0^{2,7}]pentadeca-1(11),2(7),3,5,12,14-hexaene-5-carbonitrile.
3-15: 13-chloro-10-[2,6-difluoro-4-({2-[(2-hydroxyethyl)amino]ethyl}amino)phenyl]-8-ethyl-9-oxo-3,8,10-triazatricyclo[9.4.0.0^{2,7}]pentadeca-1(11),2(7),3,5,12,14-hexaene-4-carbonitrile;
3-16: 13-chloro-10-(2,6-difluoro-4-{[2-(methylamino)ethyl]amino}phenyl)-8-ethyl-9-oxo-3,8,10-triazatricyclo[9.4.0.0^{2,7}]pentadeca-1(11),2(7),3,5,12,14-hexaene-4-carbonitrile;
3-17: 13-chloro-10-[2,6-difluoro-4-({2-[(oxan-4-yl)amino]ethyl}amino)phenyl]-8-ethyl-9-oxo-3,8,10-triazatricyclo[9.4.0.0^{2,7}]pentadeca-1(11),2(7),3,5,12,14-hexaene-4-carbonitrile;
3-18: 8-[2,6-difluoro-4-({2-[(2-hydroxyethyl)amino]ethyl}amino)phenyl]-10-ethyl-14-fluoro-9-oxo-8,10-diazatricyclo[9.4.0.0^{2,7}]pentadeca-1(11),2(7),3,5,12,14-hexaene-5-carbonitrile;
3-19: 8-(2,6-difluoro-4-{[2-(methylamino)ethyl]amino}phenyl)-10-ethyl-14-fluoro-9-oxo-8,10-diazatricyclo[9.4.0.0^{2,7}]pentadeca-1(11),2(7),3,5,12,14-hexaene-5-carbonitrile;
3-20: 10-[2,6-difluoro-4-({2-[(2-hydroxyethyl)amino]ethyl}amino)phenyl]-8-ethyl-4-fluoro-14-methyl-9-oxo-6,8,10-triazatricyclo[9.4.0.0^{2,7}]pentadeca-1(11),2(7),3,5,12,14-hexaene-13-carbonitrile;
3-21: 5,14-dichloro-8-[2,6-difluoro-4-({2-[(2-hydroxyethyl)amino]ethyl}amino)phenyl]-10-ethyl-3,8,10,12-tetraazatricyclo[9.4.0.0^{2,7}]pentadeca-1(11),2(7),3,5,12,14-hexaen-9-one;
3-22: 13-chloro-10-[2,6-difluoro-4-({2-[(2-hydroxyethyl)amino]ethyl}amino)phenyl]-8-ethyl-9-oxo-5,8,10-triazatricyclo[9.4.0.0^{2,7}]pentadeca-1(11),2(7),3,5,12,14-hexaene-4-carbonitrile;
3-23: 10-[2,6-difluoro-4-({2-[(2-hydroxyethyl)amino]ethyl}amino)phenyl]-8-ethyl-4-fluoro-14-methoxy-9-oxo-6,8,10-triazatricyclo[9.4.0.0^{2,7}]pentadeca-1(11),2(7),3,5,12,14-hexaene-13-carbonitrile;
3-24: 13-chloro-10-[2,6-difluoro-4-({2-[(oxan-4-yl)amino]ethyl}amino)phenyl]-8-ethyl-9-oxo-5,8,10-triazatricyclo[9.4.0.0^{2,7}]pentadeca-1(11),2(7),3,5,12,14-hexaene-4-carbonitrile; and
3-25: 5-chloro-8-[2,6-difluoro-4-({2-[(2-hydroxyethyl)amino]ethyl}amino)phenyl]-10-ethyl-14-fluoro-3,8,10,12-tetraazatricyclo[9.4.0.0^{2,7}]pentadeca-1(11),2(7),3,5,12,14-hexaen-9-one.

In some embodiments, provided herein is a pharmaceutically acceptable salt of a compound that is described in Table 3.

### Further Forms of Compounds

In one aspect, compounds described herein are in the form of pharmaceutically acceptable salts. As well, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

“Pharmaceutically acceptable,” as used herein, refers a material, such as a carrier or diluent, which does not abrogate the biological activity or properties of the compound, and is relatively nontoxic, i.e., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zurich:Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible and this capability can be manipulated as one aspect of delayed and sustained release behaviors. Also, because the salt-forming molecule can be in equilibrium with a neutral form, passage through biological membranes can be adjusted.

In some embodiments, pharmaceutically acceptable salts are obtained by reacting a compound of Formula (I′) or (I) with an acid. In some embodiments, the compound of Formula (I′) or (I) (i.e. free base form) is basic and is reacted with an organic acid or an inorganic acid. Inorganic acids include, but are not limited to, hydrochloric acid, hydrobromic acid, sulfuric acid, phosphoric acid, nitric acid, and metaphosphoric acid. Organic acids include, but are not limited to, 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (−L); malonic acid; mandelic acid (DL); methanesulfonic acid; naphthalene-1,5-disulfonic acid; naphthalene-2-sulfonic acid; nicotinic acid; oleic acid; oxalic acid; palmitic acid; pamoic acid; phosphoric acid; proprionic acid; pyroglutamic acid (−L); salicylic acid; sebacic acid; stearic acid; succinic acid; sulfuric acid; tartaric acid (+L); thiocyanic acid; toluenesulfonic acid (p); and undecylenic acid.

In some embodiments, a compound of Formula (I′) or (I) is prepared as a chloride salt, sulfate salt, bromide salt, mesylate salt, maleate salt, citrate salt or phosphate salt.

In some embodiments, pharmaceutically acceptable salts are obtained by reacting a compound of Formula (I′) or (I) with a base. In some embodiments, the compound of Formula (I′) or (I) is acidic and is reacted with a base. In such situations, an acidic proton of the compound of Formula (I′) or (I) is replaced by a metal ion, e.g., lithium, sodium, potassium, magnesium, calcium, or an aluminum ion. In some cases, compounds described herein coordinate with an organic base, such as, but not limited to, ethanolamine, diethanolamine, triethanolamine, tromethamine, meglumine, N-methylglucamine, dicyclohexylamine, tris(hydroxymethyl)methylamine. In other cases, compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, potassium carbonate, sodium hydroxide, lithium hydroxide, and the like. In some embodiments, the compounds provided herein are prepared as a sodium salt, calcium salt, potassium salt, magnesium salt, meglumine salt, N-methylglucamine salt or ammonium salt.

It should be understood that a reference to a pharmaceutically acceptable salt includes the solvent addition forms. In some embodiments, solvates contain either stoichiometric or non-stoichiometric amounts of a solvent, and are formed during the process of crystallization with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

The methods and formulations described herein include the use of N-oxides (if appropriate), or pharmaceutically acceptable salts of compounds having the structure of Formula (I′) or (I), as well as active metabolites of these compounds having the same type of activity.

In some embodiments, sites on the organic radicals (e.g. alkyl groups, aromatic rings) of compounds of Formula (I′) or (I) are susceptible to various metabolic reactions. Incorporation of appropriate substituents on the organic radicals will reduce, minimize or eliminate this metabolic pathway. In specific embodiments, the appropriate substituent to decrease or eliminate the susceptibility of the aromatic ring to metabolic reactions is, by way of example only, a halogen, deuterium, an alkyl group, a haloalkyl group, or a deuteroalkyl group.

In another embodiment, the compounds described herein are labeled isotopically (e.g. with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine chlorine, iodine, phosphorus, such as, for example, ²H, ³H, ¹³C, ¹⁴C, ¹⁵N, ¹⁸O, ¹⁷O, ³⁵S, ¹⁸F, ³⁶Cl, ¹²³I, ¹²⁴I, ¹²⁵I, ¹³¹I, ³²P and ³³P. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

In some embodiments, the compounds of Formula (I′) or (I) possess one or more stereocenters and each stereocenter exists independently in either the R or S configuration. In some embodiments, the compound of Formula (I′) or (I) exists in the R configuration. In some embodiments, the compound of Formula (I′) or (I) exists in the S configuration. The compounds presented herein include all diastereomeric, individual enantiomers, atropisomers, and epimeric forms as well as the appropriate mixtures thereof. The compounds and methods provided herein include all cis, trans, syn, anti, entgegen (E), and zusammen (Z) isomers as well as the appropriate mixtures thereof.

Individual stereoisomers are obtained, if desired, by methods such as, stereoselective synthesis and/or the separation of stereoisomers by chiral chromatographic columns or the separation of diastereomers by either non-chiral or chiral chromatographic columns or crystallization and recrystallization in a proper solvent or a mixture of solvents. In certain embodiments, compounds of Formula (I′) or (I) are prepared as their individual stereoisomers by reacting a racemic mixture of the compound with an optically active resolving agent to form a pair of diastereoisomeric compounds/salts, separating the diastereomers and recovering the optically pure individual enantiomers. In some embodiments, resolution of individual enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In some embodiments, stereoisomers are obtained by stereoselective synthesis.

In some embodiments, compounds described herein are prepared as prodrugs. A “prodrug” refers to an agent that is converted into the parent drug in vivo. Prodrugs are often useful because, in some situations, they are easier to administer than the parent drug. They are, for instance, bioavailable by oral administration whereas the parent is not. Further or alternatively, the prodrug also has improved solubility in pharmaceutical compositions over the parent drug. In some embodiments, the design of a prodrug increases the effective water solubility. An example, without limitation, of a prodrug is a compound described herein, which is administered as an ester (the “prodrug”) but then is metabolically hydrolyzed to provide the active entity. A further example of a prodrug is a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

Prodrugs of the compounds described herein include, but are not limited to, esters, ethers, carbonates, thiocarbonates, N-acyl derivatives, N-acyloxyalkyl derivatives, N-alkyloxyacyl derivatives, quaternary derivatives of tertiary amines, N-Mannich bases, Schiff bases, amino acid conjugates, phosphate esters, and sulfonate esters. See for example Design of Prodrugs, Bundgaard, A. Ed., Elseview, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference. In some embodiments, a hydroxyl group in the compounds disclosed herein is used to form a prodrug, wherein the hydroxyl group is incorporated into an acyloxyalkyl ester, alkoxycarbonyloxyalkyl ester, alkyl ester, aryl ester, phosphate ester, sugar ester, ether, and the like. In some embodiments, a hydroxyl group in the compounds disclosed herein is a prodrug wherein the hydroxyl is then metabolized in vivo to provide a carboxylic acid group. In some embodiments, a carboxyl group is used to provide an ester or amide (i.e. the prodrug), which is then metabolized in vivo to provide a carboxylic acid group. In some embodiments, compounds described herein are prepared as alkyl ester prodrugs.

Prodrug forms of the herein described compounds, wherein the prodrug is metabolized in vivo to produce a compound of Formula (I′) or (I) as set forth herein are included within the scope of the claims. In some cases, some of the herein-described compounds is a prodrug for another derivative or active compound.

In some embodiments, any one of the hydroxyl group(s), amino group(s) and/or carboxylic acid group(s) are functionalized in a suitable manner to provide a prodrug moiety. In some embodiments, the prodrug moiety is as described above.

In additional or further embodiments, the compounds described herein are metabolized upon administration to an organism in need to produce a metabolite that is then used to produce a desired effect, including a desired therapeutic effect.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulfhydryl groups. Metabolites of the compounds disclosed herein are optionally identified either by administration of compounds to a host and analysis of tissue samples from the host, or by incubation of compounds with hepatic cells in vitro and analysis of the resulting compounds.

In some instances, heterocyclic rings may exist in tautomeric forms. In such situations, it is understood that the structures of said compounds are illustrated or named in one tautomeric form but could be illustrated or named in the alternative tautomeric form. The alternative tautomeric forms are expressly included in this disclosure, such as, for example, the structures illustrated below. For example, benzimidazoles or imidazoles could exist in the following tautomeric forms:

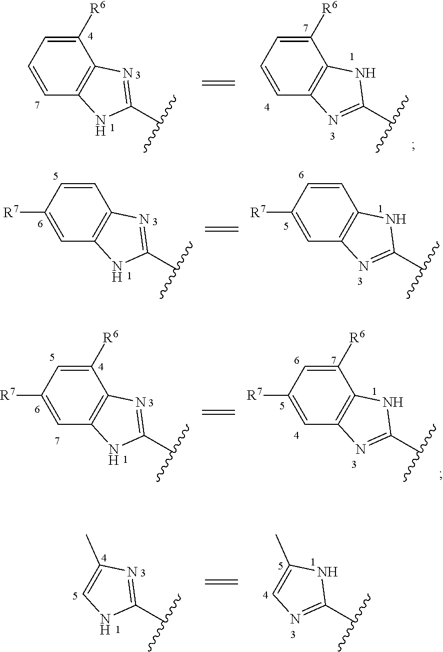

### Synthesis of Compounds

Compounds of Formula (I′) or (I) described herein are synthesized using standard synthetic techniques or using methods known in the art in combination with methods described herein.

Unless otherwise indicated, conventional methods of mass spectroscopy, NMR, HPLC, protein chemistry, biochemistry, recombinant DNA techniques and pharmacology are employed.

Compounds are prepared using standard organic chemistry techniques such as those described in, for example, March's Advanced Organic Chemistry, 6^{th} Edition, John Wiley and Sons, Inc. Alternative reaction conditions for the synthetic transformations described herein may be employed such as variation of solvent, reaction temperature, reaction time, as well as different chemical reagents and other reaction conditions.

In some embodiments, compounds described herein are prepared as described in Scheme A.

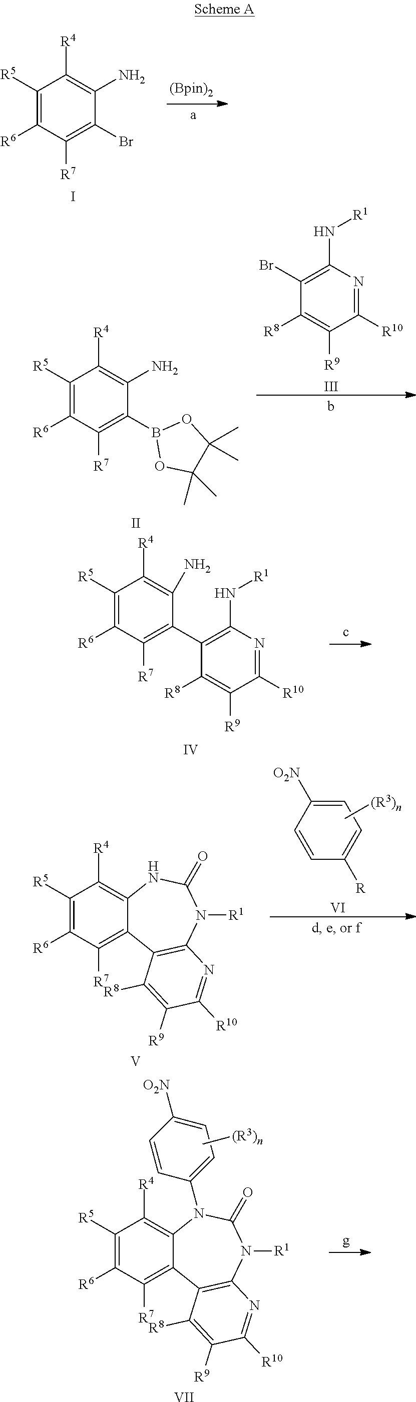

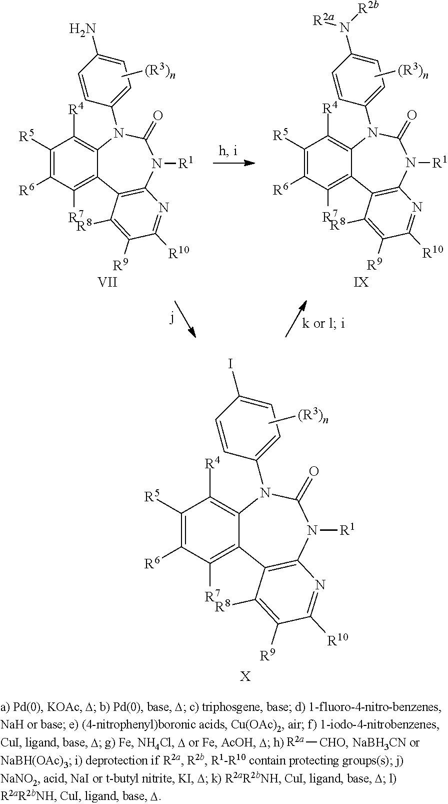

Starting from the appropriately substituted 2-bromoaniline I, formation of pinacol boronate ester under standard palladium-catalyzed conditions gives the intermediates II. Subsequent Suzuki-Miyaura reactions of II with substituted 3-bromo-2-aminopyridines III provide the coupled products IV. Treatment of IV with reagents such as triphosgene results in 7-membered cyclic urea ring formation to give the tricycles V. Intermediates V can be further derivatized in several ways. For example, SNAr of the anions of V with a 4-fluoronitrobenzene derivatives VI yields the nitroaryl intermediates VII. Likewise, Chan-Lam coupling of V with the appropriate arylboronic acid versions of VI, in the presence of copper acetate and air, provides VII. Alternatively, Ullmann reaction with 4-iodonitrobenzene derivatives VI employing copper iodide and a ligand also give VII. The nitro moieties of VII can be reduced under standard conditions, for example with iron powder, to yield the anilines VIII, which can then be subjected to common reductive alkylation procedures, followed by protecting group deprotection if necessary, to give desired compounds IX. Alternatively, anilines VII can be converted to the aryl iodides X using Sandmeyer protocols. This is followed by either Buchwald coupling with amines in the presence of palladium catalyst and phosphine ligand, or by Ullman coupling as described above, followed by deprotection if necessary, to provide products IX.

In some embodiments, compounds described herein are prepared as described in Scheme B.

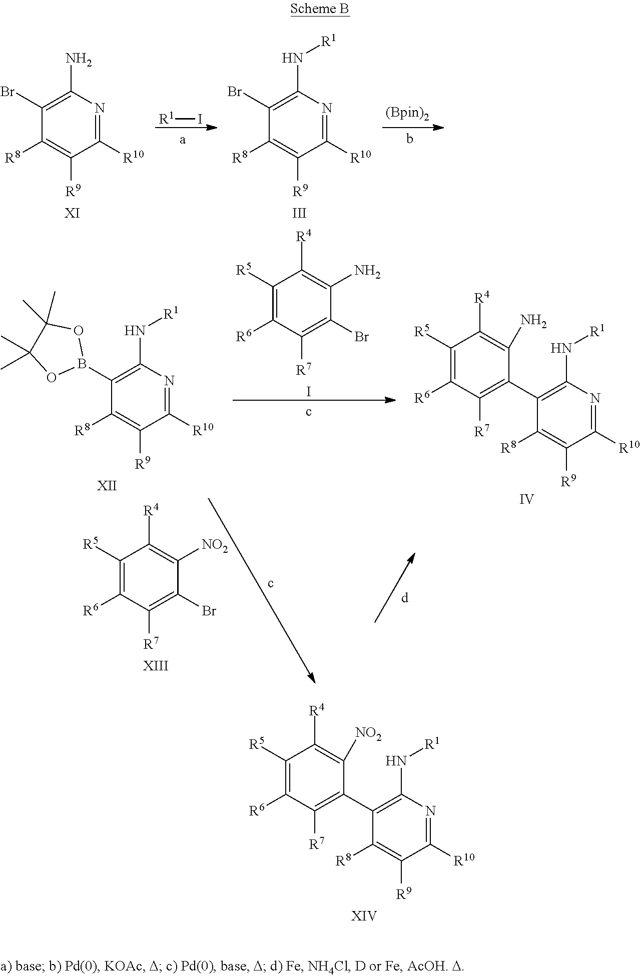

An alternative synthesis involves reversing the coupling partners of the Suzuki-Miyaura reaction. For example, the bromo-aminopyridines XI can be N-alkylated under standard conditions, providing III, which can then be subjected to pinacol boronate ester formation in the presence of palladium catalyst as described earlier. The resulting pinacol boronate coupling partners XII is reacted with the corresponding bromoanilines I, under standard Suzuki-Miyaura conditions, to give the previously-described coupled product IV. Alternatively, pinacol boronates XII can be cross-coupled with bromonitroaryls XIII to yield XIV. The nitro moieties of XIV are then reduced under standard conditions to also give IV. The intermediates IV can be further elaborated to the desired products (e.g. IX), using the same chemistry as described in Scheme A.

In some embodiments, compounds described herein are prepared as described in Scheme C.

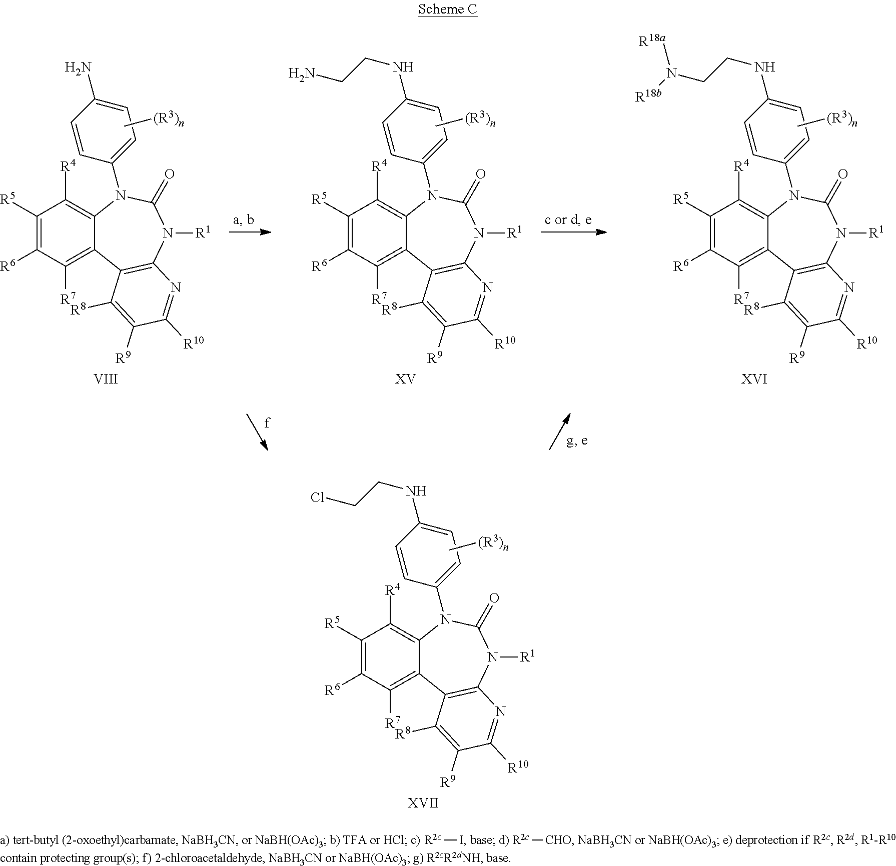

The anilines VIII, previously described in Scheme A, can undergo reductive alkylation reactions with aldehydes such as t-butyl (2-oxoethyl)carbamate, followed by Boc deprotection, to yield diamines XV. A second reductive alkylation, or simple alkylation with alkylhalides, followed by deprotection if necessary, gives the products XVI. Alternatively, the anilines VIII can be reacted with aldehydes such as chloroacetaldehyde to provide chloro intermediates XVII. The chloride moiety can then be displaced via standard S_{N}² conditions, giving the desired products XVI following deprotection.

In some embodiments, compounds described herein are prepared as described in Scheme D.

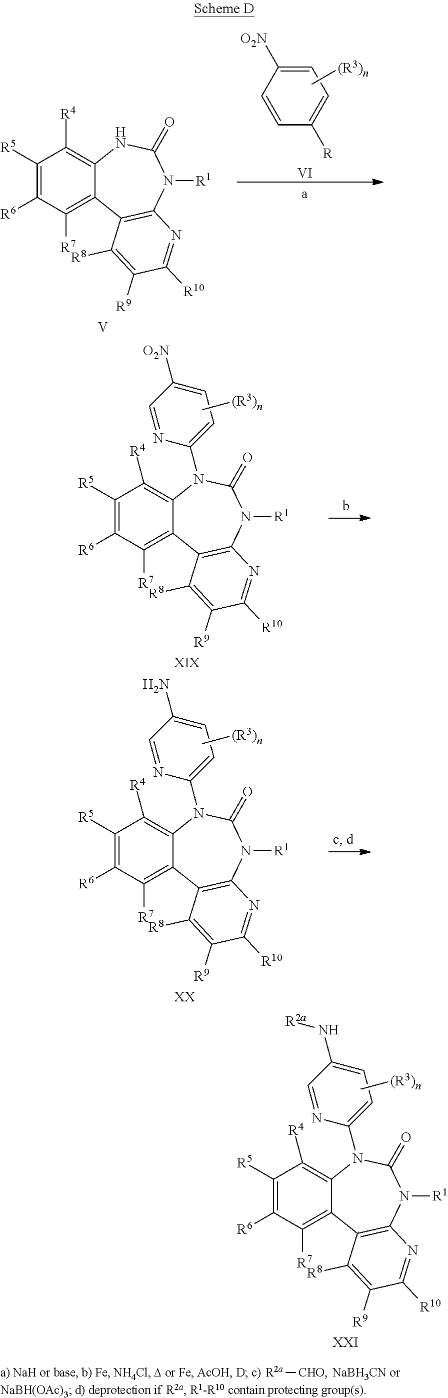

The previously described tricyclic intermediates V can be reacted with appropriately substituted 2-fluoro-5-nitropyridines in the presence of base to provide nitropyridine intermediates XIX. Nitro reduction employing iron, or other standard reductants, yields the amino intermediates XX, which can be further derivatized as described previously, e.g. via reductive alkylation and deprotection, to give the desired products XXI.

In some embodiments, compounds described herein are prepared as described in Scheme E.

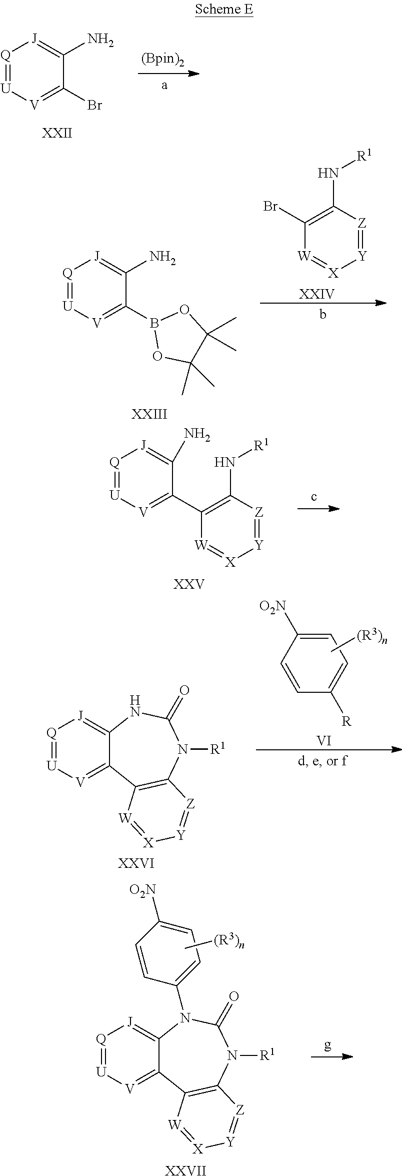

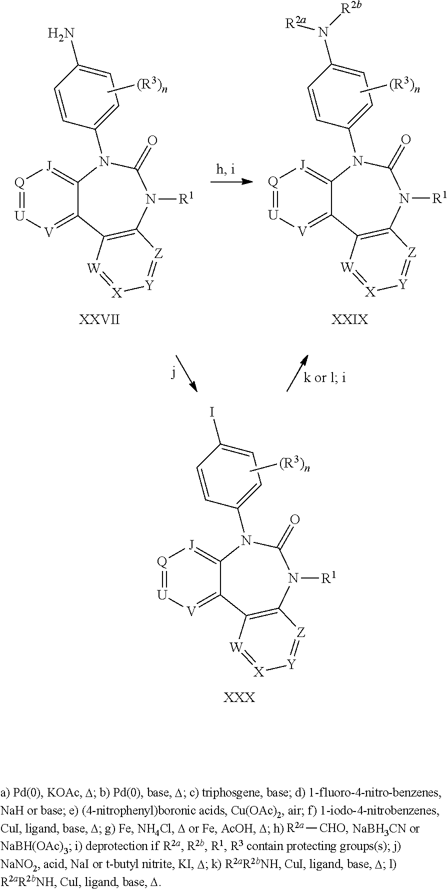

Aryl- or heteroaryl-amines XXII, e.g. aminopyridines, can be subjected to analogous chemistry to that described earlier—namely pinacol boronate ester formation to give XXIII, followed by Suzuki-Miyaura coupling with aryl- or heteroaryl-bromides XXIV, to provide the coupled products XXV. Formation of the seven-membered cyclic urea XXVI is again achieved upon treatment of XXV with triphosgene. Installation of the nitroaryl moiety can be realized using the previously described methods to give XXVIII. Finally, nitro reduction to the anilines XXVIII, and subsequent appropriate derivatizations either directly or via iodides XXX, provide the desired products XXIX.

In some embodiments, compounds described herein are prepared as described in Scheme F.

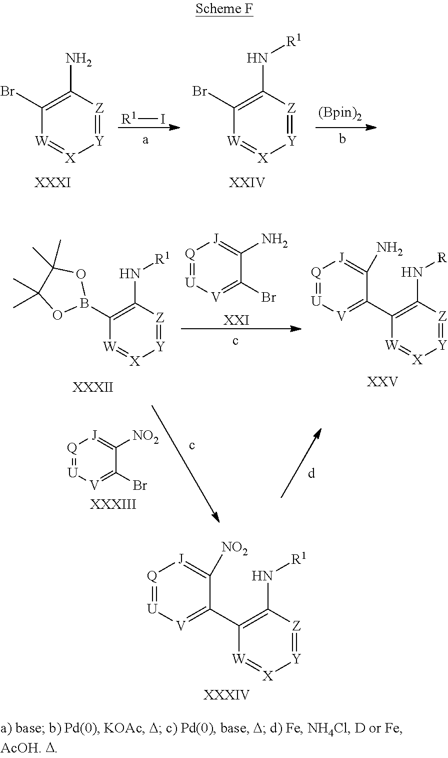

An alternative synthesis of aryl- or heteroaryl-tricylic products again involves reversing the coupling partners of the Suzuki-Miyaura reaction. In a similar manner to the steps described in Scheme B, aryl- or heteroaryl-compounds XXXI can be alkylated to give XXIV, then converted to the pinacol boronate esters XXXII as described previously. Subsequent Suzuki-Miyaura reactions with the appropriately substituted coupling partners XXII or XXXIII give the biaryl or heterobiaryl intermediates XXV or XXXIV, respectively, which can be further elaborated and derivatized employing the chemistry described in the previous schemes.

In some embodiments, compounds described herein are synthesized as outlined in the Examples.

### Certain Terminology

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

As used herein, C₁-Cₓ includes C₁-C₂, C₁-C₃ . . . C₁-Cₓ. By way of example only, a group designated as “C₁-C₆” indicates that there are one to six carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

An “alkyl” group refers to an aliphatic hydrocarbon group. The alkyl group is branched or straight chain. In some embodiments, the “alkyl” group has 1 to 10 carbon atoms, i.e. a C₁-C₁₀alkyl. Whenever it appears herein, a numerical range such as “1 to 10” refers to each integer in the given range; e.g., “1 to 10 carbon atoms” means that the alkyl group consist of 1 carbon atom, 2 carbon atoms, 3 carbon atoms, etc., up to and including 10 carbon atoms, although the present definition also covers the occurrence of the term “alkyl” where no numerical range is designated. In some embodiments, an alkyl is a C₁-C₆alkyl. In one aspect the alkyl is methyl, ethyl, propyl, iso-propyl, n-butyl, iso-butyl, sec-butyl, or t-butyl. Typical alkyl groups include, but are in no way limited to, methyl, ethyl, propyl, isopropyl, butyl, isobutyl, sec-butyl, tertiary butyl, pentyl, neopentyl, or hexyl.

An “alkylene” group refers to a divalent alkyl radical. Any of the above mentioned monovalent alkyl groups may be an alkylene by abstraction of a second hydrogen atom from the alkyl. In some embodiments, an alkylene is a C₁-C₆alkylene. In other embodiments, an alkylene is a C₁-C₄alkylene. Typical alkylene groups include, but are not limited to, —CH₂—, —CH₂CH₂—, —CH₂CH₂CH₂—, —CH₂CH₂CH₂CH₂—, and the like. In some embodiments, an alkylene is —CH₂—.

An “alkoxy” group refers to a (alkyl)O— group, where alkyl is as defined herein.

The term “alkylamine” refers to the —N(alkyl)ₓH_{y} group, where x is 0 and y is 2, or where x is 1 and y is 1, or where x is 2 and y is 0.

An “hydroxyalkyl” refers to an alkyl in which one hydrogen atom is replaced by a hydroxyl. In some embodiments, a hydroxyalkyl is a C₁-C₄hydroxyalkyl. Typical hydroxyalkyl groups include, but are not limited to, —CH₂OH, —CH₂CH₂OH, —CH₂CH₂CH₂OH, —CH₂CH₂CH₂CH₂OH, and the like.

An “aminoalkyl” refers to an alkyl in which one hydrogen atom is replaced by an amino. In some embodiments, aminoalkyl is a C₁-C₄aminoalkyl. Typical aminoalkyl groups include, but are not limited to, —CH₂NH₂, —CH₂CH₂NH₂, —CH₂CH₂CH₂NH₂, —CH₂CH₂CH₂CH₂NH₂, and the like.

The term “alkenyl” refers to a type of alkyl group in which at least one carbon-carbon double bond is present. In one embodiment, an alkenyl group has the formula —C(R)═CR₂, wherein R refers to the remaining portions of the alkenyl group, which may be the same or different. In some embodiments, R is H or an alkyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)═CH₂, —CH═CHCH₃, —C(CH₃)═CHCH₃, and —CH₂CH═CH₂.

The term “alkynyl” refers to a type of alkyl group in which at least one carbon-carbon triple bond is present. In one embodiment, an alkenyl group has the formula —C≡C—R, wherein R refers to the remaining portions of the alkynyl group. In some embodiments, R is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃—C≡CCH₂CH₃, —CH₂C≡CH.

The term “heteroalkyl” refers to an alkyl group in which one or more skeletal atoms of the alkyl are selected from an atom other than carbon, e.g., oxygen, nitrogen (e.g. —NH—, —N(alkyl)-, sulfur, or combinations thereof. A heteroalkyl is attached to the rest of the molecule at a carbon atom of the heteroalkyl. In one aspect, a heteroalkyl is a C₁-C₆heteroalkyl.

The term “aromatic” refers to a planar ring having a delocalized π-electron system containing 4n+2 π electrons, where n is an integer. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

The term “carbocyclic” or “carbocycle” refers to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic. Carbocycles include aryls and cycloalkyls.

As used herein, the term “aryl” refers to an aromatic ring wherein each of the atoms forming the ring is a carbon atom. In one aspect, aryl is phenyl or a naphthyl. In some embodiments, an aryl is a phenyl. In some embodiments, an aryl is a phenyl, naphthyl, indanyl, indenyl, or tetrahydronaphthyl. In some embodiments, an aryl is a C₆-C₁₀aryl. Depending on the structure, an aryl group is a monoradical or a diradical (i.e., an arylene group).

The term “cycloalkyl” refers to a monocyclic or polycyclic aliphatic, non-aromatic radical, wherein each of the atoms forming the ring (i.e. skeletal atoms) is a carbon atom. In some embodiments, cycloalkyls are spirocyclic or bridged compounds. In some embodiments, cycloalkyls are optionally fused with an aromatic ring, and the point of attachment is at a carbon that is not an aromatic ring carbon atom. Cycloalkyl groups include groups having from 3 to 10 ring atoms. In some embodiments, cycloalkyl groups are selected from among cyclopropyl, cyclobutyl, cyclopentyl, cyclopentenyl, cyclohexyl, cyclohexenyl, cycloheptyl, cyclooctyl, spiro[2.2]pentyl, norbornyl and bicycle[1.1.1]pentyl. In some embodiments, a cycloalkyl is a C₃-C₆cycloalkyl. In some embodiments, a cycloalkyl is a C₃-C₄cycloalkyl.

The term “halo” or, alternatively, “halogen” or “halide” means fluoro, chloro, bromo or iodo. In some embodiments, halo is fluoro, chloro, or bromo.

The term “fluoroalkyl” refers to an alkyl in which one or more hydrogen atoms are replaced by a fluorine atom. In one aspect, a fluoroalkyl is a C₁-C₆fluoroalkyl.

The term “heterocycle” or “heterocyclic” refers to heteroaromatic rings (also known as heteroaryls) and heterocycloalkyl rings containing one to four heteroatoms in the ring(s), where each heteroatom in the ring(s) is selected from O, S and N, wherein each heterocyclic group has from 3 to 10 atoms in its ring system, and with the proviso that any ring does not contain two adjacent O or S atoms. Non-aromatic heterocyclic groups (also known as heterocycloalkyls) include rings having 3 to 10 atoms in its ring system and aromatic heterocyclic groups include rings having 5 to 10 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The terms “heteroaryl” or, alternatively, “heteroaromatic” refers to an aryl group that includes one or more ring heteroatoms selected from nitrogen, oxygen and sulfur. Illustrative examples of heteroaryl groups include monocyclic heteroaryls and bicyclic heteroaryls. Monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, bicyclic heteroaryl is a C₆-C₉heteroaryl.

A “heterocycloalkyl” group refers to a cycloalkyl group that includes at least one heteroatom selected from nitrogen, oxygen and sulfur. In some embodiments, a heterocycloalkyl is fused with an aryl or heteroaryl. In some embodiments, the heterocycloalkyl is oxazolidinonyl, pyrrolidinyl, tetrahydrofuranyl, tetrahydrothienyl, tetrahydropyranyl, tetrahydrothiopyranyl, piperidinyl, morpholinyl, thiomorpholinyl, piperazinyl, piperidin-2-onyl, pyrrolidine-2,5-dithionyl, pyrrolidine-2,5-dionyl, pyrrolidinonyl, imidazolidinyl, imidazolidin-2-onyl, or thiazolidin-2-onyl. In one aspect, a heterocycloalkyl is a C₂-C₁₀heterocycloalkyl. In another aspect, a heterocycloalkyl is a C₄-C₁₀heterocycloalkyl. In some embodiments, a heterocycloalkyl is monocyclic or bicyclic. In some embodiments, a heterocycloalkyl is monocyclic and is a 3, 4, 5, 6, 7, or 8-membered ring. In some embodiments, a heterocycloalkyl is monocyclic and is a 3, 4, 5, or 6-membered ring. In some embodiments, a heterocycloalkyl is monocyclic and is a 3 or 4-membered ring. In some embodiments, a heterocycloalkyl is monocyclic and is a 4, 5, or 6-membered ring. In some embodiments, a heterocycloalkyl is monocyclic and is a 5 or 6-membered ring. In some embodiments, a heterocycloalkyl contains 0-2 N atoms in the ring. In some embodiments, a heterocycloalkyl contains 0-2 N atoms, 0-2 O atoms and 0-1 S atoms in the ring.

The term “bond” or “single bond” refers to a chemical bond between two atoms, or two moieties when the atoms joined by the bond are considered to be part of larger substructure. In one aspect, when a group described herein is a bond, the referenced group is absent thereby allowing a bond to be formed between the remaining identified groups.

The term “moiety” refers to a specific segment or functional group of a molecule. Chemical moieties are often recognized chemical entities embedded in or appended to a molecule.

Unless otherwise indicated, the term “optionally substituted” or “substituted” means that the referenced group is optionally substituted with one or more additional group(s) individually and independently selected from halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from halogen, —CN, —NH₂, —OH, —NH(CH₃), —N(CH₃)₂, —CH₃, —CH₂CH₃, —CHF₂, —CF₃, —OCH₃, —OCHF₂, and —OCF₃. In some embodiments, substituted groups are substituted with one or two of the preceding groups. In some embodiments, an optional substituent on an aliphatic carbon atom (acyclic or cyclic) includes oxo (═O).

In some embodiments, each substituted alkyl, substituted fluoroalkyl, substituted heteroalkyl, substituted carbocycle, and substituted heterocycle is substituted with one or more R^{S}groups independently selected from the group consisting of halogen, C₁-C₆alkyl, monocyclic carbocycle, monocyclic heterocycle, —CN, —OR²¹, —CO₂R²¹, —C(═O)N(R²¹)₂, —N(R²¹)₂, —NR²¹C(═O)R²², —SR²¹, —S(═O)R²², —SO₂R²², or —SO₂N(R²¹)₂; each R²¹ is independently selected from hydrogen, C₁-C₆alkyl, C₁-C₆fluoroalkyl, C₁-C₆heteroalkyl, C₃-C₆cycloalkyl, C₂-C₆heterocycloalkyl, phenyl, benzyl, 5-membered heteroaryl and 6-membered heteroaryl; or two R²¹ groups are taken together with the N atom to which they are attached to form a N-containing heterocycle; each R²² is independently selected from C₁-C₆alkyl, C₁-C₆fluoroalkyl, C₁-C₆heteroalkyl, C₃-C₆cycloalkyl, C₂-C₆heterocycloalkyl, phenyl, benzyl, 5-membered heteroaryl and 6-membered heteroaryl.

The term “acceptable” with respect to a formulation, composition or ingredient, as used herein, means having no persistent detrimental effect on the general health of the subject being treated.

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In some embodiments, a modulator is an antagonist.

The terms “administer,” “administering,” “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound of Formula (I′) or (I), or a pharmaceutically acceptable salt thereof, and a co-agent, are both administered to a patient simultaneously in the form of a single entity or dosage. The term “non-fixed combination” means that the active ingredients, e.g. a compound of Formula (I′) or (I), or a pharmaceutically acceptable salt thereof, and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The terms “article of manufacture” and “kit” are used as synonyms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the compounds described herein are administered either alone or in combination with pharmaceutically acceptable carriers, excipients or diluents, in a pharmaceutical composition. Administration of the compounds and compositions described herein can be effected by any method that enables delivery of the compounds to the site of action. These methods include, though are not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route may depend upon for example the condition and disorder of the recipient. By way of example only, compounds described herein can be administered locally to the area in need of treatment, by for example, local infusion during surgery, topical application such as creams or ointments, injection, catheter, or implant. The administration can also be by direct injection at the site of a diseased tissue or organ.

In some embodiments, pharmaceutical compositions suitable for oral administration are presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. In some embodiments, the active ingredient is presented as a bolus, electuary or paste.

Pharmaceutical compositions which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. In some embodiments, the tablets are coated or scored and are formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In some embodiments, stabilizers are added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or Dragee coatings for identification or to characterize different combinations of active compound doses.

In some embodiments, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Pharmaceutical compositions for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical compositions may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

Pharmaceutical compositions may be administered topically, that is by non-systemic administration. This includes the application of a compound of the present invention externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Pharmaceutical compositions suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient may comprise, for topical administration, from 0.001% to 10% w/w, for instance from 1% to 2% by weight of the formulation.

Pharmaceutical compositions for administration by inhalation are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, pharmaceutical preparations may take the form of a dry powder composition, for example a powder mix of the compound and a suitable powder base such as lactose or starch.

The powder composition may be presented in unit dosage form, in for example, capsules, cartridges, gelatin or blister packs from which the powder may be administered with the aid of an inhalator or insufflator.

It should be understood that in addition to the ingredients particularly mentioned above, the compounds and compositions described herein may include other agents conventional in the art having regard to the type of formulation in question, for example those suitable for oral administration may include flavoring agents.

### Methods of Dosing and Treatment Regimens

In one embodiment, the compounds of Formula (I′) or (I), or a pharmaceutically acceptable salt thereof, are used in the preparation of medicaments for the treatment of diseases or conditions in a mammal that would benefit from modulation of parathyroid hormone (PTH) receptor activity. Methods for treating any of the diseases or conditions described herein in a mammal in need of such treatment, involves administration of pharmaceutical compositions that include at least one compound of Formula (I′) or (I) or a pharmaceutically acceptable salt, active metabolite, prodrug, or pharmaceutically acceptable solvate thereof, in therapeutically effective amounts to said mammal.

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in patients, effective amounts for this use will depend on the severity and course of the disease, disorder or condition, previous therapy, the patient's health status and response to the drugs, and the judgment of the treating physician. In one aspect, prophylactic treatments include administering to a mammal, who previously experienced at least one symptom of the disease being treated and is currently in remission, a pharmaceutical composition comprising a compound of Formula (I′) or (I), or a pharmaceutically acceptable salt thereof, in order to prevent a return of the symptoms of the disease or condition.

In certain embodiments wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

Once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In general, however, doses employed for adult human treatment are typically in the range of 0.01 mg-2000 mg per day. In one embodiment, the desired dose is conveniently presented in a single dose or in divided doses administered simultaneously or at appropriate intervals, for example as two, three, four or more sub-doses per day.

In one embodiment, the daily dosages appropriate for the compound of Formula (I′) or (I), or a pharmaceutically acceptable salt thereof, described herein are from about 0.01 to about 50 mg/kg per body weight. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

In any of the aforementioned aspects are further embodiments in which the effective amount of the compound of Formula (I′) or (I), or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal.

In any of the aforementioned aspects are further embodiments comprising single administrations of the effective amount of the compound, including further embodiments in which (i) the compound is administered once a day; or (ii) the compound is administered to the mammal multiple times over the span of one day.

In any of the aforementioned aspects are further embodiments comprising multiple administrations of the effective amount of the compound, including further embodiments in which (i) the compound is administered continuously or intermittently: as in a single dose; (ii) the time between multiple administrations is every 6 hours; (iii) the compound is administered to the mammal every 8 hours; (iv) the compound is administered to the mammal every 12 hours; (v) the compound is administered to the mammal every 24 hours. In further or alternative embodiments, the method comprises a drug holiday, wherein the administration of the compound is temporarily suspended or the dose of the compound being administered is temporarily reduced; at the end of the drug holiday, dosing of the compound is resumed. In one embodiment, the length of the drug holiday varies from 2 days to 1 year.

### Combination Treatments

In certain instances, it is appropriate to administer at least one compound of Formula (I′) or (I), or a pharmaceutically acceptable salt thereof, in combination with one or more other therapeutic agents.

In one embodiment, the therapeutic effectiveness of one of the compounds described herein is enhanced by administration of an adjuvant (i.e., by itself the adjuvant has minimal therapeutic benefit, but in combination with another therapeutic agent, the overall therapeutic benefit to the patient is enhanced). Or, in some embodiments, the benefit experienced by a patient is increased by administering one of the compounds described herein with another agent (which also includes a therapeutic regimen) that also has therapeutic benefit.

In one specific embodiment, a compound of Formula (I′) or (I), or a pharmaceutically acceptable salt thereof, is co-administered with a second therapeutic agent, wherein the compound of Formula (I′) or (I), or a pharmaceutically acceptable salt thereof, and the second therapeutic agent modulate different aspects of the disease, disorder or condition being treated, thereby providing a greater overall benefit than administration of either therapeutic agent alone.

In any case, regardless of the disease, disorder or condition being treated, the overall benefit experienced by the patient is simply be additive of the two therapeutic agents or the patient experiences a synergistic benefit.

For combination therapies described herein, dosages of the co-administered compounds vary depending on the type of co-drug employed, on the specific drug employed, on the disease or condition being treated and so forth. In additional embodiments, when co-administered with one or more other therapeutic agents, the compound provided herein is administered either simultaneously with the one or more other therapeutic agents, or sequentially.

In combination therapies, the multiple therapeutic agents (one of which is one of the compounds described herein) are administered in any order or even simultaneously. If administration is simultaneous, the multiple therapeutic agents are, by way of example only, provided in a single, unified form, or in multiple forms (e.g., as a single pill or as two separate pills).

The compounds of Formula (I′) or (I), or a pharmaceutically acceptable salt thereof, as well as combination therapies, are administered before, during or after the occurrence of a disease or condition, and the timing of administering the composition containing a compound varies. Thus, in one embodiment, the compounds described herein are used as a prophylactic and are administered continuously to subjects with a propensity to develop conditions or diseases in order to prevent the occurrence of the disease or condition. In another embodiment, the compounds and compositions are administered to a subject during or as soon as possible after the onset of the symptoms. In specific embodiments, a compound described herein is administered as soon as is practicable after the onset of a disease or condition is detected or suspected, and for a length of time necessary for the treatment of the disease. In some embodiments, the length required for treatment varies, and the treatment length is adjusted to suit the specific needs of each subject.

### EXAMPLES

As used above, and throughout the description of the invention, the following abbreviations, unless otherwise indicated, shall be understood to have the following meanings:

### Abbreviations

ACN or CH₃CN: acetonitrile;

AcOH: acetic acid;

Ar: aryl or aromatic;

Binap: (±)-2,2′-Bis(diphenylphosphino)-1,1′-binaphthalene,

B(OH)₂: boronic acid;

Bpin: (pinacolato)boron;

(Bpin)₂: Bis(pinacolato)diboron;

Cs₂CO₃: cesium carbonate;

CuI: copper(I) iodide;

Cu(OAc)₂: copper(II) acetate;

DCE: dichloroethane;

DCM: dichloromethane;

DIPEA: N,N-diisopropylethylamine;

DMA: N,N-dimethylacetamide;

DMF: N,N-dimethylformamide;

DMSO: dimethyl sulfoxide;

Et₃N or TEA: triethylamine;

EtOAc: ethyl acetate;

EtOH: ethanol;

FA: formic acid;

Fe: iron (powder);

H₂: hydrogen gas;

HCl: hydrogen chloride;

Het: heterocycle or heteroaromatic;

Hex: hexane;

H₂O: water;

K₂CO₃: potassium carbonate;

KI: potassium iodide;

KOtBu: potassium tert-butoxide;

K₃PO₄: potassium phosphate tribasic;

LCMS: liquid chromatograph mass spectrometer;

LiHMDS: lithium bis(trimethylsilyl)amide;

MeOH: methanol;

MgSO₄: magnesium sulfate (anhydrous);

NaBH₄: sodium borohydride;

NaBH(OAc)₃: sodium triacetoxyborohydride;

NaBH₃CN: sodium cyanoborohydride;

NaH: sodium hydride;

NaHCO₃: sodium bicarbonate;

NaH₂PO₄: monosodium phosphate;

NaI: sodium iodide;

NaIO₄: sodium periodate;

NaNO₂: sodium nitrite;

NaOH: sodium hydroxide;

Na₂S₂O₃: sodium thiosulfate;

Na₂SO₄: sodium sulfate (anhydrous);

NBS: N-bromosuccinimide;

NH₄Cl: ammonium chloride;

NMP: N-methyl-2-pyrrolidone;

Pd: palladium (catalyst);

Pd/C: palladium on carbon;

Pd₂(dba)₃: tris(dibenzylideneacetone)dipalladium(O);

Pd₂(dba)₃-CHCl₃: tris(dibenzylideneacetone)dipalladium(O)-chloroform adduct;

Pd(dppf)Cl₂: [1,1′-Bis(diphenylphosphino)ferrocene]dichloropalladium(II);

Pd(dtbpf)Cl₂: [1,1′-Bis (di-t-butylphosphino)ferrocene]dichloropalladium(II);

Pd(OAc)₂: palladium(II) acetate;

Pd(PPh₃)₄: tetrakis(triphenylphosphine)palladium(O);

PE: petroleum ether;

Ph₃P: triphenylphosphine;

Prep-HPLC: preparative high performance liquid chromatography;

p-TsOH: p-toluenesulfonic acid;

TFA: trifluoroacetic acid;

THF: tetrahydrofuran;

Trixiephos: rac-2-(Di-tert-butylphosphino)-1,1′-binaphthyl;

Xantphos: 4,5-Bis(diphenylphosphino)-9,9-dimethylxanthene;

XPhos: 2-dicyclohexylphosphino-2′,4′,6′-triisopropylbiphenyl;

ZnCl₂: zinc(II) chloride;

Zn(CN)₂: zinc(II) cyanide;

rt: room temperature;

h: hour or hours;

Cpd: compound

### Example 1: 4,13-dichloro-10-(2,6-difluoro-4-{[2-(methylamino)ethyl]amino}phenyl)-8-ethyl-6,8,10-triazatricyclo[9.4.0.0^{2,7}]pentadeca-1(11),2(7),3,5,12,14-hexaen-9-one (cpd 1-8)

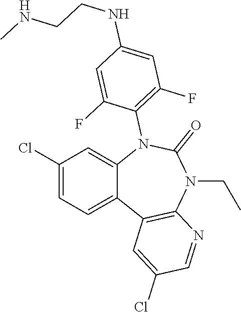 [SMILES: CCN1C(=O)N(c2c(F)cc(NCCNC)cc2F)c2cc(Cl)ccc2-c2cc(Cl)cnc21]

Step 1-1, preparation of 3-bromo-5-chloro-N-ethylpyridin-2-amine: To a solution of 3-bromo-5-chloropyridin-2-amine (9.53 g, 45.0 mmol) in anhydrous THF (100 mL) under nitrogen was added sodium hydride (2.70 g, 60% wt, 67.5 mmol) in one portion, then mixture was stirred at 40° C. for 15 min. After cooling the mixture to room temperature, iodoethane (7.96 g, 49.5 mmol) was added dropwise, and reaction mixture was stirred at 60° C. for 2 h. It was cooled to room temperature then partitioned between sat. aq. NH₄Cl and ethyl acetate. The organic layer was washed with sat. aq. NaHCO₃ and brine, then dried over sodium sulfate and concentrated. The residue was purified by silica gel column chromatography (0-100% EtOAc/hexane) to give 3-bromo-5-chloro-N-ethylpyridin-2-amine (10.4 g, 98%) as a pale yellow oil. LCMS (M+H)⁺=235.1.

Step 1-2, preparation of 5-chloro-2-(4,4,5,5-tetramethyl-1,3,2-dioxaborolan-2-yl)aniline: To a solution of 2-bromo-5-chloroaniline (9.06 g, 43.0 mmol) in DMSO (80 mL) under nitrogen was added 4,4,4′,4′,5,5,5′,5′-octamethyl-2,2′-bi(1,3,2-dioxaborolane) (13.4 g, 51.6 mmol), Pd(dppf)Cl₂ (2.5 g, 3.3 mmol), and potassium acetate (12.8 g, 129 mmol). The mixture was stirred at 85° C. for 13 h. The mixture was diluted with ethyl acetate, washed with sat. aq. NH₄Cl, sat. aq. NaHCO₃, and brine, and dried over sodium sulfate. After concentrating, the residue was purified via silica gel column chromatography (0-100% EtOAc/hexane) to give 5-chloro-2-(4,4,5,5-tetramethyl-1,3,2-dioxaborolan-2-yl)aniline as a pale green oil (9.77 g, 90%). LCMS (M+H)⁺=254.4.

Step 1-3, preparation of 3-(2-amino-4-chlorophenyl)-5-chloro-N-ethylpyridin-2-amine: To a solution of 3-bromo-5-chloro-N-ethylpyridin-2-amine (9.2 g, 39 mmol, from “Step 1-1”) in 1,4-dioxane (80 mL) under nitrogen was added 5-chloro-2-(4,4,5,5-tetramethyl-1,3,2-dioxaborolan-2-yl)aniline (9.768 g, 38.53 mmol), potassium acetate (12.8 g, 129 mmol), Pd(dppf)Cl₂ (2.5 g, 3.3 mmol), and water (8 mL). The mixture was heated at 95° C. for 1 h, then cooled, diluted with ethyl acetate, washed with sat. aq. NH₄Cl, sat. aq. NaHCO₃, and brine, and dried over sodium sulfate. After concentrating, the residue was purified via reverse-phase C18 column chromatography (10-100% water/ACN, 0.1% TFA) to give as a white solid 3-(2-amino-4-chlorophenyl)-5-chloro-N-ethylpyridin-2-amine (8.43 g, 78%). LCMS (M+H)⁺=282.3.

Step 1-4, preparation of 2,9-dichloro-5-ethyl-5,7-dihydro-6H-benzo[d]pyrido[3,2-f][1,3]diazepin-6-one: To a solution of 3-(2-amino-4-chlorophenyl)-5-chloro-N-ethylpyridin-2-amine (8.43 g, 29.9 mmol) in DCM (250 mL) at 0° C. under nitrogen was added triethylamine (7.56 g, 74.7 mmol), followed a solution of triphosgene (4.43 g, 14.9 mmol) in DCM (100 mL). After stirring at room temperature for 1 h, it was poured into 1:1 sat. aq. NaHCO₃/brine and the organic layer was washed with sat. aq. NH₄Cl and brine, then dried over sodium sulfate. It was concentrated to ¼ volume to give a thick slurry, which was filtered to isolate as a white solid 2,9-dichloro-5-ethyl-5,7-dihydro-6H-benzo[d]pyrido[3,2-f][1,3]diazepin-6-one (7.99 g, 87%). LCMS (M+H)⁺=308.2.

Step 1-5, preparation of 2,9-dichloro-7-(2,6-difluoro-4-nitrophenyl)-5-ethyl-5,7-dihydro-6Hbenzo[d]pyrido[3,2-f][1,3]diazepin-6-one: To a mixture of 2,9-dichloro-5-ethyl-5,7-dihydro-6H-benzo[d]pyrido[3,2-f][1,3]diazepin-6-one (7.99 g, 25.9 mmol) and 1,2,3-trifluoro-5-nitrobenzene (12 g, 65 mmol) in DMF (150 mL) at 0° C. under nitrogen was added NaH (3.12 g, 60% wt, 78.1 mmol), and the reaction mixture was stirred for 75 min at 0° C. It was then poured into cold sat. aq. NH₄Cl and extracted with ethyl acetate. The organic layer was washed with water, sat. aq. NaHCO₃, and brine, dried over sodium sulfate, concentrated, and purified by silica gel column chromatography (0-60% EtOAc/hexane). The resulting solid was triturated with EtOAc-hexane (1:20) and collected by filtration to give as a white solid 2,9-dichloro-7-(2,6-difluoro-4-nitrophenyl)-5-ethyl-5,7-dihydro-6Hbenzo[d]pyrido[3,2-f][1,3]diazepin-6-one (11.4 g, 94%). LCMS (M+H)³⁰=465.2.

Step 1-6, preparation of 7-(4-amino-2,6-difluorophenyl)-2,9-dichloro-5-ethyl-5,7-dihydro-6H-benzo[d]pyrido-[3,2-f][1,3]diazepin-6-one: A mixture of 2,9-dichloro-7-(2,6-difluoro-4-nitrophenyl)-5-ethyl-5,7-dihydro-6H-benzo[d]pyrido-[3,2-f][1,3]diazepin-6-one (4.00 g, 8.60 mmol) and iron powder (3.60 g, 64.5 mmol) in AcOH (40 mL) was heated with vigorous stirring at 75 deg C. overnight to give a thick off-white slurry. Water (200 mL) was added, then the mixture was filtered. The collected solid product was triturated with minimum amounts of cold ethanol (2×) and ether (2×), then dried in vacuo to give as a pale yellow solid 7-(4-amino-2,6-difluorophenyl)-2,9-dichloro-5-ethyl-5,7-dihydro-6H-benzo[d]pyrido-[3,2-f][1,3]diazepin-6-one (3.50 g, 93.5%). LCMS (M+H)⁺=435.1.

Step 1-7, preparation of tert-butyl (2-((4-(2,9-dichloro-5-ethyl-6-oxo-5,6-dihydro-7H-benzo[d]pyrido[3,2-f][1,3]diazepin-7-yl)-3,5-difluorophenyl)amino)ethyl)(methyl)carbamate: A mixture of 7-(4-amino-2,6-difluorophenyl)-2,9-dichloro-5-ethyl-5,7-dihydro-6H-benzo[d]pyrido[3,2-f][1,3]diazepin-6-one (22 mg, 51 μmol), tert-butyl methyl(2-oxoethyl)carbamate (28 mg, 0.15 mmol), and acetic acid (6.1 mg, 0.10 mmol) in MeOH (1.0 mL) was stirred at 60° C. for 15 minutes. A solution of zinc chloride (100 μL, 1.9 molar in 2-methyl-THF, 190 μmol) was added, followed by sodium cyanoborohydride (6.4 mg, 0.10 mmol). The mixture was stirred 3 h, then partitioned between DCM and sat. aq. NaHCO₃. The organic layer was washed with brine, dried over sodium sulfate, concentrated and purified by silica gel column chromatography (0-100% EtOAc/hexane) to give as a colorless oil tert-butyl (2-((4-(2,9-dichloro-5-ethyl-6-oxo-5,6-dihydro-7H-benzo[d]pyrido[3,2-f][1,3]diazepin-7-yl)-3,5-difluorophenyl)amino)ethyl)(methyl)carbamate (30 mg, 100%). LCMS (M+H)⁺=592.4.

Step 1-8, preparation of 4,13-dichloro-10-(2,6-difluoro-4-{[2-(methylamino)ethyl]amino}phenyl)-8-ethyl-6,8,10-triazatricyclo[9.4.0.0^{2,7}]pentadeca-1(11),2(7),3,5,12,14-hexaen-9-one: To the solution of tert-butyl (2-((4-(2,9-dichloro-5-ethyl-6-oxo-5,6-dihydro-7H-benzo[d]pyrido[3,2-f][1,3]diazepin-7-yl)-3,5-difluorophenyl)amino)ethyl)(methyl)carbamate (30 mg, 51 μmol) in DCM (0.7 mL) was added TFA (0.3 mL). After 40 min stirring, it was concentrated and purified by C18 reverse-phase chromatography (10-100% ACN/water, 0.1% TFA) to give the TFA salt of the title compound (25 mg, 80%). LCMS (M+H)⁺=492.3.

The following compounds were prepared similarly to Example 1 with appropriate substituting reagents and substrates at different steps and may require additional functional group modifications, via well-known chemistry with appropriate reagents. The reductive amination reaction can be carried out with other reductants such as sodium triacetoxyborohydride under appropriate conditions. Different salts, such as HCl, formic acid, or TFA salts, may be obtained.

| Compound no. | MS (M + H)⁺ |
|---|---|
| 1-1 | 444.4 |
| 1-2 | 458.1 |
| 1-4 | 528.1 |
| 1-5 | 514.1 |
| 1-12 | 562.2 |
| 1-13 | 548.4 |
| 1-14 | 476.1 |
| 1-18 | 472.2 |
| 1-19 | 546.4 |
| 1-20 | 532.2 |
| 1-21 | 472.2 |
| 1-23 | 472.2 |
| 1-24 | 498.2 |
| 1-27 | 542.4 |
| 1-28 | 528.1 |
| 1-29 | 488.0 |
| 1-30 | 478.3 |
| 1-32 | 490.2 |
| 1-35 | 548.3 |
| 1-36 | 506.2 |
| 1-38 | 510.1 |
| 1-39 | 506.2 |
| 1-40 | 506.2 |
| 1-41 | 493.1 |
| 1-45 | 546.1 |
| 1-46 | 518.2 |
| 1-50 | 507.0 |
| 1-53 | 549.2 |
| 1-57 | 525.8 |
| 1-59 | 524.3 |
| 1-61 | 510.2 |
| 1-66 | 510.1 |
| 1-75 | 476.2 |
| 1-78 | 504.2 |
| 1-87 | 506.2 |
| 1-89 | 483.2 |
| 1-94 | 494.2 |
| 1-105 | 490.2 |
| 1-113 | 508.1 |
| 1-130 | 476.2 |
| 1-135 | 458.3 |
| 2-1 | 408.4 |
| 2-2 | 426.1 |
| 2-3 | 456.5 |
| 2-4 | 440.2 |
| 2-5 | 436.0 |
| 2-6 | 452.2 |
| 2-8 | 474.1 |
| 2-11 | 475.2 |
| 2-12 | 544.3 |
| 2-13 | 526.3 |
| 2-14 | 545.3 |
| 3-1 | 472.2 |
| 3-3 | 472.2 |
| 3-5 | 506.1 |
| 3-7 | 490.2 |
| 3-9 | 510.5 |
| 3-16 | 483.2 |
| 3-17 | 553.3 |

### Example 2: 4,13-dichloro-10-[2,6-difluoro-4-({2-[(2-hydroxyethyl)amino]ethyl}amino)phenyl]-8-ethyl-6,8,10-triazatricyclo[9.4.0.0^{2,7}]pentadeca-1(11),2(7),3,5,12,14-hexaen-9-one (cpd 1-11)

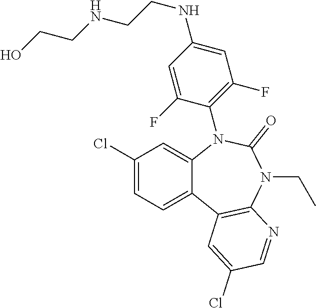 [SMILES: CCN1C(=O)N(c2c(F)cc(NCCNCCO)cc2F)c2cc(Cl)ccc2-c2cc(Cl)cnc21]

Step 2-1, preparation of tert-butyl (2-((tert-butyldimethylsilyl)oxy)ethyl)(2-hydroxyethyl)carbamate: To tert-butyl bis(2-hydroxyethyl)carbamate (5.00 g, 24.4 mmol) and triethylamine (7.15 g, 9.85 mL, 70.6 mmol) in DCM (75 mL) at 0° C. under nitrogen was added, dropwise over 1 h, a solution of tert-butylchlorodimethylsilane (4.77 g, 31.7 mmol) in DCM (25 mL). It was stirred overnight at RT, then washed with water (2×) and brine, then dried over MgSO₄, concentrated and purified by silica gel chromatography (0-40% EtOAc/hexane) to provide tert-butyl (2-((tert-butyldimethylsilyl)oxy)ethyl)(2-hydroxyethyl)carbamate (5.3 g, 68%) as a colorless oil. LCMS (M+H)⁺=320.1.

Step 2-2, preparation of tert-butyl (2-((tert-butyldimethylsilyl)oxy)ethyl)(2-oxoethyl)carbamate: To oxalyl dichloride (2.69 g, 1.85 mL, 21.2 mmol) in DCM (50 mL) at −78° C. under nitrogen was added dropwise a solution of DMSO (3.31 g, 3.00 mL, 42.3 mmol) in DCM (15 mL) via cannula, and the mixture was stirred 30 min at the same temperature. Then a solution of tert-butyl (2-((tert-butyldimethylsilyl)oxy)ethyl)(2-oxoethyl)carbamate (4.5 g, 14 mmol) in DCM (15 mL) was added dropwise via cannula and stirring was continued for 1 h. Finally, triethylamine (9.06 g, 13.0 mL, 89.5 mmol) was added dropwise. The mixture was stirred at −78° C. for 15 min then allowed to reach RT over 1 h. It was diluted with DCM, washed with water (2×) and brine, dried over MgSO₄ and concentrated to give as a pale yellow oil tert-butyl (2-((tert-butyldimethylsilyl)oxy)ethyl)(2-oxoethyl)carbamate (4.5 g, 87%), which was used without purification. LCMS (M+H)⁺=no ionization.

Step 2-3, preparation of tert-butyl (2-((tert-butyldimethylsilyl)oxy)ethyl)(2-((4-(2,9-dichloro-5-ethyl-6-oxo-5,6-dihydro-7H-benzo[d]pyrido[3,2-f][1,3]diazepin-7-yl)-3,5-difluorophenyl)amino)ethyl)carbamate: To 7-(4-amino-2,6-difluorophenyl)-2,9-dichloro-5-ethyl-5,7-dihydro-6H-benzo[d]pyrido[3,2-f][1,3]diazepin-6-one (from “Step 1-6, Example 1”) (160 mg, 0.368 mmol) in MeOH (4 mL), was added tert-butyl (2-((tert-butyldimethylsilyl)oxy)ethyl)(2-oxoethyl)carbamate (from “Step 2-2, Example 2”) (233 mg, 0.734 mmol) and sodium cyanoborohydride (92.4 mg, 1.47 mmol). The resulting solution was stirred 4 h at 60° C., then purified directly by C18 reverse-phase chromatography (30-98% ACN/water, 0.1% FA) to give tert-butyl (2-((tert-butyldimethylsilyl)oxy)ethyl)(2-((4-(2,9-dichloro-5-ethyl-6-oxo-5,6-dihydro-7H-benzo[d]pyrido[3,2-f][1,3]diazepin-7-yl)-3,5-difluorophenyl)amino)ethyl)carbamate (100 mg, 37%) as white solid. LCMS (M+H)⁺: 736.3.

Step 2-4, preparation of 4,13-dichloro-10-[2,6-difluoro-4-({2-[(2-hydroxyethyl)amino]ethyl}amino)phenyl]-8-ethyl-6,8,10-triazatricyclo[9.4.0.0^{2,7}]7pentadeca-1(11),2(7),3,5,12,14-hexaen-9-one: A solution of tert-butyl (2-((tert-butyldimethylsilyl)oxy)ethyl)(2-((4-(2,9-dichloro-5-ethyl-6-oxo-5,6-dihydro-7H-benzo[d]pyrido[3,2-f][1,3]diazepin-7-yl)-3,5-difluorophenyl)amino)ethyl)carbamate (100 mg, 136 μmol) and TFA (2 mL) was stirred 4 h at RT, then concentrated and purified by prep-HPLC under the following conditions: column, SunFire Prep C18 OBD, 5 um, 19*150 mm; mobile phase, 25-65% ACN/water, 0.1% TFA, over 10 min; detector, UV 220/254 nm. This gave the TFA salt of the title compound as a white solid (72 mg, 71%). LCMS (M+H)⁺=522.2.

The following compounds were prepared similarly to Example 2 with appropriate substituting reagents and substrates at different steps and may require additional functional group modifications, via well-known chemistry with appropriate reagents. The reductive amination reaction can be carried out with other reductants such as sodium triacetoxyborohydride under appropriate conditions. Different salts, such as HCl, formic acid, or TFA salts, may be obtained.

| Compound no. | MS (M + H)⁺ |
|---|---|
| 1-7 | 488.4 |
| 1-17 | 506.2 |
| 1-26 | 502.3 |
| 1-31 | 520.2 |
| 1-34 | 508.4 |
| 1-37 | 540.2 |
| 1-47 | 536.2 |
| 1-48 | 576.2 |
| 1-49 | 536.2 |
| 1-51 | 536.2 |
| 1-52 | 548.2 |
| 1-56 | 556.1 |
| 1-60 | 540.2 |
| 1-67 | 540.2 |
| 1-71 | 536.2 |
| 1-73 | 536.2 |
| 1-74 | 536.2 |
| 1-76 | 506.2 |
| 1-79 | 534.2 |
| 1-88 | 536.2 |
| 1-90 | 513.2 |
| 1-95 | 524.2 |
| 1-106 | 520.2 |
| 1-114 | 538.2 |
| 1-129 | 506.2 |
| 1-134 | 531.3 |
| 1-142 | 488.3 |
| 2-7 | 504.2 |
| 2-9 | 486.1 |
| 2-10 | 505.2 |
| 3-2 | 502.2 |
| 3-4 | 536.4 |
| 3-6 | 520.2 |
| 3-10 | 540.2 |
| 3-15 | 513.3 |

### Example 3: 10-{4-[(2-aminoethyl)amino]-2,6-difluorophenyl}-4,13-dichloro-8-ethyl-6,8,10-triazatricyclo[9.4.0.0^{2,7}]pentadeca-1(11),2(7),3,5,12,14-hexaen-9-one (cpd 1-9)

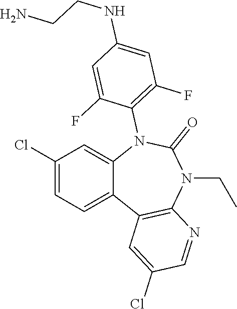 [SMILES: CCN1C(=O)N(c2c(F)cc(NCCN)cc2F)c2cc(Cl)ccc2-c2cc(Cl)cnc21]

Step 3-1, preparation of tert-butyl (2-((4-(2,9-dichloro-5-ethyl-6-oxo-5,6-dihydro-7H-benzo[d]pyrido[3,2-f][1.3]diazepin-7-yl)-3,5-difluorophenyl)amino)ethyl)carbamate: To a mixture of 7-(4-amino-2,6-difluorophenyl)-2,9-dichloro-5-ethyl-5,7-dihydro-6H-benzo[d]pyrido[3,2-f][1,3]diazepin-6-one (from “Step 1-6, Example 1”) (1.22 g, 2.80 mmol) in MeOH (25 mL) was added tert-butyl (2-oxoethyl)carbamate (1.41 g, 8.41 mmol), acetic acid (340 mg, 5.6 mmol) and zinc(II) chloride solution (3.0 mL, 1.9 molar in 2-methyl-THF, 5.6 mmol). After the mixture was heated at 60° C. for 30 min, sodium cyanoborohydride (350 mg, 5.6 mmol) was added and heating was continued for 2 h. The reaction was diluted with DCM, washed with sat. aq. NaHCO₃ and brine, dried over sodium sulfate, concentrated, and purified by C18 reverse-phase chromatography (10-100% ACN/water, 0.1% TFA) to yield tert-butyl (2-((4-(2,9-dichloro-5-ethyl-6-oxo-5,6-dihydro-7H-benzo[d]pyrido[3,2-f][1,3]diazepin-7-yl)-3,5-difluorophenyl)amino)ethyl)carbamate (1.33 g, 82%) an off-white foam. LCMS (M+H)⁺=578.3.

Step 3-2, preparation of 10-{4-[(2-aminoethyl)amino]-2,6-difluorophenyl}-4,13-dichloro-8-ethyl-6,8,10-triazatricyclo[9.4.0.0^{2,7}]pentadeca-1(11),2(7),3,5,12,14-hexaen-9-one: A solution of tert-butyl (2-((4-(2,9-dichloro-5-ethyl-6-oxo-5,6-dihydro-7H-benzo[d]pyrido[3,2-f][1,3]diazepin-7-yl)-3,5-difluorophenyl)amino)ethyl)carbamate (1.33 g, 2.30 mmol) and TFA (3 mL) in DCM (10 mL) was stirred at RT for 1 h, then concentrated and purified by C18 reverse-phase chromatography (10-100% ACN/water, 0.1% TFA) to give the TFA salt of the title compound (1.15 g, 85%) as a white foam. LCMS (M+H)⁺=478.2.

The following compounds were prepared similarly to Example 3 with appropriate substituting reagents and substrates at different steps and may require additional functional group modifications, via well-known chemistry with appropriate reagents. The reductive amination reaction can be carried out with other reductants such as sodium triacetoxyborohydride under appropriate conditions. Different salts, such as HCl, formic acid, or TFA salts, may be obtained.

| Compound no. | MS (M + H)⁺ |
|---|---|
| 1-3 | 444.4 |
| 1-15 | 462.1 |
| 1-22 | 458.4 |
| 1-33 | 464.2 |
| 3-8 | 492.3 |

### Example 4: 4-({2-[(4-{4,13-dichloro-8-ethyl-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)butanoic Acid (cod 1-10)

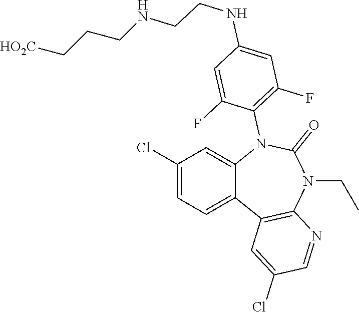 [SMILES: CCN1C(=O)N(c2c(F)cc(NCCNCCCC(=O)O)cc2F)c2cc(Cl)ccc2-c2cc(Cl)cnc21]

Step 4-1, preparation of tert-butyl 4-((2-((4-(2,9-dichloro-5-ethyl-6-oxo-5,6-dihydro-7H-benzo[d]pyrido[3,2-f][1.3]diazepin-7-yl)-3,5-difluorophenyl)amino)ethyl)amino)butanoate: To 10-{4-[(2-aminoethyl)amino]-2,6-difluorophenyl}-4,13-dichloro-8-ethyl-6,8,10-triazatricyclo[9.4.0.0^{2,7}]pentadeca-1(11),2(7),3,5,12,14-hexaen-9-one (Cpd 1-9, from “Step 3-2, Example 3”) (990 mg, 1.67 mmol) in MeOH (15 mL) was added tert-butyl 4-oxobutanoate (264 mg, 1.67 mmol), acetic acid (0.1 mL, 2 mmol), and a solution of zinc(II) chloride (880 μL, 1.9 molar in 2-Me-THF, 1.67 mmol). The reaction mixture was stirred at 50° C. for 30 min, then sodium cyanoborohydride (158 mg, 2.51 mmol) was added. The mixture was stirred 2.5 h at RT, then partitioned between DCM and sat. aq. NaHCO₃. The aqueous was extracted with DCM and the combined organics were washed with brine, dried over sodium sulfate, concentrated, and purified by C18 reverse-phase chromatography (10-100% ACN/water, 0.1% TFA) to provide tert-butyl 4-((2-((4-(2,9-dichloro-5-ethyl-6-oxo-5,6-dihydro-7H-benzo[d]pyrido[3,2-f][1,3]diazepin-7-yl)-3,5-difluorophenyl)amino)ethyl)amino)butanoate (1.07 g, 87%) as a white foam. LCMS (M+H)⁺=620.1.

Step 4-2, preparation of 4-({2-[(4-{4,13-dichloro-8-ethyl-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)butanoic acid: To tert-butyl 4-((2-((4-(2,9-dichloro-5-ethyl-6-oxo-5,6-dihydro-7H-benzo[d]pyrido[3,2-f][1,3]diazepin-7-yl)-3,5-difluorophenyl)amino)ethyl)amino)butanoate (1.07 g, 1.45 mmol) at 0° C. was added TFA (2.75 mL, 35.7 mmol) and the solution was stirred for 1 h at RT. The mixture was concentrated then purified by C18 reverse-phase chromatography (10-100% ACN/water, 0.1% TFA). The aqueous fractions containing compound were treated with 4N HCl_{(aq)} (1.5 mL), then concentrated to ½ volume. The mixture was cooled in an ice-bath and the resulting solid was collected by filtration, rinsed with water, and dried in vacuo to provide the HCl salt of the title compound (675 mg, 77%) as a white solid. LCMS (M+H)⁺=564.4.

The following compounds were prepared similarly to Example 4 with appropriate substituting reagents and substrates at different steps and may require additional functional group modifications, via well-known chemistry with appropriate reagents. The reductive amination reaction can be carried out with other reductants such as sodium triacetoxyborohydride under appropriate conditions. Different salts such as HCl formic acid or TFA salts, may be obtained.

| Compound no. | MS (M + H)⁺ |
|---|---|
| 1-6 | 530.1 |
| 1-16 | 548.2 |
| 1-25 | 544.4 |
| 1-64 | 562.2 |
| 1-69 | 582.2 |
| 1-70 | 578.3 |
| 1-72 | 550.4 |
| 1-77 | 548.2 |
| 1-80 | 576.3 |
| 1-96 | 566.2 |
| 3-11 | 582.4 |

### Example 5: 10-{4-[(3R)-3-aminopyrrolidin-1-yl]-2,6-difluorophenyl}-4,13-dichloro-8-ethyl-6,8,10-triazatricyclo[9.4.0.0^{2,7}]pentadeca-1(11),2(7),3,5,12,14-hexaen-9-one (cpd 1-42)

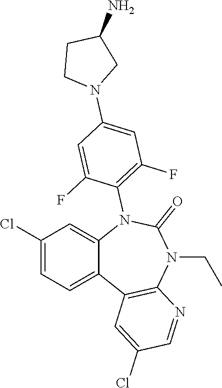 [SMILES: CCN1C(=O)N(c2c(F)cc(N3CC[C@@H](N)C3)cc2F)c2cc(Cl)ccc2-c2cc(Cl)cnc21]

Step 5-1, preparation of 2,9-dichloro-7-(2,6-difluoro-4-iodophenyl)-5-ethyl-5,7-dihydro-6H-benzo[d]pyrido[3,2-f][1,3]diazepin-6-one: To a mixture of 7-(4-amino-2,6-difluorophenyl)-2,9-dichloro-5-ethyl-5,7-dihydro-6H-benzo[d]pyrido[3,2-f][1,3]diazepin-6-one (from “Step 1-6, Example 1”) (300 mg, 689 μmol) and conc. HCl (3 mL) at 0° C. was added dropwise NaNO₂ (143 mg, 2.07 mmol) in water (2 mL). After stirring 30 min at 0° C., a solution of KI (1.14 g, 6.87 mmol) in ACN (3 mL) was added dropwise then stirring was continued 1 h at 0° C. The reaction mixture was diluted with water and extracted with ethyl acetate (3×). The combined organic layers were dried over sodium sulfate and concentrated, then the residue was purified by silica gel column chromatography (50% EtOAc/hexane) to give as a yellow oil 2,9-dichloro-7-(2,6-difluoro-4-iodophenyl)-5-ethyl-5,7-dihydro-6H-benzo[d]pyrido[3,2-f][1,3]diazepin-6-one (240 mg, 64%). LCMS (M+H)+=546.1.

Step 5-2, preparation of tert-butyl (R)-(1-(4-(2,9-dichloro-5-ethyl-6-oxo-5,6-dihydro-7H-benzo[d]pyrido[3,2-f][1,3]diazepin-7-yl)-3,5-difluorophenyl)pyrrolidin-3-yl)carbamate: A flask under nitrogen was charged with 2,9-dichloro-7-(2,6-difluoro-4-iodophenyl)-5-ethyl-5,7-dihydro-6H-benzo[d]pyrido[3,2-f][1,3]diazepin-6-one (25 mg, 46 μmol), tert-butyl (R)-pyrrolidin-3-ylcarbamate (26 mg, 0.14 mmol), xantphos (5.3 mg, 9.2 μmol), Pd₂(dba)₃ (4.2 mg, 4.6 μmol), Cs₂CO₃ (45 mg, 0.14 mmol) and 1,4-dioxane (1 mL). The resulting mixture was stirred 1 h at 100° C., then cooled, concentrated, and purified by silica gel column chromatography (50% EtOAc/hexane) to afford tert-butyl (R)-(1-(4-(2,9-dichloro-5-ethyl-6-oxo-5,6-dihydro-7H-benzo[d]pyrido[3,2-f][1,3]diazepin-7-yl)-3,5-difluorophenyl)pyrrolidin-3-yl)carbamate (20 mg, 72%) as a yellow oil. LCMS (M+H)+=604.4.

Step 5-3, preparation of 10-{4-[(3R)-3-aminopyrrolidin-1-yl]-2,6-difluorophenyl}-4,13-dichloro-8-ethyl-6,8,10-triazatricyclo[9.4.0.0^{2,7}]pentadeca-1(11),2(7),3,5,12,14-hexaen-9-one: From tert-butyl (R)-(1-(4-(2,9-dichloro-5-ethyl-6-oxo-5,6-dihydro-7H-benzo[d]pyrido[3,2-f][1,3]diazepin-7-yl)-3,5-difluorophenyl)pyrrolidin-3-yl)carbamate (20 mg, 33 μmol), the title compound (17 mg, 69%) was prepared using a similar method as described in “Step 2-4, Example 2.” LCMS (M+H)⁺=504.2.

The following compounds were prepared similarly to Example 5 with appropriate substituting reagents and substrates at different steps and may require additional functional group modifications, via well-known chemistry with appropriate reagents. The amination reaction can be carried out with other metal/catalyst systems under appropriate conditions. Different salts, such as HCl, formic acid, or TFA salts, may be obtained.

| Compound no. | MS (M + H)⁺ |
|---|---|
| 1-43 | 504.1 |
| 1-44 | 490.1 |
| 1-65 | 502.2 |

### Example 6: 4-chloro-10-(2,6-difluoro-4-{[2-(methylamino)ethyl]amino}phenyl)-8-ethyl-9-oxo-6,8,10-triazatricyclo[9.4.0.0^{2,7}]pentadeca-1(11),2(7),3,5,12,14-hexaene-13-carbonitrile (cpd 1-54)

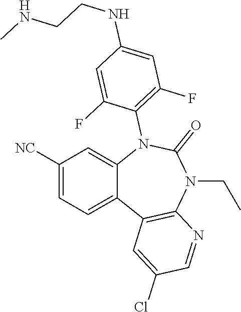 [SMILES: CCN1C(=O)N(c2c(F)cc(NCCNC)cc2F)c2cc(C#N)ccc2-c2cc(Cl)cnc21]

Step 6-1, preparation of 3-amino-4-(4,4,5,5-tetramethyl-1,3,2-dioxaborolan-2-yl)benzonitrile: To a solution of 3-amino-4-bromobenzonitrile (10.4 g, 51.7 mmol) in DMSO (80 mL) under nitrogen was added 4,4,4′,4′,5,5,5′,5′-octamethyl-2,2′-bi(1,3,2-dioxaborolane) (16.1 g, 62.1 mmol), Pd(dppf)Cl₂ (1.2 g, 51.7 mmol), and potassium acetate (15.4 g, 155 mmol). The mixture was stirred at 85° C. for 15 hours, then diluted with ethyl acetate, washed with water, sat. aq. NH₄Cl, sat. aq. NaHCO₃, and brine, then dried over magnesium sulfate. It was concentrated and purified via silica gel column chromatography (0-100% EtOAc/hexane) to give as an off-white solid 3-amino-4-(4,4,5,5-tetramethyl-1,3,2-dioxaborolan-2-yl)benzonitrile (10.4 g, 82%). LCMS (M+H)⁺=245.3.

Step 6-2, preparation of 3-amino-4-(5-chloro-2-(ethylamino)pyridin-3-Yl)benzonitrile: To a solution of 3-bromo-5-chloro-N-ethylpyridin-2-amine (from “Step 1-1, Example 1”) (7.59 g, 32.3 mmol) and 3-amino-4-(4,4,5,5-tetramethyl-1,3,2-dioxaborolan-2-yl)benzonitrile (8.68 g, 35.5 mmol) in 1,4-dioxane (90 mL) under nitrogen atmosphere was added potassium carbonate (10 g, 72 mmol), palladium tetrakis(triphenylphosphine) (1.0 g, 0.90 mmol), and water (10 mL). After the mixture was heated at 90° C. for 2.5 hours, additional 3-amino-4-(4,4,5,5-tetramethyl-1,3,2-dioxaborolan-2-yl)benzo-nitrile (350 mg), palladium tetrakis(triphenylphosphine) (380 mg), and potassium carbonate (2.8 g) were added and the reaction heated at 90° C. for a further 2 hours. It was then cooled to RT, diluted with ethyl acetate, washed sequentially with sat. aq. ammonium chloride, water, and brine, dried over anhydrous sodium sulfate, then concentrated. The resulting oil was loaded onto a silica column. During this process, a significant amount of yellow solid crashed out. These solids were collected by filtration to afford the first batch of product in an amount of 6.11 g. Silica gel column chromatography (50% EtOAc/hexane) of the remaining oil gave a second batch of the product in an amount of 1.42 g. The total amount of 3-amino-4-(5-chloro-2-(ethylamino)pyridin-3-yl)benzonitrile was 7.53 g (86%). LCMS (M+H)⁺=273.0.

Step 6-3, preparation of 2-chloro-5-ethyl-6-oxo-6,7-dihydro-5H-benzo[d]pyrido[3,2-f][1,3]diazepine-9-carbonitrile: To a suspension of 3-amino-4-(5-chloro-2-(ethylamino)pyridin-3-yl)benzonitrile (6.11 g, 22.4 mmol) in DCM (110 mL) at 0° C. under nitrogen was added triethylamine (6.80 g, 67.2 mmol). A solution of bis(trichloromethyl)carbonate (2.66 g, 8.96 mmol) in DCM (15 mL) was added dropwise, the mixture stirred 30 min at RT, then cooled to 0° C. The reaction mixture was filtered, the solids collected and rinsed with hexanes to give as a white solid 2-chloro-5-ethyl-6-oxo-6,7-dihydro-5H-benzo[d]pyrido[3,2-f][1,3]diazepine-9-carbonitrile (8.0 g, 87%). LCMS (M+H)⁺=299.3.

Step 6-4, preparation of 2-chloro-7-(2,6-difluoro-4-nitrophenyl)-5-ethyl-6-oxo-6,7-dihydro-5H-benzo[d]pyrido-[3,2-f][1,3]diazepine-9-carbonitrile: To a suspension of 2-chloro-5-ethyl-6-oxo-6,7-dihydro-5H-benzo[d]pyrido[3,2-f][1,3]diazepine-9-carbonitrile (5.68 g, 19.0 mmol) and 1,2,3-trifluoro-5-nitrobenzene (8.76 g, 47.5 mmol) in DMF (100 mL) at 0° C. under nitrogen was added portionwise sodium hydride (2.28 g, 60% wt, 57.0 mmol). After stirring at 0° C. for 1.5 h, the mixture was added to cold sat. aq. NH₄Cl, then extracted with ethyl acetate. The organics were further washed with water, sat. aq. sodium bicarbonate, brine, then dried over sodium sulfate and concentrated. Addition of DCM to the oily residue provided a solid, which was collected and rinsed with DCM to give the first batch of product (4.78 g). The solute was concentrated then purified by silica gel column chromatography (0-100% EtOAc/hexane) to give a second batch of product (3.32 g). The total amount of 2-chloro-7-(2,6-difluoro-4-nitrophenyl)-5-ethyl-6-oxo-6,7-dihydro-5H-benzo[d]pyrido-[3,2-f][1,3]diazepine-9-carbonitrile, isolated as a white solid, was 8.11 g (94%). LCMS (M+H)⁺=456.3.

Step 6-5, preparation of 7-(4-amino-2,6-difluorophenyl)-2-chloro-5-ethyl-6-oxo-6,7-dihydro-5H-benzo[d]-pyrido[3,2-f][1,3]diazepine-9-carbonitrile: A mixture of 2-chloro-7-(2,6-difluoro-4-nitrophenyl)-5-ethyl-6-oxo-6,7-dihydro-5H-benzo[d]pyrido-[3,2-f][1,3]diazepine-9-carbonitrile (8.34 g, 18.3 mmol), ammonium hydrochloride (4.89 g, 91.5 mmol), and iron powder (5.11 g, 91.5 mmol) in EtOH/water (5:1, 180 mL) was heated at 90° C. for 2 h. It was cooled to RT and filtered over celite, then the solute was concentrated. The residue was partitioned between sat. aq. NaHCO₃ and DCM; the organic layer was then washed with brine and dried over sodium sulfate. It was concentrated to ¼ volume to give a slurry, which was filtered to collect the product as a yellow solid (6.37 g). The filtrate was concentrated onto silica gel and purified by silica gel column chromatography (0-100% EtOAc/hexane) to give a second batch of product (1.24 g). The total amount of 7-(4-amino-2,6-difluorophenyl)-2-chloro-5-ethyl-6-oxo-6,7-dihydro-5H-benzo[d]-pyrido[3,2-f][1,3]diazepine-9-carbonitrile was 7.6 g (98%). LCMS (M+H)⁺=426.1.

Step 6-6, preparation of tert-butyl (2-((4-(2-chloro-9-cyano-5-ethyl-6-oxo-5,6-dihydro-7H-benzo[d]pyrido[3,2-f][1,3]diazepin-7-yl)-3,5-difluorophenyl)amino)ethyl)(methyl)carbamate: A mixture of 7-(4-amino-2,6-difluorophenyl)-2-chloro-5-ethyl-6-oxo-6,7-dihydro-5H-benzo[d]pyrido[3,2-f][1,3]diazepine-9-carbonitrile (25 mg, 59 μmol), tert-butyl methyl(2-oxoethyl)carbamate (30 mg, 0.17 mmol), ZnCl₂ (10 mg, 73 μmol) and MeOH (1 mL) was stirred at 60° C. for 2 h under nitrogen. Sodium cyanoborohydride (10 mg, 0.16 mmol) was then added and the mixture stirred at 60° C. for 1 h. The reaction mixture was cooled then purified directly by prep-HPLC with the following conditions: column, SunFire Prep C18 OBD, 19*150 mm, 5 um; mobile phase, 35-98% ACN/water, 0.1% FA over 7 min; flow rate, 70 mL/min; detector, UV 220 nm. This provided tert-butyl (2-((4-(2-chloro-9-cyano-5-ethyl-6-oxo-5,6-dihydro-7H-benzo[d]pyrido[3,2-f][1,3]diazepin-7-yl)-3,5-difluorophenyl)amino)ethyl)(methyl)carbamate (13 mg, 38%) as a yellow solid. LCMS (M+H+22)⁺=605.1.

Step 6-7, preparation of 4-chloro-10-(2,6-difluoro-4-{[2-(methylamino)ethyl]amino}phenyl)-8-ethyl-9-oxo-6,8,10-triazatricyclo[9.4.0.0^{2,7}]pentadeca-1(11),2(7),3,5,12,14-hexaene-13-carbonitrile: To a solution of tert-butyl (2-((4-(2-chloro-9-cyano-5-ethyl-6-oxo-5,6-dihydro-7H-benzo[d]pyrido[3,2-f][1,3]diazepin-7-yl)-3,5-difluorophenyl)amino)ethyl)(methyl)carbamate (13 mg, 22 μmol) in DCM (1.5 mL) was added TFA (0.5 mL) and the solution was stirred 1 h. It was concentrated and purified by prep-HPLC under the following conditions: column, SunFire Prep C18 OBD, 19*150 mm, 5 um; mobile phase, 35-65% ACN/water, 0.1% TFA over 7 min; flow rate, 20 mL/min; detector, UV 220 nm. This gave as a white solid the TFA salt of the title compound (12 mg, 75%). LCMS (M+H)⁺=483.2.

The following compounds were prepared similarly to Example 6 with appropriate substituting reagents and substrates at different steps and may require additional functional group modifications, via well-known chemistry with appropriate reagents. The reductive amination reaction can be carried out with other reductants such as sodium triacetoxyborohydride under appropriate conditions. Different salts, such as HCl, formic acid, or TFA salts, may be obtained.

| Compound no. | MS (M + H)⁺ |
|---|---|
| 1-62 | 481.3 |
| 1-81 | 467.2 |
| 1-84 | 481.2 |
| 1-91 | 497.2 |
| 1-111 | 467.2 |
| 1-117 | 481.2 |
| 1-119 | 497.2 |
| 1-121 | 483.2 |
| 1-122 | 497.2 |
| 1-133 | 497.1 |
| 1-211 | 569.2 |
| 3-19 | 466.2 |

### Example 7: 4-chloro-10-[2,6-difluoro-4-({2-[(2-hydroxyethyl)amino]ethyl}amino)phenyl]-8-ethyl-9-oxo-6,8,10-triazatricyclo[9.4.0.0^{2,7}]pentadeca-1(11),2(7),3,5,12,14-hexaene-13-carbonitrile (cpd 1-55)

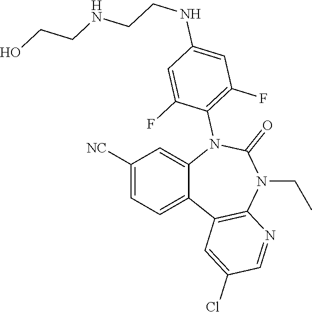 [SMILES: CCN1C(=O)N(c2c(F)cc(NCCNCCO)cc2F)c2cc(C#N)ccc2-c2cc(Cl)cnc21]

Step 7-1, preparation of tert-butyl (2-((tert-butyldimethylsilyl)oxy)ethyl)(2-((4-(2-chloro-9-cyano-5-ethyl-6-oxo-5,6-dihydro-7H-benzo[d]pyrido[3,2-f][1,3]diazepin-7-yl)-3,5-difluorophenyl)amino)ethyl)carbamate: Under nitrogen atmosphere, a mixture of 7-(4-amino-2,6-difluorophenyl)-2-chloro-5-ethyl-6-oxo-6,7-dihydro-5H-benzo[d]pyrido[3,2-f][1,3]diazepine-9-carbonitrile (from “Step 6-5, Example 6”) (25 mg, 59 μmol), tert-butyl (2-((tert-butyldimethylsilyl)oxy)ethyl)(2-oxoethyl)carbamate (from “Step 2-2, Example 2”) (60 mg, 0.19 mmol), ZnCl₂ (10 mg, 73 μmol), and MeOH (1 mL) was stirred at 60° C. for 2 h. Sodium cyanoborohydride (10 mg, 0.16 mmol) was added and the mixture was stirred at 60° C. for 2 h. After cooling, the mixture was purified directly by prep-HPLC with the following conditions: column, SunFire Prep C18 OBD, 19*150 mm, 5 um; mobile phase, 35-98% ACN/water, 0.1% FA over 7 min; flow rate 70 mL/min; detector, UV 220 nm. This yielded tert-butyl (2-((tert-butyldimethylsilyl)oxy)ethyl)(2-((4-(2-chloro-9-cyano-5-ethyl-6-oxo-5,6-dihydro-7H-benzo[d]pyrido[3,2-f][1,3]diazepin-7-yl)-3,5-difluorophenyl)amino)ethyl)carbamate (15 mg, 35%) as a yellow solid. LCMS (M+H+22)⁺=749.1.

Step 7-2, preparation of 4-chloro-10-[2,6-difluoro-4-({2-[(2-hydroxyethyl)amino]ethyl}amino)phenyl]-8-ethyl-9-oxo-6,8,10-triazatricyclo[9.4.0.0^{2,7}]pentadeca-1(11),2(7),3,5,12,14-hexaene-13-carbonitrile: To a solution of tert-butyl (2-((tert-butyldimethylsilyl)oxy)ethyl)(2-((4-(2-chloro-9-cyano-5-ethyl-6-oxo-5,6-dihydro-7H-benzo[d]pyrido[3,2-f][1,3]diazepin-7-yl)-3,5-difluorophenyl)amino)ethyl)carbamate (15 mg, 21 μmol) in DCM (0.5 mL) was added TFA (1 mL) and the solution was stirred 1 h, then concentrated. The residue was purified by prep-HPLC under the following conditions: column, SunFire Prep C18 OBD, 19*150 mm, 5 um; mobile phase, 35-68% ACN/water, 0.1% TFA over 7 min; flow rate, 20 mL/min; detector, UV 220 nm. This gave as a white solid the TFA salt of the title compound (7.4 mg, 48%). LCMS (M+H)⁺=513.2.

The following compounds were prepared similarly to Example 7 with appropriate substituting reagents and substrates at different steps and may require additional functional group modifications, via well-known chemistry with appropriate reagents. The reductive amination reaction can be carried out with other reductants such as sodium triacetoxyborohydride under appropriate conditions. Different salts, such as HCl, formic acid, or TFA salts, may be obtained.

| Compound no. | MS (M + H)⁺ |
|---|---|
| 1-63 | 511.2 |
| 1-82 | 497.2 |
| 1-85 | 511.3 |
| 1-92 | 527.2 |
| 1-112 | 497.3 |
| 1-118 | 511.2 |
| 1-120 | 527.2 |
| 1-195 | 499.1 |
| 1-201 | 541.3 |
| 1-226 | 486.3 |
| 3-12 | 498.3 |
| 3-13 | 498.2 |
| 3-14 | 498.2 |
| 3-18 | 496.3 |
| 3-20 | 511.2 |
| 3-23 | 527.3 |

### Example 8: 10-{4-[(2-aminoethyl)amino]-2,6-difluorophenyl}-4-chloro-8-ethyl-9-oxo-6,8,10-triazatricyclo[9.4.0.0^{2,7}]pentadeca-1(11),2(7),3,5,12,14-hexaene-13-carbonitrile (cpd 126)

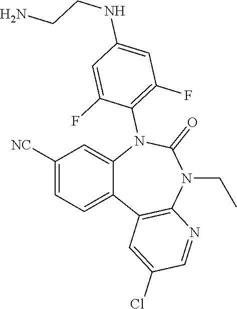 [SMILES: CCN1C(=O)N(c2c(F)cc(NCCN)cc2F)c2cc(C#N)ccc2-c2cc(Cl)cnc21]

Step 8-1, preparation of tert-butyl (2-((4-(2-chloro-9-cyano-5-ethyl-6-oxo-5,6-dihydro-7H-benzo[d]pyrido[3,2-f][1.3]diazepin-7-yl)-3,5-difluorophenyl)amino)ethyl)carbamate: Under nitrogen atmosphere, a mixture of 7-(4-amino-2,6-difluorophenyl)-2-chloro-5-ethyl-6-oxo-6,7-dihydro-5H-benzo[d]pyrido[3,2-f][1,3]diazepine-9-carbonitrile (from “Step 6-5, Example 6”) (50 mg, 0.12 mmol), tert-butyl (2-oxoethyl)carbamate (80 mg, 0.50 mmol), ZnCl₂ (20 mg, 0.15 mmol) and MeOH (1 mL) was stirred at 60° C. for 2 h. Sodium cyanoborohydride (20 mg, 0.32 mmol) was added, then the mixture was stirred at 60° C. for 1 h. The reaction mixture was cooled then purified by prep-HPLC under the following conditions: column, SunFire Prep C18 OBD, 19*150 mm, 5 um; mobile phase, 35-98% ACN/water, 0.1% FA over 7 min; flow rate 70 mL/min; detector, UV 220 nm. This yielded tert-butyl (2-((4-(2-chloro-9-cyano-5-ethyl-6-oxo-5,6-dihydro-7H-benzo[d]pyrido[3,2-f][1,3]diazepin-7-yl)-3,5-difluorophenyl)amino)ethyl)carbamate (35 mg, 52%) as a yellow solid. LCMS (M+H)⁺=591.3.

Step 8-2, preparation of 10-{4-[(2-aminoethyl)amino]-2,6-difluorophenyl}-4-chloro-8-ethyl-9-oxo-6,8,10-triazatricyclo[9.4.0.0^{2,7}]pentadeca-1(11),2(7),3,5,12,14-hexaene-13-carbonitrile: To a solution of tert-butyl (2-((4-(2-chloro-9-cyano-5-ethyl-6-oxo-5,6-dihydro-7H-benzo[d]pyrido[3,2-f][1,3]diazepin-7-yl)-3,5-difluorophenyl)amino)ethyl)carbamate (35 mg, 62 μmol) and DCM (1.5 mL) was added TFA (0.5 mL). The mixture was stirred for 1 h, then concentrated and purified by prep-HPLC under the following conditions: column, SunFire Prep C18 OBD column, 19*150 mm, 5 um; mobile phase, 35-98% ACN/water, 0.1% NH₃H₂O over 7 min; flow rate, 70 mL/min; detector, UV 220 nm. This yielded as a yellow solid the title compound (28 mg, 97%). LCMS (M+H)⁺=469.1.

The following compounds were prepared similarly to Example 8 with appropriate substituting reagents and substrates at different steps and may require additional functional group modifications, via well-known chemistry with appropriate reagents.

| Compound no. | MS (M + H)⁺ |
|---|---|
| 1-223 | 455.1 |

### Example 9: 4-({2-[(4-{4-chloro-13-cyano-8-ethyl-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)butanoic Acid (cpd 1-58)

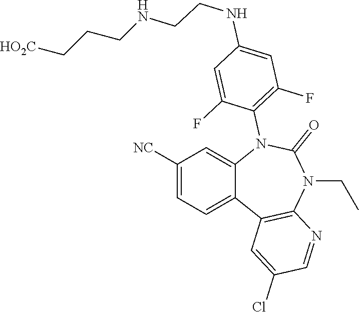 [SMILES: CCN1C(=O)N(c2c(F)cc(NCCNCCCC(=O)O)cc2F)c2cc(C#N)ccc2-c2cc(Cl)cnc21]

Step 9-1, preparation of tert-butyl 4-((2-((4-(2-chloro-9-cyano-5-ethyl-6-oxo-5,6-dihydro-7H-benzo[d]pyrido[3,2-f][1,3]diazepin-7-yl)-3,5-difluorophenyl)amino)ethyl)amino)butanoate: To a solution of 10-{4-[(2-aminoethyl)amino]-2,6-difluorophenyl}-4-chloro-8-ethyl-9-oxo-6,8,10-triazatricyclo[9.4.0.0^{2,7}]pentadeca-1(11),2(7),3,5,12,14-hexaene-13-carbonitrile (from “Step 8-2, Example 8”) (25 mg, 53 μmol) in MeOH (1 mL) was added tert-butyl 4-oxobutanoate (10 mg, 63 μmol) and sodium cyanoborohydride (10 mg, 0.16 mmol). The mixture was stirred for 1 h, then purified directly by prep-HPLC with the following conditions: column, SunFire Prep C18 OBD, 19*150 mm, 5 um; mobile phase, 35-98% ACN/water, 0.10% FA over 7 min; flow rate 70 mL/min; detector, UV 220 nm. This gave as a yellow solid tert-butyl 4-((2-((4-(2-chloro-9-cyano-5-ethyl-6-oxo-5,6-dihydro-7H-benzo[d]pyrido[3,2-f][1,3]diazepin-7-yl)-3,5-difluorophenyl)amino)ethyl)amino)butanoate (13 mg, 40%) as a yellow solid. LCMS (M+H)⁺=611.3.

Step 9-2, preparation of 4-({2-[(4-{4-chloro-13-cyano-8-ethyl-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)butanoic acid: To tert-butyl 4-((2-((4-(2-chloro-9-cyano-5-ethyl-6-oxo-5,6-dihydro-7H-benzo[d]pyrido[3,2-f][1,3]diazepin-7-yl)-3,5-difluorophenyl)amino)ethyl)amino)butanoate (13 mg, 21 μmol) in DCM (1 mL) was added TFA (2 mL) and the mixture was stirred for 4 h. The resulting solution was concentrated under vacuum then purified by prep-HPLC with the following conditions: column, SunFire Prep C18 OBD, 19*150 mm, 5 um; mobile phase, 32-68% ACN/water, 0.05% TFA over 7 min; flow rate, 20 mL/min; detector, UV 220 nm. This gave as a white solid the TFA salt of the title compound (12 mg, 72%). LCMS (M+H)⁺=555.2.

The following compounds were prepared similarly to Example 9 with appropriate substituting reagents and substrates at different steps and may require additional functional group modifications, via well-known chemistry with appropriate reagents. The reductive amination reaction can be carried out with other reductants such as sodium triacetoxyborohydride under appropriate conditions. Different salts, such as HCl, formic acid, or TFA salts, may be obtained.

| Compound no. | MS (M + H)⁺ |
|---|---|
| 1-68 | 553.3 |
| 1-83 | 539.3 |
| 1-86 | 553.3 |
| 1-93 | 569.2 |
| 1-217 | 583.0 |

### Example 10: 4-chloro-10-{2,6-difluoro-4-[(2-{[2-(methylamino)ethyl]amino}ethyl)amino]phenyl}-8-ethyl-9-oxo-6,8,10-triazatricyclo[9.4.0.0^{2,7}]pentadeca-1(11),2(7),3,5,12,14-hexaene-13-carbonitrile (cpd 1-127)

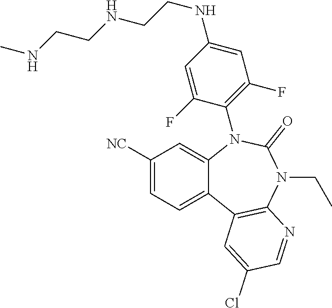 [SMILES: CCN1C(=O)N(c2c(F)cc(NCCNCCNC)cc2F)c2cc(C#N)ccc2-c2cc(Cl)cnc21]

Step 10-1, preparation of tert-butyl (2-((2-((4-(2-chloro-9-cyano-5-ethyl-6-oxo-5,6-dihydro-7H-benzo[d]pyrido[3,2-f][1,3]diazepin-7-yl)-3,5-difluorophenyl)amino)ethyl)amino)ethyl)(methyl)carbamate: To a solution of 10-{4-[(2-aminoethyl)amino]-2,6-difluorophenyl}-4-chloro-8-ethyl-9-oxo-6,8,10-triazatricyclo[9.4.0.0^{2,7}]pentadeca-1(11),2(7),3,5,12,14-hexaene-13-carbonitrile (from “Step 8-2, Example 8”) (37 mg, 63 μmol) in MeOH (0.5 mL) was added tert-butyl methyl(2-oxoethyl)carbamate (11 mg, 62 μmol) and the solution was stirred 10 min. Sodium cyanoborohydride (8 mg, 0.1 mmol) was added and the resulting mixture was stirred 40 min. It was purified directly by C18 reverse-phase chromatography (10-100% ACN/water, 0.1% TFA) to provide as the TFA salt tert-butyl (2-((2-((4-(2-chloro-9-cyano-5-ethyl-6-oxo-5,6-dihydro-7H-benzo[d]pyrido[3,2-f][1,3]diazepin-7-yl)-3,5-difluorophenyl)amino)ethyl)amino)ethyl)(methyl)carbamate (9.2 mg, 20%). LCMS (M+H)⁺=626.3.

Step 10-2, preparation of 4-chloro-10-{2,6-difluoro-4-[(2-{[2-(methylamino)ethyl]amino}ethyl)amino]phenyl}-8-ethyl-9-oxo-6,8,10-triazatricyclo[9.4.0.0^{2,7}]pentadeca-1(11),2(7),3,5,12,14-hexaene-13-carbonitrile: To tert-butyl (2-((2-((4-(2-chloro-9-cyano-5-ethyl-6-oxo-5,6-dihydro-7H-benzo[d]pyrido[3,2-f][1,3]diazepin-7-yl)-3,5-difluorophenyl)amino)ethyl)amino)ethyl)(methyl)carbamate (9.2 mg, 63 μM) in DCM (0.5 mL) was added TFA (0.2 mL) and the mixture was stirred for 40 min. The resulting solution was concentrated under vacuum then purified by C18 reverse-phase chromatography (10-100% ACN/water, 0.1% TFA) to provide the TFA salt of the title compound (6.6 mg, 70%). LCMS (M+H)⁺=526.0.

The following compound was prepared similarly to Example 10 with appropriate substituting reagents and substrates at different steps and may require additional functional group modifications, via well-known chemistry with appropriate reagents. The reductive amination reaction can be carried out with other reductants such as sodium triacetoxyborohydride under appropriate conditions. Different salts, such as HCl, formic acid, or TFA salts, may be obtained.

| Compound no. | MS (M + H)⁺ |
|---|---|
| 1-131 | 512.0 |
| 1-228 | 581.3 |

### Example 11: 4-chloro-10-{4-[(2-{[2-(dimethylamino)ethyl]amino}ethyl)amino]-2,6-difluorophenyl}-8-ethyl-9-oxo-6,8,10-triazatricyclo[9.4.0.0^{2,7}]pentadeca-1(11),2(7),3,5,12,14-hexaene-13-carbonitrile (cpd 1-132)

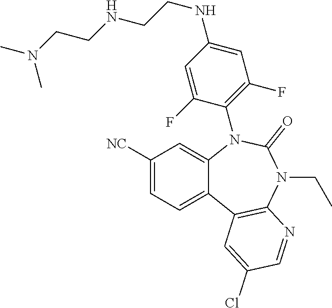 [SMILES: CCN1C(=O)N(c2c(F)cc(NCCNCCN(C)C)cc2F)c2cc(C#N)ccc2-c2cc(Cl)cnc21]

Step 11-1, preparation of 2-chloro-7-(4-((2-chloroethyl)amino)-2,6-difluorophenyl)-5-ethyl-6-oxo-6,7-dihydro-5H-benzo[d]pyrido[3,2-f][1,3]diazepine-9-carbonitrile: To a mixture of 7-(4-amino-2,6-difluorophenyl)-2-chloro-5-ethyl-6-oxo-6,7-dihydro-5H-benzo[d]pyrido[3,2-f][1,3]diazepine-9-carbonitrile (from “Step 6-5, Example 6”) (425 mg, 1.00 mmol) in MeOH (2.5 mL) was added 2-chloroacetaldehyde in water (172 mg, 50% wt, 1.10 mmol) at 0° C. and the mixture stirred 15 min. Zinc(II) chloride in 2-Me-THF (1.0 mL, 1.9 M, 1.9 mmol) was added and stirring was continued at 0° C. for 45 min, followed by addition of sodium borohydride (76 mg, 2.00 mmol). The mixture was stirred 1 h, then further MeOH (2 mL), 2-chloroacetaldehyde in water (560 mg, 50% wt, 3.6 mmol), acetic acid (0.1 mL) and zinc(II) chloride in 2-Me-THF (1 mL, 1.9 M, 1.9 mmol) were added and the reaction was heated to 60° C. for 20 min, then cooled, quenched sat. aq. NaHCO₃, and extracted with ethyl acetate. The organic layer was washed with water and brine, dried over sodium sulfate, concentrated and purified by silica gel column chromatography (0-100% EtOAc/hexane) to give as a colorless oil 2-chloro-7-(4-((2-chloroethyl)amino)-2,6-difluorophenyl)-5-ethyl-6-oxo-6,7-dihydro-5H-benzo[d]pyrido[3,2-f][1,3]diazepine-9-carbonitrile (231 mg, 47%). LCMS (M+H)⁺=488.1.

Step 11-2, preparation of 4-chloro-10-{4-[(2-{[2-(dimethylamino)ethyl]amino}ethyl)amino]-2,6-difluorophenyl}-8-ethyl-9-oxo-6,8,10-triazatricyclo[9.4.0.0^{2,7}]pentadeca-1(11),2(7),3,5,12,14-hexaene-13-carbonitrile: A mixture of 2-chloro-7-(4-((2-chloroethyl)amino)-2,6-difluorophenyl)-5-ethyl-6-oxo-6,7-dihydro-5H-benzo[d]pyrido[3,2-f][1,3]diazepine-9-carbonitrile (15 mg, 31 μmol), N1,N1-dimethylethane-1,2-diamine (4.1 mg, 46 μmol), potassium carbonate (6.4 mg, 46 μmol), and sodium iodide (6.9 mg, 46 μmol) in propionitrile (0.3 mL) was heated at 80° C. for 4 h. Additional N1,N1-dimethylethane-1,2-diamine (20 mg, 0.22 mmol) and sodium iodide (14 mg, 90 μmol) were added and the mixture was heated at 90° C. overnight. The reaction was partitioned between DCM and brine, separated, and the organic layer dried over sodium sulfate. The organic solution was concentrated then purified by C18 reverse-phase chromatography (10-100% ACN/water, 0.1% TFA) to provide the TFA salt of the title compound. LCMS (M+H)⁺=540.1.

The following compounds were prepared similarly to Example 11 with appropriate substituting reagents and substrates at different steps and may require additional functional group modifications, via well-known chemistry with appropriate reagents. The reductive amination and alkylation reactions can be carried out with other reagents under appropriate conditions. Different salts, such as HCl, formic acid, or TFA salts, may be obtained.

| Compound no. | MS (M + H)⁺ |
|---|---|
| 1-128 | 552.1 |
| 1-137 | 527.0 |
| 1-138 | 541.1 |
| 1-139 | 541.0 |
| 1-140 | 555.2 |
| 1-141 | 577.0 |
| 1-148 | 539.3 |
| 1-149 | 527.0 |
| 1-150 | 589.9 |
| 1-154 | 522.2 |
| 1-155 | 538.3 |
| 1-156 | 543.1 |
| 1-157 | 543.1 |
| 1-160 | 508.2 |
| 1-161 | 540.3 |
| 1-162 | 526.2 |
| 1-164 | 539.3 |
| 1-166 | 569.2 |
| 1-168 | 554.4 |
| 1-169 | 496.3 |
| 1-170 | 566.0 |
| 1-171 | 543.3 |
| 1-172 | 553.2 |
| 1-173 | 527.2 |
| 1-174 | 566.0 |
| 1-176 | 566.0 |
| 1-177 | 539.2 |
| 1-178 | 541.2 |
| 1-179 | 539.2 |
| 1-180 | 527.2 |
| 1-181 | 557.0 |
| 1-183 | 543.3 |
| 1-184 | 573.1 |
| 1-185 | 527.3 |
| 1-186 | 527.2 |
| 1-187 | 539.2 |
| 1-188 | 642.1 |
| 1-189 | 556.0 |
| 1-190 | 527.1 |
| 1-191 | 582.0 |
| 1-192 | 539.2 |
| 1-193 | 595.1 |
| 1-194 | 527.3 |
| 1-196 | 573.1 |
| 1-197 | 513.2 |
| 1-198 | 525.3 |
| 1-199 | 511.4 |
| 1-200 | 527.4 |
| 1-202 | 571.2 |
| 1-203 | 511.3 |
| 1-204 | 523.3 |
| 1-205 | 513.2 |
| 1-206 | 566.9 |
| 1-207 | 573.0 |
| 1-209 | 582.9 |
| 1-210 | 556.8 |
| 1-213 | 511.1 |
| 1-214 | 510.1 |
| 1-215 | 527.2 |
| 1-218 | 523.3 |
| 1-219 | 540.9 |
| 1-220 | 557.0 |
| 1-222 | 523.3 |
| 1-224 | 573.0 |
| 1-225 | 541.9 |
| 1-227 | 560.9 |
| 1-229 | 585.1 |
| 1-230 | 589.0 |
| 1-231 | 539.2 |
| 1-232 | 523.3 |
| 1-233 | 601.0 |
| 1-236 | 541.1 |
| 1-237 | 496.3 |
| 1-238 | 552.3 |
| 1-239 | 589.0 |
| 1-240 | 582.9 |
| 1-242 | 536.2 |

### Example 12: 4-chloro-10-[2,6-difluoro-4-(piperazin-1-yl)phenyl]-8-ethyl-9-oxo-6,8,10-triazatricyclo[9.4.0.0^{2,7}]pentadeca-1(11),2(7),3,5,12,14-hexaene-13-carbonitrile (cpd 1-136)

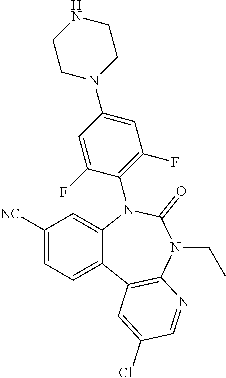 [SMILES: CCN1C(=O)N(c2c(F)cc(N3CCNCC3)cc2F)c2cc(C#N)ccc2-c2cc(Cl)cnc21]

Step 12-1, preparation of 2-chloro-7-(2,6-difluoro-4-iodophenyl)-5-ethyl-6-oxo-6,7-dihydro-5H-benzo[d]pyrido[3,2-f][1,3]diazepine-9-carbonitrile: To a solution of 7-(4-amino-2,6-difluorophenyl)-2-chloro-5-ethyl-6-oxo-6,7-dihydro-5H-benzo[d]pyrido[3,2-f][1,3]diazepine-9-carbonitrile (from “Step 6-5, Example 6”) (2.0 g, 4.7 mmol) in ACN (60 mL) under nitrogen was added KI (3.9 g, 23 mmol) and tert-butyl nitrite (4.8 g, 47 mmol). The resulting mixture was stirred at 60° C. for 3 h, then quenched with water, and extracted with EtOAc (3×). The organic layers were combined, dried over Na₂SO₄, filtered and concentrated. The residue was purified by silica gel column chromatography (15% EtOAc/PE) to give as a yellow solid 2-chloro-7-(2,6-difluoro-4-iodophenyl)-5-ethyl-6-oxo-6,7-dihydro-5H-benzo[d]pyrido[3,2-f][1,3]diazepine-9-carbonitrile (1.8 g, 71%). LCMS (M+H)⁺=537.0.

Step 12-2, preparation of 4-(4-(2-chloro-9-cyano-5-ethyl-6-oxo-5,6-dihydro-7H-benzo[d]pyrido[3,2-f][1,3]diazepin-7-yl)-3,5-difluorophenyl)piperazine-1-carboxylate: To a mixture of 2-chloro-7-(2,6-difluoro-4-iodophenyl)-5-ethyl-6-oxo-6,7-dihydro-5H-benzo[d]pyrido[3,2-f][1,3]diazepine-9-carbonitrile (30 mg, 56 μmol), tert-butyl piperazine-1-carboxylate (31 mg, 0.17 mmol), KOtBu (9.4 mg, 84 μmol) and toluene (2 mL) under nitrogen was added Pd₂(dba)₃ (5.1 mg, 5.6 μmol) and xantphos (6.5 mg, 11 μmol). The mixture was stirred at 50° C. for 1 h, then quenched with water, extracted with EtOAc (3×) and the combined organic layers were dried over Na₂SO₄ and concentrated. The residue was a purified by silica gel column chromatography (20% EtOAc/PE) to provide as a yellow oil tert-butyl 4-(4-(2-chloro-9-cyano-5-ethyl-6-oxo-5,6-dihydro-7H-benzo[d]pyrido[3,2-f][1,3]diazepin-7-yl)-3,5-difluorophenyl)piperazine-1-carboxylate (25 mg, 42 μmol, 75%). LCMS (M+H)⁺=595.3.

Step 12-3, preparation of 4-chloro-10-[2,6-difluoro-4-(piperazin-1-yl)phenyl]-8-ethyl-9-oxo-6,8,10-triazatricyclo[9.4.0.0^{2,7}]pentadeca-1(11),2(7),3,5,12,14-hexaene-13-carbonitrile: To a solution of tert-butyl 4-(4-(2-chloro-9-cyano-5-ethyl-6-oxo-5,6-dihydro-7H-benzo[d]pyrido[3,2-f][1,3]diazepin-7-yl)-3,5-difluorophenyl)piperazine-1-carboxylate (25 mg, 42 μmol) in DCM (5 mL) was added TFA (1 mL) and the solution was stirred 2 h then concentrated. The residue was purified via prep-HPLC with the following conditions: column, SunFire Prep C18 OBD column, 19*150 mm, 5 μm; mobile phase, 35-55% ACN/water, 0.05% TFA over 8 min; total flow, 20 mL/min; detector, UV 254 nm. This gave the TFA salt of the title compound (19 mg, 64%) as a white solid. LCMS (M+H)⁺=495.3.

The following compounds were prepared similarly to Example 12 with appropriate substituting reagents and substrates at different steps and may require additional functional group modifications, via well-known chemistry with appropriate reagents. The amination reaction can be carried out with other metal/catalyst systems under appropriate conditions. Different salts, such as HCl, formic acid, or TFA salts, may be obtained.

| Compound no. | MS (M + H)⁺ |
|---|---|
| 1-123 | 479.2 |
| 1-124 | 509.2 |
| 1-143 | 509.2 |
| 1-144 | 539.3 |
| 1-145 | 495.2 |
| 1-146 | 497.3 |
| 1-147 | 525.3 |
| 1-151 | 525.2 |
| 1-152 | 525.2 |
| 1-153 | 525.2 |
| 1-158 | 525.2 |
| 1-159 | 497.2 |
| 1-165 | 497.2 |
| 1-182 | 537.0 |
| 1-212 | 511.2 |
| 1-234 | 483.1 |
| 1-235 | 509.1 |
| 1-241 | 495.7 |

### Example 13: 10-(2,6-difluoro-4-{[2-(methylamino)ethyl]amino}phenyl)-4,15-difluoro-9-oxo-8-(propan-2-yl)-6,8,10-triazatricyclo[9.4.0.0^{2,7}]pentadeca-1(11),2(7),3,5,12,14-hexaene-13-carbonitrile (cpd 1-107)

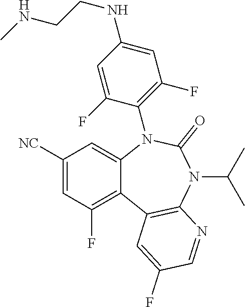 [SMILES: CNCCNc1cc(F)c(N2C(=O)N(C(C)C)c3ncc(F)cc3-c3c(F)cc(C#N)cc32)c(F)c1]

Step 13-1, preparation of 4-amino-3-fluoro-5-nitrobenzonitrile: To 4-bromo-2-fluoro-6-nitroaniline (5.0 g, 21 mmol) in DMF (50 mL) under nitrogen was added zinc cyanide (2.0 g, 17 mmol), Pd₂(dba)₃-CHCl₃ (0.66 g, 0.64 mmol), and 4,5-bis(diphenylphosphino)-9,9-dimethylxanthene (0.9 g, 2 mmol). The mixture was heated at 120° C. for 4 h. After cooling, the solids were filtered off and the filtrate was concentrated under vacuum. The residue was purified by silica gel column chromatography (25% EtOAc/PE) to give as a yellow oil 4-amino-3-fluoro-5-nitrobenzonitrile (4.0 g, 97%). GCMS (M)⁺: 181.1.

Step 13-2, preparation of 4-bromo-3-fluoro-5-nitrobenzonitrile: To 4-amino-3-fluoro-5-nitrobenzonitrile (4.0 g, 22 mmol) in ACN (50 mL) at 0° C. was added cuprous bromide (9.5 g, 66 mmol), then t-butyl nitrite (6.8 g, 66 mmol) over 10 min. The mixture was then heated at 60° C. for 1 h, then diluted with water and extracted with ethyl acetate (3×). The combined organic layers were washed with brine, dried over sodium sulfate and concentrated. The residue was purified by silica gel column chromatography (15% EtOAc/PE) to give as a yellow solid 4-bromo-3-fluoro-5-nitrobenzonitrile (2.4 g, 44%). GCMS (M)⁺: 243.9.

Step 13-3, preparation of 3-bromo-5-fluoro-N-isopropylpyridin-2-amine: To a solution of 3-bromo-5-fluoropyridin-2-amine (5.0 g, 26 mmol) in DMF (50 mL) under nitrogen at 0° C. was added portionwise NaH (1.6 g, 60% wt., 40 mmol). The mixture was stirred 20 min at 0° C., then was added dropwise 2-iodopropane (6.7 g, 39 mmol) and stirring was continued for 5 h at 20° C. The mixture was diluted with water, then extracted with ethyl acetate (3×). The combined organic layers were washed with brine (2×), dried over sodium sulfate, and concentrated. The residue was purified via silica gel column chromatography (33% EtOAc/PE) to give as a yellow oil 3-bromo-5-fluoro-N-isopropylpyridin-2-amine (4.8 g, 79%). LCMS (M+H)⁺=233.0.

Step 13-4, preparation of 5-fluoro-N-isopropyl-3-(4,4,5,5-tetramethyl-1,3,2-dioxaborolan-2-yl)pyridin-2-amine: A mixture of 3-bromo-5-fluoro-N-isopropylpyridin-2-amine (2.0 g, 8.6 mmol), bis(pinacolato)diborane (3.3 g, 13 mmol), potassium acetate (2.5 g, 25 mmol), and [1,1′-bis(diphenylphosphino)ferrocene]dichloropalladium(II)-CH₂Cl₂ (0.35 g, 0.43 mmol) in dioxane (25 mL) under nitrogen was heated at 80° C. overnight. After cooling, the solids were removed by filtration and the filtrate was concentrated under vacuum. The residue was purified via silica gel column chromatography (25% EtOAc/PE) to give as a yellow oil 5-fluoro-N-isopropyl-3-(4,4,5,5-tetramethyl-1,3,2-dioxaborolan-2-yl)pyridin-2-amine (3.0 g, 95%). LCMS (M+H)⁺=281.2.

Step 13-5, preparation of 3-fluoro-4-(5-fluoro-2-(isopropylamino)pyridin-3-yl)-5-nitrobenzonitrile: A mixture of 5-fluoro-N-isopropyl-3-(4,4,5,5-tetramethyl-1,3,2-dioxaborolan-2-yl)pyridin-2-amine (515 mg, 1.84 mmol), 4-bromo-3-fluoro-5-nitrobenzonitrile (from “Step 13-2, Example 13”) (300 mg, 1.22 mmol), K₂CO₃ (508 mg, 3.68 mmol), and Pd(dtbpf)Cl₂ (40 mg, 61 μmol) in dioxane/water (4 mL/0.4 mL) under nitrogen was heated at 90° C. for 2 h. After cooling, the solids were filtered off and the filtrate was concentrated under vacuum. The residue was purified via silica gel column chromatography (25% EtOAc/PE) to give as a yellow solid 3-fluoro-4-(5-fluoro-2-(isopropylamino)pyridin-3-yl)-5-nitrobenzonitrile (290 mg, 74%). LCMS (M+H)⁺=319.1.

Step 13-6, preparation of 3-amino-5-fluoro-4-(5-fluoro-2-(isopropylamino)pyridin-3-yl)benzonitrile: A mixture of 3-fluoro-4-(5-fluoro-2-(isopropylamino)pyridin-3-yl)-5-nitrobenzonitrile (290 mg, 0.91 mmol), iron powder (360 mg, 6.4 mmol), and ammonium chloride (340 mg, 6.4 mmol) in EtOH (5 mL) and water (1 mL) was heated at 80° C. for 2 h. After cooling, the solids were filtered off and the filtrate was concentrated under vacuum. The residue was purified via C18 reverse-phase flash chromatography (30-98% ACN/water, 0.1% FA) to give as a yellow solid 3-amino-5-fluoro-4-(5-fluoro-2-(isopropylamino)pyridin-3-yl)benzonitrile (240 mg, 91%). LCMS (M+H)⁺=289.1.

Step 13-7, preparation of 2,11-difluoro-5-isopropyl-6-oxo-6,7-dihydro-5H-benzo[d]pyrido[3,2-f][1,3]diazepine-9-carbonitrile: To 3-amino-5-fluoro-4-(5-fluoro-2-(isopropylamino)pyridin-3-yl)benzonitrile (85 mg, 0.29 mmol) in DCM (2 mL) under nitrogen 0° C. at was added DIEA (0.15 g, 1.2 mmol), followed by dropwise addition of a solution of triphosgene (70 mg, 0.24 mmol) in DCM (0.3 mL). The mixture was stirred at 0° C. for 2 h, then concentrated and purified via C18 reverse-phase flash chromatography (30-98% ACN/water, 0.1% FA) to give as a yellow solid 2,11-difluoro-5-isopropyl-6-oxo-6,7-dihydro-5H-benzo[d]pyrido[3,2-f][1,3]diazepine-9-carbonitrile (89 mg, 96%). LCMS (M+H)⁺=315.1.

Step 13-8, preparation of 7-(2,6-difluoro-4-nitrophenyl)-2,11-difluoro-5-isopropyl-6-oxo-6,7-dihydro-5H-benzo[d]pyrido[3,2-f][1,3]diazepine-9-carbonitrile: To a solution of 2,11-difluoro-5-isopropyl-6-oxo-6,7-dihydro-5H-benzo[d]pyrido[3,2-f][1,3]diazepine-9-carbonitrile (89 mg, 0.28 mmol) in DMF (2 mL) under nitrogen was added NaH (17 mg, 60% wt, 0.43 mmol), and 1,2,3-trifluoro-5-nitrobenzene (60 mg, 0.34 mmol). The resulting solution was stirred for 1 h, then purified directly via C18 reverse-phase flash chromatography (30-98% ACN/water, 0.1% FA) to give as a yellow solid 7-(2,6-difluoro-4-nitrophenyl)-2,11-difluoro-5-isopropyl-6-oxo-6,7-dihydro-5H-benzo[d]pyrido[3,2-f][1,3]diazepine-9-carbonitrile (95 mg, 71%). LCMS (M+H)⁺=472.1.

Step 13-9, preparation of 7-(4-amino-2,6-difluorophenyl)-2,11-difluoro-5-isopropyl-6-oxo-6,7-dihydro-5H-benzo[d]pyrido[3,2-f][1,3]diazepine-9-carbonitrile: To 7-(2,6-difluoro-4-nitrophenyl)-2,11-difluoro-5-isopropyl-6-oxo-6,7-dihydro-5H-benzo[d]pyrido[3,2-f][1,3]diazepine-9-carbonitrile (80 mg, 0.17 mmol) in EtOH (3 mL) and water (0.6 mL) was added ammonium chloride (76 mg, 1.4 mmol) and iron powder (76 mg, 1.4 mmol). The mixture was heated at 80° C. for 1 h, then cooled, filtered and purified directly via C18 reverse-phase flash chromatography (30-98% ACN/water, 0.1% FA) to give as a pale yellow solid 7-(4-amino-2,6-difluorophenyl)-2,11-difluoro-5-isopropyl-6-oxo-6,7-dihydro-5H-benzo[d]pyrido[3,2-f][1,3]diazepine-9-carbonitrile (70 mg, 93%). LCMS (M+H)⁺=442.1.

Step 13-10, preparation of tert-butyl (2-((4-(9-cyano-2,11-difluoro-5-isopropyl-6-oxo-5,6-dihydro-7H-benzo[d]pyrido[3,2-f][1,3]diazepin-7-yl)-3,5-difluorophenyl)amino)ethyl)(methyl)carbamate: A mixture of 7-(4-amino-2,6-difluorophenyl)-2,11-difluoro-5-isopropyl-6-oxo-6,7-dihydro-5H-benzo[d]pyrido[3,2-f][1,3]diazepine-9-carbonitrile (30 mg, 68 μmol), tert-butyl methyl(2-oxoethyl)carbamate (35 mg, 0.20 mmol), ZnCl₂ (28 mg, 0.21 mmol), and NaBH₃CN (17 mg, 4.0 Eq, 0.27 mmol) in MeOH (0.6 mL) under nitrogen was heated at 60° C. for 2 h, then cooled and then then purified directly via C18 reverse-phase flash chromatography (30-98% ACN/water, 0.1% FA) to give as a tan solid tert-butyl (2-((4-(9-cyano-2,11-difluoro-5-isopropyl-6-oxo-5,6-dihydro-7H-benzo[d]pyrido[3,2-f][1,3]diazepin-7-yl)-3,5-difluorophenyl)amino)ethyl)(methyl)carbamate (15 mg, 37%). LCMS (M+H)⁺=599.2.

Step 13-11, preparation of 10-(2,6-difluoro-4-{[2-(methylamino)ethyl]amino}phenyl)-4,15-difluoro-9-oxo-8-(propan-2-yl)-6,8,10-triazatricyclo[9.4.0.0^{2,7}]pentadeca-1(11),2(7),3,5,12,14-hexaene-13-carbonitrile: To tert-butyl (2-((4-(9-cyano-2,11-difluoro-5-isopropyl-6-oxo-5,6-dihydro-7H-benzo[d]pyrido[3,2-f][1,3]diazepin-7-yl)-3,5-difluorophenyl)amino)ethyl)(methyl)carbamate (15 mg, 25 μmol) in DCM (2 mL) was added TFA (0.4 mL). The resulting solution was stirred for 2 h, then concentrated and purified via prep-HPLC under the following conditions: column, SunFire Prep C18 OBD column, 19*150 mm, 5 μm; mobile phase, 20-50% ACN/water, 0.05% TFA over 10 min; total flow, 20 mL/min; detector, UV 220/254 nm. This gave the TFA salt of the title compound (11 mg, 61%) as white solid. LCMS (M+H)⁺=499.2.

The following compounds were prepared similarly to Example 13 with appropriate substituting reagents and substrates at different steps and may require additional functional group modifications, via well-known chemistry with appropriate reagents. The reductive amination reaction can be carried out with other reductants such as sodium triacetoxyborohydride under appropriate conditions. Different salts, such as HCl, formic acid, or TFA salts, may be obtained.

| Compound no. | MS (M + H)⁺ |
|---|---|
| 1-99 | 501.2 |
| 1-102 | 485.2 |
| 1-109 | 471.2 |
| 1-115 | 487.1 |

### Example 14: 10-[2,6-difluoro-4-({2-[(2-hydroxyethyl)amino]ethyl}amino)phenyl]-4,15-difluoro-9-oxo-8-(propan-2-yl)-6,8,10-triazatricyclo[9.4.0.0^{2,7}]pentadeca-1(11),2(7),3,5,12,14-hexaene-13-carbonitrile (cpd 1-108)

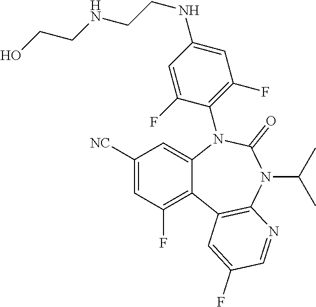 [SMILES: CC(C)N1C(=O)N(c2c(F)cc(NCCNCCO)cc2F)c2cc(C#N)cc(F)c2-c2cc(F)cnc21]

Step 14-1, preparation of tert-butyl (2-((tert-butyldimethylsilyl)oxy)ethyl)(2-((4-(9-cyano-2,11-difluoro-5-isopropyl-6-oxo-5,6-dihydro-7H-benzo[d]pyrido[3,2-f][1,3]diazepin-7-yl)-3,5-difluorophenyl)amino)ethyl)carbamate: To a mixture of 7-(4-amino-2,6-difluorophenyl)-2,11-difluoro-5-isopropyl-6-oxo-6,7-dihydro-5H-benzo[d]pyrido[3,2-f][1,3]diazepine-9-carbonitrile (from “Step 13-9, Example 13”) (30 mg, 68 μmol), ZnCl₂ (28 mg, 0.21 mmol), and tert-butyl (2-((tert-butyldimethylsilyl)oxy)ethyl)(2-oxoethyl)carbamate (from “Step 2-2, Example 2”) (65 mg, 0.20 mmol) in MeOH (2 mL) under nitrogen was added NaBH₃CN (17 mg, 0.27 mmol). The reaction mixture was stirred at 60° C. for 2 h, then cooled and purified directly via C18 reverse-phase flash chromatography (30-98% ACN/water, 0.1% FA) to give as a yellow solid tert-butyl (2-((tert-butyldimethylsilyl)oxy)ethyl)(2-((4-(9-cyano-2,11-difluoro-5-isopropyl-6-oxo-5,6-dihydro-7H-benzo[d]pyrido[3,2-f][1,3]diazepin-7-yl)-3,5-difluorophenyl)amino)ethyl)carbamate (28 mg, 55%). LCMS (M+H)⁺=743.3.

Step 14-2, preparation of 10-[2,6-difluoro-4-({2-[(2-hydroxyethyl)amino]ethyl}amino)phenyl]-4,15-difluoro-9-oxo-8-(propan-2-yl)-6,8,10-triazatricyclo[9.4.0.0^{2,7}]pentadeca-1(11),2(7),3,5,12,14-hexaene-13-carbonitrile: A solution of tert-butyl (2-((tert-butyldimethylsilyl)oxy)ethyl)(2-((4-(9-cyano-2,11-difluoro-5-isopropyl-6-oxo-5,6-dihydro-7H-benzo[d]pyrido[3,2-f][1,3]diazepin-7-yl)-3,5-difluorophenyl)amino)ethyl)carbamate (28 mg, 38 μmol) in TFA (2 mL) was stirred 2 h, then concentrated and purified via prep-HPLC under the following conditions: column, SunFire Prep C18 OBD column, 19*150 mm, 5 μm; mobile phase, 20-51% ACN/water, 0.05% TFA over 10 min; total flow, 20 mL/min; detector, UV 220/254 nm. This gave the TFA salt of the title compound (15 mg, 53%) as white solid. LCMS (M+H)⁺=529.3.

The following compounds were prepared similarly to Example 14 with appropriate substituting reagents and substrates at different steps and may require additional functional group modifications, via well-known chemistry with appropriate reagents. The reductive amination reaction can be carried out with other reductants such as sodium triacetoxyborohydride under appropriate conditions. Different salts, such as HCl, formic acid, or TFA salts, may be obtained.

| Compound no. | MS (M + H)⁺ |
|---|---|
| 1-100 | 531.2 |
| 1-103 | 515.2 |
| 1-110 | 501.2 |
| 1-116 | 517.1 |
| 1-125 | 529.3 |

### Example 15: 4-{[2-({4-[13-cyano-4,15-difluoro-9-oxo-8-(propan-2-yl)-6,8,10-triazatricyclo[9.4.0.0^{2,7}]pentadeca-1(15),2(7),3,5,11,13-hexaen-10-yl]-3,5-difluorophenyl}amino)ethyl]amino}butanoic Acid (cpd 1-163)

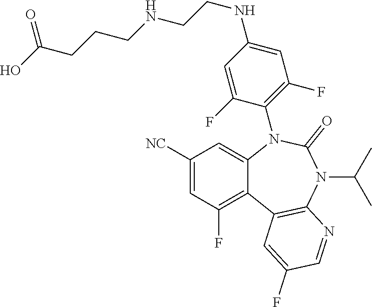 [SMILES: CC(C)N1C(=O)N(c2c(F)cc(NCCNCCCC(=O)O)cc2F)c2cc(C#N)cc(F)c2-c2cc(F)cnc21]

Step 15-1, preparation of tert-butyl 4-((2-hydroxyethyl)amino)butanoate: To 2-aminoethan-1-ol (7.0 g, 0.11 mol) and potassium carbonate (25 g, 0.18 mol) in ACN (130 mL) was added dropwise tert-butyl 4-bromobutanoate (13 g, 58 mmol), then stirred 6 h. The reaction was then diluted with water and extracted with EtOAc (3×). The organic layers were combined, washed with brine, dried over sodium sulfate, and concentrated under vacuum. The crude product tert-butyl 4-((2-hydroxyethyl)amino)butanoate (9.0 g, 80%) was used directly in the next step. LCMS (M+H)⁺=204.2.

Step 15-2, preparation of tert-butyl 4-((tert-butoxycarbonyl)(2-hydroxyethyl)amino)butanoate: A mixture of tert-butyl 4-((2-hydroxyethyl)amino)butanoate (9.0 g, 35 mmol), potassium carbonate (18.0 g, 130 mmol) and boc-anhydride (13 g, 60 mmol) was stirred in acetonitrile (100 mL) for 1 h. The reaction was then diluted with water and extracted with EtOAc (3×). The organic layers were combined, washed with brine, dried over sodium sulfate, and concentrated under vacuum. The residue was purified via silica gel column chromatography (25% EtOAc/PE) to afford tert-butyl 4-((tert-butoxycarbonyl)(2-hydroxyethyl)amino)butanoate (7.5 g, 63%) as a yellow solid. LCMS (M+H)⁺=304.2.

Step 15-3, preparation of tert-butyl 4-((tert-butoxycarbonyl)(2-oxoethyl)amino)butanoate: To tert-butyl 4-((tert-butoxycarbonyl)(2-hydroxyethyl)amino)butanoate (7.5 g, 25 mmol) in acetonitrile (100 mL) at 0° C. was added portionwise Dess-Martin periodinane (15 g, 35 mmol). It was stirred 2 h, then quenched with 1:1 sat. NaHCO₃(aq)/Na₂S₂O₃(aq), and extracted with DCM (3×). The organic layers were combined, washed with brine, dried over sodium sulfate, and concentrated under vacuum. The residue was purified via silica gel column chromatography (25% EtOAc/PE) to give tert-butyl 4-((tert-butoxycarbonyl)(2-oxoethyl)amino)butanoate (6.1 g, 82%) as a yellow oil. LCMS (M+H)⁺=302.2.

Step 15-4, preparation of tert-butyl 4-((tert-butoxycarbonyl)(2-((4-(9-cyano-2,11-difluoro-5-isopropyl-6-oxo-5,6-dihydro-7H-benzo[d]pyrido[3,2-f][1,3]diazepin-7-yl)-3,5-difluorophenyl)amino)ethyl)amino)butanoate: To a mixture of 7-(4-amino-2,6-difluorophenyl)-2,11-difluoro-5-isopropyl-6-oxo-6,7-dihydro-5H-benzo[d]pyrido[3,2-f][1,3]diazepine-9-carbonitrile (from “Step 13-9, Example 13”) (20 mg, 45 μmol), tert-butyl 4-((tert-butoxycarbonyl)(2-oxoethyl)amino)butanoate (45 mg, 0.15 mmol) in EtOH (1 mL), was added ZnCl₂ (10 mg, 73 μmol), and the suspension was stirred 1 h at 60° C. The mixture was cooled to RT and was added NaBH₃CN (10 mg, 0.16 mmol), then the reaction was heated an additional 1 h at 60° C., then cooled and purified via prep-HPLC under the following conditions: column, SunFire Prep C18 OBD column, 19*150 mm, 5 μm; mobile phase, 35-98% ACN/water, 0.1% FA over 7 min; total flow, 70 mL/min; detector, UV 220/254 nm. This yielded tert-butyl 4-((tert-butoxycarbonyl)(2-((4-(9-cyano-2,11-difluoro-5-isopropyl-6-oxo-5,6-dihydro-7H-benzo[d]pyrido[3,2-f][1,3]diazepin-7-yl)-3,5-difluorophenyl)amino)ethyl)amino)butanoate (25 mg, 76%) as a tan solid. LCMS (M+H)⁺=727.5.

Step 15-5, preparation of 4-{[2-({4-[13-cyano-4,15-difluoro-9-oxo-8-(propan-2-yl)-6,8,10-triazatricyclo[9.4.0.0^{2,7}]pentadeca-1(15),2(7),3,5,11,13-hexaen-10-yl]-3,5-difluorophenyl}amino)ethyl]amino}butanoic acid: From tert-butyl 4-((tert-butoxycarbonyl)(2-((4-(9-cyano-2,11-difluoro-5-isopropyl-6-oxo-5,6-dihydro-7H-benzo[d]pyrido[3,2-f][1,3]diazepin-7-yl)-3,5-difluorophenyl)amino)ethyl)amino)butanoate (25 mg, 34 μmol), the TFA salt of the title compound (16 mg, 57%) was prepared using a similar method as described in “Step 9-2, Example 9”. LCMS (M+H)⁺=571.2.

The following compounds were prepared similarly to Example 15 with appropriate substituting reagents and substrates at different steps and may require additional functional group modifications, via well-known chemistry with appropriate reagents. The reductive amination reaction can be carried out with other reductants such as sodium triacetoxyborohydride under appropriate conditions. Different salts, such as HCl, formic acid, or TFA salts, may be obtained.

| Compound no. | MS (M + H)⁺ |
|---|---|
| 1-101 | 573.3 |
| 1-104 | 557.2 |

### Example 16: 10-[2,6-difluoro-4-({2-[(2-hydroxyethyl)amino]ethyl}amino)phenyl]-8-ethyl-9-oxo-4-(trifluoromethyl)-6,8,10-triazatricyclo[9.4.0.0^{2,7}]pentadeca-1(11),2(7),3,5,12,14-hexaene-13-carbonitrile (cpd 1-98)

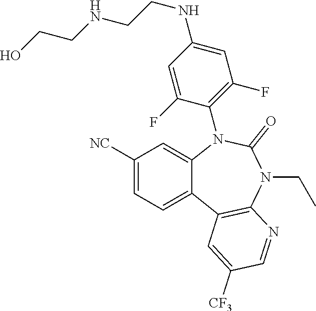 [SMILES: CCN1C(=O)N(c2c(F)cc(NCCNCCO)cc2F)c2cc(C#N)ccc2-c2cc(C(F)(F)F)cnc21]

Step 16-1, preparation of 3-bromo-N-ethyl-5-(trifluoromethyl)pyridin-2-amine: From 3-bromo-5-(trifluoromethyl)pyridin-2-amine (10.0 g, 41.5 mmol), the title compound (8.5 g, 76%) was prepared using a similar method as described in “Step 1-1, Example 1”. LCMS (M+H)⁺=369.0.

Step 16-2, preparation of 3-amino-4-(2-(ethylamino)-5-(trifluoromethyl)pyridin-3-yl)benzonitrile: A mixture of 3-bromo-N-ethyl-5-(trifluoromethyl)pyridin-2-amine (7.0 g, 26 mmol), 3-amino-4-(4,4,5,5-tetramethyl-1,3,2-dioxaborolan-2-yl)benzonitrile (from “Step 6-1, Example 6”) (9.0 g, 37 mmol), K₂CO₃ (12 g, 87 mmol) and Pd(dtbpf)Cl₂ (1.0 g, 1.5 mmol) in 1,4-dioxane/water (100 mL/10 mL) under nitrogen was heated at 80° C. for 2 h. The mixture was concentrated, then purified by silica gel column chromatography (25% EtOAc/PE) to afford 3-amino-4-(2-(ethylamino)-5-(trifluoromethyl)pyridin-3-yl)benzonitrile (6.5 g, 82%) as a yellow solid. LCMS (M+H)⁺=307.1.

Step 16-3, preparation of 5-ethyl-6-oxo-2-(trifluoromethyl)-6,7-dihydro-5H-benzo[d]pyrido[3,2-f][1,3]diazepine-9-carbonitrile: From 3-amino-4-(2-(ethylamino)-5-(trifluoromethyl)pyridin-3-yl)benzonitrile (6.1 g, 20 mmol), the title compound (5.2 g, 79%) was prepared using a similar method as described in “Step 6-3, Example 6”.

Step 16-4, preparation of 7-(2,6-difluoro-4-nitrophenyl)-5-ethyl-6-oxo-2-(trifluoromethyl)-6,7-dihydro-5H-benzo[d]pyrido[3,2-f][1,3]diazepine-9-carbonitrile: From 5-ethyl-6-oxo-2-(trifluoromethyl)-6,7-dihydro-5H-benzo[d]pyrido-[3,2-f][1,3]diazepine-9-carbonitrile (5.2 g, 16 mmol), the title compound (7.2 g, 94%) was prepared using a similar method as described in “Step 6-4, Example 6”.

Step 16-5, preparation of 7-(4-amino-2,6-difluorophenyl)-5-ethyl-6-oxo-2-(trifluoromethyl)-6,7-dihydro-5H-benzo[d]pyrido[3,2-f][1,3]diazepine-9-carbonitrile: From 7-(2,6-difluoro-4-nitrophenyl)-5-ethyl-6-oxo-2-(trifluoromethyl)-6,7-dihydro-5H-benzo[d]pyrido[3,2-f][1,3]diazepine-9-carbonitrile (7.2 g, 15 mmol), the title compound (6.0 g, 89%) was prepared using a similar method as described in “Step 6-5, Example 6”.

Step 16-6, preparation of tert-butyl (2-((tert-butyldimethylsilyl)oxy)ethyl)(2-((4-(9-cyano-5-ethyl-6-oxo-2-(trifluoromethyl)-5,6-dihydro-7H-benzo[d]pyrido[3,2-f][1,3]diazepin-7-yl)-3,5-difluorophenyl)amino)ethyl)carbamate: From 7-(4-amino-2,6-difluorophenyl)-5-ethyl-6-oxo-2-(trifluoromethyl)-6,7-dihydro-5H-benzo[d]pyrido[3,2-f][1,3]diazepine-9-carbonitrile (20 mg, 44 μmol), the title compound (20 mg, 60%) was prepared using a similar method as described in “Step 7-1, Example 7”.

Step 16-7, preparation of 10-[2,6-difluoro-4-({2-[(2-hydroxyethyl)amino]ethyl}amino)phenyl]-8-ethyl-9-oxo-4-(trifluoromethyl)-6,8,10-triazatricyclo[9.4.0.0^{2,7}]pentadeca-1(11),2(7),3,5,12,14-hexaene-13-carbonitrile: From tert-butyl (2-((tert-butyldimethylsilyl)oxy)ethyl)(2-((4-(9-cyano-5-ethyl-6-oxo-2-(trifluoromethyl)-5,6-dihydro-7H-benzo[d]pyrido[3,2-f][1,3]diazepin-7-yl)-3,5-difluorophenyl)amino)ethyl)carbamate (20 mg, 26 μmol), the title compound (11 mg, 74%) was prepared using a similar method as described in “Step 7-2, Example 7”.

The following compound was prepared similarly to Example 16 with appropriate substituting reagents and substrates at different steps and may require additional functional group modifications, via well-known chemistry with appropriate reagents.

| Compound no. | MS (M + H)⁺ |
|---|---|
| 1-208 | 493.3 |

### Example 17: 10-(2,6-difluoro-4-{[2-(methylamino)ethyl]amino}phenyl)-8-ethyl-9-oxo-4-(trifluoromethyl)-6,8,10-triazatricyclo[9.4.0.0^{2,7}]pentadeca-1(11),2(7),3,5,12,14-hexaene-13-carbonitrile (cpd 1-97)

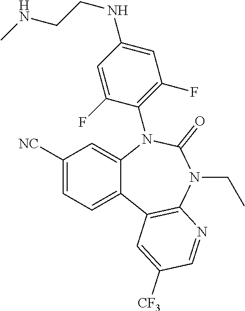 [SMILES: CCN1C(=O)N(c2c(F)cc(NCCNC)cc2F)c2cc(C#N)ccc2-c2cc(C(F)(F)F)cnc21]

Step 17-1, preparation of tert-butyl (2-((4-(9-cyano-5-ethyl-6-oxo-2-(trifluoromethyl)-5,6-dihydro-7H-benzo[d]pyrido[3,2-f][1,3]diazepin-7-yl)-3,5-difluorophenyl)amino)ethyl)(methyl)carbamate: From 7-(4-amino-2,6-difluorophenyl)-5-ethyl-6-oxo-2-(trifluoromethyl)-6,7-dihydro-5H-benzo[d]pyrido[3,2-f][1,3]diazepine-9-carbonitrile (from “Step 16-5, Example 16”) (20 mg, 44 μmol), the title compound (20 mg, 75%) was prepared using a similar method as described in “Step 6-6, Example 6”.

Step 17-2, preparation of 10-(2,6-difluoro-4-{[2-(methylamino)ethyl]amino}phenyl)-8-ethyl-9-oxo-4-(trifluoromethyl)-6,8,10-triazatricyclo[9.4.0.0^{2,7}]pentadeca-1(11),2(7),3,5,12,14-hexaene-13-carbonitrile: From tert-butyl (2-((4-(9-cyano-5-ethyl-6-oxo-2-(trifluoromethyl)-5,6-dihydro-7H-benzo[d]pyrido[3,2-f][1,3]diazepin-7-yl)-3,5-difluorophenyl)amino)ethyl)(methyl)carbamate (20 mg, 32 μmol), the title compound (14 mg, 57%) was prepared using a similar method as described in “Step 6-7, Example 6”.

### Example 18: Methyl N-[2-({2-[(4-{4-chloro-13-cyano-8-ethyl-9-oxo-6,8,10-triazatricyclo[9.4.0.0^{2,7}]pentadeca-1(11),2(7),3,5,12,14-hexaen-10-yl}-3,5-difluorophenyl)amino]ethyl}amino)ethyl]carbamate (cpd 1-167)

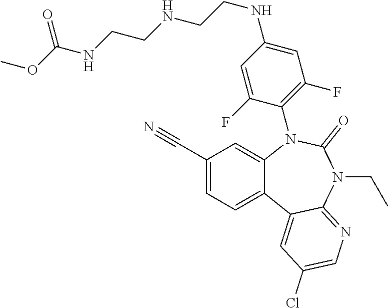 [SMILES: CCN1C(=O)N(c2c(F)cc(NCCNCCNC(=O)OC)cc2F)c2cc(C#N)ccc2-c2cc(Cl)cnc21]

Step 18-1, preparation of tert-butyl (2-aminoethyl)(2-((4-(2-chloro-9-cyano-5-ethyl-6-oxo-5,6-dihydro-7H-benzo[d]pyrido[3,2-f][1,3]diazepin-7-yl)-3,5-difluorophenyl)amino)ethyl)carbamate: A solution of 2-chloro-7-(2,6-difluoro-4-iodophenyl)-5-ethyl-6-oxo-6,7-dihydro-5H-benzo[d]pyrido[3,2-f][1,3]diazepine-9-carbonitrile (from “Step 12-1, Example 12) (100.0 mg, 186.3 μmol), CuI (36.0 mg, 189 μmol), tert-butyl bis(2-aminoethyl)carbamate (150.0 mg, 737.9 μmol), K₂CO₃ (75.0 mg, 543 μmol), 2-isobutyrylcyclohexan-1-one (32.0 mg, 190 μmol) was prepared in DMSO (1 mL), and stirred for 2 hour at 20° C. in an oil bath. The residue was applied onto a C18 gel column with H₂O(0.1% FA)/ACN=(65/35). The collected fractions were combined and concentrated under vacuum to provide tert-butyl (2-aminoethyl)(2-((4-(2-chloro-9-cyano-5-ethyl-6-oxo-5,6-dihydro-7H-benzo[d]pyrido[3,2-f][1,3]diazepin-7-yl)-3,5-difluorophenyl)amino)ethyl)carbamate (20 mg, 18%). (M+H)⁺=612.2.

Step 18-2, preparation of tert-butyl (2-((4-(2-chloro-9-cyano-5-ethyl-6-oxo-5,6-dihydro-7H-benzo[d]pyrido[3,2-]H[1,3]diazepin-7-yl)-3,5-difluorophenyl)amino)ethyl)(2-((methoxycarbonyl)amino)ethyl)carbamate: A solution of tert-butyl (2-aminoethyl)(2-((4-(2-chloro-9-cyano-5-ethyl-6-oxo-5,6-dihydro-7H-benzo[d]pyrido[3,2-f][1,3]diazepin-7-yl)-3,5-difluorophenyl)amino)ethyl)carbamate (20.0 mg, 32.7 μmol), TEA (124 μL, 889 μmol), and methyl carbonochloridate (2.8 mg, 30 μmol) in DCM (2 mL) was prepare, and stirred for 1 hour at 0° C. The reaction was then quenched by the addition of 8 mL of water/ice. The resulting solution was extracted with 3×3 mL of DCM, and concentrated under vacuum providing tert-butyl (2-((4-(2-chloro-9-cyano-5-ethyl-6-oxo-5,6-dihydro-7H-benzo[d]pyrido[3,2-f][1,3]diazepin-7-yl)-3,5-difluorophenyl)amino)ethyl)(2-((methoxycarbonyl)amino)ethyl)carbamate (12.0 mg, 54.8%). (M+H)⁺=670.2.

Step 18-preparation of methyl (2-((2-((4-(2-chloro-9-cyano-5-ethyl-6-oxo-5,6-dihydro-7H-benzo[d]pyrido[3,2-f][1,3]diazepin-7-yl)-3,5-difluorophenyl)amino)ethyl)amino)ethyl)carbamate: A solution of tert-butyl (2-((4-(2-chloro-9-cyano-5-ethyl-6-oxo-5,6-dihydro-7H-benzo[d]pyrido[3,2-f][1,3]diazepin-7-yl)-3,5-difluorophenyl)amino)ethyl)(2-((methoxycarbonyl)amino)ethyl)carbamate (12.0 mg, 17.9 μmol) in DCM (5 mL) and TFA (1 mL) was prepared, and stirred for 1 hour at 25° C. The resulting mixture was concentrated under vacuum. The crude product was purified by Prep-HPLC with the following conditions (Prep-HPLC-013): Column, SunFire Prep C18 OBD Column, 19*150 mm 5 um; mobile phase, Water (0.05% TFA) and ACN (35.0% ACN up to 55.0% in 7 min); Total flow rate, 20 mL/min; Detector, UV 220 nm. The collected fractions were combined and concentrated under vacuum, then lyophilization, providing the TFA salt of the title compound (5.6 mg, 39%). (M+H)⁺=570.2.

The following compounds were prepared similarly to Example 18 with appropriate substituting reagents and substrates at different steps and may require additional functional group modifications, via well-known chemistry with appropriate reagents.

| Compound no. | MS (M + H)⁺ |
|---|---|
| 1-175 | 591.2 |
| 1-216 | 554.2 |
| 1-221 | 575.1 |

### Example 19: 5,14-dichloro-8-[2,6-difluoro-4-({2-[(2-hydroxyethyl)amino]ethyl}amino)phenyl]-10-ethyl-3,8,10,12-tetraazatricyclo[9.4.0.0^{2,7}]pentadeca-1(11),2(7),3,5,12,14-hexaen-9-one (cpd 3-21)

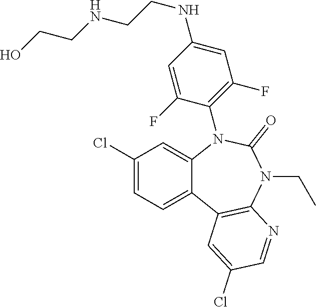 [SMILES: CCN1C(=O)N(c2c(F)cc(NCCNCCO)cc2F)c2cc(Cl)ccc2-c2cc(Cl)cnc21]

Step 19-1, preparation of 5,5′-dichloro-N2′-ethyl-[2,3′-bipyridine]-2′,3-diamine: To a solution of 2-bromo-5-chloropyridin-3-amine (300 mg, 1.45 mmol), 5-chloro-N-ethyl-3-(4,4,5,5-tetramethyl-1,3,2-dioxaborolan-2-yl)pyridin-2-amine (817 mg, 2.89 mmol) in 1,4-dioxane (8 mL) and water (2 mL), was added potassium carbonate (400 mg, 2.89 mmol), Pd(dtbpf)Cl₂ (94.2 mg, 145 μmol). The resulting mixture was stirred at 80° C. for 2 hour under nitrogen. The resulting mixture was quenched with NH₄Cl aq. (20 mL), extracted with EtOAc (3*20 mL). The organic layers combined, dried over Na₂SO₄, filtered and concentrated. The residue was applied onto a silica gel column with ethyl acetate/petroleum ether (20:80), providing 5,5′-dichloro-N2′-ethyl-[2,3′-bipyridine]-2′,3-diamine (280 mg, 68.4%). (M+H)⁺=283.0.

Step 19-2, preparation of 3,10-dichloro-7-ethyl-5,7-dihydro-6H-dipyrido[2,3-d:2′,3′-f][1,3]diazepin-6-one: To a solution of 5,5′-dichloro-N2′-ethyl-[2,3′-bipyridine]-2′,3-diamine (260 mg, 918 μmol), N-ethyl-N-isopropylpropan-2-amine (356 mg, 2.75 mmol) in DCM (6 mL), was added bis(trichloromethyl) carbonate (109 mg, 367 μmol) in DCM (2 mL) dropwise with stirring at 0° C. The resulting mixture was stirred at 0° C. for 1 hour. The resulting mixture was quenched with NH₄Cl aq. (20 mL), extracted with EtOAc (3*20 mL). The organic layers combined, dried over Na₂SO₄, filtered and concentrated. The residue was applied onto a silica gel column with ethyl acetate/petroleum ether (30:70), providing 3,10-dichloro-7-ethyl-5,7-dihydro-6H-dipyrido[2,3-d:2′,3′-f][1,3]diazepin-6-one (80 mg, 28%). (M+H)⁺=309.0.

Step 19-3, preparation of 3,10-dichloro-5-(2,6-difluoro-4-nitrophenyl)-7-ethyl-5,7-dihydro-6H-dipyrido[2,3-d:2′,3′-f][1,3]diazepin-6-one: To a solution of 3,10-dichloro-7-ethyl-5,7-dihydro-6H-dipyrido[2,3-d:2′,3′-f][1,3]diazepin-6-one (80 mg, 0.26 mmol) in DMF (4 mL), was added sodium hydride (21 mg, 60% Wt, 0.52 mmol),1,2,3-trifluoro-5-nitrobenzene (92 mg, 0.52 mmol). The resulting mixture was stirred at 25° C. for 1 hour. The resulting mixture was quenched with NH₄Cl aq. (15 mL), extracted with MTBE (3*15 mL). The organic layers combined, dried over Na₂SO₄, filtered and concentrated. The residue was applied onto a silica gel column with ethyl acetate/petroleum ether (20:80), providing 3,10-dichloro-5-(2,6-difluoro-4-nitrophenyl)-7-ethyl-5,7-dihydro-6H-dipyrido[2,3-d:2′,3′-f][1,3]diazepin-6-one (85 mg 70%). (M+H)⁺=466.1.

Step 19-4, preparation of 5-(4-amino-2,6-difluorophenyl)-3,10-dichloro-7-ethyl-5,7-dihydro-6H-dipyrido[2,3-d:2′,3′-f][1,3]diazepin-6-one: To a solution of 3,10-dichloro-5-(2,6-difluoro-4-nitrophenyl)-7-ethyl-5,7-dihydro-6H-dipyrido[2,3-d:2′,3′-f][1,3]diazepin-6-one (85 mg, 0.18 mmol) in EtOH (5 mL) and water (1 mL), was added ammonium chloride (49 mg, 0.91 mmol), iron (51 mg, 0.91 mmol). The resulting mixture was stirred at 70° C. for 1 hour. The resulting mixture was quenched with water (20 mL), extracted with EtOAc (3*20 mL). The organic layers combined, dried over Na₂SO₄, filtered and concentrated. The residue was applied onto a silica gel column with ethyl acetate/petroleum ether (30:70), providing 5-(4-amino-2,6-difluorophenyl)-3,10-dichloro-7-ethyl-5,7-dihydro-6H-dipyrido[2,3-d:2′,3′-f][1,3]diazepin-6-one (70 mg, 88%). (M+H)⁺=436.0.

Step 19-5, preparation of tert-butyl (2-((tert-butyldimethylsilyl)oxy)ethyl)(2-((4-(3,10-dichloro-7-ethyl-6-oxo-6,7-dihydro-5H-dipyrido[2,3-d:2′,3′-f][1.3]diazepin-5-yl)-3,5-difluorophenyl)amino)ethyl)carbamate: To a solution of 5-(4-amino-2,6-difluorophenyl)-3,10-dichloro-7-ethyl-5,7-dihydro-6H-dipyrido[2,3-d:2′,3′-f][1,3]diazepin-6-one (20 mg, 46 μmol) in EtOH (2 mL), was added tert-butyl (2-((tert-butyldimethylsilyl)oxy)ethyl)(2-oxoethyl)carbamate (44 mg, 3 Eq, 0.14 mmol), zinc(II) chloride (12 mg, 92 μmol), sodium cyanotrihydroborate (8.6 mg, 0.14 mmol). The resulting mixture was stirred at 60° C. for 2 hour, then quenched with NH₄Cl aq. (10 mL), and extracted with EtOAc (3*10 mL). The organic layers were combined, dried over Na₂SO₄, filtered and concentrated. The residue was applied onto a silica gel column with ethyl acetate/petroleum ether (20:80), providing tert-butyl (2-((tert-butyldimethylsilyl)oxy)ethyl)(2-((4-(3,10-dichloro-7-ethyl-6-oxo-6,7-dihydro-5H-dipyrido[2,3-d:2′,3′-f][1,3]diazepin-5-yl)-3,5-difluorophenyl)amino)ethyl)carbamate (20 mg, 59%). (M+Na)⁺=759.3.

Step 19-6, preparation of 5,14-dichloro-8-[2,6-difluoro-4-({2-[(2-hydroxyethyl)amino]ethyl}amino)phenyl]-10-ethyl-3,8,10,12-tetraazatricyclo[9.4.0.0^{2,7}]pentadeca-1(11),2(7),3,5,12,14-hexaen-9-one: To a solution of tert-butyl (2-((tert-butyldimethylsilyl)oxy)ethyl)(2-((4-(3,10-dichloro-7-ethyl-6-oxo-6,7-dihydro-5H-dipyrido[2,3-d:2′,3′-f][1,3]diazepin-5-yl)-3,5-difluorophenyl)amino)ethyl)carbamate (20 mg, 27 μmol) in DCM (5 mL) was added TFA (1 mL). The resulting mixture was stirred at 25° C. for 1 hour. The resulting solution was concentrated to afford crude product. The title compound was isolated by Prep-HPLC with the following conditions: Column, SunFire Prep C18 OBD Column, 19*150 mm 5 um 10 nm; mobile phase, water(0.1% FA) and CAN(35% Phase B up to 55% in 8 min); total flow 20 mL/min; detector UV 254 nm (3.0 mg, 19%). (M+H)⁺=523.2.

The following compound was prepared similarly to Example 19 with appropriate substituting reagents and substrates at different steps and may require additional functional group modifications, via well-known chemistry with appropriate reagents.

| Compound no. | MS (M + H)⁺ |
|---|---|
| 3-25 | 507.2 |

### Example 20: 13-chloro-10-[2,6-difluoro-4-({2-[(2-hydroxyethyl)amino]ethyl}amino)phenyl]-8-ethyl-9-oxo-5,8,10-triazatricyclo[9.4.0.0^{2,7}]pentadeca-1(11),2(7),3,5,12,14-hexaene-4-carbonitrile (cpd 3-22)

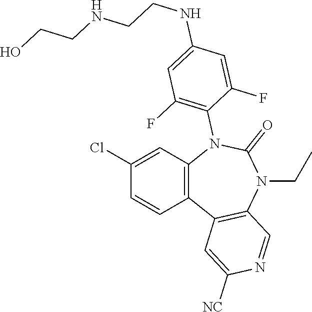 [SMILES: CCN1C(=O)N(c2c(F)cc(NCCNCCO)cc2F)c2cc(Cl)ccc2-c2cc(C#N)ncc21]

Step 20-1, preparation of tert-butyl (6-chloro-4-iodopyridin-3-yl)(ethyl)carbamate: A mixture of tert-butyl (6-chloro-4-iodopyridin-3-yl)carbamate (2 g, 6 mmol) in DMF (80 mL) was cooled to 0° C., followed by the addition NaH (0.5 g, 60% Wt, 0.01 mol). The resulting reaction mixture was stirred for 0.5 h at 0° C., then iodoethane was added (1 g, 6 mmol). The resulting reaction mixture was stirred for 0.5 h at 5° C. The reaction was then quenched by water (30 mL). The resulting solution was extracted with ethyl acetate (3×30 mL). The organic layers were dried over anhydrous sodium sulfate and concentrated under vacuum. The residue was applied onto a silica gel column with ethyl acetate/petroleum ether (1:1), providing tert-butyl (6-chloro-4-iodopyridin-3-yl)(ethyl)carbamate (1.5 g, 70%). (M+H)⁺=383.0.

Step 20-2, preparation of 5-chloro-2-(4,4,5,5-tetramethyl-1,3,2-dioxaborolan-2-yl)aniline: A solution of 2-bromo-5-chloroaniline (2 g, 0.01 mol), 1,1′-Bis(diphenylphosphino)ferrocene-palladium(II) dichloride (0.35 g, 0.48 mmol), BPD (4.3 g, 17 mmol), and KOAc (3.2 g, 33 mmol) in 1,4-dioxane (20 mL) was stirred at 80° C. for 16 hour in an oil bath. The resulting mixture was concentrated under vacuum. The residue was applied onto a silica gel column with ethyl acetate/petroleum ether (20/80). The collected fractions were combined and concentrated under vacuum, providing 5-chloro-2-(4,4,5,5-tetramethyl-1,3,2-dioxaborolan-2-yl)aniline (1.6 g, 70%). (M+H)⁺=253.0.

Step 20-3: preparation of tert-butyl (4-(2-amino-4-chlorophenyl)-6-chloropyridin-3-yl)(ethyl)carbamate: A solution of tert-butyl (6-chloro-4-iodopyridin-3-yl)(ethyl)carbamate (1 g, 3 mmol), Pd(DTBPF)Cl₂ (0.08 g, 0.1 mmol), 5-chloro-2-(4,4,5,5-tetramethyl-1,3,2-dioxaborolan-2-yl)aniline (1.1 g, 4.3 mmol), and K₂CO₃ (1.2 g, 8.7 mmol) in 1,4-dioxane (60 mL) and water (6 mL) was stirred for 60° C. for 2 hour in an oil bath. The resulting mixture was concentrated under vacuum. The residue was applied onto a silica gel column with ethyl acetate/petroleum ether (20/80). The collected fractions were combined and concentrated under vacuum to provide tert-butyl (4-(2-amino-4-chlorophenyl)-6-chloropyridin-3-yl)(ethyl)carbamate (800 mg, 80%). (M+H)⁺=423.1.

Step 20-4: preparation of 4-(2-amino-4-chlorophenyl)-6-chloro-N-ethylpyridin-3-amine: A solution of tert-butyl (4-(2-amino-4-chlorophenyl)-6-chloropyridin-3-yl)(ethyl)carbamate (700 mg, 1.83 mmol) and TEA (10 mL) in DCM (20 mL) was stirred at 25° C. for 1 hour. The solids were filtered and the resulting solution was concentrated under vacuum. The residue was applied onto a silica gel column with ethyl acetate/petroleum ether (20/80). The collected fractions were combined and concentrated under vacuum, providing 4-(2-amino-4-chlorophenyl)-6-chloro-N-ethylpyridin-3-amine (510 mg, 98.7%). (M+H)⁺=282.0.

Step 20-5: preparation of 2,9-dichloro-5-ethyl-5,7-dihydro-6H-benzo[d]pyrido[4,3-f][13]diazepin-6-one: Triphosgene (610 mg, 2.06 mmol) in DCM (1 mL) was added dropwise to a solution of 4-(2-amino-4-chlorophenyl)-6-chloro-N-ethylpyridin-3-amine (480 mg, 1.70 mmol) and TEA (520 mg, 716 μL, 5.14 mmol) in DCM (10 mL) with stirring at 0° C. The resulting mixture was stirred at 0° C. for 3 hour. The resulting solution was diluted with water (20 mL) and extracted with ethyl acetate (3×20 mL) and the organic layers combined. The resulting mixture was washed with saturated NaHCO₃aq.brine (20 mL). The mixture was dried over anhydrous sodium sulfate and concentrated under vacuum, providing 2,9-dichloro-5-ethyl-5,7-dihydro-6H-benzo[d]pyrido[4,3-f][1,3]diazepin-6-one (410 mg, 78.2%). (M+H)⁺=308.1.

Step 20-6: preparation of 9-chloro-5-ethyl-6-oxo-6,7-dihydro-5H-benzo[d]pyrido[4,3-f][1,3]diazepine-2-carbonitrile: A solution of 2,9-dichloro-5-ethyl-5,7-dihydro-6H-benzo[d]pyrido[4,3-f][1,3]diazepin-6-one (300 mg, 974 μmol), Pd₂(dba)₃ (45 mg, 49 μmol), dicyanozinc (240 mg, 2.04 mmol), XantPhos (57 mg, 99 μmol), and NMP (8 mL) was stirred at 150° C. for 1 hour under microwave radiation. The resulting mixture was concentrated under vacuum. The residue was applied onto a silica gel column with ethyl acetate/petroleum ether (30/80). The collected fractions were combined and concentrated under vacuum, providing 9-chloro-5-ethyl-6-oxo-6,7-dihydro-5H-benzo[d]pyrido[4,3-f][1,3]diazepine-2-carbonitrile (100 mg, 34.4%). (M+H)⁺=299.1.

Step 20-7: preparation of 9-chloro-7-(2,6-difluoro-4-nitrophenyl)-5-ethyl-6-oxo-6,7-dihydro-5H-benzo[d]pyrido[4,3-f][1,3]diazepine-2-carbonitrile: A mixture of 9-chloro-5-ethyl-6-oxo-6,7-dihydro-5H-benzo[d]pyrido[4,3-f][1,3]diazepine-2-carbonitrile (80 mg, 0.27 mmol) in DMF (5 mL) was cooled to 0° C., followed by the addition of NaH (25 mg, 60% Wt, 0.63 mmol). The resulting reaction mixture was stirred for 0.5 h at 0° C. 1,2,3-trifluoro-5-nitrobenzene (70 mg, 0.40 mmol) was then added, and the resulting reaction mixture was stirred for 0.5 h at 0° C. The reaction was then quenched by water (30 mL). The resulting solution was extracted with ethyl acetate (3×30 mL). The organic layers were dried over anhydrous sodium sulfate and concentrated under vacuum. The residue was applied onto a silica gel column with ethyl acetate/petroleum ether (1:1), providing 9-chloro-7-(2,6-difluoro-4-nitrophenyl)-5-ethyl-6-oxo-6,7-dihydro-5H-benzo[d]pyrido[4,3-f][1,3]diazepine-2-carbonitrile (90 mg, 74%). (M+H)⁺=456.1.

Step 20-8: preparation of 7-(4-amino-2,6-difluorophenyl)-9-chloro-5-ethyl-6-oxo-6,7-dihydro-5H-benzo[d]pyrido[4,3-f][1,3]diazepine-2-carbonitrile: A solution of 9-chloro-7-(2,6-difluoro-4-nitrophenyl)-5-ethyl-6-oxo-6,7-dihydro-5H-benzo[d]pyrido[4,3-f][1,3]diazepine-2-carbonitrile (80 mg, 0.18 mmol), EtOH (10 mL), water (1 mL), Fe (50 mg, 0.90 mmol), NH₄Cl (49 mg, 0.92 mmol) was stirred for 80° C. for 1 hour. The solids were then filtered out. The resulting mixture was concentrated under vacuum. The residue was applied onto a silica gel column with ethyl acetate/petroleum ether (20/80). The collected fractions were combined and concentrated under vacuum providing 7-(4-amino-2,6-difluorophenyl)-9-chloro-5-ethyl-6-oxo-6,7-dihydro-5H-benzo[d]pyrido[4,3-f][1,3]diazepine-2-carbonitrile (50 mg, 67%). (M+H)⁺=426.1.

Step 20-9: preparation of tert-butyl (2-((tert-butyldimethylsilyl)oxy)ethyl)(2-((4-(9-chloro-2-cyano-5-ethyl-6-oxo-5,6-dihydro-7H-benzo[d]pyrido[4,3-f][1,3]diazepin-7-yl)-3,5-difluorophenyl)amino)ethyl)carbamate: A mixture of 7-(4-amino-2,6-difluorophenyl)-9-chloro-5-ethyl-6-oxo-6,7-dihydro-5H-benzo[d]pyrido[4,3-f][1,3]diazepine-2-carbonitrile (20 mg, 47 μmol), ZnCl₂ (20 mg, 0.15 mmol), tert-butyl (2-((tert-butyldimethylsilyl)oxy)ethyl)(2-oxoethyl)carbamate (40 mg, 0.13 mmol), 1,4-dioxane (2 mL) was stirred at 25° C. for 0.5 hour. Then NaBH₃CN (6 mg, 0.1 mmol) was added, and the mixture was stirred at 25° C. for 0.5 hour. The reaction mixture was cooled to room temperature and concentrated under vacuum. The residue was applied onto a silica gel column with ethyl acetate/petroleum ether (1:2), providing tert-butyl (2-((tert-butyldimethylsilyl)oxy)ethyl)(2-((4-(9-chloro-2-cyano-5-ethyl-6-oxo-5,6-dihydro-7H-benzo[d]pyrido[4,3-f][1,3]diazepin-7-yl)-3,5-difluorophenyl)amino)ethyl)carbamate (17 mg, 50%). (M+H)⁺=727.5.

Step 20-10: preparation of 13-chloro-10-[2,6-difluoro-4-({2-[(2-hydroxyethyl)amino]ethyl}amino)phenyl]-8-ethyl-9-oxo-5,8,10-triazatricyclo[9.4.0.0^{2,7}]pentadeca-1(11),2(7),3,5,12,14-hexaene-4-carbonitrile: A mixture of tert-butyl (2-((tert-butyldimethylsilyl)oxy)ethyl)(2-((4-(9-chloro-2-cyano-5-ethyl-6-oxo-5,6-dihydro-7H-benzo[d]pyrido[4,3-f][1,3]diazepin-7-yl)-3,5-difluorophenyl)amino)ethyl)carbamate (14 mg, 19 μmol) and HCl (1 mL) in Et₂O (5 mL) was stirred at 25° C. for 1 hour. The resulting solution was concentrated under vacuum. The crude product was purified by Prep-HPLC with the following conditions (Prep-HPLC-007): Column, SunFire Prep C18 OBD Column, 19*150 mm 5 um 10 nm; mobile phase, water (0.05% TFA) and ACN (27% ACN up to 59% in 10 min); Total flow rate, 20 mL/min; Detector, UV 220 nm. This gave the TFA salt of the title compound (4.2 mg, 29%). (M+H)⁺=513.2.

The following compound was prepared similarly to Example 20 with appropriate substituting reagents and substrates at different steps and may require additional functional group modifications, via well-known chemistry with appropriate reagents.

| Compound no. | MS (M + H)⁺ |
|---|---|
| 3-24 | 553.2 |

### Example A-1: Parenteral Pharmaceutical Composition

To prepare a parenteral pharmaceutical composition suitable for administration by injection (subcutaneous, intravenous), 1-100 mg of a water-soluble salt of a compound Formula (I′) or (I), or a pharmaceutically acceptable salt or solvate thereof, is dissolved in sterile water and then mixed with 10 mL of 0.9% sterile saline. A suitable buffer is optionally added as well as optional acid or base to adjust the pH. The mixture is incorporated into a dosage unit form suitable for administration by injection

### Example A-2: Oral Solution

To prepare a pharmaceutical composition for oral delivery, a sufficient amount of a compound of Formula (I′) or (I), or a pharmaceutically acceptable salt thereof, is added to water (with optional solubilizer(s), optional buffer(s) and taste masking excipients) to provide a 20 mg/mL solution.

### Example A-3: Oral Tablet

A tablet is prepared by mixing 20-50% by weight of a compound of Formula (I′) or (I), or a pharmaceutically acceptable salt thereof, 20-50% by weight of microcrystalline cellulose, 1-10% by weight of low-substituted hydroxypropyl cellulose, and 1-10% by weight of magnesium stearate or other appropriate excipients. Tablets are prepared by direct compression. The total weight of the compressed tablets is maintained at 100-500 mg.

### Example A-4: Oral Capsule

To prepare a pharmaceutical composition for oral delivery, 10-500 mg of a compound of Formula (I′) or (I), or a pharmaceutically acceptable salt thereof, is mixed with starch or other suitable powder blend. The mixture is incorporated into an oral dosage unit such as a hard gelatin capsule, which is suitable for oral administration.

In another embodiment, 10-500 mg of a compound of Formula (I′) or (I), or a pharmaceutically acceptable salt thereof, is placed into Size 4 capsule, or size 1 capsule (hypromellose or hard gelatin) and the capsule is closed.

### Example B-1: PTH Receptor Assays

Competitive Antagonist Binding Assay Protocol for hPTH-R1 Antagonists:

The inhibition of binding to the human PTHR1 receptor was measured in a radioactive competition binding assay using the radiolabeled [¹²⁵I]-[NLE^{8,18}, TYR³⁴]-parathyroid hormone (1-34) ligand (PerkinElmer Cat# NEX244, custom synthesis) as the probe ligand for the receptor. Briefly, membranes from cells expressing the human PTHR1 (Eurofins Cat# HTS030M) were incubated with SPA beads (PerkinElmer Cat# RPNQ0001) in binding assay buffer [50 mM HEPES (Biopioneer Cat# C0113), 5 mM MgCl₂ (Sigma Cat# M8266), 10 mM CaCl₂ (Fisher Scientific Cat# BP510), 0.1% BSA (Fisher Scientific Cat# BP1600), 0.01% TritonX-100 (Bio-Rad Cat#161-0407), and protease inhibitors (Pierce Cat# A32963); pH 7.4]. Membranes bound to SPA bead were treated with various dilutions of compounds (final concentrations typically 0-10,000 nM), and 0.2 nM radiolabeled ligand in 96-well isoplates (PerkinElmer Cat#6005040) for 90 minutes at room temperature. The radioactive signal was detected using a MicroBetaTriLux 1450 LSC (PerkinElmer). All data manipulations to determine the Kᵢ values were performed using GraphPad v8 (GraphPad, San Diego Calif.).

Table A demonstrates illustrative binding affinity of compounds, where “a” means Kᵢ is <100 nM; “b” means Kᵢ is between 100 to 1000 nM; “c” means Kᵢ is between 1000 to 10000 nM; “d” means Kᵢ is >10000 nM.

TABLE A
Representative PTH Antagonist Activity

| Compound No. | hPTHR1 Kᵢ |
|---|---|
| 1-1 | a |
| 1-2 | a |
| 1-3 | a |
| 1-4 | a |
| 1-5 | a |
| 1-6 | a |
| 1-7 | a |
| 1-8 | a |
| 1-9 | a |
| 1-10 | a |
| 1-11 | a |
| 1-12 | a |
| 1-13 | a |
| 1-14 | a |
| 1-15 | a |
| 1-16 | a |
| 1-17 | a |
| 1-18 | a |
| 1-19 | a |
| 1-20 | a |
| 1-21 | a |
| 1-22 | a |
| 1-23 | a |
| 1-24 | a |
| 1-25 | a |
| 1-26 | a |
| 1-27 | a |
| 1-28 | a |
| 1-29 | a |
| 1-30 | a |
| 1-31 | a |
| 1-32 | a |
| 1-33 | a |
| 1-34 | a |
| 1-35 | a |
| 1-36 | a |
| 1-37 | a |
| 1-38 | a |
| 1-39 | a |
| 1-40 | a |
| 1-41 | d |
| 1-42 | a |
| 1-43 | a |
| 1-44 | a |
| 1-45 | a |
| 1-46 | a |
| 1-47 | a |
| 1-48 | a |
| 1-49 | a |
| 1-50 | d |
| 1-51 | a |
| 1-52 | a |
| 1-53 | c |
| 1-54 | a |
| 1-55 | a |
| 1-56 | a |
| 1-57 | a |
| 1-58 | a |
| 1-59 | a |
| 1-60 | a |
| 1-61 | a |
| 1-62 | a |
| 1-63 | a |
| 1-64 | a |
| 1-65 | a |
| 1-66 | a |
| 1-67 | a |
| 1-68 | a |
| 1-69 | a |
| 1-70 | a |
| 1-71 | a |
| 1-72 | a |
| 1-73 | a |
| 1-74 | a |
| 1-75 | a |
| 1-76 | a |
| 1-77 | a |
| 1-78 | a |
| 1-79 | a |
| 1-80 | a |
| 1-81 | a |
| 1-82 | a |
| 1-83 | a |
| 1-84 | a |
| 1-85 | a |
| 1-86 | a |
| 1-87 | a |
| 1-88 | a |
| 1-89 | a |
| 1-90 | a |
| 1-91 | a |
| 1-92 | a |
| 1-93 | a |
| 1-94 | a |
| 1-95 | a |
| 1-96 | a |
| 1-97 | a |
| 1-98 | a |
| 1-99 | a |
| 1-100 | a |
| 1-101 | a |
| 1-102 | a |
| 1-103 | a |
| 1-104 | a |
| 1-105 | a |
| 1-106 | a |
| 1-107 | a |
| 1-108 | a |
| 1-109 | a |
| 1-110 | a |
| 1-111 | a |
| 1-112 | a |
| 1-113 | a |
| 1-114 | a |
| 1-115 | a |
| 1-116 | a |
| 1-117 | a |
| 1-118 | a |
| 1-119 | a |
| 1-120 | a |
| 1-121 | b |
| 1-122 | a |
| 1-123 | a |
| 1-124 | b |
| 1-125 | a |
| 1-126 | a |
| 1-127 | a |
| 1-128 | b |
| 1-129 | a |
| 1-130 | a |
| 1-131 | a |
| 1-132 | a |
| 1-133 | a |
| 1-134 | a |
| 1-135 | a |
| 1-136 | b |
| 1-137 | a |
| 1-138 | a |
| 1-139 | a |
| 1-140 | a |
| 1-141 | a |
| 1-142 | a |
| 1-143 | a |
| 1-144 | a |
| 1-145 | a |
| 1-146 | a |
| 1-147 | a |
| 1-148 | b |
| 1-149 | a |
| 1-150 | a |
| 1-151 | a |
| 1-152 | b |
| 1-153 | a |
| 1-154 | a |
| 1-155 | a |
| 1-156 | a |
| 1-157 | a |
| 1-158 | b |
| 1-159 | a |
| 1-160 | b |
| 1-161 | a |
| 1-162 | a |
| 1-163 | a |
| 1-164 | a |
| 1-165 | a |
| 1-166 | a |
| 1-167 | a |
| 1-168 | a |
| 1-169 | a |
| 1-170 | a |
| 1-171 | a |
| 1-172 | a |
| 1-173 | a |
| 1-174 | a |
| 1-175 | a |
| 1-176 | a |
| 1-177 | a |
| 1-178 | a |
| 1-179 | a |
| 1-180 | a |
| 1-181 | a |
| 1-182 | a |
| 1-183 | a |
| 1-184 | a |
| 1-185 | a |
| 1-186 | a |
| 1-187 | a |
| 1-188 | a |
| 1-189 | a |
| 1-190 | a |
| 1-191 | a |
| 1-192 | a |
| 1-193 | a |
| 1-194 | a |
| 1-195 | a |
| 1-196 | a |
| 1-197 | a |
| 1-198 | a |
| 1-199 | a |
| 1-200 | a |
| 1-201 | a |
| 1-202 | a |
| 1-203 | a |
| 1-204 | a |
| 1-205 | a |
| 1-206 | a |
| 1-207 | a |
| 1-208 | a |
| 1-209 | a |
| 1-210 | a |
| 1-211 | a |
| 1-212 | a |
| 1-213 | a |
| 1-214 | a |
| 1-215 | a |
| 1-216 | a |
| 1-217 | a |
| 1-218 | a |
| 1-219 | a |
| 1-220 | a |
| 1-221 | a |
| 1-222 | a |
| 1-223 | a |
| 1-224 | a |
| 1-225 | a |
| 1-226 | a |
| 1-227 | a |
| 1-228 | a |
| 1-229 | a |
| 1-230 | a |
| 1-231 | a |
| 1-232 | a |
| 1-233 | a |
| 1-234 | a |
| 1-235 | a |
| 1-236 | a |
| 1-237 | a |
| 1-238 | a |
| 1-239 | a |
| 1-240 | a |
| 1-241 | a |
| 1-242 | a |
| 2-1 | b |
| 2-2 | b |
| 2-3 | a |
| 2-4 | a |
| 2-5 | a |
| 2-6 | a |
| 2-7 | a |
| 2-8 | a |
| 2-9 | a |
| 2-10 | a |
| 2-11 | a |
| 2-12 | a |
| 2-13 | a |
| 2-14 | a |
| 3-1 | a |
| 3-2 | a |
| 3-3 | a |
| 3-4 | a |
| 3-5 | a |
| 3-6 | a |
| 3-7 | a |
| 3-8 | b |
| 3-9 | a |
| 3-10 | a |
| 3-11 | a |
| 3-12 | a |
| 3-13 | a |
| 3-14 | a |
| 3-15 | c |
| 3-16 | c |
| 3-17 | c |
| 3-18 | a |
| 3-19 | a |
| 3-20 | a |
| 3-21 | a |
| 3-22 | a |
| 3-23 | a |
| 3-24 | a |
| 3-25 | a |

The examples and embodiments described herein are for illustrative purposes only and various modifications or changes suggested to persons skilled in the art are to be included within the spirit and purview of this application and scope of the appended claims.

1. A compound of Formula (I′), or a pharmaceutically acceptable salt or solvate thereof: 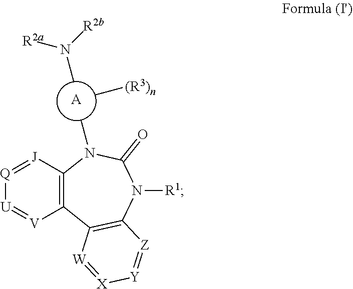
wherein: 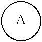
is selected from phenyl and a 6-membered heteroaryl ring comprising 1 or 2 nitrogen atoms;
J is C(R⁴) or N;
Q is C(R⁵) or N;
U is C(R⁶) or N;
V is C(R⁷) or N; wherein no more than two of J, Q, U, and V are N;
W is C(R⁸) or N;
X is C(R⁹) or N;
Y is C(R¹⁰) or N;
Z is C(R¹¹) or N; wherein no more than two of W, X, Y, and Z are N;
R¹ is hydrogen, C₁₋₆alkyl, C₂₋₆alkenyl, C₂₋₆alkynyl, C₃₋₆cycloalkyl, or C₂₋₉heterocycloalkyl, wherein C₁₋₆alkyl, C₂₋₆alkenyl, C₂₋₆alkynyl, C₃₋₆cycloalkyl, and C₂₋₉heterocycloalkyl are optionally substituted with one, two, three, four, or five R¹⁹;
R^{2a} is hydrogen, C₁₋₆alkyl, C₂₋₆alkenyl, C₂₋₆alkynyl, C₃₋₆cycloalkyl, or —(C(R¹⁶)(R¹⁷))ₘ—N(R^{18a})(R^{18b});
R^{2b} is hydrogen, C₁₋₆alkyl, C₂₋₆alkenyl, C₂₋₆alkynyl, or C₃₋₆cycloalkyl, wherein C₁₋₆alkyl, C₂₋₆alkenyl, C₂₋₆alkynyl, and C₃₋₆cycloalkyl are optionally substituted with one, two, three, four, or five R²⁰;
or R^{2a} and R^{2b} are taken together with the nitrogen atom to which they are attached to form a C₂₋₉heterocycloalkyl optionally substituted with one, two, three, four, or five R²⁰;
each R³ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —N(R¹⁴)C(O)R¹⁵, —S(O)₂R¹⁵, and —S(O)₂N(R¹²)(R¹³)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, —OR¹², —N(R¹²)(R¹³), and —C(O)OR¹²;
R⁴, R⁵, R⁶, R⁷, R⁸, R⁹, R¹⁰, and R¹¹ are each independently selected from hydrogen, halogen, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, —CN, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —N(R¹⁴)C(O)R¹⁵, —S(O)₂R¹⁵, and —S(O)₂N(R¹²)(R¹³)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, three, four, or five R²¹;
each R¹² is independently selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, —C(O)OH, C₃₋₆cycloalkyl, C₂₋₉heterocycloalkyl, C₆₋₁₀aryl, and C₁₋₉heteroaryl;
each R¹³ is independently selected from hydrogen, C₁₋₆alkyl, and C₁₋₆haloalkyl;
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹⁶ and each R¹⁷ are each independently selected from hydrogen, halogen, hydroxy, C₁₋₆alkyl, C₁₋₆haloalkyl, and C₁₋₆alkoxy; or R¹⁶ and R¹⁷ are taken together to form a C₃₋₆cycloalkyl;
R^{18a} and R^{18b} are independently selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, three, four, or five R²⁰; or R^{18a} and R^{18b} are taken together with the nitrogen atom to which they are attached to form a C₂₋₉heterocycloalkyl optionally substituted with one, two, three, four, or five R²⁰;
each R¹⁹, R²⁰, and R²¹ are each independently selected from halogen, oxo, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —N(R¹⁴)C(O)R¹⁵, —S(O)₂R¹⁵, and —S(O)₂N(R¹²)(R¹³)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, oxo, C₁₋₆alkyl, C₁₋₆haloalkyl, —OR¹², —S(O)₂OH, —N(R¹²)(R¹³), and —C(O)OR¹²;
m is 2, 3, or 4; and
n is 0, 1, 2, 3, or 4.

2-6. (canceled)

7. The compound of claim 1, or a pharmaceutically acceptable salt or solvate thereof, having the structure of Formula (Ia):

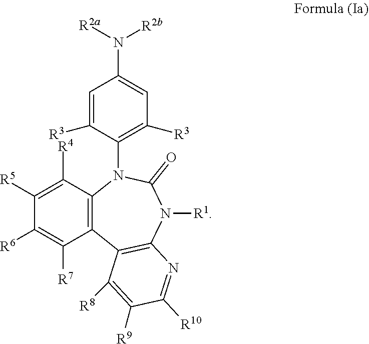

8. The compound of claim 1, or a pharmaceutically acceptable salt or solvate thereof, wherein R¹ is C₁₋₆alkyl optionally substituted with one, two, three, four, or five R¹⁹.

9-11. (canceled)

12. The compound of claim 1, or a pharmaceutically acceptable salt or solvate thereof, wherein R¹ is an unsubstituted C₁₋₆alkyl.

13-31. (canceled)

32. The compound of claim 1, or a pharmaceutically acceptable salt or solvate thereof, wherein R^{2a} and R^{2b} are taken together with the nitrogen atom to which they are attached to form a C₂₋₉heterocycloalkyl optionally substituted with one, two, three, four, or five R²⁰.

33. The compound of claim 32, or a pharmaceutically acceptable salt or solvate thereof, wherein R^{2a} and R^{2b} are taken together with the nitrogen atom to which they are attached to form a C₂₋₉heterocycloalkyl optionally substituted with one, two, three, four, or five R²⁰, and each R²⁰ is independently selected from halogen, —OR¹², —N(R¹²)(R¹³), and C₁₋₆alkyl optionally substituted with —N(R¹²)(R¹³).

34. The compound of claim 32, or a pharmaceutically acceptable salt or solvate thereof, wherein R^{2a} and R^{2b} are taken together with the nitrogen atom to which they are attached to form a substituted C₂₋₉heterocycloalkyl selected from

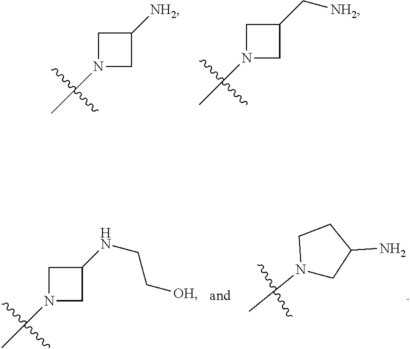 [SMILES: *N1CC(N)C1 ; *N1CC(CN)C1 ; *N1CC(NCCO)C1 ; *N1CCC(N)C1 ; "."]

35. (canceled)

36. The compound of claim 1, or a pharmaceutically acceptable salt or solvate thereof, wherein each R³ is independently selected from halogen.

37. The compound of claim 36, or a pharmaceutically acceptable salt or solvate thereof, wherein each R³ is —F.

38. The compound of claim 1, or a pharmaceutically acceptable salt or solvate thereof, wherein R⁵ is selected from hydrogen, halogen, C₁₋₆alkyl, C₃₋₆cycloalkyl, C₂₋₉heterocycloalkyl, —CN, —C(O)N(R¹²)(R¹³), and —OR¹², wherein C₁₋₆alkyl, C₃₋₆cycloalkyl, and C₂₋₉heterocycloalkyl are optionally substituted with one, two, three, four, or five R²¹.

39. The compound of claim 1, or a pharmaceutically acceptable salt or solvate thereof, wherein R⁵ is selected from hydrogen, halogen, C₁₋₆alkyl, —CN, and —OR¹², wherein C₁₋₆alkyl is optionally substituted with one, two, three, four, or five R²¹.

40. The compound of claim 39, or a pharmaceutically acceptable salt or solvate thereof, wherein R⁵ is selected from hydrogen, halogen, C₁₋₆alkyl, —CN, and —OR¹², wherein C₁₋₆alkyl is optionally substituted with one, two, three halogen, and R¹² is selected from hydrogen and C₁₋₆alkyl.

41. The compound of claim 40, or a pharmaceutically acceptable salt or solvate thereof, wherein R⁵ is selected from hydrogen, —F, —Cl, —CH₃, —CF₃, —CN, and —OCH₃.

42. The compound of claim 1, or a pharmaceutically acceptable salt or solvate thereof, wherein R⁷ is selected from hydrogen, halogen, C₁₋₆alkyl, —CN, and —OR¹², wherein C₁₋₆alkyl is optionally substituted with one, two, three, four, or five R²¹.

43. The compound of claim 42, or a pharmaceutically acceptable salt or solvate thereof, wherein R⁷ is selected from hydrogen, halogen, C₁₋₆alkyl, —CN, and —OR¹², wherein C₁₋₆alkyl is optionally substituted with one, two, three halogen, and R¹² is selected from hydrogen and C₁₋₆alkyl.

44. The compound of claim 43, or a pharmaceutically acceptable salt or solvate thereof, wherein R⁷ is selected from hydrogen, —F, —Cl, —CH₃, —CF₃, —CN, and —OCH₃.

45. The compound of claim 1, or a pharmaceutically acceptable salt or solvate thereof, wherein R⁹ is selected from hydrogen, halogen, C₁₋₆alkyl, C₃₋₆cycloalkyl, —CN, and —OR¹², wherein C₁₋₆alkyl and C₃₋₆cycloalkyl are optionally substituted with one, two, three, four, or five R²¹.

46. (canceled)

47. The compound of claim 45, or a pharmaceutically acceptable salt or solvate thereof, wherein R⁹ is selected from hydrogen, —F, —Cl, —CH₃, —CF₃, —CN, —OCH₃, and

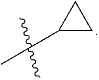 [SMILES: *C1CC1]

48. The compound of claim 1, or a pharmaceutically acceptable salt or solvate thereof, wherein R⁴, R⁶, R⁸, and R¹⁰ are each independently selected from hydrogen, halogen, and unsubstituted C₁₋₆alkyl.

49-55. (canceled)

56. A method of treating a disease or condition in a mammal that would benefit from the modulation of parathyroid hormone (PTH) receptor activity comprising administering a compound of claim 1, or a pharmaceutically acceptable salt, or solvate thereof, to the mammal in need thereof.

57-59. (canceled)
